(19) United States
(12) Patent Application Publication
(10) Pub. No.: US 2009/0137396 A1
(43) Pub. Date: May 28, 2009

(54) Piperazine Compounds with a Herbicidal Action

(75) Inventors: Eike Hupe, Ludwigshafen (DE); Cyrill Zagar, Hong Kong (CN); Matthias Witschel, Bad Duerkheim (DE); Toralf Kuehn, Mannheim (DE); William Karl Moberg, Hassloch (DE); Liliana Parra Rapado, Offenburg (DE); Frank Stelzer, Mannheim (DE); Andrea Vescovi, Mannheim (DE); Robert Reinhard, Limburgerhof (DE); Bernd Sievernich, Hassloch (DE); Klaus Grossmann, Neuhofen (DE); Thomas Ehrhardt, Speyer (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/159,686

(22) PCT Filed: Jan. 4, 2007
(86) PCT No.: PCT/EP07/50067

§ 371 Date: Jun. 30, 2008

(30) Foreign Application Priority Data
Jan. 5, 2006 (EP) 06000185.6

(51) Int. Cl. A01N 43/84; A01N 43/60; C07D 241/04; A01P 21/00; C07D 413/08

(52) U.S. Cl. 504/225; 504/235; 544/383; 544/121

(57) ABSTRACT

The present invention relates to the use of piperazine compounds of the formula I

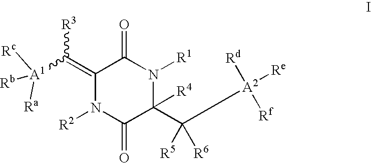

or of the agriculturally useful salts of piperazine compounds of the formula I as herbicides, where in formula I the variables are as defined in the claims and the description.

Moreover, the invention relates to compositions comprising, in addition to auxiliaries customary for formulating crop protection agents, piperazine compounds of the formula I or agriculturally useful salts of I, and to a process for preparing these compositions. Furthermore, the invention relates to certain piperazine compounds of the formula I, a process for preparing piperazine compounds of the formula I and a method for controlling unwanted vegetation which comprises treating plants, their seeds and/or their habitat with at least one piperazine compound of the formula I.

The present invention relates to the use of piperazine compounds of the formula I

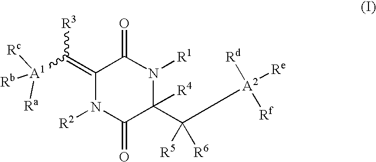

or of the agriculturally useful salts of piperazine compounds of the formula I as herbicides, where in formula I the variables are as defined below:
R¹ and R² independently of one another are:
   cyano, C₁-C₆-alkyl, C₃-C₆-cycloalkyl, C₃-C₈-alkenyl, C₃-C₈-cycloalkenyl, C₃-C₆-alkynyl, C₃-C₆-cycloalkynyl, phenyl, phenyl-(C₁-C₆)-alkyl, heterocyclyl, heterocyclyl-(C₁-C₆)-alkyl; phenyl-[C₁-C₆-alkoxycarbonyl]-(C₁-C₆)-alkyl or phenylheterocyclyl-(C₁-C₆)-alkyl; or
   COR²¹, where
      R²¹ is hydrogen, C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, hydroxyl, C₁-C₆-alkoxy, C₃-C₆-alkenyloxy, C₃-C₆-alkynyloxy, amino, C₁-C₆-alkylamino, [di-(C₁-C₆)-alkyl]amino, C₃-C₆-alkenylamino, C₃-C₆-alkynylamino, C₁-C₆-alkylsulfonylamino, N—(C₂-C₆-alkenyl)-N—(C₁-C₆-alkyl)amino, N—(C₂-C₆-alkynyl)-N—(C₁-C₆-alkyl)amino, N—(C₁-C₆-alkoxy)-N—(C₁-C₆-alkyl)amino, N—(C₂-C₆-alkenyl)-N—(C₁-C₆-alkoxy)amino, N—(C₂-C₆-alkynyl)-N—(C₁-C₆-alkoxy)-amino, phenyl, phenylamino, phenoxy, naphthyl or heterocyclyl; or
   NR²²R²³ where
      R²² and R²³ independently of one another are hydrogen, C₁-C₆-alkyl, C₃-C₆-cycloalkyl, C₃-C₆-alkenyl, C₃-C₆-cycloalkenyl, C₃-C₆-alkynyl, C₃-C₆-cycloalkynyl or C₁-C₆-alkylcarbonyl; or
   OR²⁴, where
      R²⁴ is C₁-C₆-alkyl, C₃-C₆-cycloalkyl, C₃-C₆-alkenyl, C₃-C₆-cycloalkenyl C₃-C₆-alkynyl, C₃-C₆-cycloalkynyl, phenyl or phenyl-(C₁-C₆)-alkyl; or
   SO₂R²⁵, where R²⁵ is C₁-C₆-alkyl or phenyl;
   where the abovementioned aliphatic, cyclic or aromatic moieties of the substituents of R¹ and R² may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl or C₁-C₄-alkylcarbonyloxy;
   and where R¹ may additionally be hydrogen;
R³ is hydrogen, halogen, cyano, C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, phenyl, phenyl-(C₁-C₆)-alkyl, heterocyclyl, heterocyclyl-(C₁-C₆)-alkyl; phenyl-[C₁-C₆-alkoxycarbonyl]-(C₁-C₆)-alkyl or phenylheterocyclyl-(C₁-C₆)-alkyl; or a radical COR²⁶, NR²⁷R²⁸, OR²⁹, SO₂R³⁰ or N(OR³¹)R³², where
   R²⁶ is hydrogen, C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, hydroxyl, C₁-C₆-alkoxy, C₃-C₆-alkenyloxy, C₃-C₆-alkynyloxy, amino, C₁-C₆-alkylamino, [di-(C₁-C₆)-alkyl]amino, C₃-C₆-alkenylamino, C₃-C₆-alkynylamino, C₁-C₆-alkylsulfonylamino, N—(C₂-C₆-alkenyl)-N—(C₁-C₆-alkyl)amino, N—(C₂-C₆-alkynyl)-N—(C₁-C₆-alkyl)amino, N—(C₁-C₆-alkoxy)-N—(C₁-C₆-alkyl)amino, N—(C₂-C₆-alkenyl)-N—(C₁-C₆-alkoxy)amino, N—(C₂-C₆-alkynyl)-N—(C₁-C₆-alkoxy)-amino, phenyl, phenylamino, phenoxy, naphthyl or heterocyclyl;
   R²⁷ and R²⁸ independently of one another are hydrogen, C₁-C₆-alkyl, aryl or heteroaryl;
   R²⁹ is C₁-C₆-alkyl;
   R³⁰ is C₁-C₆-alkyl or phenyl;
   R³¹ is hydrogen, C₁-C₁₀-alkyl, phenyl or phenyl-(C₁-C₆)-alkyl;
   R³² is C₁-C₆-alkyl, phenyl or phenyl-(C₁-C₆)-alkyl;
   where the abovementioned aliphatic, cyclic or aromatic moieties of the substituents of R³ or R²⁶, R²⁷, R²⁸, R²⁹, R³⁰, R³¹ and R³² may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl or C₁-C₄-alkylcarbonyloxy;
R⁴, R⁵, R⁶ independently of one another are hydrogen, hydroxyl, C₁-C₆-alkyl, C₁-C₆-alkoxy,
   where the abovementioned aliphatic moieties of the substituents of R⁴, R⁵ or R⁶ may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄₀-alkyl]aminocarbonyl or C₁-C₄-alkylcarbonyloxy;
A¹ is aryl or heteroaryl;
A² is aryl or heteroaryl, except for indolyl;
R^{a} is halogen, cyano, nitro, C₁-C₆-alkyl, C₃-C₆-cycloalkyl, C₂-C₆-alkenyl, C₃-C₆-cycloalkenyl, C₄-C₁₀-alkadienyl, C₂-C₆-alkynyl, [tri-(C₁-C₆)-alkylsilyl]-(C₂-C₆)-alkynyl, C₃-C₆-cycloalkynyl, C₁-C₆-alkylthio, C₁-C₆-alkylsulfinyl, aryl, phenyl-(C₁-C₆)-alkyl, phenyl-(C₂-C₆)-alkenyl, phenylsulfonyl-(C₁-C₆)-alkyl, heterocyclyl, heterocyclyl-(C₁-C₆)-alkyl or phenyl-[C₁-C₆-alkoxycarbonyl]-(C₁-C₆)-alkyl,
   Z¹P(O)(OR⁹)₂, Z²B(OR¹⁰)₂, where
      R⁹ and R¹⁰ are each hydrogen or C₁-C₆-alkyl and the radicals R¹⁰ in Z²B(OR¹⁰)₂ together may form a C₂-C₄-alkylene chain; or
   Z³COR¹¹, where
      R¹¹ is hydrogen, C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, hydroxyl, C₁-C₆-alkoxy, C₃-C₆-alkenyloxy, C₃-C₆-alkynyloxy, amino, C₁-C₆-alkylamino, [di-(C₁-C₆)-alkyl]amino, C₁-C₆-alkoxyamino, [di-(C₁-C₆)-alkoxy]amino, C₁-C₆-alkylsulfonylamino, C₁-C₆-alkylaminosulfonylamino, [di-(C₁-C₆)-alkylamino]sulfonylamino, C₃-C₈-alkenylamino, C₃-C₆-alkynylamino, N—(C₂-C₆-alkenyl)-N—(C₁-C₆-alkyl)amino, N—(C₂-C₆-alkynyl)-N—(C₁-C₆-alkyl)amino, N—(C₁-C₆-alkoxy)-N—(C₁-C₆-alkyl)amino, N—(C₂-C₆-alkenyl)-N—(C₁-C₆-alkoxy)-amino, N—(C₂-C₆-alkynyl)-N—(C₁-C₆-alkoxy)amino, phenyl, phenoxy, phenylamino, naphthyl or heterocyclyl; or
   Z⁴NR¹²R¹³, where
      R¹² and R¹³ independently of one another are hydrogen, C₁-C₆-alkyl, C₃-C₆-cycloalkyl, C₃-C₆-alkenyl, C₃-C₆-cycloalkenyl, C₃-C₆-alkynyl, C₃-C₆-cycloalkynyl, C₁-C₆-alkylcarbonyl, C₃-C₆-cycloalkylcarbonyl, [di-(C₁-C₆)-alkylamino]carbonyl, C₁-C₆-alkoxycarbonyl, C₁-C₆-alkoxycarbonyl-(C₁-C₆)-alkyl, C₁-C₆-alkylsulfonyl, C₁-C₆-alkylaminosulfonyl, [di-(C₁-C₆)-alkylamino]sulfonyl, phenylcarbonyl, phenylaminocarbonyl, phenylsulfonyl, phenylsulfonylaminocarbonyl or heterocyclylcarbonyl; or
   Z⁵CH═N—O—R¹⁴, where R¹⁴ is hydrogen or C₁-C₆-alkyl; or
   Z⁶OR¹⁵, where
      R¹⁵ is hydrogen, C₁-C₆-alkyl, C₃-C₆-cycloalkyl, C₃-C₆-alkenyl, C₃-C₆-cycloalkenyl, C₃-C₆-alkynyl, C₃-C₆-cycloalkynyl, C₁-C₆-alkylcarbonyl, C₁-C₈-alkoxycarbonyl-(C₁-C₆)-alkyl, [di-(C₁-C₆)-alkoxycarbonyl]-(C₁-C₈)-alkyl, phenyl or phenyl-(C₁-C₆)-alkyl; or
   Z⁷SO₂R¹⁶, where R¹⁶ is C₁-C₆-alkyl or phenyl; and where
   Z¹, Z², Z³, Z⁴, Z⁵, Z⁶, Z⁷ independently of one another are a bond, —CH₂—, —CH₂—CH₂—, —O—CH(R¹⁷)—, —S—CH(R¹⁸)—, —S(O)—CH(R¹⁹)— or —SO₂CH(R²⁰)—, and
   where R¹⁷, R¹⁸, R¹⁹ and R²⁰ independently of one another are hydrogen or C₁-C₆-alkyl; and
   where the abovementioned aliphatic, cyclic or aromatic moieties of the substituent R^{a} may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-haloalkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl or C₁-C₄-alkylcarbonyloxy; and
R^{b}, R^{c}, R^{d}, R^{e} and R^{f} are each independently of one another hydrogen or have one of the meanings given for R^{a}; and
   where two radicals R^{a}, R^{b} or R^{c} attached to adjacent ring atoms of A¹ or two radicals R^{d}, R^{e} or R^{f} attached to adjacent ring atoms of A² may also be straight-chain C₃-C₆-alkylene which may be partially or fully halogenated and which may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl or C₁-C₄-alkylcarbonyloxy, where one CH₂ group in C₃-C₆-alkylene may be replaced by a carbonyl group, thiocarbonyl group or sulfonyl group and in which one or two non-adjacent CH₂ groups in C₃-C₆-alkylene may in each case be replaced by oxygen, sulfur or a group NR³⁴, where R³⁴ has one of the meanings given for R¹².

The thaxtomins A and B produced by the plant pathogen S. scabies (King R. R. et al., J. Agric. Food Chem. (1992) 40, 834-837) are natural products having a central piperazine-2,5-dione ring which carries a 4-nitroindol-3-ylmethyl radical in the 3-position and an optionally OH-substituted benzyl radical in the 2-position. Owing to its plant-damaging action, this compound class was also investigated for a possible use as herbicides (King R. R. et al., J. Agric. Food Chem. (2001) 49, 2298-2301).

In the context of synthetic studies about the preparation of thaxtomin A and B, J. Gelin et al., J. Org. Chem. 58, 1993, pp. 3473-3475, and J. Moyroud et al. Tetrahedron 52, 1996, pp. 8525-8543 describe dehydrothaxtomin derivatives. Described are, inter alia, compounds of the formula

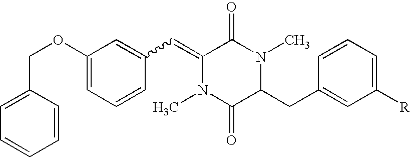

in which R is hydrogen or NO₂.

N. Saito et al., J. Chem. Soc. Perkin Trans 1997, pp. 53-69 describe, inter alia, compounds of the formula below

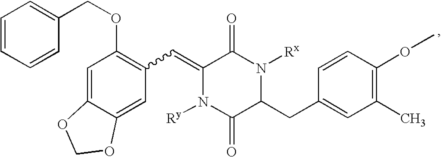

in which R^{y} is hydrogen or benzyl and R^{x} is hydrogen, acetyl or isopropyloxycarbonyl as precursors for preparing ecteinascidins.

In the context of synthetic studies about the preparation of phthalascidin, Z. Z. Liu et al., Chinese Chem. Lett. 13(8) 2002, pp. 701-704 describe an intermediate of the formula below in which Bn is benzyl:

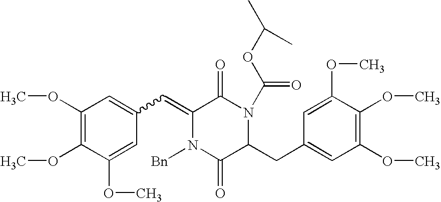

J. Bryans et al., Journal of Antibiotics 49(10), 1996, pp. 1014-1021 describe the compound of the formula below:

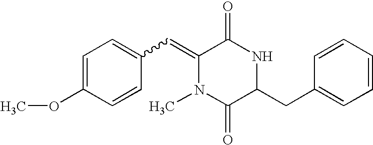

WO 99/48889, WO 01/53290 and WO 2005/011699 describe 2,5-diketopiperazine compounds which have a 4-imidazolyl radical attached via a methylene or methyne group in the 3- or 6-position and in the other 3- or 6-position a benzyl or benzylidene radical. These compounds are antitumor compounds.

It is an object of the present invention to provide compounds having herbicidal action. In particular, the intention is to provide compounds having high herbicidal activity, in particular even at low application rates, and whose compatibility with crop plants is sufficient for commercial use.

This and further objects are achieved by the compounds of the formula I defined at the outset and by their agriculturally useful salts.

Accordingly, the present invention relates to the use of piperazine compounds of the general formula I or of the agriculturally useful salts of piperazine compounds of the formula I as herbicides, i.e. for controlling harmful plants.

The invention also relates to compositions comprising a herbicidally effective amount of at least one piperazine compound of the formula I or an agriculturally useful salt of I and auxiliaries customary for formulating crop protection agents.

Moreover, the invention relates to a method for controlling unwanted vegetation which comprises allowing a herbicidally effective amount of at least one piperazine compound of the formula I or an agriculturally useful salt of I to act on plants, their seeds and/or their habitat.

The piperazine compounds of the formula I are novel and also form part of the subject matter of the present invention,
except for compounds of the formula I in which A¹ is phenyl and A² is 4-imidazolyl or A¹ is 4-imidazolyl and A² is phenyl,
furthermore except for the compound of the formula I in which R¹ is hydrogen and R² is methyl, R³, R⁴, R⁵ and R⁶ are hydrogen and the group A¹(R^{a}R^{b}R^{c}) is 4-methoxyphenyl and the group A²(R^{d}R^{e}R^{f}) is phenyl.
furthermore except for compounds of the formula I in which A¹ is phenyl, R¹ and R² are methyl, R³, R⁴, R⁵ and R⁶ are hydrogen, R^{a} is benzyloxy which is attached in the 3-position, R^{b} and R^{c} are hydrogen and the group A²(R^{d}R^{e}R^{f}) is phenyl or 3-nitrophenyl,
furthermore except for compounds of the formula I in which R¹ is hydrogen, acetyl or isopropyloxycarbonyl, R² is hydrogen or benzyl, R³, R⁴, R⁵ and R⁸ are hydrogen, the group A¹(R^{a}R^{b}R^{c}) is a radical of the formula

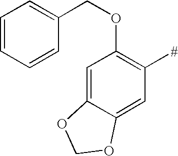

where # indicates the attachment to the methyne carbon which carries R³ and the group A²(R^{d}R^{e}R^{f}) is 3-methyl-4-methoxyphenyl,
furthermore except for the compound of the formula I in which R¹ is isopropyloxycarbonyl and R² is benzyl, R³, R⁴, R⁵ and R⁶ are hydrogen, and the groups A¹(R^{a}R^{b}R^{c}) and A²(R^{d}R^{e}R^{f}) are each 3,4,5-trimethoxyphenyl.

Moreover, the invention relates to processes and intermediates for preparing compounds of the formula I.

Further embodiments of the present invention are evident from the claims, the description and the examples. It is to be understood that the features mentioned above and still to be illustrated below of the subject matter of the invention can be applied not only in the combination given in each particular case but also in other combinations, without leaving the scope of the invention.

Depending on the substitution pattern, the compounds of the formula I may comprise one or more centers of chirality, in which case they are present as enantiomer or diastereomer mixtures. The invention provides both the pure enantiomers or diastereomers and their mixtures.

With respect to the exocyclic double bond, the compounds of the formula I can be present as E isomer or as Z isomer. The invention provides both the pure E isomers and Z isomers and mixtures thereof.

The compounds of the formula I may also be present in the form of their agriculturally useful salts, the nature of the salt generally being immaterial. Suitable salts are, in general, the salts of those cations or the acid addition salts of those acids whose cations and anions, respectively, have no adverse effect on the herbicidal action of the compounds I.

Suitable cations are in particular ions of the alkali metals, preferably lithium, sodium or potassium, of the alkaline earth metals, preferably calcium or magnesium, and of the transition metals, preferably manganese, copper, zinc or iron. Ammonium can likewise be used as cation, where, if desired, one to four hydrogen atoms may be replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, dimethylammonium, diisopropylammonium, tetramethylammonium, tetrabutylammonium, 2-(2-hydroxyeth-1-oxy)eth-1-ylammonium, di(2-hydroxyeth-1-yl)ammonium, trimethylbenzylammonium. Furthermore phosphonium ions, sulfonium ions, preferably tri(C₁-C₄-alkyl)sulfonium, and sulfoxonium ions, preferably tri(C₁-C₄-alkyl)sulfoxonium may be mentioned.

Anions of useful acid addition salts are primarily chloride, bromide, fluoride, hydrogensulfate, sulfate, dihydrogenphosphate, hydrogenphosphate, nitrate, bicarbonate, carbonate, hexafluorosilicate, hexafluorophosphate, benzoate, and the anions of C₁-C₄-alkanoic acids, preferably formate, acetate, propionate and butyrate.

The organic moieties mentioned for the substituents of the compounds according to the invention are collective terms for individual enumerations of the specific group members. All hydrocarbon chains, such as
alkyl, haloalkyl, and also the alkyl moieties in cyanoalkyl, alkoxy, haloalkoxy, alkylthio, haloalkylthio, alkylsulfinyl, haloalkylsulfinyl, alkylsulfonyl, haloalkylsulfonyl, N-alkylaminosulfonyl, N,N-dialkylaminosulfonyl, dialkylamino, N-alkylsulfonylamino, N-haloalkylsulfonylamino, N-alkyl-N-alkylsulfonylamino, N-alkyl-N-haloalkylsulfonylamino, alkylcarbonyl, haloalkylcarbonyl, alkoxycarbonyl, haloalkoxycarbonyl, alkylcarbonyloxy, alkylaminocarbonyl, dialkylaminocarbonyl, dialkylaminothiocarbonyl, alkoxyalkyl, dialkoxyalkyl, alkylthioalkyl, dialkylaminoalkyl, dialkylhydrazinoalkyl, alkyliminooxyalkyl, alkylcarbonylalkyl, alkoxyiminoalkyl, N-(alkylamino)iminoalkyl, N-(dialkylamino)iminoalkyl, alkoxycarbonylalkyl, dialkylaminocarbonylalkyl, phenylalkenylcarbonyl, heterocyclylalkenylcarbonyl, N-alkoxy-N-alkylaminocarbonyl, N-alkyl-N-phenylaminocarbonyl, N-alkyl-N-heterocyclylaminocarbonyl, phenylalkyl, heterocyclylalkyl, phenylcarbonylalkyl, heterocyclylcarbonylalkyl, dialkylaminoalkoxycarbonyl, alkoxyalkoxycarbonyl, alkenylcarbonyl, alkenyloxycarbonyl, alkenylaminocarbonyl, N-alkenyl-N-alkylaminocarbonyl, N-alkenyl-N-alkoxyaminocarbonyl, alkynylcarbonyl, alkynyloxycarbonyl, alkynylaminocarbonyl, N-alkynyl-N-alkylaminocarbonyl, N-alkynyl-N-alkoxyaminocarbonyl, alkenyl, alkynyl, haloalkenyl, haloalkynyl and alkoxyalkoxy moieties
   may be straight-chain or branched. The prefix Cₙ-Cₘ— indicates the respective carbon number of the hydrocarbon moiety. Unless indicated otherwise, halogenated substituents preferably carry one to five identical or different halogen atoms, in particular fluorine atoms or chlorine atoms.

The term halogen denotes in each case fluorine, chlorine, bromine or iodine.

Examples of other meanings are:

alkyl and also the alkyl moieties, for example, in alkoxy, alkylthio, alkylsulfinyl and alkylsulfonyl, alkylcarbonyl, alkylamino, alkylsilyl, phenylalkyl, phenylsulfonylalkyl, heterocyclylalkyl: saturated straight-chain or branched hydrocarbon radicals having one or more carbon atoms, for example 1 to 2, 1 to 4 or 1 to 6 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl. In one embodiment according to the invention, alkyl denotes small alkyl groups such as C₁-C₄-alkyl. In another embodiment according to the invention, alkyl denotes relatively large alkyl groups such as C₅-C₆-alkyl.

Haloalkyl: an alkyl radical as mentioned above whose hydrogen atoms are partially or fully substituted by halogen atoms such as fluorine, chlorine, bromine and/or iodine, for example chloromethyl, dichloromethyl, trichloromethyl, fluoromethyl, difluoromethyl, trifluoromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl and nonafluorobutyl.

Cycloalkyl and also the cycloalkyl moieties, for example, in cycloalkoxy or cycloalkylcarbonyl: monocyclic saturated hydrocarbon groups having three or more carbon atoms, for example 3 to 6 carbon ring members, such as cyclopropyl, cyclobutyl, cyclopentyl and cyclohexyl.

Alkenyl and also alkenyl moieties, for example, in phenyl-(C₂-C₆)-alkenyl or alkenylamino: monounsaturated straight-chain or branched hydrocarbon radicals having two or more carbon atoms, for example 2 to 4, 2 to 6, or 3 to 6 carbon atoms, and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-tri-methyl-2-propenyl, 1-ethyl-1-methyl-2-propenyl, 1-ethyl-2-methyl-1-propenyl, 1-ethyl-2-methyl-2-propenyl.

In one embodiment according to the invention, alkenyl groups such as C₂-C₆-alkenyl are employed. In another embodiment according to the invention, use is made of alkenyl groups such as C₃-C₆-alkenyl.

Cycloalkenyl and also cycloalkenyl moieties: monocyclic, monounsaturated hydrocarbon groups having three or more carbon atoms, for example 3 to 6, preferably 5 to 6, carbon ring members, such as cyclopenten-1-yl, cyclopenten-3-yl, cyclohexen-1-yl, cyclohexen-3-yl, cyclohexen-4-yl.

Alkynyl and also alkynyl moieties, for example, in [tri-(C₁-C₆)-alkylsilyl-(C₂-C₆)-alkynyl or alkynylamino: straight-chain or branched hydrocarbon groups having two or more carbon atoms, for example 2 to 4, 2 to 6, or 3 to 6 carbon atoms, and one or two triple bonds in any position, but not adjacent to one another, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl.

Cycloalkynyl and also cycloalkynyl moieties: monocyclic hydrocarbon groups having three or more carbon atoms, for example 3 to 6, preferably 5 to 6, carbon ring members and one triple bond, such as cyclohexyn-1-yl, cyclohexyn-3-yl, cyclohexyn-4-yl.

C₄-C₁₀-alkadienyl: doubly unsaturated straight-chain or branched hydrocarbon radicals having four or more carbon atoms and two double bonds in any position, but not adjacent to one another, for example 4 to 10 carbon atoms and two double bonds in any position, but not adjacent to one another, for example 1,3-butadienyl, 1-methyl-1,3-butadienyl, 2-methyl-1,3-butadienyl, penta-1,3-dien-1-yl, hexa-1,4-dien-1-yl, hexa-1,4-dien-3-yl, hexa-1,4-dien-6-yl, hexa-1,5-dien-1-yl, hexa-1,5-dien-3-yl, hexa-1,5-dien-4-yl, hepta-1,4-dien-1-yl, hepta-1,4-dien-3-yl, hepta-1,4-dien-6-yl, hepta-1,4-dien-7-yl, hepta-1,5-dien-1-yl, hepta-1,5-dien-3-yl, hepta-1,5-dien-4-yl, hepta-1,5-dien-7-yl, hepta-1,6-dien-1-yl, hepta-1,6-dien-3-yl, hepta-1,6-dien-4-yl, hepta-1,6-dien-5-yl, hepta-1,6-dien-2-yl, octa-1,4-dien-1-yl, octa-1,4-dien-2-yl, octa-1,4-dien-3-yl, octa-1,4-dien-6-yl, octa-1,4-dien-7-yl, octa-1,5-dien-1-yl, octa-1,5-dien-3-yl, octa-1,5-dien-4-yl, octa-1,5-dien-7-yl, octa-1,6-dien-1-yl, octa-1,6-dien-3-yl, octa-1,6-dien-4-yl, octa-1,6-dien-5-yl, octa-1,6-dien-2-yl, deca-1,4-dienyl, deca-1,5-dienyl, deca-1,6-dienyl, deca-1,7-dienyl, deca-1,8-dienyl, deca-2,5-dienyl, deca-2,6-dienyl, deca-2,7-dienyl, deca-2,8-dienyl.

Alkoxy or alkoxy moieties, for example, in phenylalkoxy, alkoxyamino, alkoxycarbonyl: alkyl, as defined above, which is attached via an oxygen atom: for example methoxy, ethoxy, n-propoxy, 1-methylethoxy, butoxy, 1-methylpropoxy, 2-methylpropoxy or 1,1-dimethylethoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy.

In one embodiment according to the invention, small alkoxy groups such as C₁-C₄-alkoxy are employed. In another embodiment according to the invention, use is made of relatively large alkoxy groups such as C₅-C₆-alkoxy.

Alkenyloxy: alkenyl as mentioned above which is attached via an oxygen atom, for example C₃-C₆-alkenyloxy, such as 1-propenyloxy, 2-propenyloxy, 1-methylethenyloxy, 1-butenyloxy, 2-butenyloxy, 3-butenyloxy, 1-methyl-1-propenyloxy, 2-methyl-1-propenyloxy, 1-methyl-2-propenyloxy, 2-methyl-2-propenyloxy, 1-pentenyloxy, 2-pentenyloxy, 3-pentenyloxy, 4-pentenyloxy, 1-methyl-1-butenyloxy, 2-methyl-1-butenyloxy, 3-methyl-1-butenyloxy, 1-methyl-2-butenyloxy, 2-methyl-2-butenyloxy, 3-methyl-2-butenyloxy, 1-methyl-3-butenyloxy, 2-methyl-3-butenyloxy, 3-methyl-3-butenyloxy, 1,1-dimethyl-2-propenyloxy, 1,2-dimethyl-1-propenyloxy, 1,2-dimethyl-2-propenyloxy, 1-ethyl-1-propenyloxy, 1-ethyl-2-propenyloxy, 1-hexenyloxy, 2-hexenyloxy, 3-hexenyloxy, 4-hexenyloxy, 5-hexenyloxy, 1-methyl-1-pentenyloxy, 2-methyl-1-pentenyloxy, 3-methyl-1-pentenyloxy, 4-methyl-1-pentenyloxy, 1-methyl-2-pentenyloxy, 2-methyl-2-pentenyloxy, 3-methyl-2-pentenyloxy, 4-methyl-2-pentenyloxy, 1-methyl-3-pentenyloxy, 2-methyl-3-pentenyloxy, 3-methyl-3-pentenyloxy, 4-methyl-3-pentenyloxy, 1-methyl-4-pentenyloxy, 2-methyl-4-pentenyloxy, 3-methyl-4-pentenyloxy, 4-methyl-4-pentenyloxy, 1,1-dimethyl-2-butenyloxy, 1,1-dimethyl-3-butenyloxy, 1,2-dimethyl-1-butenyloxy, 1,2-dimethyl-2-butenyloxy, 1,2-dimethyl-3-butenyloxy, 1,3-dimethyl-1-butenyloxy, 1,3-dimethyl-2-butenyloxy, 1,3-dimethyl-3-butenyloxy, 2,2-dimethyl-3-butenyloxy, 2,3-dimethyl-1-butenyloxy, 2,3-dimethyl-2-butenyloxy, 2,3-dimethyl-3-butenyloxy, 3,3-dimethyl-1-butenyloxy, 3,3-dimethyl-2-butenyloxy, 1-ethyl-1-butenyloxy, 1-ethyl-2-butenyloxy, 1-ethyl-3-butenyloxy, 2-ethyl-1-butenyloxy, 2-ethyl-2-butenyloxy, 2-ethyl-3-butenyloxy, 1,1,2-trimethyl-2-propenyloxy, 1-ethyl-1-methyl-2-propenyloxy, 1-ethyl-2-methyl-1-propenyloxy and 1-ethyl-2-methyl-2-propenyloxy. In one embodiment according to the invention, small alkenyloxy groups such as C₃-C₄-alkenyloxy are employed. In another embodiment according to the invention, use is made of relatively large alkenyloxy groups such as C₅-C₆-alkenyloxy.

Alkynyloxy: alkynyl as mentioned above which is attached via an oxygen atom, for example C₃-C₆-alkynyloxy, such as 2-propynyloxy, 2-butynyloxy, 3-butynyloxy, 1-methyl-2-propynyloxy, 2-pentynyloxy, 3-pentynyloxy, 4-pentynyloxy, 1-methyl-2-butynyloxy, 1-methyl-3-butynyloxy, 2-methyl-3-butynyloxy, 1-ethyl-2-propynyloxy, 2-hexynyloxy, 3-hexynyloxy, 4-hexynyloxy, 5-hexynyloxy, 1-methyl-2-pentynyloxy, 1-methyl-3-pentynyloxy. In one embodiment according to the invention, small alkynyloxy groups such as C₃-C₄-alkynyloxy are employed. In another embodiment according to the invention, use is made of relatively large alkynyloxy groups such as C₅-C₆-alkynyloxy.

Alkylthio: alkyl as defined above which is attached via a sulfur atom.

Alkylsulfinyl: alkyl as defined above which is attached via an SO group.

Alkylsulfonyl: alkyl as defined above which is attached via an S(O)₂ group.

Alkylcarbonyl: alkyl as defined above which is attached via a (C═O) group, for example methylcarbonyl, ethylcarbonyl, propylcarbonyl, 1-methylethylcarbonyl, butylcarbonyl, 1-methylpropylcarbonyl, 2-methylpropylcarbonyl or 1,1-dimethylethylcarbonyl, pentylcarbonyl, 1-methylbutylcarbonyl, 2-methylbutylcarbonyl, 3-methylbutylcarbonyl, 2,2-dimethylpropylcarbonyl, 1-ethylpropylcarbonyl, hexylcarbonyl, 1,1-dimethylpropylcarbonyl, 1,2-dimethylpropylcarbonyl, 1-methylpentylcarbonyl, 2-methylpentylcarbonyl, 3-methylpentylcarbonyl, 4-methylpentylcarbonyl, 1,1-dimethylbutylcarbonyl, 1,2-dimethylbutylcarbonyl, 1,3-dimethylbutylcarbonyl, 2,2,-dimethylbutylcarbonyl, 2,3-dimethylbutylcarbonyl, 3,3-dimethylbutylcarbonyl, 1-ethylbutylcarbonyl, 2-ethylbutylcarbonyl, 1,1,2-trimethylpropylcarbonyl, 1,2,2-trimethylpropylcarbonyl, 1-ethyl-1-methylpropylcarbonyl or 1-ethyl-2-methylpropylcarbonyl.

Alkenylcarbonyl: alkenyl as defined above which is attached via a (C═O) group, for example 1-ethenylcarbonyl.

Alkynylcarbonyl: alkynyl as defined above which is attached via a (C═O) group, for example 1-propynylcarbonyl.

Heterocyclyl: a mono- or bicyclic saturated, partially unsaturated or aromatic heterocyclic ring having three or more, for example 3 to 10, ring atoms, for example a monocyclic 3-, 4-, 5-, 6- or 7-membered heterocyclic ring which contains one to four identical or different heteroatoms selected from the group consisting of oxygen, sulfur and nitrogen and which may be attached via carbon or nitrogen, for example
3- or 4-membered saturated or unsaturated rings attached via carbon, such as 2-oxiranyl, 2-oxetanyl, 3-oxetanyl, 2-aziridinyl, 3-thiethanyl, 1-azetidinyl, 2-azetidinyl.
5-membered saturated rings attached via carbon, such as tetrahydrofuran-2-yl, tetrahydrofuran-3-yl, tetrahydrothien-2-yl, tetrahydrothien-3-yl, tetrahydropyrrol-2-yl, tetrahydropyrrol-3-yl, tetrahydropyrazol-3-yl, tetrahydropyrazol-4-yl, tetrahydroisoxazol-3-yl, tetrahydroisoxazol-4-yl, tetrahydroisoxazol-5-yl, 1,2-oxathiolan-3-yl, 1,2-oxathiolan-4-yl, 1,2-oxathiolan-5-yl, tetrahydroisothiazol-3-yl, tetrahydroisothiazol-4-yl, tetrahydroisothiazol-5-yl, 1,2-dithiolan-3-yl, 1,2-dithiolan-4-yl, tetrahydroimidazol-2-yl, tetrahydroimidazol-4-yl, tetrahydrooxazol-2-yl, tetrahydrooxazol-4-yl, tetrahydrooxazol-5-yl, tetrahydrothiazol-2-yl, tetrahydrothiazol-4-yl, tetrahydrothiazol-5-yl, 1,3-dioxolan-2-yl, 1,3-dioxolan-4-yl, 1,3-oxathiolan-2-yl, 1,3-oxathiolan-4-yl, 1,3-oxathiolan-5-yl, 1,3-dithiolan-2-yl, 1,3-dithiolan-4-yl, 1,3,2-dioxathiolan-4-yl.
6-membered saturated rings attached via carbon, such as: tetrahydropyran-2-yl, tetrahydropyran-3-yl, tetrahydropyran-4-yl, piperidin-2-yl, piperidin-3-yl, piperidin-4-yl, tetrahydrothiopyran-2-yl, tetrahydrothiopyran-3-yl, tetrahydrothiopyran-4-yl, 1,3-dioxan-2-yl, 1,3-dioxan-4-yl, 1,3-dioxan-5-yl, 1,4-dioxan-2-yl, 1,3-dithian-2-yl, 1,3-dithian-4-yl, 1,3-dithian-5-yl, 1,4-dithian-2-yl, 1,3-oxathian-2-yl, 1,3-oxathian-4-yl, 1,3-oxathian-5-yl, 1,3-oxathian-6-yl, 1,4-oxathian-2-yl, 1,4-oxathian-3-yl, 1,2-dithian-3-yl, 1,2-dithian-4-yl, hexahydropyrimidin-2-yl, hexahydropyrimidin-4-yl, hexahydropyrimidin-5-yl, hexahydropyrazin-2-yl, hexahydropyridazin-3-yl, hexahydropyridazin-4-yl, tetrahydro-1,3-oxazin-2-yl, tetrahydro-1,3-oxazin-4-yl, tetrahydro-1,3-oxazin-5-yl, tetrahydro-1,3-oxazin-6-yl, tetrahydro-1,3-thiazin-2-yl, tetrahydro-1,3-thiazin-4-yl, tetrahydro-1,3-thiazin-5-yl, tetrahydro-1,3-thiazin-6-yl, tetrahydro-1,4-thiazin-2-yl, tetrahydro-1,4-thiazin-3-yl, tetrahydro-1,4-oxazin-2-yl, tetrahydro-1,4-oxazin-3-yl, tetrahydro-1,2-oxazin-3-yl, tetrahydro-1,2-oxazin-4-yl, tetrahydro-1,2-oxazin-5-yl, tetrahydro-1,2-oxazin-6-yl.
5-membered saturated rings attached via nitrogen, such as: tetrahydropyrrol-1-yl, tetrahydropyrazol-1-yl, tetrahydroisoxazol-2-yl, tetrahydroisothiazol-2-yl, tetrahydroimidazol-1-yl, tetrahydrooxazol-3-yl, tetrahydrothiazol-3-yl.
6-membered-saturated rings attached via nitrogen, such as: piperidin-1-yl, hexahydropyrimidin-1-yl, hexahydropyrazin-1-yl, hexahydropyridazin-1-yl, tetrahydro-1,3-oxazin-3-yl, tetrahydro-1,3-thiazin-3-yl, tetrahydro-1,4-thiazin-4-yl, tetrahydro-1,4-oxazin-4-yl, tetrahydro-1,2-oxazin-2-yl.
5-membered partially unsaturated rings attached via carbon, such as: 2,3-dihydrofuran-2-yl, 2,3-dihydrofuran-3-yl, 2,5-dihydrofuran-2-yl, 2,5-dihydrofuran-3-yl, 4,5-dihydrofuran-2-yl, 4,5-dihydrofuran-3-yl, 2,3-dihydrothien-2-yl, 2,3-dihydrothien-3-yl, 2,5-dihydrothien-2-yl, 2,5-dihydrothien-3-yl, 4,5-dihydrothien-2-yl, 4,5-dihydrothien-3-yl, 2,3-dihydro-1H-pyrrol-2-yl, 2,3-dihydro-1H-pyrrol-3-yl, 2,5-dihydro-1H-pyrrol-2-yl, 2,5-dihydro-1H-pyrrol-3-yl, 4,5-dihydro-1H-pyrrol-2-yl, 4,5-dihydro-1H-pyrrol-3-yl, 3,4-dihydro-2H-pyrrol-2-yl, 3,4-dihydro-2H-pyrrol-3-yl, 3,4-dihydro-5H-pyrrol-2-yl, 3,4-dihydro-5H-pyrrol-3-yl, 4,5-dihydro-1H-pyrazol-3-yl, 4,5-dihydro-1H-pyrazol-4-yl, 4,5-dihydro-1H-pyrazol-5-yl, 2,5-dihydro-1H-pyrazol-3-yl, 2,5-dihydro-1H-pyrazol-4-yl, 2,5-dihydro-1H-pyrazol-5-yl, 4,5-dihydroisoxazol-3-yl, 4,5-dihydroisoxazol-4-yl, 4,5-dihydroisoxazol-5-yl, 2,5-dihydroisoxazol-3-yl, 2,5-dihydroisoxazol-4-yl, 2,5-dihydroisoxazol-5-yl, 2,3-dihydroisoxazol-3-yl, 2,3-dihydroisoxazol-4-yl, 2,3-dihydroisoxazol-5-yl, 4,5-dihydroisothiazol-3-yl, 4,5-dihydroisothiazol-4-yl, 4,5-dihydroisothiazol-5-yl, 2,5-dihydroisothiazol-3-yl, 2,5-dihydroisothiazol-4-yl, 2,5-dihydroisothiazol-5-yl, 2,3-dihydroisothiazol-3-yl, 2,3-dihydroisothiazol-4-yl, 2,3-dihydroisothiazol-5-yl, Δ³-1,2-dithiol-3-yl, Δ³-1,2-dithiol-4-yl, Δ³-1,2-dithiol-5-yl, 4,5-dihydro-1H-imidazol-2-yl, 4,5-dihydro-1H-imidazol-4-yl, 4,5-dihydro-1H-imidazol-5-yl, 2,5-dihydro-1H-imidazol-2-yl, 2,5-dihydro-1H-imidazol-4-yl, 2,5-dihydro-1H-imidazol-5-yl, 2,3-dihydro-1H-imidazol-2-yl, 2,3-dihydro-1H-imidazol-4-yl, 4,5-dihydrooxazol-2-yl, 4,5-dihydrooxazol-4-yl, 4,5-dihydrooxazol-5-yl, 2,5-dihydrooxazol-2-yl, 2,5-dihydrooxazol-4-yl, 2,5-dihydrooxazol-5-yl, 2,3-dihydrooxazol-2-yl, 2,3-dihydrooxazol-4-yl, 2,3-dihydrooxazol-5-yl, 4,5-dihydrothiazol-2-yl, 4,5-dihydrothiazol-4-yl, 4,5-dihydrothiazol-5-yl, 2,5-dihydrothiazol-2-yl, 2,5-dihydrothiazol-4-yl, 2,5-dihydrothiazol-5-yl, 2,3-dihydrothiazol-2-yl, 2,3-dihydrothiazol-4-yl, 2,3-dihydrothiazol-5-yl, 1,3-dioxol-2-yl, 1,3-dioxol-4-yl, 1,3-dithiol-2-yl, 1,3-dithiol-4-yl, 1,3-oxathiol-2-yl, 1,3-oxathiol-4-yl, 1,3-oxathiol-5-yl, 1,2,3-Δ²-oxadiazolin-4-yl, 1,2,3-Δ²-oxadiazolin-5-yl, 1,2,4-Δ⁴-oxadiazolin-3-yl, 1,2,4-Δ⁴-oxadiazolin-5-yl, 1,2,4-Δ²-oxadiazolin-3-yl, 1,2,4-Δ²-oxadiazolin-5-yl, 1,2,4-Δ³-oxadiazolin-3-yl, 1,2,4-Δ³-oxadiazolin-5-yl, 1,3,4-Δ²-oxadiazolin-2-yl, 1,3,4-Δ²-oxadiazolin-5-yl, 1,3,4-Δ³-oxadiazolin-2-yl, 1,3,4-oxadiazolin-2-yl, 1,2,4-Δ⁴-thiadiazolin-3-yl, 1,2,4-Δ⁴-thiadiazolin-5-yl, 1,2,4-Δ³-thiadiazolin-3-yl, 1,2,4-Δ³-thiadiazolin-5-yl, 1,2,4-Δ²-thiadiazolin-3-yl, 1,2,4-Δ²-thiadiazolin-5-yl, 1,3,4-Δ²-thiadiazolin-2-yl, 1,3,4-Δ²-thiadiazolin-5-yl, 1,3,4-Δ³-thiadiazolin-2-yl, 1,3,4-thiadiazolin-2-yl, 1,2,3-Δ²-triazolin-4-yl, 1,2,3-Δ²-triazolin-5-yl, 1,2,4-Δ²-triazolin-3-yl, 1,2,4-Δ²-triazolin-5-yl, 1,2,4-Δ³-triazolin-3-yl, 1,2,4-Δ³-triazolin-5-yl, 1,2,4-Δ¹-triazolin-2-yl, 1,2,4-triazolin-3-yl, 3H-1,2,4-dithiazol-5-yl, 2H-1,3,4-dithiazol-5-yl, 2H-1,3,4-oxathiazol-5-yl.
6-membered partially unsaturated rings attached via carbon, such as: 2H-3,4-dihydropyran-6-yl, 2H-3,4-dihydropyran-5-yl, 2H-3,4-dihydropyran-4-yl, 2H-3,4-dihydropyran-3-yl, 2H-3,4-dihydropyran-2-yl, 2H-3,4-dihydrothiopyran-6-yl, 2H-3,4-dihydrothiopyran-5-yl, 2H-3,4-dihydrothiopyran-4-yl, 2H-3,4-dihydrothiopyran-3-yl, 2H-3,4-dihydrothiopyran-2-yl, 1,2,3,4-tetrahydropyridin-6-yl, 1,2,3,4-tetrahydropyridin-5-yl, 1,2,3,4-tetrahydropyridin-4-yl, 1,2,3,4-tetrahydropyridin-3-yl, 1,2,3,4-tetrahydropyridin-2-yl, 2H-5,6-dihydropyran-2-yl, 2H-5,6-dihydropyran-3-yl, 2H-5,6-dihydropyran-4-yl, 2H-5,6-dihydropyran-5-yl, 2H-5,6-dihydropyran-6-yl, 2H-5,6-dihydrothiopyran-2-yl, 2H-5,6-dihydrothiopyran-3-yl, 2H-5,6-dihydrothiopyran-4-yl, 2H-5,6-dihydrothiopyran-5-yl, 2H-5,6-dihydrothiopyran-6-yl, 1,2,5,6-tetrahydropyridin-2-yl, 1,2,5,6-tetrahydropyridin-3-yl, 1,2,5,6-tetrahydropyridin-4-yl, 1,2,5,6-tetrahydropyridin-5-yl, 1,2,5,6-tetrahydropyridin-6-yl, 2,3,4,5-tetrahydropyridin-2-yl, 2,3,4,5-tetrahydropyridin-3-yl, 2,3,4,5-tetrahydropyridin-4-yl, 2,3,4,5-tetrahydropyridin-5-yl, 2,3,4,5-tetrahydropyridin-6-yl, 4H-pyran-2-yl, 4H-pyran-3-yl, 4H-pyran-4-yl, 4H-thiopyran-2-yl, 4H-thiopyran-3-yl, 4H-thiopyran-4-yl, 1,4-dihydropyridin-2-yl, 1,4-dihydropyridin-3-yl, 1,4-dihydropyridin-4-yl, 2H-pyran-2-yl, 2H-pyran-3-yl, 2H-pyran-4-yl, 2H-pyran-5-yl, 2H-pyran-6-yl, 2H-thiopyran-2-yl, 2H-thiopyran-3-yl, 2H-thiopyran-4-yl, 2H-thiopyran-5-yl, 2H-thiopyran-6-yl, 1,2-dihydropyridin-2-yl, 1,2-dihydropyridin-3-yl, 1,2-dihydropyridin-4-yl, 1,2-dihydropyridin-5-yl, 1,2-dihydropyridin-6-yl, 3,4-dihydropyridin-2-yl, 3,4-dihydropyridin-3-yl, 3,4-dihydropyridin-4-yl, 3,4-dihydropyridin-5-yl, 3,4-dihydropyridin-6-yl, 2,5-dihydropyridin-2-yl, 2,5-dihydropyridin-3-yl, 2,5-dihydropyridin-4-yl, 2,5-dihydropyridin-5-yl, 2,5-dihydropyridin-6-yl, 2,3-dihydropyridin-2-yl, 2,3-dihydropyridin-3-yl, 2,3-dihydropyridin-4-yl, 2,3-dihydropyridin-5-yl, 2,3-dihydropyridin-6-yl, 2H-5,6-dihydro-1,2-oxazin-3-yl, 2H-5,6-dihydro-1,2-oxazin-4-yl, 2H-5,6-dihydro-1,2-oxazin-5-yl, 2H-5,6-dihydro-1,2-oxazin-6-yl, 2H-5,6-dihydro-1,2-thiazin-3-yl, 2H-5,6-dihydro-1,2-thiazin-4-yl, 2H-5,6-dihydro-1,2-thiazin-5-yl, 2H-5,6-dihydro-1,2-thiazin-6-yl, 4H-5,6-dihydro-1,2-oxazin-3-yl, 4H-5,6-dihydro-1,2-oxazin-4-yl, 4H-5,6-dihydro-1,2-oxazin-5-yl, 4H-5,6-dihydro-1,2-oxazin-6-yl, 4H-5,6-dihydro-1,2-thiazin-3-yl, 4H-5,6-dihydro-1,2-thiazin-4-yl, 4H-5,6-dihydro-1,2-thiazin-5-yl, 4H-5,6-dihydro-1,2-thiazin-6-yl, 2H-3,6-dihydro-1,2-oxazin-3-yl, 2H-3,6-dihydro-1,2-oxazin-4-yl, 2H-3,6-dihydro-1,2-oxazin-5-yl, 2H-3,6-dihydro-1,2-oxazin-6-yl, 2H-3,6-dihydro-1,2-thiazin-3-yl, 2H-3,6-dihydro-1,2-thiazin-4-yl, 2H-3,6-dihydro-1,2-thiazin-5-yl, 2H-3,6-dihydro-1,2-thiazin-6-yl, 2H-3,4-dihydro-1,2-oxazin-3-yl, 2H-3,4-dihydro-1,2-oxazin-4-yl, 2H-3,4-dihydro-1,2-oxazin-5-yl, 2H-3,4-dihydro-1,2-oxazin-6-yl, 2H-3,4-dihydro-1,2-thiazin-3-yl, 2H-3,4-dihydro-1,2-thiazin-4-yl, 2H-3,4-dihydro-1,2-thiazin-5-yl, 2H-3,4-dihydro-1,2-thiazin-6-yl, 2,3,4,5-tetrahydropyridazin-3-yl, 2,3,4,5-tetrahydropyridazin-4-yl, 2,3,4,5-tetrahydropyridazin-5-yl, 2,3,4,5-tetrahydropyridazin-6-yl, 3,4,5,6-tetrahydropyridazin-3-yl, 3,4,5,6-tetrahydropyridazin-4-yl, 1,2,5,6-tetrahydropyridazin-3-yl, 1,2,5,6-tetrahydropyridazin-4-yl, 1,2,5,6-tetrahydropyridazin-5-yl, 1,2,5,6-tetrahydropyridazin-6-yl, 1,2,3,6-tetrahydropyridazin-3-yl, 1,2,3,6-tetrahydropyridazin-4-yl, 4H-5,6-dihydro-1,3-oxazin-2-yl, 4H-5,6-dihydro-1,3-oxazin-4-yl, 4H-5,6-dihydro-1,3-oxazin-5-yl, 4H-5,6-dihydro-1,3-oxazin-6-yl, 4H-5,6-dihydro-1,3-thiazin-2-yl, 4H-5,6-dihydro-1,3-thiazin-4-yl, 4H-5,6-dihydro-1,3-thiazin-5-yl, 4H-5,6-dihydro-1,3-thiazin-6-yl, 3,4,5,6-tetrahydropyrimidin-2-yl, 3,4,5,6-tetrahydropyrimidin-4-yl, 3,4,5,6-tetrahydropyrimidin-5-yl, 3,4,5,6-tetrahydropyrimidin-6-yl, 1,2,3,4-tetrahydropyrazin-2-yl, 1,2,3,4-tetrahydropyrazin-5-yl, 1,2,3,4-tetrahydropyrimidin-2-yl, 1,2,3,4-tetrahydropyrimidin-4-yl, 1,2,3,4-tetrahydropyrimidin-5-yl, 1,2,3,4-tetrahydropyrimidin-6-yl, 2,3-dihydro-1,4-thiazin-2-yl, 2,3-dihydro-1,4-thiazin-3-yl, 2,3-dihydro-1,4-thiazin-5-yl, 2,3-dihydro-1,4-thiazin-6-yl, 2H-1,2-oxazin-3-yl, 2H-1,2-oxazin-4-yl, 2H-1,2-oxazin-5-yl, 2H-1,2-oxazin-6-yl, 2H-1,2-thiazin-3-yl, 2H-1,2-thiazin-4-yl, 2H-1,2-thiazin-5-yl, 2H-1,2-thiazin-6-yl, 4H-1,2-oxazin-3-yl, 4H-1,2-oxazin-4-yl, 4H-1,2-oxazin-5-yl, 4H-1,2-oxazin-6-yl, 4H-1,2-thiazin-3-yl, 4H-1,2-thiazin-4-yl, 4H-1,2-thiazin-5-yl, 4H-1,2-thiazin-6-yl, 6H-1,2-oxazin-3-yl, 6H-1,2-oxazin-4-yl, 6H-1,2-oxazin-5-yl, 6H-1,2-oxazin-6-yl, 6H-1,2-thiazin-3-yl, 6H-1,2-thiazin-4-yl, 6H-1,2-thiazin-5-yl, 6H-1,2-thiazin-6-yl, 2H-1,3-oxazin-2-yl, 2H-1,3-oxazin-4-yl, 2H-1,3-oxazin-5-yl, 2H-1,3-oxazin-6-yl, 2H-1,3-thiazin-2-yl, 2H-1,3-thiazin-4-yl, 2H-1,3-thiazin-5-yl, 2H-1,3-thiazin-6-yl, 4H-1,3-oxazin-2-yl, 4H-1,3-oxazin-4-yl, 4H-1,3-oxazin-5-yl, 4H-1,3-oxazin-6-yl, 4H-1,3-thiazin-2-yl, 4H-1,3-thiazin-4-yl, 4H-1,3-thiazin-5-yl, 4H-1,3-thiazin-6-yl, 6H-1,3-oxazin-2-yl, 6H-1,3-oxazin-4-yl, 6H-1,3-oxazin-5-yl, 6H-1,3-oxazin-6-yl, 6H-1,3-thiazin-2-yl, 6H-1,3-oxazin-4-yl, 6H-1,3-oxazin-5-yl, 6H-1,3-thiazin-6-yl, 2H-1,4-oxazin-2-yl, 2H-1,4-oxazin-3-yl, 2H-1,4-oxazin-5-yl, 2H-1,4-oxazin-6-yl, 2H-1,4-thiazin-2-yl, 2H-1,4-thiazin-3-yl, 2H-1,4-thiazin-5-yl, 2H-1,4-thiazin-6-yl, 4H-1,4-oxazin-2-yl, 4H-1,4-oxazin-3-yl, 4H-1,4-thiazin-2-yl, 4H-1,4-thiazin-3-yl, 1,4-dihydropyridazin-3-yl, 1,4-dihydropyridazin-4-yl, 1,4-dihydropyridazin-5-yl, 1,4-dihydropyridazin-6-yl, 1,4-dihydropyrazin-2-yl, 1,2-dihydropyrazin-2-yl, 1,2-dihydropyrazin-3-yl, 1,2-dihydropyrazin-5-yl, 1,2-dihydropyrazin-6-yl, 1,4-dihydropyrimidin-2-yl, 1,4-dihydropyrimidin-4-yl, 1,4-dihydropyrimidin-5-yl, 1,4-dihydropyrimidin-6-yl, 3,4-dihydropyrimidin-2-yl, 3,4-dihydropyrimidin-4-yl, 3,4-dihydropyrimidin-5-yl or 3,4-dihydropyrimidin-6-yl.
5-membered partially unsaturated rings attached via nitrogen, such as: 2,3-dihydro-1H-pyrrol-1-yl, 2,5-dihydro-1H-pyrrol-1-yl, 4,5-dihydro-1H-pyrazol-1-yl, 2,5-dihydro-1H-pyrazol-1-yl, 2,3-dihydro-1H-pyrazol-1-yl, 2,5-dihydroisoxazol-2-yl, 2,3-dihydroisoxazol-2-yl, 2,5-dihydroisothiazol-2-yl, 2,3-dihydroisoxazol-2-yl, 4,5-dihydro-1H-imidazol-1-yl, 2,5-dihydro-1H-imidazol-1-yl, 2,3-dihydro-1H-5 imidazol-1-yl, 2,3-dihydrooxazol-3-yl, 2,3-dihydrothiazol-3-yl, 1,2,4-Δ⁴-oxadiazolin-2-yl, 1,2,4-Δ²-oxadiazolin-4-yl, 1,2,4-Δ³-oxadiazolin-2-yl, 1,3,4-Δ²-oxadiazolin-4-yl, 1,2,4-Δ⁵-thiadiazolin-2-yl, 1,2,4-Δ³-thiadiazolin-2-yl, 1,2,4-Δ²-thiadiazolin-4-yl, 1,3,4-Δ²-thiadiazolin-4-yl, 1,2,3-Δ²-triazolin-1-yl, 1,2,4-Δ²-triazolin-1-yl, 1,2,4-Δ²-triazolin-4-yl, 1,2,4-Δ³-triazolin-1-yl, 1,2,4-Δ¹-triazolin-4-yl.
6-membered partially unsaturated rings attached via nitrogen, such as: 1,2,3,4-tetrahydropyridin-1-yl, 1,2,5,6-tetrahydropyridin-1-yl, 1,4-dihydropyridin-1-yl, 1,2-dihydropyridin-1-yl, 2H-5,6-dihydro-1,2-oxazin-2-yl, 2H-5,6-dihydro-1,2-thiazin-2-yl, 2H-3,6-dihydro-1,2-oxazin-2-yl, 2H-3,6-dihydro-1,2-thiazin-2-yl, 2H-3,4-dihydro-1,2-oxazin-2-yl, 2H-3,4-dihydro-1,2-thiazin-2-yl, 2,3,4,5-tetrahydropyridazin-2-yl, 1,2,5,6-tetrahydropyridazin-1-yl, 1,2,5,6-tetrahydropyridazin-2-yl, 1,2,3,6-tetrahydropyridazin-1-yl, 3,4,5,6-tetrahydropyrimidin-3-yl, 1,2,3,4-tetrahydropyrazin-1-yl, 1,2,3,4-tetrahydropyrimidin-1-yl, 1,2,3,4-tetrahydropyrimidin-3-yl, 2,3-dihydro-1,4-thiazin-4-yl, 2H-1,2-oxazin-2-yl, 2H-1,2-thiazin-2-yl, 4H-1,4-oxazin-4-yl, 4H-1,4-thiazin-4-yl, 1,4-dihydropyridazin-1-yl, 1,4-dihydropyrazin-1-yl, 1,2-dihydropyrazin-1-yl, 1,4-dihydropyrimidin-1-yl or 3,4-dihydropyrimidin-3-yl.
5-membered heteroaromatic rings, attached via carbon, having generally 1, 2, 3 or 4 nitrogen atoms or a heteroatom selected from oxygen and sulfur and, if appropriate, 1, 2 or 3 nitrogen atoms as ring members, such as: 2-furyl, 3-furyl, 2-thienyl, 3-thienyl, pyrrol-2-yl, pyrrol-3-yl, pyrazol-3-yl, pyrazol-4-yl, isoxazol-3-yl, isoxazol-4-yl, isoxazol-5-yl, isothiazol-3-yl, isothiazol-4-yl, isothiazol-5-yl, imidazol-2-yl, imidazol-4-yl, oxazol-2-yl, oxazol-4-yl, oxazol-5-yl, thiazol-2-yl, thiazol-4-yl, thiazol-5-yl, 1,2,3-oxadiazol-4-yl, 1,2,3-oxadiazol-5-yl, 1,2,4-oxadiazol-3-yl, 1,2,4,-oxadiazol-5-yl, 1,3,4-oxadiazol-2-yl, 1,2,3-thiadiazol-4-yl, 1,2,3-thiadiazol-5-yl, 1,2,4-thiadiazol-3-yl, 1,2,4-thiadiazol-5-yl, 1,3,4-thiadiazolyl-2-yl, 1,2,3-triazol-4-yl, 1,2,4-triazol-3-yl, tetrazol-5-yl.
6-membered heteroaromatic rings, attached via carbon, having generally 1, 2, 3 or 4 nitrogen atoms as ring members, such as
pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl, 1,3,5-triazin-2-yl, 1,2,4-triazin-3-yl, 1,2,4-triazin-5-yl, 1,2,4-triazin-6-yl, 1,2,4,5-tetrazin-3-yl.
5-membered heteroaromatic rings, attached via nitrogen, having generally 1, 2, 3 or 4 nitrogen atoms as ring members, such as:
pyrrol-1-yl, pyrazol-1-yl, imidazol-1-yl, 1,2,3-triazol-1-yl, 1,2,4-triazol-1-yl, tetrazol-1-yl.
   or a bicyclic heterocycle which comprises one of the abovementioned 5- or 6-membered heterocyclic rings and a further fused-on saturated, unsaturated or aromatic carbocycle, for example a benzene, cyclohexane, cyclohexene or cyclohexadiene ring, or a further fused-on 5- or 6-membered heterocyclic ring, where the latter may likewise be saturated, unsaturated or aromatic.

A sulfur atom in the heterocycles mentioned may be oxidized to S═O or S(═O)₂.

Accordingly, hetaryl and/or heteroaryl are a 5- or 6-membered heteroaromatic radical which has 1, 2, 3 or 4 identical or different heteroatoms selected from the group consisting of oxygen, sulfur and nitrogen as ring members, which may be attached via carbon or nitrogen and which, together with a further fused-on benzene ring or a 5- to 6-membered heteroaromatic may form a bicyclic ring system. Examples of hetaryl are the abovementioned 5- and 6-membered heteroaromatic rings attached via carbon, the abovementioned 5-membered heteroaromatic rings attached via nitrogen and bicyclic heteroaromatic radicals such as quinolinyl, isoquinolinyl, quinazolinyl, quinoxalinyl, indolyl, benzothienyl, benzofuryl, benzoxazolyl, benzothiazolyl, benzimidazolyl, benzopyrazolyl, benzotriazole, indolizinyl, 1,2,4-triazolo[1,5-a]pyrimidinyl, 1,2,4-triazolo[4,3-a]pyridinyl, pyrazolo[3,4-b]pyridinyl, 1,2,4-triazolo[1,5-a]pyridinyl, imidazo[1,2-a]pyridyl, imidazo[3,4-a]pyrimidinyl, and the like.

Aryl: a mono- or polycyclic aromatic carbocycle, for example a mono- or bicyclic or a mono- to tricyclic aromatic carbocycle having 6 to 14 ring members, such as, for example, phenyl, naphthyl or anthracenyl.

Arylalkyl: an aryl radical attached via an alkylene group, in particular via a methylene, 1,1-ethylene or 1,2-ethylene group, for example benzyl, 1-phenylethyl and 2-phenylethyl.

Heterocyclylalkyl and also hetarylalkyl: a heterocyclyl or hetaryl radical attached via an alkylene group, in particular via a methylene, 1,1-ethylene or 1,2-ethylene group.

In a particular embodiment, the variables of the compounds of the formula I have the meanings below, these meanings—both on their own and in combination with one another—being particular embodiments of the compounds of the formula I:
R¹ is hydrogen, amino, cyano, C₁-C₆-alkyl, C₃-C₆-alkenyl, C₃-C₆-alkynyl, phenyl-(C₁-C₆)-alkyl, heterocyclyl-(C₁-C₆)-alkyl or COR²¹, where R²¹ is as defined above and is in particular C₁-C₆-alkyl, C₁-C₁₀-alkoxy, phenyl, phenylamino or heterocyclyl; where the abovementioned aliphatic, cyclic or aromatic moieties of the substituents may be partially or fully halogenated. With particular preference, R¹ has the meanings hydrogen or C₁-C₆-alkyl, in particular methyl.
R² is amino, cyano, C₁-C₆-alkyl, C₃-C₆-alkenyl, C₃-C₆-alkynyl, phenyl-(C₁-C₆)-alkyl, heterocyclyl-(C₁-C₆)-alkyl or COR²¹, where R²¹ is as defined above and is in particular C₁-C₆-alkyl, C₁-C₆-alkoxy, phenyl, phenylamino or heterocyclyl; where the abovementioned aliphatic, cyclic or aromatic moieties of the substituents may be partially or fully halogenated. With particular preference R² has the meaning C₁-C₆-alkyl, in particular methyl.
R³ is hydrogen, halogen, C₁-C₆-alkyl or halo-C₁-C₆-alkyl.
R⁴, R⁵ and/or R⁶ are hydrogen.
A¹ is aryl or heteroaryl selected from the group consisting of phenyl, naphthyl, furyl, thienyl, pyrrolyl, pyrazolyl, thiazolyl, isothiazolyl, oxazolyl, isoxazolyl, triazolyl, tetrazolyl, pyridinyl, pyridazinyl, pyrimidinyl, pyrazinyl, triazinyl or tetrazinyl.
   In particular, A¹ is selected from the group consisting of phenyl, furyl, thienyl, triazolyl, tetrazolyl or pyridinyl.
   With particular preference, A¹ has the meaning phenyl or pyridinyl, in particular phenyl.
   In another preferred embodiment, A¹ is a bicyclic aromatic radical, in particular napthyl or one of the bicyclic heteroaromatic radicals mentioned above, such as quinolinyl, isoquinolinyl, quinazolinyl, quinoxalinyl, indolyl, benzothienyl, benzofuryl, benzoxazolyl, benzothiazolyl, benzimidazolyl, benzopyrazolyl, benzotriazole, indolizinyl, 1,2,4-triazolo[1,5-a]pyrimidinyl, 1,2,4-triazolo[4,3-a]pyridinyl, pyrazolo[3,4-b]pyridinyl, 1,2,4-triazolo[1,5-a]pyridinyl, imidazo[1,2-a]pyridyl, imidazo[3,4-a]pyrimidinyl, especially indolyl, very especially 3-indolyl.
A² is aryl or heteroaryl selected from the group consisting of phenyl, naphthyl, furyl, thienyl, pyrrolyl, pyrazolyl, thiazolyl, isothiazolyl, oxazolyl, isoxazolyl, triazolyl, tetrazolyl, pyridinyl, pyridazinyl, pyrimidinyl, pyrazinyl, triazinyl or tetrazinyl, in particular selected from the group consisting of phenyl, furyl, thienyl, triazolyl, tetrazolyl or pyridinyl. Particularly preferably, A² has the meaning phenyl or thienyl, in particular phenyl.

According to the invention, A¹ carries a radical R^{a} different from hydrogen, as mentioned above. Preferably, this radical is attached in the ortho-position to the point of attachment of A¹ to a nitrogen or in particular to a carbon atom of A¹; this ortho-position is preferred independently of whether A¹ is a mono- or polycyclic aromatic or heteroaromatic ring.

In an alternatively preferred embodiment of the invention, if A¹ is a polycyclic aromatic or heteroaromatic ring, the radical R^{a} is preferably not attached to the cycle of the polycyclic ring system which comprises the point of attachment of A¹, i.e. via which A¹ is attached to the exocyclic double bond which leads to the piperazine ring (=“linking cycle”). In a ring system with three or more cycles, R^{a} is preferably attached to a cycle which is directly adjacent to the “linking cycle”. With particular preference, R^{a} is attached as closely as possible to the point of attachment of A¹. Here, R^{a} is in particular in the α-position to the bridgehead atom which is closest to the point of attachment of A¹ to the exocyclic double bond.

If R^{a} is attached to a nitrogen atom, R^{a} is preferably different from halogen, C₁-C₆-alkylthio, C₁-C₆-alkylsulfinyl, Z¹P(O)(OR⁹)₂, where Z¹ is a bond. In a preferred embodiment of the invention, R^{a} is attached to a carbon atom.

R^{a} has preferably one of the following meanings:
halogen, cyano, nitro, C₁-C₆-alkyl, C₃-C₆-cycloalkyl, C₂-C₆-alkenyl, C₂-C₆-alkynyl, [tri-(C₁-C₆)-alkylsilyl]-(C₂-C₆)-alkynyl, C₁-C₆-alkylthio, C₁-C₆-alkylsulfinyl, aryl, heterocyclyl, in particular 5- or 6-membered heterocyclyl, where aryl and heterocyclyl are unsubstituted or may have one or 2 radicals selected from the group consisting of C₁-C₄-alkyl, C₁-C₄-haloalkyl, C₁-C₄-alkoxy, C₁-C₄-haloalkoxy, CN and halogen;
Z¹P(O)(OR⁹)₂, where Z¹ is a bond or —CH₂— and R⁹ are each hydrogen or C₁-C₆-alkyl;
Z³COR¹¹, where Z³ is a bond and R¹¹ has the meanings mentioned above and in particular is hydrogen, C₁-C₆-alkyl, hydroxyl, C₁-C₁₀-alkoxy, C₃-C₆-alkenyloxy, C₃-C₆-alkynyloxy, amino, C₁-C₁₀-alkylamino, [di-(C₁-C₆)-alkyl]amino, C₁-C₆-alkoxyamino, N—C₁-C₆-alkoxy-N—C₁-C₆-alkylamino, [di-(C₁-C₆)-alkoxy]amino, C₁-C₆-alkylsulfonylamino, C₁-C₆-alkylaminosulfonylamino, [di-(C₁-C₆)-alkylamino]sulfonylamino, phenyl, phenoxy, phenylamino, naphthyl or heterocyclyl, especially 5- or 6-membered heteroaryl attached via carbon;
Z⁴NR¹²R¹³, where Z⁴ is a bond or —CH₂— and R¹² and R¹³ have the meanings mentioned above and in particular independently of one another are hydrogen, C₁-C₆-alkyl, C₃-C₆-cycloalkyl, C₃-C₆-alkenyl, C₃-C₆-alkynyl, C₁-C₆-alkylcarbonyl, [di-(C₁-C₆)-alkylamino]carbonyl, C₁-C₆-alkoxycarbonyl, C₁-C₆-alkylsulfonyl, C₁-C₆-alkylaminosulfonyl, [di-(C₁-C₆)-alkylamino]sulfonyl, C₃-C₆-cycloalkylcarbonyl, phenylcarbonyl, phenylaminocarbonyl, phenylsulfonyl, phenylsulfonylaminocarbonyl or heterocyclylcarbonyl, especially 5- or 6-membered heteroarylcarbonyl attached via carbon;
Z⁵CH═N—O—R¹⁴, where Z⁵ is a bond and R¹⁴ is hydrogen or C₁-C₆-alkyl; or
Z⁶OR¹⁵, where Z⁶ is a bond or —CH₂— and R¹⁵ has the meanings mentioned above and in particular is C₃-C₆-cycloalkyl, C₃-C₆-alkenyl, C₃-C₆-alkynyl, C₁-C₆-alkylcarbonyl, C₁-C₆-alkoxycarbonyl-(C₁-C₆)-alkyl, di-(C₁-C₆-alkoxycarbonyl)-C₁-C₆-alkyl, phenylcarbonyl or phenyl, R¹⁵ may also be hydrogen, C₁-C₁₀-alkyl or phenyl-C₁-C₆-alkyl; or
Z⁷SO₂R¹⁶, where Z⁷ is a bond or CH₂ and R¹⁶ is C₁-C₆-alkyl or phenyl;
   and where the abovementioned aliphatic, cyclic or aromatic moieties of the substituents R^{a} may be partially or fully halogenated.

If R^{a} is attached to a nitrogen atom, R^{a} is preferably different from halogen, C₁-C₆-alkylthio, C₁-C₆-alkylsulfinyl, Z¹P(O)(OR⁹)₂, where Z¹ is a bond. In a preferred embodiment of the invention, R^{a} is attached to a carbon atom.

R^{a} has in particular one of the following meanings:
halogen, cyano, nitro, C₁-C₆-alkylthio, C₁-C₆-alkylsulfinyl, aryl, heterocyclyl, where the two last-mentioned radicals are unsubstituted or may have one or 2 radicals selected from the group consisting of C₁-C₄-alkyl, C₁-C₄-haloalkyl, C₁-C₄-alkoxy, C₁-C₄-haloalkoxy, CN and halogen,
C₂-C₆-alkenyl, C₂-C₆-alkynyl,
Z¹P(O)(OR⁹)₂, where Z¹ is a bond or —CH₂— and R⁹ is in each case hydrogen or C₁-C₆-alkyl; or
Z³COR¹¹, where Z³ is a bond and R¹¹ is hydrogen, C₁-C₆-alkyl, hydroxyl, C₁-C₆-alkoxy, C₃-C₆-alkenyloxy, C₃-C₆-alkynyloxy, amino, C₁-C₆-alkylamino, [di-(C₁-C₆)-alkyl]amino, C₁-C₆-alkoxyamino, N—C₁-C₆-alkoxy-N—C₁-C₆-alkylamino, C₁-C₆-alkylsulfonylamino, C₁-C₆-alkylaminosulfonylamino, [di-(C₁-C₆)-alkyl-amino]sulfonylamino, phenyl, phenoxy, phenylamino, naphthyl or heterocyclyl, especially 5- or 6-membered heteroaryl which is attached via carbon; or
Z⁴NR¹²R¹³, where Z⁴ is a bond or —CH₂— and R¹² and R¹³ independently of one another are hydrogen, C₁-C₆-alkyl, C₃-C₆-cycloalkyl, C₃-C₆-alkenyl, C₃-C₆-alkynyl, C₁-C₆-alkylcarbonyl, [di-(C₁-C₆)-alkylamino]carbonyl, C₁-C₆-alkoxycarbonyl, C₁-C₆-alkylsulfonyl, phenylcarbonyl, phenylsulfonyl, or heterocyclylcarbonyl, especially 5- or 6-membered heteroarylcarbonyl which is attached via carbon; or
Z⁵CH═N—O—R¹⁴, where Z⁵ is a bond and R¹⁴ is hydrogen or C₁-C₆-alkyl; or
Z⁶OR¹⁵, where Z⁶ is a bond or —CH₂— and R¹⁵ is hydrogen, C₁-C₆-alkyl, C₃-C₆-cycloalkyl, C₃-C₆-alkenyl, C₃-C₆-alkynyl, C₁-C₆-alkylcarbonyl, C₁-C₆-alkoxy-carbonyl-(C₁-C₆)-alkyl, phenyl or phenyl-(C₁-C₆)-alkyl; or
Z⁷SO₂R¹⁶, where Z⁷ is a bond or CH₂ and R¹⁶ is C₁-C₆-alkyl or phenyl;
   and where the aliphatic, cyclic or aromatic parts mentioned of the substituents R^{a} may be partially or fully halogenated.

Very particularly preferably, R^{a} is a radical selected from the group consisting of halogen, cyano, nitro, C₂-C₄-alkenyl and C₂-C₄-alkynyl, NH—C(O)—C₁-C₆-alkyl, NH—S(O)₂—C₁-C₆-alkyl and 5-membered heteroaryl, for example oxazolyl, thiazolyl, isoxazolyl, isothiazolyl, where the heteroaryl radicals mentioned above may have one or 2 radicals selected from the group consisting of C₁-C₂-alkyl, C₁-C₂-haloalkyl and halogen, and which is in particular attached in one of the ortho-positions of A¹.

R^{b}, R^{c}, R^{d}, R^{e} and R^{f} are preferably hydrogen or independently of one another have one of the meanings mentioned as being preferred or particularly preferred for R^{a}.

In particular, the radicals R^{b}, R^{c}, R^{d}, R^{e} and R^{f} independently of one another are selected from the group consisting of hydrogen, halogen, C₁-C₄-alkyl, C₂-C₄-alkenyl, C₂-C₄-alkynyl, C₁-C₄-alkoxy, C₁-C₄-haloalkyl and C₁-C₄-haloalkoxy, where two groups R^{b}, R^{c}, R^{d}, R^{e} or R^{f} attached to adjacent carbon atoms of A¹ or A² may also be a group O—CH₂—O.

R^{b} is in particular a radical different from hydrogen. Preferably, R^{b} is a radical attached in the ortho-position of A¹, i.e. if R^{a} is likewise attached in the ortho-position, R^{b} is in the second ortho-position.

If one or both radicals R^{b}, R^{c} are substituents different from hydrogen, they are selected in particular from among the substituents stated as being preferred, and especially from the group consisting of halogen, C₁-C₄-alkyl, C₁-C₄-alkoxy, C₁-C₄-haloalkyl, C₂-C₄-alkenyl, C₂-C₄-alkynyl and C₁-C₄-haloalkoxy, or R^{b} and R^{c} together are a group O—CH₂—O.

A² is in particular unsubstituted, or one or two of the substituents R^{d}, R^{e} and R^{f} are substituents different from hydrogen. If 1 or 2 of the substituents R^{d}, R^{e} and R^{f} are different from hydrogen, they are selected in particular from the group consisting of halogen, C₁-C₄-alkyl, C₁-C₄-alkoxy, C₁-C₄-haloalkyl and C₁-C₄-haloalkoxy.

The meaning that a substituent may assume in the context of the invention is completely independent of the meaning which another substituent may assume in the context of the invention. However, particular preference is given to compounds of the formula I in which a plurality or in particular all variables have a meaning given as being preferred or particularly preferred.

Furthermore, it has been found to be advantageous for the exocyclic double bond at the piperazine ring to have the Z-conformation, i.e. the group A¹(R^{a}R^{b}R^{c}) and the ring nitrogen which carries the group R² are in a cis arrangement with respect to this double bond. Accordingly, a preferred embodiment of the invention relates to piperazine compounds of the formula (Z)-I in which the variables A¹, A², R¹-R⁶ and R^{a}-R^{f} are as defined above, and also to mixtures of the Z-isomer (Z)-I with the E-isomer of the formula (E)-I, where the E/Z ratio is <1:1, in particular <1:2, particularly preferably <1:4 and especially <1:10. In formula (E)-I, the variables A¹, A², R¹-R⁶ and R^{a}-R^{f} are also as defined above.

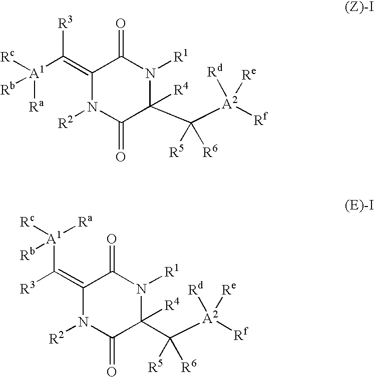

Furthermore, it has been found to be advantageous for the carbon atom which carries R⁴ to have the S-configuration. Accordingly, a preferred embodiment of the invention relates to piperazine compounds of the formula I-S in which the variables A¹, A², R¹-R⁶ and R^{a}-R^{f} are as defined above, and also to mixtures of the S-enantiomer (S)-I with the R-enantiomer of the formula (R)-I in which the R/S ratio is <1:1, in particular <1:2, particularly preferably <1:4 and especially <1:10. In formula (R)-I, the variables A¹, A², R¹-R⁶ and R^{a}-R^{f} are also as defined above.

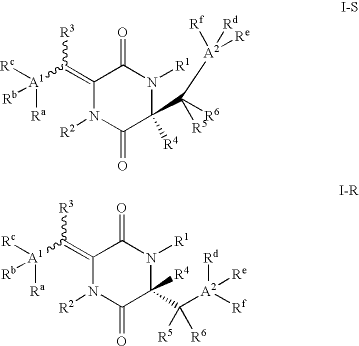

Preference is given in particular to compounds of the formula I in which both the exocyclic double bond has the Z-conformation and the carbon atom which carries R⁴ has the S-configuration. Hereinbelow, these compounds are also referred to as compounds (Z,S)-I.

Preference is given in particular to the compounds of the formula I.1 in which R¹, R³, R⁴, R⁵, and R⁶ are hydrogen and R² is CH₃, particularly preferably the compounds (Z,S)-I.1 which have the Z- or S-configuration at the indicated positions (1) and (2). Examples of preferred compounds I.1 and (Z,S)-I.1 are those in which A¹, R^{a}, R^{b} and R^{c} are as defined in Table 1:

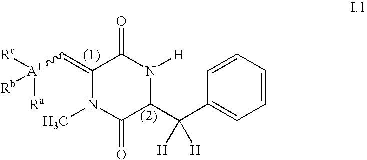

The compounds I.1.1 to I.3434 listed below are per se preferred embodiments of the invention.

TABLE 1

| Comp. No. | A¹ | R^{a} | R^{b} | R^{c} |
|---|---|---|---|---|
| I.1.1 | phenyl | 2-NO₂ | H | H |
| I.1.2 | phenyl | 2-NO₂ | 3-Cl | H |
| I.1.3 | phenyl | 2-NO₂ | 4-Cl | H |
| I.1.4 | phenyl | 2-NO₂ | 5-Cl | H |
| I.1.5 | phenyl | 2-NO₂ | 6-Cl | H |
| I.1.6 | phenyl | 2-NO₂ | 3-F | H |
| I.1.7 | phenyl | 2-NO₂ | 4-F | H |
| I.1.8 | phenyl | 2-NO₂ | 5-F | H |
| I.1.9 | phenyl | 2-NO₂ | 6-F | H |
| I.1.10 | phenyl | 2-NO₂ | 3-CH₃ | H |
| I.1.11 | phenyl | 2-NO₂ | 4-CH₃ | H |
| I.1.12 | phenyl | 2-NO₂ | 5-CH₃ | H |
| I.1.13 | phenyl | 2-NO₂ | 6-CH₃ | H |
| I.1.14 | phenyl | 2-NO₂ | 5-vinyl | H |
| I.1.15 | phenyl | 2-NO₂ | 6-vinyl | H |
| I.1.16 | phenyl | 2-NO₂ | 5-CF₃ | 3-Cl |
| I.1.17 | phenyl | 2-NO₂ | 5-CF₃ | 4-Cl |
| I.1.18 | phenyl | 2-NO₂ | 4-CF₃ | 5-Cl |
| I.1.19 | phenyl | 2-NO₂ | 5-CF₃ | 6-Cl |
| I.1.20 | phenyl | 2-NO₂ | 5-CF₃ | 3-F |
| I.1.21 | phenyl | 2-NO₂ | 5-CF₃ | 4-F |
| I.1.22 | phenyl | 2-NO₂ | 4-CF₃ | 5-F |
| I.1.23 | phenyl | 2-NO₂ | 5-CF₃ | 6-F |
| I.1.24 | phenyl | 2-NO₂ | 5-CF₃ | 3-CH₃ |
| I.1.25 | phenyl | 2-NO₂ | 5-CF₃ | 4-CH₃ |
| I.1.26 | phenyl | 2-NO₂ | 4-CF₃ | 5-CH₃ |
| I.1.27 | phenyl | 2-NO₂ | 5-CF₃ | 6-CH₃ |
| I.1.28 | phenyl | 2-NO₂ | 4-CF₃ | 5-vinyl |
| I.1.29 | phenyl | 2-NO₂ | 4-CF₃ | 6-vinyl |
| I.1.30 | phenyl | 2-NO₂ | 3-Cl | 4-F |
| I.1.31 | phenyl | 2-NO₂ | 3-Cl | 5-F |
| I.1.32 | phenyl | 2-NO₂ | 3-Cl | 6-F |
| I.1.33 | phenyl | 2-NO₂ | 3-Cl | 4-Cl |
| I.1.34 | phenyl | 2-NO₂ | 3-Cl | 5-Cl |
| I.1.35 | phenyl | 2-NO₂ | 3-Cl | 6-Cl |
| I.1.36 | phenyl | 2-NO₂ | 3-Cl | 4-CH₃ |
| I.1.37 | phenyl | 2-NO₂ | 3-Cl | 5-CH₃ |
| I.1.38 | phenyl | 2-NO₂ | 3-Cl | 6-CH₃ |
| I.1.39 | phenyl | 2-NO₂ | 3-Cl | 5-vinyl |
| I.1.40 | phenyl | 2-NO₂ | 3-Cl | 6-vinyl |
| I.1.41 | phenyl | 2-NO₂ | 3-F | 4-F |
| I.1.42 | phenyl | 2-NO₂ | 3-F | 5-F |
| I.1.43 | phenyl | 2-NO₂ | 3-F | 6-F |
| I.1.44 | phenyl | 2-NO₂ | 3-F | 4-Cl |
| I.1.45 | phenyl | 2-NO₂ | 3-F | 5-Cl |
| I.1.46 | phenyl | 2-NO₂ | 3-F | 6-Cl |
| I.1.47 | phenyl | 2-NO₂ | 3-F | 4-CH₃ |
| I.1.48 | phenyl | 2-NO₂ | 3-F | 5-CH₃ |
| I.1.49 | phenyl | 2-NO₂ | 3-F | 6-CH₃ |
| I.1.50 | phenyl | 2-NO₂ | 3-F | 5-vinyl |
| I.1.51 | phenyl | 2-NO₂ | 3-F | 6-vinyl |
| I.1.52 | phenyl | 2-NO₂ | 3-CH₃ | 4-F |
| I.1.53 | phenyl | 2-NO₂ | 3-CH₃ | 5-F |
| I.1.54 | phenyl | 2-NO₂ | 3-CH₃ | 6-F |
| I.1.55 | phenyl | 2-NO₂ | 3-CH₃ | 4-Cl |
| I.1.56 | phenyl | 2-NO₂ | 3-CH₃ | 5-Cl |
| I.1.57 | phenyl | 2-NO₂ | 3-CH₃ | 6-Cl |
| I.1.58 | phenyl | 2-NO₂ | 3-CH₃ | 4-CH₃ |
| I.1.59 | phenyl | 2-NO₂ | 3-CH₃ | 5-CH₃ |
| I.1.60 | phenyl | 2-NO₂ | 3-CH₃ | 6-CH₃ |
| I.1.61 | phenyl | 2-NO₂ | 3-CH₃ | 5-vinyl |
| I.1.62 | phenyl | 2-NO₂ | 3-CH₃ | 6-vinyl |
| I.1.63 | phenyl | 2-NO₂ | 4-Cl | 5-F |
| I.1.64 | phenyl | 2-NO₂ | 4-Cl | 6-F |
| I.1.65 | phenyl | 2-NO₂ | 4-Cl | 5-Cl |
| I.1.66 | phenyl | 2-NO₂ | 4-Cl | 6-Cl |
| I.1.67 | phenyl | 2-NO₂ | 4-Cl | 5-CH₃ |
| I.1.68 | phenyl | 2-NO₂ | 4-Cl | 6-CH₃ |
| I.1.69 | phenyl | 2-NO₂ | 4-Cl | 5-vinyl |
| I.1.70 | phenyl | 2-NO₂ | 4-Cl | 6-vinyl |
| I.1.71 | phenyl | 2-NO₂ | 4-F | 5-F |
| I.1.72 | phenyl | 2-NO₂ | 4-F | 6-F |
| I.1.73 | phenyl | 2-NO₂ | 4-F | 5-Cl |
| I.1.74 | phenyl | 2-NO₂ | 4-F | 6-Cl |
| I.1.75 | phenyl | 2-NO₂ | 4-F | 5-CH₃ |
| I.1.76 | phenyl | 2-NO₂ | 4-F | 6-CH₃ |
| I.1.77 | phenyl | 2-NO₂ | 4-F | 5-vinyl |
| I.1.78 | phenyl | 2-NO₂ | 4-F | 6-vinyl |
| I.1.79 | phenyl | 2-NO₂ | 4-CH₃ | 5-F |
| I.1.80 | phenyl | 2-NO₂ | 4-CH₃ | 6-F |
| I.1.81 | phenyl | 2-NO₂ | 4-CH₃ | 5-Cl |
| I.1.82 | phenyl | 2-NO₂ | 4-CH₃ | 6-Cl |
| I.1.83 | phenyl | 2-NO₂ | 4-CH₃ | 5-CH₃ |
| I.1.84 | phenyl | 2-NO₂ | 4-CH₃ | 6-CH₃ |
| I.1.85 | phenyl | 2-NO₂ | 4-CH₃ | 5-vinyl |
| I.1.86 | phenyl | 2-NO₂ | 4-CH₃ | 6-vinyl |
| I.1.87 | phenyl | 2-NO₂ | 5-Cl | 6-Cl |
| I.1.88 | phenyl | 2-NO₂ | 5-Cl | 6-F |
| I.1.89 | phenyl | 2-NO₂ | 5-Cl | 6-CH₃ |
| I.1.90 | phenyl | 2-NO₂ | 5-Cl | 6-vinyl |
| I.1.91 | phenyl | 2-NO₂ | 5-F | 6-Cl |
| I.1.92 | phenyl | 2-NO₂ | 5-F | 6-F |
| I.1.93 | phenyl | 2-NO₂ | 5-F | 6-CH₃ |
| I.1.94 | phenyl | 2-NO₂ | 5-F | 6-vinyl |
| I.1.95 | phenyl | 2-NO₂ | 5-CH₃ | 6-Cl |
| I.1.96 | phenyl | 2-NO₂ | 5-CH₃ | 6-F |
| I.1.97 | phenyl | 2-NO₂ | 5-CH₃ | 6-CH₃ |
| I.1.98 | phenyl | 2-NO₂ | 5-CH₃ | 6-vinyl |
| I.1.99 | phenyl | 2-NO₂ | 5-vinyl | 6-Cl |
| I.1.100 | phenyl | 2-NO₂ | 5-vinyl | 6-F |
| I.1.101 | phenyl | 2-NO₂ | 5-vinyl | 6-CH₃ |
| I.1.102 | phenyl | 2-NO₂ | 5-vinyl | 6-vinyl |
| I.1.103 | phenyl | 2-CN | H | H |
| I.1.104 | phenyl | 2-CN | 3-Cl | H |
| I.1.105 | phenyl | 2-CN | 4-Cl | H |
| I.1.106 | phenyl | 2-CN | 5-Cl | H |
| I.1.107 | phenyl | 2-CN | 6-Cl | H |
| I.1.108 | phenyl | 2-CN | 3-F | H |
| I.1.109 | phenyl | 2-CN | 4-F | H |
| I.1.110 | phenyl | 2-CN | 5-F | H |
| I.1.111 | phenyl | 2-CN | 6-F | H |
| I.1.112 | phenyl | 2-CN | 3-CH₃ | H |
| I.1.113 | phenyl | 2-CN | 4-CH₃ | H |
| I.1.114 | phenyl | 2-CN | 5-CH₃ | H |
| I.1.115 | phenyl | 2-CN | 6-CH₃ | H |
| I.1.116 | phenyl | 2-CN | 5-vinyl | H |
| I.1.117 | phenyl | 2-CN | 6-vinyl | H |
| I.1.118 | phenyl | 2-CN | 5-CF₃ | 3-Cl |
| I.1.119 | phenyl | 2-CN | 5-CF₃ | 4-Cl |
| I.1.120 | phenyl | 2-CN | 4-CF₃ | 5-Cl |
| I.1.121 | phenyl | 2-CN | 5-CF₃ | 6-Cl |
| I.1.122 | phenyl | 2-CN | 5-CF₃ | 3-F |
| I.1.123 | phenyl | 2-CN | 5-CF₃ | 4-F |
| I.1.124 | phenyl | 2-CN | 4-CF₃ | 5-F |
| I.1.125 | phenyl | 2-CN | 5-CF₃ | 6-F |
| I.1.126 | phenyl | 2-CN | 5-CF₃ | 3-CH₃ |
| I.1.127 | phenyl | 2-CN | 5-CF₃ | 4-CH₃ |
| I.1.128 | phenyl | 2-CN | 4-CF₃ | 5-CH₃ |
| I.1.129 | phenyl | 2-CN | 5-CF₃ | 6-CH₃ |
| I.1.130 | phenyl | 2-CN | 4-CF₃ | 5-vinyl |
| I.1.131 | phenyl | 2-CN | 4-CF₃ | 6-vinyl |
| I.1.132 | phenyl | 2-CN | 3-Cl | 4-F |
| I.1.133 | phenyl | 2-CN | 3-Cl | 5-F |
| I.1.134 | phenyl | 2-CN | 3-Cl | 6-F |
| I.1.135 | phenyl | 2-CN | 3-Cl | 4-Cl |
| I.1.136 | phenyl | 2-CN | 3-Cl | 5-Cl |
| I.1.137 | phenyl | 2-CN | 3-Cl | 6-Cl |
| I.1.138 | phenyl | 2-CN | 3-Cl | 4-CH₃ |
| I.1.139 | phenyl | 2-CN | 3-Cl | 5-CH₃ |
| I.1.140 | phenyl | 2-CN | 3-Cl | 6-CH₃ |
| I.1.141 | phenyl | 2-CN | 3-Cl | 5-vinyl |
| I.1.142 | phenyl | 2-CN | 3-Cl | 6-vinyl |
| I.1.143 | phenyl | 2-CN | 3-F | 4-F |
| I.1.144 | phenyl | 2-CN | 3-F | 5-F |
| I.1.145 | phenyl | 2-CN | 3-F | 6-F |
| I.1.146 | phenyl | 2-CN | 3-F | 4-Cl |
| I.1.147 | phenyl | 2-CN | 3-F | 5-Cl |
| I.1.148 | phenyl | 2-CN | 3-F | 6-Cl |
| I.1.149 | phenyl | 2-CN | 3-F | 4-CH₃ |
| I.1.150 | phenyl | 2-CN | 3-F | 5-CH₃ |
| I.1.151 | phenyl | 2-CN | 3-F | 6-CH₃ |
| I.1.152 | phenyl | 2-CN | 3-F | 5-vinyl |
| I.1.153 | phenyl | 2-CN | 3-F | 6-vinyl |
| I.1.154 | phenyl | 2-CN | 3-CH₃ | 4-F |
| I.1.155 | phenyl | 2-CN | 3-CH₃ | 5-F |
| I.1.156 | phenyl | 2-CN | 3-CH₃ | 6-F |
| I.1.157 | phenyl | 2-CN | 3-CH₃ | 4-Cl |
| I.1.158 | phenyl | 2-CN | 3-CH₃ | 5-Cl |
| I.1.159 | phenyl | 2-CN | 3-CH₃ | 6-Cl |
| I.1.160 | phenyl | 2-CN | 3-CH₃ | 4-CH₃ |
| I.1.161 | phenyl | 2-CN | 3-CH₃ | 5-CH₃ |
| I.1.162 | phenyl | 2-CN | 3-CH₃ | 6-CH₃ |
| I.1.163 | phenyl | 2-CN | 3-CH₃ | 5-vinyl |
| I.1.164 | phenyl | 2-CN | 3-CH₃ | 6-vinyl |
| I.1.165 | phenyl | 2-CN | 4-Cl | 5-F |
| I.1.166 | phenyl | 2-CN | 4-Cl | 6-F |
| I.1.167 | phenyl | 2-CN | 4-Cl | 5-Cl |
| I.1.168 | phenyl | 2-CN | 4-Cl | 6-Cl |
| I.1.169 | phenyl | 2-CN | 4-Cl | 5-CH₃ |
| I.1.170 | phenyl | 2-CN | 4-Cl | 6-CH₃ |
| I.1.171 | phenyl | 2-CN | 4-Cl | 5-vinyl |
| I.1.172 | phenyl | 2-CN | 4-Cl | 6-vinyl |
| I.1.173 | phenyl | 2-CN | 4-F | 5-F |
| I.1.174 | phenyl | 2-CN | 4-F | 6-F |
| I.1.175 | phenyl | 2-CN | 4-F | 5-Cl |
| I.1.176 | phenyl | 2-CN | 4-F | 6-Cl |
| I.1.177 | phenyl | 2-CN | 4-F | 5-CH₃ |
| I.1.178 | phenyl | 2-CN | 4-F | 6-CH₃ |
| I.1.179 | phenyl | 2-CN | 4-F | 5-vinyl |
| I.1.180 | phenyl | 2-CN | 4-F | 6-vinyl |
| I.1.181 | phenyl | 2-CN | 4-CH₃ | 5-F |
| I.1.182 | phenyl | 2-CN | 4-CH₃ | 6-F |
| I.1.183 | phenyl | 2-CN | 4-CH₃ | 5-Cl |
| I.1.184 | phenyl | 2-CN | 4-CH₃ | 6-Cl |
| I.1.185 | phenyl | 2-CN | 4-CH₃ | 5-CH₃ |
| I.1.186 | phenyl | 2-CN | 4-CH₃ | 6-CH₃ |
| I.1.187 | phenyl | 2-CN | 4-CH₃ | 5-vinyl |
| I.1.188 | phenyl | 2-CN | 4-CH₃ | 6-vinyl |
| I.1.189 | phenyl | 2-CN | 5-Cl | 6-Cl |
| I.1.190 | phenyl | 2-CN | 5-Cl | 6-F |
| I.1.191 | phenyl | 2-CN | 5-Cl | 6-CH₃ |
| I.1.192 | phenyl | 2-CN | 5-Cl | 6-vinyl |
| I.1.193 | phenyl | 2-CN | 5-F | 6-Cl |
| I.1.194 | phenyl | 2-CN | 5-F | 6-F |
| I.1.195 | phenyl | 2-CN | 5-F | 6-CH₃ |
| I.1.196 | phenyl | 2-CN | 5-F | 6-vinyl |
| I.1.197 | phenyl | 2-CN | 5-CH₃ | 6-Cl |
| I.1.198 | phenyl | 2-CN | 5-CH₃ | 6-F |
| I.1.199 | phenyl | 2-CN | 5-CH₃ | 6-CH₃ |
| I.1.200 | phenyl | 2-CN | 5-CH₃ | 6-vinyl |
| I.1.201 | phenyl | 2-CN | 5-vinyl | 6-Cl |
| I.1.202 | phenyl | 2-CN | 5-vinyl | 6-F |
| I.1.203 | phenyl | 2-CN | 5-vinyl | 6-CH₃ |
| I.1.204 | phenyl | 2-CN | 5-vinyl | 6-vinyl |
| I.1.205 | phenyl | 2-ethynyl | H | H |
| I.1.206 | phenyl | 2-ethynyl | 3-Cl | H |
| I.1.207 | phenyl | 2-ethynyl | 4-Cl | H |
| I.1.208 | phenyl | 2-ethynyl | 5-Cl | H |
| I.1.209 | phenyl | 2-ethynyl | 6-Cl | H |
| I.1.210 | phenyl | 2-ethynyl | 3-F | H |
| I.1.211 | phenyl | 2-ethynyl | 4-F | H |
| I.1.212 | phenyl | 2-ethynyl | 5-F | H |
| I.1.213 | phenyl | 2-ethynyl | 6-F | H |
| I.1.214 | phenyl | 2-ethynyl | 3-CH₃ | H |
| I.1.215 | phenyl | 2-ethynyl | 4-CH₃ | H |
| I.1.216 | phenyl | 2-ethynyl | 5-CH₃ | H |
| I.1.217 | phenyl | 2-ethynyl | 6-CH₃ | H |
| I.1.218 | phenyl | 2-ethynyl | 5-vinyl | H |
| I.1.219 | phenyl | 2-ethynyl | 6-vinyl | H |
| I.1.220 | phenyl | 2-ethynyl | 5-CF₃ | 3-Cl |
| I.1.221 | phenyl | 2-ethynyl | 5-CF₃ | 4-Cl |
| I.1.222 | phenyl | 2-ethynyl | 4-CF₃ | 5-Cl |
| I.1.223 | phenyl | 2-ethynyl | 5-CF₃ | 6-Cl |
| I.1.224 | phenyl | 2-ethynyl | 5-CF₃ | 3-F |
| I.1.225 | phenyl | 2-ethynyl | 5-CF₃ | 4-F |
| I.1.226 | phenyl | 2-ethynyl | 4-CF₃ | 5-F |
| I.1.227 | phenyl | 2-ethynyl | 5-CF₃ | 6-F |
| I.1.228 | phenyl | 2-ethynyl | 5-CF₃ | 3-CH₃ |
| I.1.229 | phenyl | 2-ethynyl | 5-CF₃ | 4-CH₃ |
| I.1.230 | phenyl | 2-ethynyl | 4-CF₃ | 5-CH₃ |
| I.1.231 | phenyl | 2-ethynyl | 5-CF₃ | 6-CH₃ |
| I.1.232 | phenyl | 2-ethynyl | 4-CF₃ | 5-vinyl |
| I.1.233 | phenyl | 2-ethynyl | 4-CF₃ | 6-vinyl |
| I.1.234 | phenyl | 2-ethynyl | 3-Cl | 4-F |
| I.1.235 | phenyl | 2-ethynyl | 3-Cl | 5-F |
| I.1.236 | phenyl | 2-ethynyl | 3-Cl | 6-F |
| I.1.237 | phenyl | 2-ethynyl | 3-Cl | 4-Cl |
| I.1.238 | phenyl | 2-ethynyl | 3-Cl | 5-Cl |
| I.1.239 | phenyl | 2-ethynyl | 3-Cl | 6-Cl |
| I.1.240 | phenyl | 2-ethynyl | 3-Cl | 4-CH₃ |
| I.1.241 | phenyl | 2-ethynyl | 3-Cl | 5-CH₃ |
| I.1.242 | phenyl | 2-ethynyl | 3-Cl | 6-CH₃ |
| I.1.243 | phenyl | 2-ethynyl | 3-Cl | 5-vinyl |
| I.1.244 | phenyl | 2-ethynyl | 3-Cl | 6-vinyl |
| I.1.245 | phenyl | 2-ethynyl | 3-F | 4-F |
| I.1.246 | phenyl | 2-ethynyl | 3-F | 5-F |
| I.1.247 | phenyl | 2-ethynyl | 3-F | 6-F |
| I.1.248 | phenyl | 2-ethynyl | 3-F | 4-Cl |
| I.1.249 | phenyl | 2-ethynyl | 3-F | 5-Cl |
| I.1.250 | phenyl | 2-ethynyl | 3-F | 6-Cl |
| I.1.251 | phenyl | 2-ethynyl | 3-F | 4-CH₃ |
| I.1.252 | phenyl | 2-ethynyl | 3-F | 5-CH₃ |
| I.1.253 | phenyl | 2-ethynyl | 3-F | 6-CH₃ |
| I.1.254 | phenyl | 2-ethynyl | 3-F | 5-vinyl |
| I.1.255 | phenyl | 2-ethynyl | 3-F | 6-vinyl |
| I.1.256 | phenyl | 2-ethynyl | 3-CH₃ | 4-F |
| I.1.257 | phenyl | 2-ethynyl | 3-CH₃ | 5-F |
| I.1.258 | phenyl | 2-ethynyl | 3-CH₃ | 6-F |
| I.1.259 | phenyl | 2-ethynyl | 3-CH₃ | 4-Cl |
| I.1.260 | phenyl | 2-ethynyl | 3-CH₃ | 5-Cl |
| I.1.261 | phenyl | 2-ethynyl | 3-CH₃ | 6-Cl |
| I.1.262 | phenyl | 2-ethynyl | 3-CH₃ | 4-CH₃ |
| I.1.263 | phenyl | 2-ethynyl | 3-CH₃ | 5-CH₃ |
| I.1.264 | phenyl | 2-ethynyl | 3-CH₃ | 6-CH₃ |
| I.1.265 | phenyl | 2-ethynyl | 3-CH₃ | 5-vinyl |
| I.1.266 | phenyl | 2-ethynyl | 3-CH₃ | 6-vinyl |
| I.1.267 | phenyl | 2-ethynyl | 4-Cl | 5-F |
| I.1.268 | phenyl | 2-ethynyl | 4-Cl | 6-F |
| I.1.269 | phenyl | 2-ethynyl | 4-Cl | 5-Cl |
| I.1.270 | phenyl | 2-ethynyl | 4-Cl | 6-Cl |
| I.1.271 | phenyl | 2-ethynyl | 4-Cl | 5-CH₃ |
| I.1.272 | phenyl | 2-ethynyl | 4-Cl | 6-CH₃ |
| I.1.273 | phenyl | 2-ethynyl | 4-Cl | 5-vinyl |
| I.1.274 | phenyl | 2-ethynyl | 4-Cl | 6-vinyl |
| I.1.275 | phenyl | 2-ethynyl | 4-F | 5-F |
| I.1.276 | phenyl | 2-ethynyl | 4-F | 6-F |
| I.1.277 | phenyl | 2-ethynyl | 4-F | 5-Cl |
| I.1.278 | phenyl | 2-ethynyl | 4-F | 6-Cl |
| I.1.279 | phenyl | 2-ethynyl | 4-F | 5-CH₃ |
| I.1.280 | phenyl | 2-ethynyl | 4-F | 6-CH₃ |
| I.1.281 | phenyl | 2-ethynyl | 4-F | 5-vinyl |
| I.1.282 | phenyl | 2-ethynyl | 4-F | 6-vinyl |
| I.1.283 | phenyl | 2-ethynyl | 4-CH₃ | 5-F |
| I.1.284 | phenyl | 2-ethynyl | 4-CH₃ | 6-F |
| I.1.285 | phenyl | 2-ethynyl | 4-CH₃ | 5-Cl |
| I.1.286 | phenyl | 2-ethynyl | 4-CH₃ | 6-Cl |
| I.1.287 | phenyl | 2-ethynyl | 4-CH₃ | 5-CH₃ |
| I.1.288 | phenyl | 2-ethynyl | 4-CH₃ | 6-Cl |
| I.1.289 | phenyl | 2-ethynyl | 4-CH₃ | 5-vinyl |
| I.1.290 | phenyl | 2-ethynyl | 4-CH₃ | 6-vinyl |
| I.1.291 | phenyl | 2-ethynyl | 5-Cl | 6-Cl |
| I.1.292 | phenyl | 2-ethynyl | 5-Cl | 6-F |
| I.1.293 | phenyl | 2-ethynyl | 5-Cl | 6-CH₃ |
| I.1.294 | phenyl | 2-ethynyl | 5-Cl | 6-vinyl |
| I.1.295 | phenyl | 2-ethynyl | 5-F | 6-Cl |
| I.1.296 | phenyl | 2-ethynyl | 5-F | 6-F |
| I.1.297 | phenyl | 2-ethynyl | 5-F | 6-CH₃ |
| I.1.298 | phenyl | 2-ethynyl | 5-F | 6-vinyl |
| I.1.299 | phenyl | 2-ethynyl | 5-CH₃ | 6-Cl |
| I.1.300 | phenyl | 2-ethynyl | 5-CH₃ | 6-F |
| I.1.301 | phenyl | 2-ethynyl | 5-CH₃ | 6-CH₃ |
| I.1.302 | phenyl | 2-ethynyl | 5-CH₃ | 6-vinyl |
| I.1.303 | phenyl | 2-ethynyl | 5-vinyl | 6-Cl |
| I.1.304 | phenyl | 2-ethynyl | 5-vinyl | 6-F |
| I.1.305 | phenyl | 2-ethynyl | 5-vinyl | 6-CH₃ |
| I.1.306 | phenyl | 2-ethynyl | 5-vinyl | 6-vinyl |
| I.1.307 | phenyl | 2-COOCH₃ | H | H |
| I.1.308 | phenyl | 2-COOCH₃ | 3-Cl | H |
| I.1.309 | phenyl | 2-COOCH₃ | 4-Cl | H |
| I.1.310 | phenyl | 2-COOCH₃ | 5-Cl | H |
| I.1.311 | phenyl | 2-COOCH₃ | 6-Cl | H |
| I.1.312 | phenyl | 2-COOCH₃ | 3-F | H |
| I.1.313 | phenyl | 2-COOCH₃ | 4-F | H |
| I.1.314 | phenyl | 2-COOCH₃ | 5-F | H |
| I.1.315 | phenyl | 2-COOCH₃ | 6-F | H |
| I.1.316 | phenyl | 2-COOCH₃ | 3-CH₃ | H |
| I.1.317 | phenyl | 2-COOCH₃ | 4-CH₃ | H |
| I.1.318 | phenyl | 2-COOCH₃ | 5-CH₃ | H |
| I.1.319 | phenyl | 2-COOCH₃ | 6-CH₃ | H |
| I.1.320 | phenyl | 2-COOCH₃ | 5-vinyl | H |
| I.1.321 | phenyl | 2-COOCH₃ | 6-vinyl | H |
| I.1.322 | phenyl | 2-COOCH₃ | 5-CF₃ | 3-Cl |
| I.1.323 | phenyl | 2-COOCH₃ | 5-CF₃ | 4-Cl |
| I.1.324 | phenyl | 2-COOCH₃ | 4-CF₃ | 5-Cl |
| I.1.325 | phenyl | 2-COOCH₃ | 5-CF₃ | 6-Cl |
| I.1.326 | phenyl | 2-COOCH₃ | 5-CF₃ | 3-F |
| I.1.327 | phenyl | 2-COOCH₃ | 5-CF₃ | 4-F |
| I.1.328 | phenyl | 2-COOCH₃ | 4-CF₃ | 5-F |
| I.1.329 | phenyl | 2-COOCH₃ | 5-CF₃ | 6-F |
| I.1.330 | phenyl | 2-COOCH₃ | 5-CF₃ | 3-CH₃ |
| I.1.331 | phenyl | 2-COOCH₃ | 5-CF₃ | 4-CH₃ |
| I.1.332 | phenyl | 2-COOCH₃ | 4-CF₃ | 5-CH₃ |
| I.1.333 | phenyl | 2-COOCH₃ | 5-CF₃ | 6-CH₃ |
| I.1.334 | phenyl | 2-COOCH₃ | 4-CF₃ | 5-vinyl |
| I.1.335 | phenyl | 2-COOCH₃ | 4-CF₃ | 6-vinyl |
| I.1.336 | phenyl | 2-COOCH₃ | 3-Cl | 4-F |
| I.1.337 | phenyl | 2-COOCH₃ | 3-Cl | 5-F |
| I.1.338 | phenyl | 2-COOCH₃ | 3-Cl | 6-F |
| I.1.339 | phenyl | 2-COOCH₃ | 3-Cl | 4-Cl |
| I.1.340 | phenyl | 2-COOCH₃ | 3-Cl | 5-Cl |
| I.1.341 | phenyl | 2-COOCH₃ | 3-Cl | 6-Cl |
| I.1.342 | phenyl | 2-COOCH₃ | 3-Cl | 4-CH₃ |
| I.1.343 | phenyl | 2-COOCH₃ | 3-Cl | 5-CH₃ |
| I.1.344 | phenyl | 2-COOCH₃ | 3-Cl | 6-CH₃ |
| I.1.345 | phenyl | 2-COOCH₃ | 3-Cl | 5-vinyl |
| I.1.346 | phenyl | 2-COOCH₃ | 3-Cl | 6-vinyl |
| I.1.347 | phenyl | 2-COOCH₃ | 3-F | 4-F |
| I.1.348 | phenyl | 2-COOCH₃ | 3-F | 5-F |
| I.1.349 | phenyl | 2-COOCH₃ | 3-F | 6-F |
| I.1.350 | phenyl | 2-COOCH₃ | 3-F | 4-Cl |
| I.1.351 | phenyl | 2-COOCH₃ | 3-F | 5-Cl |
| I.1.352 | phenyl | 2-COOCH₃ | 3-F | 6-Cl |
| I.1.353 | phenyl | 2-COOCH₃ | 3-F | 4-CH₃ |
| I.1.354 | phenyl | 2-COOCH₃ | 3-F | 5-CH₃ |
| I.1.355 | phenyl | 2-COOCH₃ | 3-F | 6-CH₃ |
| I.1.356 | phenyl | 2-COOCH₃ | 3-F | 5-vinyl |
| I.1.357 | phenyl | 2-COOCH₃ | 3-F | 6-vinyl |
| I.1.358 | phenyl | 2-COOCH₃ | 3-CH₃ | 4-F |
| I.1.359 | phenyl | 2-COOCH₃ | 3-CH₃ | 5-F |
| I.1.360 | phenyl | 2-COOCH₃ | 3-CH₃ | 6-F |
| I.1.361 | phenyl | 2-COOCH₃ | 3-CH₃ | 4-Cl |
| I.1.362 | phenyl | 2-COOCH₃ | 3-CH₃ | 5-Cl |
| I.1.363 | phenyl | 2-COOCH₃ | 3-CH₃ | 6-Cl |
| I.1.364 | phenyl | 2-COOCH₃ | 3-CH₃ | 4-CH₃ |
| I.1.365 | phenyl | 2-COOCH₃ | 3-CH₃ | 5-CH₃ |
| I.1.366 | phenyl | 2-COOCH₃ | 3-CH₃ | 6-CH₃ |
| I.1.367 | phenyl | 2-COOCH₃ | 3-CH₃ | 5-vinyl |
| I.1.368 | phenyl | 2-COOCH₃ | 3-CH₃ | 6-vinyl |
| I.1.369 | phenyl | 2-COOCH₃ | 4-Cl | 5-F |
| I.1.370 | phenyl | 2-COOCH₃ | 4-Cl | 6-F |
| I.1.371 | phenyl | 2-COOCH₃ | 4-Cl | 5-Cl |
| I.1.372 | phenyl | 2-COOCH₃ | 4-Cl | 6-Cl |
| I.1.373 | phenyl | 2-COOCH₃ | 4-Cl | 5-CH₃ |
| I.1.374 | phenyl | 2-COOCH₃ | 4-Cl | 6-CH₃ |
| I.1.375 | phenyl | 2-COOCH₃ | 4-Cl | 5-vinyl |
| I.1.376 | phenyl | 2-COOCH₃ | 4-Cl | 6-vinyl |
| I.1.377 | phenyl | 2-COOCH₃ | 4-F | 5-F |
| I.1.378 | phenyl | 2-COOCH₃ | 4-F | 6-F |
| I.1.379 | phenyl | 2-COOCH₃ | 4-F | 5-Cl |
| I.1.380 | phenyl | 2-COOCH₃ | 4-F | 6-Cl |
| I.1.381 | phenyl | 2-COOCH₃ | 4-F | 5-CH₃ |
| I.1.382 | phenyl | 2-COOCH₃ | 4-F | 6-CH₃ |
| I.1.383 | phenyl | 2-COOCH₃ | 4-F | 5-vinyl |
| I.1.384 | phenyl | 2-COOCH₃ | 4-F | 6-vinyl |
| I.1.385 | phenyl | 2-COOCH₃ | 4-CH₃ | 5-F |
| I.1.386 | phenyl | 2-COOCH₃ | 4-CH₃ | 6-F |
| I.1.387 | phenyl | 2-COOCH₃ | 4-CH₃ | 5-Cl |
| I.1.388 | phenyl | 2-COOCH₃ | 4-CH₃ | 6-Cl |
| I.1.389 | phenyl | 2-COOCH₃ | 4-CH₃ | 5-CH₃ |
| I.1.390 | phenyl | 2-COOCH₃ | 4-CH₃ | 6-CH₃ |
| I.1.391 | phenyl | 2-COOCH₃ | 4-CH₃ | 5-vinyl |
| I.1.392 | phenyl | 2-COOCH₃ | 4-CH₃ | 6-vinyl |
| I.1.393 | phenyl | 2-COOCH₃ | 5-Cl | 6-Cl |
| I.1.394 | phenyl | 2-COOCH₃ | 5-Cl | 6-F |
| I.1.395 | phenyl | 2-COOCH₃ | 5-Cl | 6-CH₃ |
| I.1.396 | phenyl | 2-COOCH₃ | 5-Cl | 6-vinyl |
| I.1.397 | phenyl | 2-COOCH₃ | 5-F | 6-Cl |
| I.1.398 | phenyl | 2-COOCH₃ | 5-F | 6-F |
| I.1.399 | phenyl | 2-COOCH₃ | 5-F | 6-CH₃ |
| I.1.400 | phenyl | 2-COOCH₃ | 5-F | 6-vinyl |
| I.1.401 | phenyl | 2-COOCH₃ | 5-CH₃ | 6-Cl |
| I.1.402 | phenyl | 2-COOCH₃ | 5-CH₃ | 6-F |
| I.1.403 | phenyl | 2-COOCH₃ | 5-CH₃ | 6-CH₃ |
| I.1.404 | phenyl | 2-COOCH₃ | 5-CH₃ | 6-vinyl |
| I.1.405 | phenyl | 2-COOCH₃ | 5-vinyl | 6-Cl |
| I.1.406 | phenyl | 2-COOCH₃ | 5-vinyl | 6-F |
| I.1.407 | phenyl | 2-COOCH₃ | 5-vinyl | 6-CH₃ |
| I.1.408 | phenyl | 2-COOCH₃ | 5-vinyl | 6-vinyl |
| I.1.409 | phenyl | 2-COOH | H | H |
| I.1.410 | phenyl | 2-COOH | 3-Cl | H |
| I.1.411 | phenyl | 2-COOH | 4-Cl | H |
| I.1.412 | phenyl | 2-COOH | 5-Cl | H |
| I.1.413 | phenyl | 2-COOH | 6-Cl | H |
| I.1.414 | phenyl | 2-COOH | 3-F | H |
| I.1.415 | phenyl | 2-COOH | 4-F | H |
| I.1.416 | phenyl | 2-COOH | 5-F | H |
| I.1.417 | phenyl | 2-COOH | 6-F | H |
| I.1.418 | phenyl | 2-COOH | 3-CH₃ | H |
| I.1.419 | phenyl | 2-COOH | 4-CH₃ | H |
| I.1.420 | phenyl | 2-COOH | 5-CH₃ | H |
| I.1.421 | phenyl | 2-COOH | 6-CH₃ | H |
| I.1.422 | phenyl | 2-COOH | 5-vinyl | H |
| I.1.423 | phenyl | 2-COOH | 6-vinyl | H |
| I.1.424 | phenyl | 2-COOH | 5-CF₃ | 3-Cl |
| I.1.425 | phenyl | 2-COOH | 5-CF₃ | 4-Cl |
| I.1.426 | phenyl | 2-COOH | 4-CF₃ | 5-Cl |
| I.1.427 | phenyl | 2-COOH | 5-CF₃ | 6-Cl |
| I.1.428 | phenyl | 2-COOH | 5-CF₃ | 3-F |
| I.1.429 | phenyl | 2-COOH | 5-CF₃ | 4-F |
| I.1.430 | phenyl | 2-COOH | 4-CF₃ | 5-F |
| I.1.431 | phenyl | 2-COOH | 5-CF₃ | 6-F |
| I.1.432 | phenyl | 2-COOH | 5-CF₃ | 3-CH₃ |
| I.1.433 | phenyl | 2-COOH | 5-CF₃ | 4-CH₃ |
| I.1.434 | phenyl | 2-COOH | 4-CF₃ | 5-CH₃ |
| I.1.435 | phenyl | 2-COOH | 5-CF₃ | 6-CH₃ |
| I.1.436 | phenyl | 2-COOH | 4-CF₃ | 5-vinyl |
| I.1.437 | phenyl | 2-COOH | 4-CF₃ | 6-vinyl |
| I.1.438 | phenyl | 2-COOH | 3-Cl | 4-F |
| I.1.439 | phenyl | 2-COOH | 3-Cl | 5-F |
| I.1.440 | phenyl | 2-COOH | 3-Cl | 6-F |
| I.1.441 | phenyl | 2-COOH | 3-Cl | 4-Cl |
| I.1.442 | phenyl | 2-COOH | 3-Cl | 5-Cl |
| I.1.443 | phenyl | 2-COOH | 3-Cl | 6-Cl |
| I.1.444 | phenyl | 2-COOH | 3-Cl | 4-CH₃ |
| I.1.445 | phenyl | 2-COOH | 3-Cl | 5-CH₃ |
| I.1.446 | phenyl | 2-COOH | 3-Cl | 6-CH₃ |
| I.1.447 | phenyl | 2-COOH | 3-Cl | 5-vinyl |
| I.1.448 | phenyl | 2-COOH | 3-Cl | 6-vinyl |
| I.1.449 | phenyl | 2-COOH | 3-F | 4-F |
| I.1.450 | phenyl | 2-COOH | 3-F | 5-F |
| I.1.451 | phenyl | 2-COOH | 3-F | 6-F |
| I.1.452 | phenyl | 2-COOH | 3-F | 4-Cl |
| I.1.453 | phenyl | 2-COOH | 3-F | 5-Cl |
| I.1.454 | phenyl | 2-COOH | 3-F | 6-Cl |
| I.1.455 | phenyl | 2-COOH | 3-F | 4-CH₃ |
| I.1.456 | phenyl | 2-COOH | 3-F | 5-CH₃ |
| I.1.457 | phenyl | 2-COOH | 3-F | 6-CH₃ |
| I.1.458 | phenyl | 2-COOH | 3-F | 5-vinyl |
| I.1.459 | phenyl | 2-COOH | 3-F | 6-vinyl |
| I.1.460 | phenyl | 2-COOH | 3-CH₃ | 4-F |
| I.1.461 | phenyl | 2-COOH | 3-CH₃ | 5-F |
| I.1.462 | phenyl | 2-COOH | 3-CH₃ | 6-F |
| I.1.463 | phenyl | 2-COOH | 3-CH₃ | 4-Cl |
| I.1.464 | phenyl | 2-COOH | 3-CH₃ | 5-Cl |
| I.1.465 | phenyl | 2-COOH | 3-CH₃ | 6-Cl |
| I.1.466 | phenyl | 2-COOH | 3-CH₃ | 4-CH₃ |
| I.1.467 | phenyl | 2-COOH | 3-CH₃ | 5-CH₃ |
| I.1.468 | phenyl | 2-COOH | 3-CH₃ | 6-CH₃ |
| I.1.469 | phenyl | 2-COOH | 3-CH₃ | 5-vinyl |
| I.1.470 | phenyl | 2-COOH | 3-CH₃ | 6-vinyl |
| I.1.471 | phenyl | 2-COOH | 4-Cl | 5-F |
| I.1.472 | phenyl | 2-COOH | 4-Cl | 6-F |
| I.1.473 | phenyl | 2-COOH | 4-Cl | 5-Cl |
| I.1.474 | phenyl | 2-COOH | 4-Cl | 6-Cl |
| I.1.475 | phenyl | 2-COOH | 4-Cl | 5-CH₃ |
| I.1.476 | phenyl | 2-COOH | 4-Cl | 6-CH₃ |
| I.1.477 | phenyl | 2-COOH | 4-Cl | 5-vinyl |
| I.1.478 | phenyl | 2-COOH | 4-Cl | 6-vinyl |
| I.1.479 | phenyl | 2-COOH | 4-F | 5-F |
| I.1.480 | phenyl | 2-COOH | 4-F | 6-F |
| I.1.481 | phenyl | 2-COOH | 4-F | 5-Cl |
| I.1.482 | phenyl | 2-COOH | 4-F | 6-Cl |
| I.1.483 | phenyl | 2-COOH | 4-F | 5-CH₃ |
| I.1.484 | phenyl | 2-COOH | 4-F | 6-CH₃ |
| I.1.485 | phenyl | 2-COOH | 4-F | 5-vinyl |
| I.1.486 | phenyl | 2-COOH | 4-F | 6-vinyl |
| I.1.487 | phenyl | 2-COOH | 4-CH₃ | 5-F |
| I.1.488 | phenyl | 2-COOH | 4-CH₃ | 6-F |
| I.1.489 | phenyl | 2-COOH | 4-CH₃ | 5-Cl |
| I.1.490 | phenyl | 2-COOH | 4-CH₃ | 6-Cl |
| I.1.491 | phenyl | 2-COOH | 4-CH₃ | 5-CH₃ |
| I.1.492 | phenyl | 2-COOH | 4-CH₃ | 6-CH₃ |
| I.1.493 | phenyl | 2-COOH | 4-CH₃ | 5-vinyl |
| I.1.494 | phenyl | 2-COOH | 4-CH₃ | 6-vinyl |
| I.1.495 | phenyl | 2-COOH | 5-Cl | 6-Cl |
| I.1.496 | phenyl | 2-COOH | 5-Cl | 6-F |
| I.1.497 | phenyl | 2-COOH | 5-Cl | 6-CH₃ |
| I.1.498 | phenyl | 2-COOH | 5-Cl | 6-vinyl |
| I.1.499 | phenyl | 2-COOH | 5-F | 6-Cl |
| I.1.500 | phenyl | 2-COOH | 5-F | 6-F |
| I.1.501 | phenyl | 2-COOH | 5-F | 6-CH₃ |
| I.1.502 | phenyl | 2-COOH | 5-F | 6-vinyl |
| I.1.503 | phenyl | 2-COOH | 5-CH₃ | 6-Cl |
| I.1.504 | phenyl | 2-COOH | 5-CH₃ | 6-F |
| I.1.505 | phenyl | 2-COOH | 5-CH₃ | 6-CH₃ |
| I.1.506 | phenyl | 2-COOH | 5-CH₃ | 6-vinyl |
| I.1.507 | phenyl | 2-COOH | 5-vinyl | 6-Cl |
| I.1.508 | phenyl | 2-COOH | 5-vinyl | 6-F |
| I.1.509 | phenyl | 2-COOH | 5-vinyl | 6-CH₃ |
| I.1.510 | phenyl | 2-COOH | 5-vinyl | 6-vinyl |
| I.1.511 | phenyl | 2-CON(CH₃)₂ | H | H |
| I.1.512 | phenyl | 2-CON(CH₃)₂ | 3-Cl | H |
| I.1.513 | phenyl | 2-CON(CH₃)₂ | 4-Cl | H |
| I.1.514 | phenyl | 2-CON(CH₃)₂ | 5-Cl | H |
| I.1.515 | phenyl | 2-CON(CH₃)₂ | 6-Cl | H |
| I.1.516 | phenyl | 2-CON(CH₃)₂ | 3-F | H |
| I.1.517 | phenyl | 2-CON(CH₃)₂ | 4-F | H |
| I.1.518 | phenyl | 2-CON(CH₃)₂ | 5-F | H |
| I.1.519 | phenyl | 2-CON(CH₃)₂ | 6-F | H |
| I.1.520 | phenyl | 2-CON(CH₃)₂ | 3-CH₃ | H |
| I.1.521 | phenyl | 2-CON(CH₃)₂ | 4-CH₃ | H |
| I.1.522 | phenyl | 2-CON(CH₃)₂ | 5-CH₃ | H |
| I.1.523 | phenyl | 2-CON(CH₃)₂ | 6-CH₃ | H |
| I.1.524 | phenyl | 2-CON(CH₃)₂ | 5-vinyl | H |
| I.1.525 | phenyl | 2-CON(CH₃)₂ | 6-vinyl | H |
| I.1.526 | phenyl | 2-CON(CH₃)₂ | 5-CF₃ | 3-Cl |
| I.1.527 | phenyl | 2-CON(CH₃)₂ | 5-CF₃ | 4-Cl |
| I.1.528 | phenyl | 2-CON(CH₃)₂ | 4-CF₃ | 5-Cl |
| I.1.529 | phenyl | 2-CON(CH₃)₂ | 5-CF₃ | 6-Cl |
| I.1.530 | phenyl | 2-CON(CH₃)₂ | 5-CF₃ | 3-F |
| I.1.531 | phenyl | 2-CON(CH₃)₂ | 5-CF₃ | 4-F |
| I.1.532 | phenyl | 2-CON(CH₃)₂ | 4-CF₃ | 5-F |
| I.1.533 | phenyl | 2-CON(CH₃)₂ | 5-CF₃ | 6-F |
| I.1.534 | phenyl | 2-CON(CH₃)₂ | 5-CF₃ | 3-CH₃ |
| I.1.535 | phenyl | 2-CON(CH₃)₂ | 5-CF₃ | 4-CH₃ |
| I.1.536 | phenyl | 2-CON(CH₃)₂ | 4-CF₃ | 5-CH₃ |
| I.1.537 | phenyl | 2-CON(CH₃)₂ | 5-CF₃ | 6-CH₃ |
| I.1.538 | phenyl | 2-CON(CH₃)₂ | 4-CF₃ | 5-vinyl |
| I.1.539 | phenyl | 2-CON(CH₃)₂ | 4-CF₃ | 6-vinyl |
| I.1.540 | phenyl | 2-CON(CH₃)₂ | 3-Cl | 4-F |
| I.1.541 | phenyl | 2-CON(CH₃)₂ | 3-Cl | 5-F |
| I.1.542 | phenyl | 2-CON(CH₃)₂ | 3-Cl | 6-F |
| I.1.543 | phenyl | 2-CON(CH₃)₂ | 3-Cl | 4-Cl |
| I.1.544 | phenyl | 2-CON(CH₃)₂ | 3-Cl | 5-Cl |
| I.1.545 | phenyl | 2-CON(CH₃)₂ | 3-Cl | 6-Cl |
| I.1.546 | phenyl | 2-CON(CH₃)₂ | 3-Cl | 4-CH₃ |
| I.1.547 | phenyl | 2-CON(CH₃)₂ | 3-Cl | 5-CH₃ |
| I.1.548 | phenyl | 2-CON(CH₃)₂ | 3-Cl | 6-CH₃ |
| I.1.549 | phenyl | 2-CON(CH₃)₂ | 3-Cl | 5-vinyl |
| I.1.550 | phenyl | 2-CON(CH₃)₂ | 3-Cl | 6-vinyl |
| I.1.551 | phenyl | 2-CON(CH₃)₂ | 3-F | 4-F |
| I.1.552 | phenyl | 2-CON(CH₃)₂ | 3-F | 5-F |
| I.1 553 | phenyl | 2-CON(CH₃)₂ | 3-F | 6-F |
| I.1.554 | phenyl | 2-CON(CH₃)₂ | 3-F | 4-Cl |
| I.1.555 | phenyl | 2-CON(CH₃)₂ | 3-F | 5-Cl |
| I.1.556 | phenyl | 2-CON(CH₃)₂ | 3-F | 6-Cl |
| I.1.557 | phenyl | 2-CON(CH₃)₂ | 3-F | 4-CH₃ |
| I.1.558 | phenyl | 2-CON(CH₃)₂ | 3-F | 5-CH₃ |
| I.1.559 | phenyl | 2-CON(CH₃)₂ | 3-F | 6-CH₃ |
| I.1.560 | phenyl | 2-CON(CH₃)₂ | 3-F | 5-vinyl |
| I.1.561 | phenyl | 2-CON(CH₃)₂ | 3-F | 6-vinyl |
| I.1.562 | phenyl | 2-CON(CH₃)₂ | 3-CH₃ | 4-F |
| I.1.563 | phenyl | 2-CON(CH₃)₂ | 3-CH₃ | 5-F |
| I.1.564 | phenyl | 2-CON(CH₃)₂ | 3-CH₃ | 6-F |
| I.1.565 | phenyl | 2-CON(CH₃)₂ | 3-CH₃ | 4-Cl |
| I.1.566 | phenyl | 2-CON(CH₃)₂ | 3-CH₃ | 5-Cl |
| I.1.557 | phenyl | 2-CON(CH₃)₂ | 3-CH₃ | 6-Cl |
| I.1.568 | phenyl | 2-CON(CH₃)₂ | 3-CH₃ | 4-CH₃ |
| I.1.569 | phenyl | 2-CON(CH₃)₂ | 3-CH₃ | 5-CH₃ |
| I.1.570 | phenyl | 2-CON(CH₃)₂ | 3-CH₃ | 6-CH₃ |
| I.1.571 | phenyl | 2-CON(CH₃)₂ | 3-CH₃ | 5-vinyl |
| I.1.572 | phenyl | 2-CON(CH₃)₂ | 3-CH₃ | 6-vinyl |
| I.1.573 | phenyl | 2-CON(CH₃)₂ | 4-Cl | 5-F |
| I.1.574 | phenyl | 2-CON(CH₃)₂ | 4-Cl | 6-F |
| I.1.575 | phenyl | 2-CON(CH₃)₂ | 4-Cl | 5-Cl |
| I.1.576 | phenyl | 2-CON(CH₃)₂ | 4-Cl | 6-Cl |
| I.1.577 | phenyl | 2-CON(CH₃)₂ | 4-Cl | 5-CH₃ |
| I.1.578 | phenyl | 2-CON(CH₃)₂ | 4-Cl | 6-CH₃ |
| I.1.579 | phenyl | 2-CON(CH₃)₂ | 4-Cl | 5-vinyl |
| I.1.580 | phenyl | 2-CON(CH₃)₂ | 4-Cl | 6-vinyl |
| I.1.581 | phenyl | 2-CON(CH₃)₂ | 4-F | 5-F |
| I.1.582 | phenyl | 2-CON(CH₃)₂ | 4-F | 6-F |
| I.1.583 | phenyl | 2-CON(CH₃)₂ | 4-F | 5-Cl |
| I.1.584 | phenyl | 2-CON(CH₃)₂ | 4-F | 6-Cl |
| I.1.585 | phenyl | 2-CON(CH₃)₂ | 4-F | 5-CH₃ |
| I.1.586 | phenyl | 2-CON(CH₃)₂ | 4-F | 6-CH₃ |
| I.1.587 | phenyl | 2-CON(CH₃)₂ | 4-F | 5-vinyl |
| I.1.588 | phenyl | 2-CON(CH₃)₂ | 4-F | 6-vinyl |
| I.1.589 | phenyl | 2-CON(CH₃)₂ | 4-CH₃ | 5-F |
| I.1.590 | phenyl | 2-CON(CH₃)₂ | 4-CH₃ | 6-F |
| I.1.591 | phenyl | 2-CON(CH₃)₂ | 4-CH₃ | 5-Cl |
| I.1.592 | phenyl | 2-CON(CH₃)₂ | 4-CH₃ | 6-Cl |
| I.1.593 | phenyl | 2-CON(CH₃)₂ | 4-CH₃ | 5-CH₃ |
| I.1.594 | phenyl | 2-CON(CH₃)₂ | 4-CH₃ | 6-CH₃ |
| I.1.595 | phenyl | 2-CON(CH₃)₂ | 4-CH₃ | 5-vinyl |
| I.1.596 | phenyl | 2-CON(CH₃)₂ | 4-CH₃ | 6-vinyl |
| I.1.597 | phenyl | 2-CON(CH₃)₂ | 5-Cl | 6-Cl |
| I.1.598 | phenyl | 2-CON(CH₃)₂ | 5-Cl | 6-F |
| I.1.599 | phenyl | 2-CON(CH₃)₂ | 5-Cl | 6-CH₃ |
| I.1.600 | phenyl | 2-CON(CH₃)₂ | 5-Cl | 6-vinyl |
| I.1.601 | phenyl | 2-CON(CH₃)₂ | 5-F | 6-Cl |
| I.1.602 | phenyl | 2-CON(CH₃)₂ | 5-F | 6-F |
| I.1.603 | phenyl | 2-CON(CH₃)₂ | 5-F | 6-CH₃ |
| I.1.604 | phenyl | 2-CON(CH₃)₂ | 5-F | 6-vinyl |
| I.1.605 | phenyl | 2-CON(CH₃)₂ | 5-CH₃ | 6-Cl |
| I.1.606 | phenyl | 2-CON(CH₃)₂ | 5-CH₃ | 6-F |
| I.1.607 | phenyl | 2-CON(CH₃)₂ | 5-CH₃ | 6-CH₃ |
| I.1.608 | phenyl | 2-CON(CH₃)₂ | 5-CH₃ | 6-vinyl |
| I.1.609 | phenyl | 2-CON(CH₃)₂ | 5-vinyl | 6-Cl |
| I.1.610 | phenyl | 2-CON(CH₃)₂ | 5-vinyl | 6-F |
| I.1.611 | phenyl | 2-CON(CH₃)₂ | 5-vinyl | 6-CH₃ |
| I.1.612 | phenyl | 2-CON(CH₃)₂ | 5-vinyl | 6-vinyl |
| I.1.613 | phenyl | 2-I | H | H |
| I.1.614 | phenyl | 2-I | 3-Cl | H |
| I.1.615 | phenyl | 2-I | 4-Cl | H |
| I.1.616 | phenyl | 2-I | 5-Cl | H |
| I.1.617 | phenyl | 2-I | 6-Cl | H |
| I.1.618 | phenyl | 2-I | 3-F | H |
| I.1.619 | phenyl | 2-I | 4-F | H |
| I.1.620 | phenyl | 2-I | 5-F | H |
| I.1.621 | phenyl | 2-I | 6-F | H |
| I.1.622 | phenyl | 2-I | 3-CH₃ | H |
| I.1.623 | phenyl | 2-I | 4-CH₃ | H |
| I.1.624 | phenyl | 2-I | 5-CH₃ | H |
| I.1.625 | phenyl | 2-I | 6-CH₃ | H |
| I.1.626 | phenyl | 2-I | 5-vinyl | H |
| I.1.627 | phenyl | 2-I | 6-vinyl | H |
| I.1.628 | phenyl | 2-I | 5-CF₃ | 3-Cl |
| I.1.629 | phenyl | 2-I | 5-CF₃ | 4-Cl |
| I.1.630 | phenyl | 2-I | 4-CF₃ | 5-Cl |
| I.1.631 | phenyl | 2-I | 5-CF₃ | 6-Cl |
| I.1.632 | phenyl | 2-I | 5-CF₃ | 3-F |
| I.1.633 | phenyl | 2-I | 5-CF₃ | 4-F |
| I.1.634 | phenyl | 2-I | 4-CF₃ | 5-F |
| I.1.635 | phenyl | 2-I | 5-CF₃ | 6-F |
| I.1.636 | phenyl | 2-I | 5-CF₃ | 3-CH₃ |
| I.1.637 | phenyl | 2-I | 5-CF₃ | 4-CH₃ |
| I.1.638 | phenyl | 2-I | 4-CF₃ | 5-CH₃ |
| I.1.639 | phenyl | 2-I | 5-CF₃ | 6-CH₃ |
| I.1.640 | phenyl | 2-I | 4-CF₃ | 5-vinyl |
| I.1.641 | phenyl | 2-I | 4-CF₃ | 6-vinyl |
| I.1.642 | phenyl | 2-I | 3-Cl | 4-F |
| I.1.643 | phenyl | 2-I | 3-Cl | 5-F |
| I.1.644 | phenyl | 2-I | 3-Cl | 6-F |
| I.1.645 | phenyl | 2-I | 3-Cl | 4-Cl |
| I.1.646 | phenyl | 2-I | 3-Cl | 5-Cl |
| I.1.647 | phenyl | 2-I | 3-Cl | 6-Cl |
| I.1.648 | phenyl | 2-I | 3-Cl | 4-CH₃ |
| I.1.649 | phenyl | 2-I | 3-Cl | 5-CH₃ |
| I.1.650 | phenyl | 2-I | 3-Cl | 6-CH₃ |
| I.1.651 | phenyl | 2-I | 3-Cl | 5-vinyl |
| I.1.652 | phenyl | 2-I | 3-Cl | 6-vinyl |
| I.1.653 | phenyl | 2-I | 3-F | 4-F |
| I.1.654 | phenyl | 2-I | 3-F | 5-F |
| I.1.655 | phenyl | 2-I | 3-F | 6-F |
| I.1.656 | phenyl | 2-I | 3-F | 4-Cl |
| I.1.657 | phenyl | 2-I | 3-F | 5-Cl |
| I.1.658 | phenyl | 2-I | 3-F | 6-Cl |
| I.1.659 | phenyl | 2-I | 3-F | 4-CH₃ |
| I.1.660 | phenyl | 2-I | 3-F | 5-CH₃ |
| I.1.661 | phenyl | 2-I | 3-F | 6-CH₃ |
| I.1.662 | phenyl | 2-I | 3-F | 5-vinyl |
| I.1.663 | phenyl | 2-I | 3-F | 6-vinyl |
| I.1.664 | phenyl | 2-I | 3-CH₃ | 4-F |
| I.1.665 | phenyl | 2-I | 3-CH₃ | 5-F |
| I.1.666 | phenyl | 2-I | 3-CH₃ | 6-F |
| I.1.667 | phenyl | 2-I | 3-CH₃ | 4-Cl |
| I.1.668 | phenyl | 2-I | 3-CH₃ | 5-Cl |
| I.1.669 | phenyl | 2-I | 3-CH₃ | 6-Cl |
| I.1.670 | phenyl | 2-I | 3-CH₃ | 4-CH₃ |
| I.1.671 | phenyl | 2-I | 3-CH₃ | 5-CH₃ |
| I.1.672 | phenyl | 2-I | 3-CH₃ | 6-CH₃ |
| I.1.673 | phenyl | 2-I | 3-CH₃ | 5-vinyl |
| I.1.674 | phenyl | 2-I | 3-CH₃ | 6-vinyl |
| I.1-675 | phenyl | 2-I | 4-Cl | 5-F |
| I.1.676 | phenyl | 2-I | 4-Cl | 6-F |
| I.1.677 | phenyl | 2-I | 4-Cl | 5-Cl |
| I.1.678 | phenyl | 2-I | 4-Cl | 6-Cl |
| I.1.679 | phenyl | 2-I | 4-Cl | 5-CH₃ |
| I.1.680 | phenyl | 2-I | 4-Cl | 6-CH₃ |
| I.1.681 | phenyl | 2-I | 4-Cl | 5-vinyl |
| I.1.682 | phenyl | 2-I | 4-Cl | 6-vinyl |
| I.1.683 | phenyl | 2-I | 4-F | 5-F |
| I.1.684 | phenyl | 2-I | 4-F | 6-F |
| I.1.685 | phenyl | 2-I | 4-F | 5-Cl |
| I.1.686 | phenyl | 2-I | 4-F | 6-Cl |
| I.1.687 | phenyl | 2-I | 4-F | 5-CH₃ |
| I.1.688 | phenyl | 2-I | 4-F | 6-CH₃ |
| I.1.689 | phenyl | 2-I | 4-F | 5-vinyl |
| I.1.690 | phenyl | 2-I | 4-F | 6-vinyl |
| I.1.691 | phenyl | 2-I | 4-CH₃ | 5-F |
| I.1.692 | phenyl | 2-I | 4-CH₃ | 6-F |
| I.1.693 | phenyl | 2-I | 4-CH₃ | 5-Cl |
| I.1.694 | phenyl | 2-I | 4-CH₃ | 6-Cl |
| I.1.695 | phenyl | 2-I | 4-CH₃ | 5-CH₃ |
| I.1.696 | phenyl | 2-I | 4-CH₃ | 6-CH₃ |
| I.1.697 | phenyl | 2-I | 4-CH₃ | 5-vinyl |
| I.1.698 | phenyl | 2-I | 4-CH₃ | 6-vinyl |
| I.1.699 | phenyl | 2-I | 5-Cl | 6-Cl |
| I.1.700 | phenyl | 2-I | 5-Cl | 6-F |
| I.1.701 | phenyl | 2-I | 5-Cl | 6-CH₃ |
| I.1.702 | phenyl | 2-I | 5-Cl | 6-vinyl |
| I.1.703 | phenyl | 2-I | 5-F | 6-Cl |
| I.1.704 | phenyl | 2-I | 5-F | 6-F |
| I.1.705 | phenyl | 2-I | 5-F | 6-CH₃ |
| I.1.706 | phenyl | 2-I | 5-F | 6-vinyl |
| I.1.707 | phenyl | 2-I | 5-CH₃ | 6-Cl |
| I.1.708 | phenyl | 2-I | 5-CH₃ | 6-F |
| I.1.709 | phenyl | 2-I | 5-CH₃ | 6-CH₃ |
| I.1.710 | phenyl | 2-I | 5-CH₃ | 6-vinyl |
| I.1.711 | phenyl | 2-I | 5-vinyl | 6-Cl |
| I.1.712 | phenyl | 2-I | 5-vinyl | 6-F |
| I.1.713 | phenyl | 2-I | 5-vinyl | 6-CH₃ |
| I.1.714 | phenyl | 2-I | 5-vinyl | 6-vinyl |
| I.1.715 | phenyl | 2-Br | H | H |
| I.1.716 | phenyl | 2-Br | 3-Cl | H |
| I.1.717 | phenyl | 2-Br | 4-Cl | H |
| I.1.718 | phenyl | 2-Br | 5-Cl | H |
| I.1.719 | phenyl | 2-Br | 6-Cl | H |
| I.1.720 | phenyl | 2-Br | 3-F | H |
| I.1.721 | phenyl | 2-Br | 4-F | H |
| I.1.722 | phenyl | 2-Br | 5-F | H |
| I.1.723 | phenyl | 2-Br | 6-F | H |
| I.1.724 | phenyl | 2-Br | 3-CH₃ | H |
| I.1.725 | phenyl | 2-Br | 4-CH₃ | H |
| I.1.726 | phenyl | 2-Br | 5-CH₃ | H |
| I.1.727 | phenyl | 2-Br | 6-CH₃ | H |
| I.1.728 | phenyl | 2-Br | 5-vinyl | H |
| I.1.729 | phenyl | 2-Br | 6-vinyl | H |
| I.1.730 | phenyl | 2-Br | 5-CF₃ | 3-Cl |
| I.1.731 | phenyl | 2-Br | 5-CF₃ | 4-Cl |
| I.1.732 | phenyl | 2-Br | 4-CF₃ | 5-Cl |
| I.1.733 | phenyl | 2-Br | 5-CF₃ | 6-Cl |
| I.1.734 | phenyl | 2-Br | 5-CF₃ | 3-F |
| I.1.735 | phenyl | 2-Br | 5-CF₃ | 4-F |
| I.1.736 | phenyl | 2-Br | 4-CF₃ | 5-F |
| I.1.737 | phenyl | 2-Br | 5-CF₃ | 6-F |
| I.1.738 | phenyl | 2-Br | 5-CF₃ | 3-CH₃ |
| I.1.739 | phenyl | 2-Br | 5-CF₃ | 4-CH₃ |
| I.1.740 | phenyl | 2-Br | 4-CF₃ | 5-CH₃ |
| I.1.741 | phenyl | 2-Br | 5-CF₃ | 6-CH₃ |
| I.1.742 | phenyl | 2-Br | 4-CF₃ | 5-vinyl |
| I.1.743 | phenyl | 2-Br | 4-CF₃ | 6-vinyl |
| I.1.744 | phenyl | 2-Br | 3-Cl | 4-F |
| I.1.745 | phenyl | 2-Br | 3-Cl | 5-F |
| I.1.746 | phenyl | 2-Br | 3-Cl | 6-F |
| I.1.747 | phenyl | 2-Br | 3-Cl | 4-Cl |
| I.1.748 | phenyl | 2-Br | 3-Cl | 5-Cl |
| I.1.749 | phenyl | 2-Br | 3-Cl | 6-Cl |
| I.1.750 | phenyl | 2-Br | 3-Cl | 4-CH₃ |
| I.1.751 | phenyl | 2-Br | 3-Cl | 5-CH₃ |
| I.1.752 | phenyl | 2-Br | 3-Cl | 6-CH₃ |
| I.1.753 | phenyl | 2-Br | 3-Cl | 5-vinyl |
| I.1.754 | phenyl | 2-Br | 3-Cl | 6-vinyl |
| I.1.755 | phenyl | 2-Br | 3-F | 4-F |
| I.1.756 | phenyl | 2-Br | 3-F | 5-F |
| I.1.757 | phenyl | 2-Br | 3-F | 6-F |
| I.1.758 | phenyl | 2-Br | 3-F | 4-Cl |
| I.1.759 | phenyl | 2-Br | 3-F | 5-Cl |
| I.1.760 | phenyl | 2-Br | 3-F | 6-Cl |
| I.1.761 | phenyl | 2-Br | 3-F | 4-CH₃ |
| I.1.762 | phenyl | 2-Br | 3-F | 5-CH₃ |
| I.1.763 | phenyl | 2-Br | 3-F | 6-CH₃ |
| I.1.764 | phenyl | 2-Br | 3-F | 5-vinyl |
| I.1.765 | phenyl | 2-Br | 3-F | 6-vinyl |
| I.1.766 | phenyl | 2-Br | 3-CH₃ | 4-F |
| I.1.767 | phenyl | 2-Br | 3-CH₃ | 5-F |
| I.1.768 | phenyl | 2-Br | 3-CH₃ | 6-F |
| I.1.769 | phenyl | 2-Br | 3-CH₃ | 4-Cl |
| I.1.770 | phenyl | 2-Br | 3-CH₃ | 5-Cl |
| I.1.771 | phenyl | 2-Br | 3-CH₃ | 6-Cl |
| I.1.772 | phenyl | 2-Br | 3-CH₃ | 4-CH₃ |
| I.1.773 | phenyl | 2-Br | 3-CH₃ | 5-CH₃ |
| I.1.774 | phenyl | 2-Br | 3-CH₃ | 6-CH₃ |
| I.1.775 | phenyl | 2-Br | 3-CH₃ | 5-vinyl |
| I.1.776 | phenyl | 2-Br | 3-CH₃ | 6-vinyl |
| I.1.777 | phenyl | 2-Br | 4-Cl | 5-F |
| I.1.778 | phenyl | 2-Br | 4-Cl | 6-F |
| I.1.779 | phenyl | 2-Br | 4-Cl | 5-Cl |
| I.1.780 | phenyl | 2-Br | 4-Cl | 6-Cl |
| I.1.781 | phenyl | 2-Br | 4-Cl | 5-CH₃ |
| I.1.782 | phenyl | 2-Br | 4-Cl | 6-CH₃ |
| I.1.783 | phenyl | 2-Br | 4-Cl | 5-vinyl |
| I.1.784 | phenyl | 2-Br | 4-Cl | 6-vinyl |
| I.1.785 | phenyl | 2-Br | 4-F | 5-F |
| I.1.786 | phenyl | 2-Br | 4-F | 6-F |
| I.1.787 | phenyl | 2-Br | 4-F | 5-Cl |
| I.1.788 | phenyl | 2-Br | 4-F | 6-Cl |
| I.1.789 | phenyl | 2-Br | 4-F | 5-CH₃ |
| I.1.790 | phenyl | 2-Br | 4-F | 6-CH₃ |
| I.1.791 | phenyl | 2-Br | 4-F | 5-vinyl |
| I.1.792 | phenyl | 2-Br | 4-F | 6-vinyl |
| I.1.793 | phenyl | 2-Br | 4-CH₃ | 5-F |
| I.1.794 | phenyl | 2-Br | 4-CH₃ | 6-F |
| I.1.795 | phenyl | 2-Br | 4-CH₃ | 5-Cl |
| I.1.796 | phenyl | 2-Br | 4-CH₃ | 6-Cl |
| I.1.797 | phenyl | 2-Br | 4-CH₃ | 5-CH₃ |
| I.1.798 | phenyl | 2-Br | 4-CH₃ | 6-CH₃ |
| I.1.799 | phenyl | 2-Br | 4-CH₃ | 5-vinyl |
| I.1.800 | phenyl | 2-Br | 4-CH₃ | 6-vinyl |
| I.1.801 | phenyl | 2-Br | 5-Cl | 6-Cl |
| I.1.802 | phenyl | 2-Br | 5-Cl | 6-F |
| I.1.803 | phenyl | 2-Br | 5-Cl | 6-CH₃ |
| I.1.804 | phenyl | 2-Br | 5-Cl | 6-vinyl |
| I.1.805 | phenyl | 2-Br | 5-F | 6-Cl |
| I.1.806 | phenyl | 2-Br | 5-F | 6-F |
| I.1.807 | phenyl | 2-Br | 5-F | 6-CH₃ |
| I.1.808 | phenyl | 2-Br | 5-F | 6-vinyl |
| I.1.809 | phenyl | 2-Br | 5-CH₃ | 6-Cl |
| I.1.810 | phenyl | 2-Br | 5-CH₃ | 6-F |
| I.1.811 | phenyl | 2-Br | 5-CH₃ | 6-CH₃ |
| I.1.812 | phenyl | 2-Br | 5-CH₃ | 6-vinyl |
| I.1.813 | phenyl | 2-Br | 5-vinyl | 6-Cl |
| I.1.814 | phenyl | 2-Br | 5-vinyl | 6-F |
| I.1.815 | phenyl | 2-Br | 5-vinyl | 6-CH₃ |
| I.1.816 | phenyl | 2-Br | 5-vinyl | 6-vinyl |
| I.1.817 | phenyl | 2-Cl | H | H |
| I.1.818 | phenyl | 2-Cl | 3-Cl | H |
| I.1.819 | phenyl | 2-Cl | 4-Cl | H |
| I.1.820 | phenyl | 2-Cl | 5-Cl | H |
| I.1.821 | phenyl | 2-Cl | 6-Cl | H |
| I.1.822 | phenyl | 2-Cl | 3-F | H |
| I.1.823 | phenyl | 2-Cl | 4-F | H |
| I.1.824 | phenyl | 2-Cl | 5-F | H |
| I.1.825 | phenyl | 2-Cl | 6-F | H |
| I.1.826 | phenyl | 2-Cl | 3-CH₃ | H |
| I.1.827 | phenyl | 2-Cl | 4-CH₃ | H |
| I.1.828 | phenyl | 2-Cl | 5-CH₃ | H |
| I.1.829 | phenyl | 2-Cl | 6-CH₃ | H |
| I.1.830 | phenyl | 2-Cl | 5-vinyl | H |
| I.1.831 | phenyl | 2-Cl | 6-vinyl | H |
| I.1.832 | phenyl | 2-Cl | 5-CF₃ | 3-Cl |
| I.1.833 | phenyl | 2-Cl | 5-CF₃ | 4-Cl |
| I.1.834 | phenyl | 2-Cl | 4-CF₃ | 5-Cl |
| I.1.835 | phenyl | 2-Cl | 5-CF₃ | 6-Cl |
| I.1.836 | phenyl | 2-Cl | 5-CF₃ | 3-F |
| I.1.837 | phenyl | 2-Cl | 5-CF₃ | 4-F |
| I.1.838 | phenyl | 2-Cl | 4-CF₃ | 5-F |
| I.1.839 | phenyl | 2-Cl | 5-CF₃ | 6-F |
| I.1.840 | phenyl | 2-Cl | 5-CF₃ | 3-CH₃ |
| I.1.841 | phenyl | 2-Cl | 5-CF₃ | 4-CH₃ |
| I.1.842 | phenyl | 2-Cl | 4-CF₃ | 5-CH₃ |
| I.1.843 | phenyl | 2-Cl | 5-CF₃ | 6-CH₃ |
| I.1.844 | phenyl | 2-Cl | 4-CF₃ | 5-vinyl |
| I.1.845 | phenyl | 2-Cl | 4-CF₃ | 6-vinyl |
| I.1.846 | phenyl | 2-Cl | 3-Cl | 4-F |
| I.1.847 | phenyl | 2-Cl | 3-Cl | 5-F |
| I.1.848 | phenyl | 2-Cl | 3-Cl | 6-F |
| I.1.849 | phenyl | 2-Cl | 3-Cl | 4-Cl |
| I.1.850 | phenyl | 2-Cl | 3-Cl | 5-Cl |
| I.1.851 | phenyl | 2-Cl | 3-Cl | 6-Cl |
| I.1.852 | phenyl | 2-Cl | 3-Cl | 4-CH₃ |
| I.1.853 | phenyl | 2-Cl | 3-Cl | 5-CH₃ |
| I.1.854 | phenyl | 2-Cl | 3-Cl | 6-CH₃ |
| I.1.855 | phenyl | 2-Cl | 3-Cl | 5-vinyl |
| I.1.856 | phenyl | 2-Cl | 3-Cl | 6-vinyl |
| I.1.857 | phenyl | 2-Cl | 3-F | 4-F |
| I.1.858 | phenyl | 2-Cl | 3-F | 5-F |
| I.1.859 | phenyl | 2-Cl | 3-F | 6-F |
| I.1.860 | phenyl | 2-Cl | 3-F | 4-Cl |
| I.1.861 | phenyl | 2-Cl | 3-F | 5-Cl |
| I.1.862 | phenyl | 2-Cl | 3-F | 6-Cl |
| I.1.863 | phenyl | 2-Cl | 3-F | 4-CH₃ |
| I.1.864 | phenyl | 2-Cl | 3-F | 5-CH₃ |
| I.1.865 | phenyl | 2-Cl | 3-F | 6-CH₃ |
| I.1.866 | phenyl | 2-Cl | 3-F | 5-vinyl |
| I.1.867 | phenyl | 2-Cl | 3-F | 6-vinyl |
| I.1.868 | phenyl | 2-Cl | 3-CH₃ | 4-F |
| I.1.869 | phenyl | 2-Cl | 3-CH₃ | 5-F |
| I.1.870 | phenyl | 2-Cl | 3-CH₃ | 6-F |
| I.1.871 | phenyl | 2-Cl | 3-CH₃ | 4-Cl |
| I.1.872 | phenyl | 2-Cl | 3-CH₃ | 5-Cl |
| I.1.873 | phenyl | 2-Cl | 3-CH₃ | 6-Cl |
| I.1.874 | phenyl | 2-Cl | 3-CH₃ | 4-CH₃ |
| I.1.875 | phenyl | 2-Cl | 3-CH₃ | 5-CH₃ |
| I.1.876 | phenyl | 2-Cl | 3-CH₃ | 6-CH₃ |
| I.1.877 | phenyl | 2-Cl | 3-CH₃ | 5-vinyl |
| I.1.878 | phenyl | 2-Cl | 3-CH₃ | 6-vinyl |
| I.1.879 | phenyl | 2-Cl | 4-Cl | 5-F |
| I.1.880 | phenyl | 2-Cl | 4-Cl | 6-F |
| I.1.881 | phenyl | 2-Cl | 4-Cl | 5-Cl |
| I.1.882 | phenyl | 2-Cl | 4-Cl | 6-Cl |
| I.1.883 | phenyl | 2-Cl | 4-Cl | 5-CH₃ |
| I.1.884 | phenyl | 2-Cl | 4-Cl | 6-CH₃ |
| I.1.885 | phenyl | 2-Cl | 4-Cl | 5-vinyl |
| I.1.886 | phenyl | 2-Cl | 4-Cl | 6-vinyl |
| I.1.887 | phenyl | 2-Cl | 4-F | 5-F |
| I.1.888 | phenyl | 2-Cl | 4-F | 6-F |
| I.1.889 | phenyl | 2-Cl | 4-F | 5-Cl |
| I.1.890 | phenyl | 2-Cl | 4-F | 6-Cl |
| I.1.891 | phenyl | 2-Cl | 4-F | 5-CH₃ |
| I.1.892 | phenyl | 2-Cl | 4-F | 6-CH₃ |
| I.1.893 | phenyl | 2-Cl | 4-F | 5-vinyl |
| I.1.894 | phenyl | 2-Cl | 4-F | 6-vinyl |
| I.1.895 | phenyl | 2-Cl | 4-CH₃ | 5-F |
| I.1.896 | phenyl | 2-Cl | 4-CH₃ | 6-F |
| I.1.897 | phenyl | 2-Cl | 4-CH₃ | 5-Cl |
| I.1.898 | phenyl | 2-Cl | 4-CH₃ | 6-Cl |
| I.1.899 | phenyl | 2-Cl | 4-CH₃ | 5-CH₃ |
| I.1.900 | phenyl | 2-Cl | 4-CH₃ | 6-CH₃ |
| I.1.901 | phenyl | 2-Cl | 4-CH₃ | 5-vinyl |
| I.1.902 | phenyl | 2-Cl | 4-CH₃ | 6-vinyl |
| I.1.903 | phenyl | 2-Cl | 5-Cl | 6-Cl |
| I.1.904 | phenyl | 2-Cl | 5-Cl | 6-F |
| I.1.905 | phenyl | 2-Cl | 5-Cl | 6-CH₃ |
| I.1.906 | phenyl | 2-Cl | 5-Cl | 6-vinyl |
| I.1.907 | phenyl | 2-Cl | 5-F | 6-Cl |
| I.1.908 | phenyl | 2-Cl | 5-F | 6-F |
| I.1.909 | phenyl | 2-Cl | 5-F | 6-CH₃ |
| I.1.910 | phenyl | 2-Cl | 5-F | 6-vinyl |
| I.1.911 | phenyl | 2-Cl | 5-CH₃ | 6-Cl |
| I.1.912 | phenyl | 2-Cl | 5-CH₃ | 6-F |
| I.1.913 | phenyl | 2-Cl | 5-CH₃ | 6-CH₃ |
| I.1.914 | phenyl | 2-Cl | 5-CH₃ | 6-vinyl |
| I.1.915 | phenyl | 2-Cl | 5-vinyl | 6-Cl |
| I.1.916 | phenyl | 2-Cl | 5-vinyl | 6-F |
| I.1.917 | phenyl | 2-Cl | 5-vinyl | 6-CH₃ |
| I.1.918 | phenyl | 2-Cl | 5-vinyl | 6-vinyl |
| I.1.919 | phenyl | 2-COOCH₂CH₃ | H | H |
| I.1.920 | phenyl | 2-COOCH₂CH₃ | 3-Cl | H |
| I.1.921 | phenyl | 2-COOCH₂CH₃ | 4-Cl | H |
| I.1.922 | phenyl | 2-COOCH₂CH₃ | 5-Cl | H |
| I.1.923 | phenyl | 2-COOCH₂CH₃ | 6-Cl | H |
| I.1.924 | phenyl | 2-COOCH₂CH₃ | 3-F | H |
| I.1.925 | phenyl | 2-COOCH₂CH₃ | 4-F | H |
| I.1.926 | phenyl | 2-COOCH₂CH₃ | 5-F | H |
| I.1.927 | phenyl | 2-COOCH₂CH₃ | 6-F | H |
| I.1.928 | phenyl | 2-COOCH₂CH₃ | 3-CH₃ | H |
| I.1.929 | phenyl | 2-COOCH₂CH₃ | 4-CH₃ | H |
| I.1.930 | phenyl | 2-COOCH₂CH₃ | 5-CH₃ | H |
| I.1.931 | phenyl | 2-COOCH₂CH₃ | 6-CH₃ | H |
| I.1.932 | phenyl | 2-COOCH₂CH₃ | 5-vinyl | H |
| I.1.933 | phenyl | 2-COOCH₂CH₃ | 6-vinyl | H |
| I.1.934 | phenyl | 2-COOCH₂CH₃ | 5-CF₃ | 3-Cl |
| I.1.935 | phenyl | 2-COOCH₂CH₃ | 5-CF₃ | 4-Cl |
| I.1.936 | phenyl | 2-COOCH₂CH₃ | 4-CF₃ | 5-Cl |
| I.1.937 | phenyl | 2-COOCH₂CH₃ | 5-CF₃ | 6-Cl |
| I.1.938 | phenyl | 2-COOCH₂CH₃ | 5-CF₃ | 3-F |
| I.1.939 | phenyl | 2-COOCH₂CH₃ | 5-CF₃ | 4-F |
| I.1.940 | phenyl | 2-COOCH₂CH₃ | 4-CF₃ | 5-F |
| I.1.941 | phenyl | 2-COOCH₂CH₃ | 5-CF₃ | 6-F |
| I.1.942 | phenyl | 2-COOCH₂CH₃ | 5-CF₃ | 3-CH₃ |
| I.1.943 | phenyl | 2-COOCH₂CH₃ | 5-CF₃ | 4-CH₃ |
| I.1.944 | phenyl | 2-COOCH₂CH₃ | 4-CF₃ | 5-CH₃ |
| I.1.945 | phenyl | 2-COOCH₂CH₃ | 5-CF₃ | 6-CH₃ |
| I.1.946 | phenyl | 2-COOCH₂CH₃ | 4-CF₃ | 5-vinyl |
| I.1.947 | phenyl | 2-COOCH₂CH₃ | 4-CF₃ | 6-vinyl |
| I.1.948 | phenyl | 2-COOCH₂CH₃ | 3-Cl | 4-F |
| I.1.949 | phenyl | 2-COOCH₂CH₃ | 3-Cl | 5-F |
| I.1.950 | phenyl | 2-COOCH₂CH₃ | 3-Cl | 6-F |
| I.1.951 | phenyl | 2-COOCH₂CH₃ | 3-Cl | 4-Cl |
| I.1.952 | phenyl | 2-COOCH₂CH₃ | 3-Cl | 5-Cl |
| I.1.953 | phenyl | 2-COOCH₂CH₃ | 3-Cl | 6-Cl |
| I.1.954 | phenyl | 2-COOCH₂CH₃ | 3-Cl | 4-CH₃ |
| I.1.955 | phenyl | 2-COOCH₂CH₃ | 3-Cl | 5-CH₃ |
| I.1.956 | phenyl | 2-COOCH₂CH₃ | 3-Cl | 6-CH₃ |
| I.1.957 | phenyl | 2-COOCH₂CH₃ | 3-Cl | 5-vinyl |
| I.1.958 | phenyl | 2-COOCH₂CH₃ | 3-Cl | 6-vinyl |
| I.1.959 | phenyl | 2-COOCH₂CH₃ | 3-F | 4-F |
| I.1.960 | phenyl | 2-COOCH₂CH₃ | 3-F | 5-F |
| I.1.961 | phenyl | 2-COOCH₂CH₃ | 3-F | 6-F |
| I.1.962 | phenyl | 2-COOCH₂CH₃ | 3-F | 4-Cl |
| I.1.963 | phenyl | 2-COOCH₂CH₃ | 3-F | 5-Cl |
| I.1.964 | phenyl | 2-COOCH₂CH₃ | 3-F | 6-Cl |
| I.1.965 | phenyl | 2-COOCH₂CH₃ | 3-F | 4-CH₃ |
| I.1.966 | phenyl | 2-COOCH₂CH₃ | 3-F | 5-CH₃ |
| I.1.967 | phenyl | 2-COOCH₂CH₃ | 3-F | 6-CH₃ |
| I.1.968 | phenyl | 2-COOCH₂CH₃ | 3-F | 5-vinyl |
| I.1.969 | phenyl | 2-COOCH₂CH₃ | 3-F | 6-vinyl |
| I.1.970 | phenyl | 2-COOCH₂CH₃ | 3-CH₃ | 4-F |
| I.1.971 | phenyl | 2-COOCH₂CH₃ | 3-CH₃ | 5-F |
| I.1.972 | phenyl | 2-COOCH₂CH₃ | 3-CH₃ | 6-F |
| I.1.973 | phenyl | 2-COOCH₂CH₃ | 3-CH₃ | 4-Cl |
| I.1.974 | phenyl | 2-COOCH₂CH₃ | 3-CH₃ | 5-Cl |
| I.1.975 | phenyl | 2-COOCH₂CH₃ | 3-CH₃ | 6-Cl |
| I.1.976 | phenyl | 2-COOCH₂CH₃ | 3-CH₃ | 4-CH₃ |
| I.1.977 | phenyl | 2-COOCH₂CH₃ | 3-CH₃ | 5-CH₃ |
| I.1.978 | phenyl | 2-COOCH₂CH₃ | 3-CH₃ | 6-CH₃ |
| I.1.979 | phenyl | 2-COOCH₂CH₃ | 3-CH₃ | 5-vinyl |
| I.1.980 | phenyl | 2-COOCH₂CH₃ | 3-CH₃ | 6-vinyl |
| I.1.981 | phenyl | 2-COOCH₂CH₃ | 4-Cl | 5-F |
| I.1.982 | phenyl | 2-COOCH₂CH₃ | 4-Cl | 6-F |
| I.1.983 | phenyl | 2-COOCH₂CH₃ | 4-Cl | 5-Cl |
| I.1.984 | phenyl | 2-COOCH₂CH₃ | 4-Cl | 6-Cl |
| I.1.985 | phenyl | 2-COOCH₂CH₃ | 4-Cl | 5-CH₃ |
| I.1.986 | phenyl | 2-COOCH₂CH₃ | 4-Cl | 6-CH₃ |
| I.1.987 | phenyl | 2-COOCH₂CH₃ | 4-Cl | 5-vinyl |
| I.1.988 | phenyl | 2-COOCH₂CH₃ | 4-Cl | 6-vinyl |
| I.1.989 | phenyl | 2-COOCH₂CH₃ | 4-F | 5-F |
| I.1.990 | phenyl | 2-COOCH₂CH₃ | 4-F | 6-F |
| I.1.991 | phenyl | 2-COOCH₂CH₃ | 4-F | 5-Cl |
| I.1.992 | phenyl | 2-COOCH₂CH₃ | 4-F | 6-Cl |
| I.1.993 | phenyl | 2-COOCH₂CH₃ | 4-F | 5-CH₃ |
| I.1.994 | phenyl | 2-COOCH₂CH₃ | 4-F | 6-CH₃ |
| I.1.995 | phenyl | 2-COOCH₂CH₃ | 4-F | 5-vinyl |
| I.1.996 | phenyl | 2-COOCH₂CH₃ | 4-F | 6-vinyl |
| I.1.997 | phenyl | 2-COOCH₂CH₃ | 4-CH₃ | 5-F |
| I.1.998 | phenyl | 2-COOCH₂CH₃ | 4-CH₃ | 6-F |
| I.1.999 | phenyl | 2-COOCH₂CH₃ | 4-CH₃ | 5-Cl |
| I.1.1000 | phenyl | 2-COOCH₂CH₃ | 4-CH₃ | 6-Cl |
| I.1.1001 | phenyl | 2-COOCH₂CH₃ | 4-CH₃ | 5-CH₃ |
| I.1.1002 | phenyl | 2-COOCH₂CH₃ | 4-CH₃ | 6-CH₃ |
| I.1.1003 | phenyl | 2-COOCH₂CH₃ | 4-CH₃ | 5-vinyl |
| I.1.1004 | phenyl | 2-COOCH₂CH₃ | 4-CH₃ | 6-vinyl |
| I.1.1005 | phenyl | 2-COOCH₂CH₃ | 5-Cl | 6-Cl |
| I.1.1006 | phenyl | 2-COOCH₂CH₃ | 5-Cl | 6-F |
| I.1.1007 | phenyl | 2-COOCH₂CH₃ | 5-Cl | 6-CH₃ |
| I.1.1008 | phenyl | 2-COOCH₂CH₃ | 5-Cl | 6-vinyl |
| I.1.1009 | phenyl | 2-COOCH₂CH₃ | 5-F | 6-Cl |
| I.1.1010 | phenyl | 2-COOCH₂CH₃ | 5-F | 6-F |
| I.1.1011 | phenyl | 2-COOCH₂CH₃ | 5-F | 6-CH₃ |
| I.1.1012 | phenyl | 2-COOCH₂CH₃ | 5-F | 6-vinyl |
| I.1.1013 | phenyl | 2-COOCH₂CH₃ | 5-CH₃ | 6-Cl |
| I.1.1014 | phenyl | 2-COOCH₂CH₃ | 5-CH₃ | 6-F |
| I.1.1015 | phenyl | 2-COOCH₂CH₃ | 5-CH₃ | 6-CH₃ |
| I.1.1016 | phenyl | 2-COOCH₂CH₃ | 5-CH₃ | 6-vinyl |
| I.1.1017 | phenyl | 2-COOCH₂CH₃ | 5-vinyl | 6-Cl |
| I.1.1018 | phenyl | 2-COOCH₂CH₃ | 5-vinyl | 6-F |
| I.1.1019 | phenyl | 2-COOCH₂CH₃ | 5-vinyl | 6-CH₃ |
| I.1.1020 | phenyl | 2-COOCH₂CH₃ | 5-vinyl | 6-vinyl |
| I.1.1021 | phenyl | 2-COOCH₂CH₃ | H | H |
| I.1.1022 | phenyl | 2-COOCH₂CH₃ | 3-Cl | H |
| I.1.1023 | phenyl | 2-COOCH₂CH₃ | 4-Cl | H |
| I.1.1024 | phenyl | 2-COOCH₂CH₃ | 5-Cl | H |
| I.1.1025 | phenyl | 2-COOCH₂CH₃ | 6-Cl | H |
| I.1.1026 | phenyl | 2-COOCH₂CH₃ | 3-F | H |
| I.1.1027 | phenyl | 2-COOCH₂CH₃ | 4-F | H |
| I.1.1028 | phenyl | 2-COOCH₂CH₃ | 5-F | H |
| I.1.1029 | phenyl | 2-COOCH₂CH₃ | 6-F | H |
| I.1.1030 | phenyl | 2-COOCH₂CH₃ | 3-CH₃ | H |
| I.1.1031 | phenyl | 2-COOCH₂CH₃ | 4-CH₃ | H |
| I.1.1032 | phenyl | 2-COOCH₂CH₃ | 5-CH₃ | H |
| I.1.1033 | phenyl | 2-COOCH₂CH₃ | 6-CH₃ | H |
| I.1.1034 | phenyl | 2-COOCH₂CH₃ | 5-vinyl | H |
| I.1.1035 | phenyl | 2-COOCH₂CH₃ | 6-vinyl | H |
| I.1.1036 | phenyl | 2-COOCH₂CH₃ | 5-CF₃ | 3-Cl |
| I.1.1037 | phenyl | 2-COOCH₂CH₃ | 5-CF₃ | 4-Cl |
| I.1.1038 | phenyl | 2-COOCH₂CH₃ | 4-CF₃ | 5-Cl |
| I.1.1039 | phenyl | 2-COOCH₂CH₃ | 5-CF₃ | 6-Cl |
| I.1.1040 | phenyl | 2-COOCH₂CH₃ | 5-CF₃ | 3-F |
| I.1.1041 | phenyl | 2-COOCH₂CH₃ | 5-CF₃ | 4-F |
| I.1.1042 | phenyl | 2-COOCH₂CH₃ | 4-CF₃ | 5-F |
| I.1.1043 | phenyl | 2-COOCH₂CH₃ | 5-CF₃ | 6-F |
| I.1.1044 | phenyl | 2-COOCH₂CH₃ | 5-CF₃ | 3-CH₃ |
| I.1.1045 | phenyl | 2-COOCH₂CH₃ | 5-CF₃ | 4-CH₃ |
| I.1.1046 | phenyl | 2-COOCH₂CH₃ | 4-CF₃ | 5-CH₃ |
| I.1.1047 | phenyl | 2-COOCH₂CH₃ | 5-CF₃ | 6-CH₃ |
| I.1.1048 | phenyl | 2-COOCH₂CH₃ | 5-CF₃ | 5-vinyl |
| I.1.1049 | phenyl | 2-COOCH₂CH₃ | 4-CF₃ | 6-vinyl |
| I.1.1050 | phenyl | 2-COOCH₂CH₃ | 3-Cl | 4-F |
| I.1.1051 | phenyl | 2-COOCH₂CH₃ | 3-Cl | 5-F |
| I.1.1052 | phenyl | 2-COOCH₂CH₃ | 3-Cl | 6-F |
| I.1.1053 | phenyl | 2-COOCH₂CH₃ | 3-Cl | 4-Cl |
| I.1.1054 | phenyl | 2-COOCH₂CH₃ | 3-Cl | 5-Cl |
| I.1.1055 | phenyl | 2-COOCH₂CH₃ | 3-Cl | 6-Cl |
| I.1.1056 | phenyl | 2-COOCH₂CH₃ | 3-Cl | 4-CH₃ |
| I.1.1057 | phenyl | 2-COOCH₂CH₃ | 3-Cl | 5-CH₃ |
| I.1.1058 | phenyl | 2-COOCH₂CH₃ | 3-Cl | 6-CH₃ |
| I.1.1059 | phenyl | 2-COOCH₂CH₃ | 3-Cl | 5-vinyl |
| I.1.1060 | phenyl | 2-COOCH₂CH₃ | 3-Cl | 6-vinyl |
| I.1.1061 | phenyl | 2-COOCH₂CH₃ | 3-F | 4-F |
| I.1.1062 | phenyl | 2-COOCH₂CH₃ | 3-F | 5-F |
| I.1.1063 | phenyl | 2-COOCH₂CH₃ | 3-F | 6-F |
| I.1.1064 | phenyl | 2-COOCH₂CH₃ | 3-F | 4-Cl |
| I.1.1065 | phenyl | 2-COOCH₂CH₃ | 3-F | 5-Cl |
| I.1.1066 | phenyl | 2-COOCH₂CH₃ | 3-F | 6-Cl |
| I.1.1067 | phenyl | 2-COOCH₂CH₃ | 3-F | 4-CH₃ |
| I.1.1068 | phenyl | 2-COOCH₂CH₃ | 3-F | 5-CH₃ |
| I.1.1069 | phenyl | 2-COOCH₂CH₃ | 3-F | 6-CH₃ |
| I.1.1070 | phenyl | 2-COOCH₂CH₃ | 3-F | 5-vinyl |
| I.1.1071 | phenyl | 2-COOCH₂CH₃ | 3-F | 6-vinyl |
| I.1.1072 | phenyl | 2-COOCH₂CH₃ | 3-CH₃ | 4-F |
| I.1.1073 | phenyl | 2-COOCH₂CH₃ | 3-CH₃ | 5-F |
| I.1.1074 | phenyl | 2-COOCH₂CH₃ | 3-CH₃ | 6-F |
| I.1.1075 | phenyl | 2-COOCH₂CH₃ | 3-CH₃ | 4-Cl |
| I.1.1076 | phenyl | 2-COOCH₂CH₃ | 3-CH₃ | 5-Cl |
| I.1.1077 | phenyl | 2-COOCH₂CH₃ | 3-CH₃ | 6-Cl |
| I.1.1078 | phenyl | 2-COOCH₂CH₃ | 3-CH₃ | 4-CH₃ |
| I.1.1079 | phenyl | 2-COOCH₂CH₃ | 3-CH₃ | 5-CH₃ |
| I.1.1080 | phenyl | 2-COOCH₂CH₃ | 3-CH₃ | 6-CH₃ |
| I.1.1081 | phenyl | 2-COOCH₂CH₃ | 3-CH₃ | 5-vinyl |
| I.1.1082 | phenyl | 2-COOCH₂CH₃ | 3-CH₃ | 6-vinyl |
| I.1.1083 | phenyl | 2-COOCH₂CH₃ | 4-Cl | 5-F |
| I.1.1084 | phenyl | 2-COOCH₂CH₃ | 4-Cl | 6-F |
| I.1.1085 | phenyl | 2-COOCH₂CH₃ | 4-Cl | 5-Cl |
| I.1.1086 | phenyl | 2-COOCH₂CH₃ | 4-Cl | 6-Cl |
| I.1.1087 | phenyl | 2-COOCH₂CH₃ | 4-Cl | 5-CH₃ |
| I.1.1088 | phenyl | 2-COOCH₂CH₃ | 4-Cl | 6-CH₃ |
| I.1.1089 | phenyl | 2-COOCH₂CH₃ | 4-Cl | 5-vinyl |
| I.1.1090 | phenyl | 2-COOCH₂CH₃ | 4-Cl | 6-vinyl |
| I.1.1091 | phenyl | 2-COOCH₂CH₃ | 4-F | 5-F |
| I.1.1092 | phenyl | 2-COOCH₂CH₃ | 4-F | 6-F |
| I.1.1093 | phenyl | 2-COOCH₂CH₃ | 4-F | 5-Cl |
| I.1.1094 | phenyl | 2-COOCH₂CH₃ | 4-F | 6-Cl |
| I.1.1095 | phenyl | 2-COOCH₂CH₃ | 4-F | 5-CH₃ |
| I.1.1096 | phenyl | 2-COOCH₂CH₃ | 4-F | 6-CH₃ |
| I.1.1097 | phenyl | 2-COOCH₂CH₃ | 4-F | 5-vinyl |
| I.1.1098 | phenyl | 2-COOCH₂CH₃ | 4-F | 6-vinyl |
| I.1.1099 | phenyl | 2-COOCH₂CH₃ | 4-CH₃ | 5-F |
| I.1.1100 | phenyl | 2-COOCH₂CH₃ | 4-CH₃ | 6-F |
| I.1.1101 | phenyl | 2-COOCH₂CH₃ | 4-CH₃ | 5-Cl |
| I.1.1102 | phenyl | 2-COOCH₂CH₃ | 4-CH₃ | 6-Cl |
| I.1.1103 | phenyl | 2-COOCH₂CH₃ | 4-CH₃ | 5-CH₃ |
| I.1.1104 | phenyl | 2-COOCH₂CH₃ | 4-CH₃ | 6-CH₃ |
| I.1.1105 | phenyl | 2-COOCH₂CH₃ | 4-CH₃ | 5-vinyl |
| I.1.1106 | phenyl | 2-COOCH₂CH₃ | 4-CH₃ | 6-vinyl |
| I.1.1107 | phenyl | 2-COOCH₂CH₃ | 5-Cl | 6-Cl |
| I.1.1108 | phenyl | 2-COOCH₂CH₃ | 5-Cl | 6-F |
| I.1.1109 | phenyl | 2-COOCH₂CH₃ | 5-Cl | 6-CH₃ |
| I.1.1110 | phenyl | 2-COOCH₂CH₃ | 5-Cl | 6-vinyl |
| I.1.1111 | phenyl | 2-COOCH₂CH₃ | 5-F | 6-Cl |
| I.1.1112 | phenyl | 2-COOCH₂CH₃ | 5-F | 6-F |
| I.1.1113 | phenyl | 2-COOCH₂CH₃ | 5-F | 6-CH₃ |
| I.1.1114 | phenyl | 2-COOCH₂CH₃ | 5-F | 6-vinyl |
| I.1.1115 | phenyl | 2-COOCH₂CH₃ | 5-CH₃ | 6-Cl |
| I.1.1116 | phenyl | 2-COOCH₂CH₃ | 5-CH₃ | 6-F |
| I.1.1117 | phenyl | 2-COOCH₂CH₃ | 5-CH₃ | 6-CH₃ |
| I.1.1118 | phenyl | 2-COOCH₂CH₃ | 5-CH₃ | 6-vinyl |
| I.1.1119 | phenyl | 2-COOCH₂CH₃ | 5-vinyl | 6-Cl |
| I.1.1120 | phenyl | 2-COOCH₂CH₃ | 5-vinyl | 6-F |
| I.1.1121 | phenyl | 2-COOCH₂CH₃ | 5-vinyl | 6-CH₃ |
| I.1.1122 | phenyl | 2-COOCH₂CH₃ | 5-vinyl | 6-vinyl |
| I.1.1123 | pyridin-2-yl | 3-NO₂ | 4-Cl | 5-F |
| I.1.1124 | pyridin-2-yl | 3-NO₂ | 4-Cl | H |
| I.1.1125 | pyridin-2-yl | 3-NO₂ | 5-Cl | H |
| I.1.1126 | pyridin-2-yl | 3-NO₂ | 6-Cl | H |
| I.1.1127 | pyridin-2-yl | 3-NO₂ | 4-F | H |
| I.1.1128 | pyridin-2-yl | 3-NO₂ | 5-F | H |
| I.1.1129 | pyridin-2-yl | 3-NO₂ | 6-F | H |
| I.1.1130 | pyridin-2-yl | 3-NO₂ | 4-Br | H |
| I.1.1131 | pyridin-2-yl | 3-NO₂ | 5-Br | H |
| I.1.1132 | pyridin-2-yl | 3-NO₂ | 6-Br | H |
| I.1.1133 | pyridin-2-yl | 3-NO₂ | 4-CH₃ | H |
| I.1.1134 | pyridin-2-yl | 3-NO₂ | 5-CH₃ | H |
| I.1.1135 | pyridin-2-yl | 3-NO₂ | 6-CH₃ | H |
| I.1.1136 | pyridin-2-yl | 3-NO₂ | 4-CF₃ | H |
| I.1.1137 | pyridin-2-yl | 3-NO₂ | 5-CF₃ | H |
| I.1.1138 | pyridin-2-yl | 3-NO₂ | 6-CF₃ | H |
| I.1.1139 | pyridin-2-yl | 3-NO₂ | 4-vinyl | H |
| I.1.1140 | pyridin-2-yl | 3-NO₂ | 5-vinyl | H |
| I.1.1141 | pyridin-2-yl | 3-NO₂ | 6-vinyl | H |
| I.1.1142 | pyridin-2-yl | 3-NO₂ | 4-Cl | 6-F |
| I.1.1143 | pyridin-2-yl | 3-NO₂ | 5-Cl | 6-F |
| I.1.1144 | pyridin-2-yl | 3-CN | 4-Cl | 5-F |
| I.1.1145 | pyridin-2-yl | 3-CN | 4-Cl | H |
| I.1.1146 | pyridin-2-yl | 3-CN | 5-Cl | H |
| I.1.1147 | pyridin-2-yl | 3-CN | 6-Cl | H |
| I.1.1148 | pyridin-2-yl | 3-CN | 4-F | H |
| I.1.1149 | pyridin-2-yl | 3-CN | 5-F | H |
| I.1.1150 | pyridin-2-yl | 3-CN | 6-F | H |
| I.1.1151 | pyridin-2-yl | 3-CN | 4-Br | H |
| I.1.1152 | pyridin-2-yl | 3-CN | 5-Br | H |
| I.1.1153 | pyridin-2-yl | 3-CN | 6-Br | H |
| I.1.1154 | pyridin-2-yl | 3-CN | 4-CH₃ | H |
| I.1.1155 | pyridin-2-yl | 3-CN | 5-CH₃ | H |
| I.1.1156 | pyridin-2-yl | 3-CN | 6-CH₃ | H |
| I.1.1157 | pyridin-2-yl | 3-CN | 4-CF₃ | H |
| I.1.1158 | pyridin-2-yl | 3-CN | 5-CF₃ | H |
| I.1.1159 | pyridin-2-yl | 3-CN | 6-CF₃ | H |
| I.1.1160 | pyridin-2-yl | 3-CN | 4-vinyl | H |
| I.1.1161 | pyridin-2-yl | 3-CN | 5-vinyl | H |
| I.1.1162 | pyridin-2-yl | 3-CN | 6-vinyl | H |
| I.1.1163 | pyridin-2-yl | 3-CN | 4-Cl | 6-F |
| I.1.1164 | pyridin-2-yl | 3-CN | 5-Cl | 6-F |
| I.1.1165 | pyridin-2-yl | 3-Br | 4-Cl | 5-F |
| I.1.1166 | pyridin-2-yl | 3-Br | 4-Cl | H |
| I.1.1167 | pyridin-2-yl | 3-Br | 5-Cl | H |
| I.1.1168 | pyridin-2-yl | 3-Br | 6-Cl | H |
| I.1.1169 | pyridin-2-yl | 3-Br | 4-F | H |
| I.1.1170 | pyridin-2-yl | 3-Br | 5-F | H |
| I.1.1171 | pyridin-2-yl | 3-Br | 6-F | H |
| I.1.1172 | pyridin-2-yl | 3-Br | 4-Br | H |
| I.1.1173 | pyridin-2-yl | 3-Br | 5-Br | H |
| I.1.1174 | pyridin-2-yl | 3-Br | 6-Br | H |
| I.1.1175 | pyridin-2-yl | 3-Br | 4-CH₃ | H |
| I.1.1176 | pyridin-2-yl | 3-Br | 5-CH₃ | H |
| I.1.1177 | pyridin-2-yl | 3-Br | 6-CH₃ | H |
| I.1.1178 | pyridin-2-yl | 3-Br | 4-CF₃ | H |
| I.1.1179 | pyridin-2-yl | 3-Br | 5-CF₃ | H |
| I.1.1180 | pyridin-2-yl | 3-Br | 6-CF₃ | H |
| I.1.1181 | pyridin-2-yl | 3-Br | 4-vinyl | H |
| I.1.1182 | pyridin-2-yl | 3-Br | 5-vinyl | H |
| I.1.1183 | pyridin-2-yl | 3-Br | 6-vinyl | H |
| I.1.1184 | pyridin-2-yl | 3-Br | 4-Cl | 6-F |
| I.1.1185 | pyridin-2-yl | 3-Br | 5-Cl | 6-F |
| I.1.1186 | pyridin-2-yl | 3-Cl | 4-Cl | 5-F |
| I.1.1187 | pyridin-2-yl | 3-Cl | 4-Cl | H |
| I.1.1188 | pyridin-2-yl | 3-Cl | 5-Cl | H |
| I.1.1189 | pyridin-2-yl | 3-Cl | 6-Cl | H |
| I.1.1190 | pyridin-2-yl | 3-Cl | 4-F | H |
| I.1.1191 | pyridin-2-yl | 3-Cl | 5-F | H |
| I.1.1192 | pyridin-2-yl | 3-Cl | 6-F | H |
| I.1.1193 | pyridin-2-yl | 3-Cl | 4-Br | H |
| I.1.1194 | pyridin-2-yl | 3-Cl | 5-Br | H |
| I.1.1195 | pyridin-2-yl | 3-Cl | 6-Br | H |
| I.1.1196 | pyridin-2-yl | 3-Cl | 4-CH₃ | H |
| I.1.1197 | pyridin-2-yl | 3-Cl | 5-CH₃ | H |
| I.1.1198 | pyridin-2-yl | 3-Cl | 6-CH₃ | H |
| I.1.1199 | pyridin-2-yl | 3-Cl | 4-CF₃ | H |
| I.1.1200 | pyridin-2-yl | 3-Cl | 5-CF₃ | H |
| I.1.1201 | pyridin-2-yl | 3-Cl | 6-CF₃ | H |
| I.1.1202 | pyridin-2-yl | 3-Cl | 4-vinyl | H |
| I.1.1203 | pyridin-2-yl | 3-Cl | 5-vinyl | H |
| I.1.1204 | pyridin-2-yl | 3-Cl | 6-vinyl | H |
| I.1.1205 | pyridin-2-yl | 3-Cl | 4-Cl | 6-F |
| I.1.1206 | pyridin-2-yl | 3-Cl | 5-Cl | 6-F |
| I.1.1207 | pyridin-2-yl | 3-ethynyl | 4-Cl | 5-F |
| I.1.1208 | pyridin-2-yl | 3-ethynyl | 4-Cl | H |
| I.1.1209 | pyridin-2-yl | 3-ethynyl | 5-Cl | H |
| I.1.1210 | pyridin-2-yl | 3-ethynyl | 6-Cl | H |
| I.1.1211 | pyridin-2-yl | 3-ethynyl | 4-F | H |
| I.1.1212 | pyridin-2-yl | 3-ethynyl | 5-F | H |
| I.1.1213 | pyridin-2-yl | 3-ethynyl | 6-F | H |
| I.1.1214 | pyridin-2-yl | 3-ethynyl | 4-Br | H |
| I.1.1215 | pyridin-2-yl | 3-ethynyl | 5-Br | H |
| I.1.1216 | pyridin-2-yl | 3-ethynyl | 6-Br | H |
| I.1.1217 | pyridin-2-yl | 3-ethynyl | 4-CH₃ | H |
| I.1.1218 | pyridin-2-yl | 3-ethynyl | 5-CH₃ | H |
| I.1.1219 | pyridin-2-yl | 3-ethynyl | 6-CH₃ | H |
| I.1.1220 | pyridin-2-yl | 3-ethynyl | 4-CF₃ | H |
| I.1.1221 | pyridin-2-yl | 3-ethynyl | 5-CF₃ | H |
| I.1.1222 | pyridin-2-yl | 3-ethynyl | 6-CF₃ | H |
| I.1.1223 | pyridin-2-yl | 3-ethynyl | 4-vinyl | H |
| I.1.1224 | pyridin-2-yl | 3-ethynyl | 5-vinyl | H |
| I.1.1225 | pyridin-2-yl | 3-ethynyl | 6-vinyl | H |
| I.1.1226 | pyridin-2-yl | 3-ethynyl | 4-Cl | 6-F |
| I.1.1227 | pyridin-2-yl | 3-ethynyl | 5-Cl | 6-F |
| I.1.1228 | pyridin-2-yl | 3-I | 4-Cl | 5-F |
| I.1.1229 | pyridin-2-yl | 3-I | 4-Cl | H |
| I.1.1230 | pyridin-2-yl | 3-I | 5-Cl | H |
| I.1.1231 | pyridin-2-yl | 3-I | 6-Cl | H |
| I.1.1232 | pyridin-2-yl | 3-I | 4-F | H |
| I.1.1233 | pyridin-2-yl | 3-I | 5-F | H |
| I.1.1234 | pyridin-2-yl | 3-I | 6-F | H |
| I.1.1235 | pyridin-2-yl | 3-I | 4-Br | H |
| I.1.1236 | pyridin-2-yl | 3-I | 5-Br | H |
| I.1.1237 | pyridin-2-yl | 3-I | 6-Br | H |
| I.1.1238 | pyridin-2-yl | 3-I | 4-CH₃ | H |
| I.1.1239 | pyridin-2-yl | 3-I | 5-CH₃ | H |
| I.1.1240 | pyridin-2-yl | 3-I | 6-CH₃ | H |
| I.1.1241 | pyridin-2-yl | 3-I | 4-CF₃ | H |
| I.1.1242 | pyridin-2-yl | 3-I | 5-CF₃ | H |
| I.1.1243 | pyridin-2-yl | 3-I | 6-CF₃ | H |
| I.1.1244 | pyridin-2-yl | 3-I | 4-vinyl | H |
| I.1.1245 | pyridin-2-yl | 3-I | 5-vinyl | H |
| I.1.1246 | pyridin-2-yl | 3-I | 6-vinyl | H |
| I.1.1247 | pyridin-2-yl | 3-I | 4-Cl | 6-F |
| I.1.1248 | pyridin-2-yl | 3-I | 5-Cl | 6-F |
| I.1.1249 | pyridin-2-yl | 3-COOH | 4-Cl | 5-F |
| I.1.1250 | pyridin-2-yl | 3-COOH | 4-Cl | H |
| I.1.1251 | pyridin-2-yl | 3-COOH | 5-Cl | H |
| I.1.1252 | pyridin-2-yl | 3-COOH | 6-Cl | H |
| I.1.1253 | pyridin-2-yl | 3-COOH | 4-F | H |
| I.1.1254 | pyridin-2-yl | 3-COOH | 5-F | H |
| I.1.1255 | pyridin-2-yl | 3-COOH | 6-F | H |
| I.1.1256 | pyridin-2-yl | 3-COOH | 4-Br | H |
| I.1.1257 | pyridin-2-yl | 3-COOH | 5-Br | H |
| I.1.1258 | pyridin-2-yl | 3-COOH | 6-Br | H |
| I.1.1259 | pyridin-2-yl | 3-COOH | 4-CH₃ | H |
| I.1.1260 | pyridin-2-yl | 3-COOH | 5-CH₃ | H |
| I.1.1261 | pyridin-2-yl | 3-COOH | 6-CH₃ | H |
| I.1.1262 | pyridin-2-yl | 3-COOH | 4-CF₃ | H |
| I.1.1263 | pyridin-2-yl | 3-COOH | 5-CF₃ | H |
| I.1.1264 | pyridin-2-yl | 3-COOH | 6-CF₃ | H |
| I.1.1265 | pyridin-2-yl | 3-COOH | 4-vinyl | H |
| I.1.1266 | pyridin-2-yl | 3-COOH | 5-vinyl | H |
| I.1.1267 | pyridin-2-yl | 3-COOH | 6-vinyl | H |
| I.1.1268 | pyridin-2-yl | 3-COOH | 4-Cl | 6-F |
| I.1.1269 | pyridin-2-yl | 3-COOH | 5-Cl | 6-F |
| I.1.1270 | pyridin-2-yl | 3-COOCH₃ | 4-Cl | 5-F |
| I.1.1271 | pyridin-2-yl | 3-COOCH₃ | 4-Cl | H |
| I.1.1272 | pyridin-2-yl | 3-COOCH₃ | 5-Cl | H |
| I.1.1273 | pyridin-2-yl | 3-COOCH₃ | 6-Cl | H |
| I.1.1274 | pyridin-2-yl | 3-COOCH₃ | 4-F | H |
| I.1.1275 | pyridin-2-yl | 3-COOCH₃ | 5-F | H |
| I.1.1276 | pyridin-2-yl | 3-COOCH₃ | 6-F | H |
| I.1.1277 | pyridin-2-yl | 3-COOCH₃ | 4-Br | H |
| I.1.1278 | pyridin-2-yl | 3-COOCH₃ | 5-Br | H |
| I.1.1279 | pyridin-2-yl | 3-COOCH₃ | 6-Br | H |
| I.1.1280 | pyridin-2-yl | 3-COOCH₃ | 4-CH₃ | H |
| I.1.1281 | pyridin-2-yl | 3-COOCH₃ | 5-CH₃ | H |
| I.1.1282 | pyridin-2-yl | 3-COOCH₃ | 6-CH₃ | H |
| I.1.1283 | pyridin-2-yl | 3-COOCH₃ | 4-CF₃ | H |
| I.1.1284 | pyridin-2-yl | 3-COOCH₃ | 5-CF₃ | H |
| I.1.1285 | pyridin-2-yl | 3-COOCH₃ | 6-CF₃ | H |
| I.1.1286 | pyridin-2-yl | 3-COOCH₃ | 4-vinyl | H |
| I.1.1287 | pyridin-2-yl | 3-COOCH₃ | 5-vinyl | H |
| I.1.1288 | pyridin-2-yl | 3-COOCH₃ | 6-vinyl | H |
| I.1.1289 | pyridin-2-yl | 3-COOCH₃ | 4-Cl | 6-F |
| I.1.1290 | pyridin-2-yl | 3-COOCH₃ | 5-Cl | 6-F |
| I.1.1291 | pyridin-2-yl | 3-COOCH₂CH₃ | 4-Cl | 5-F |
| I.1.1292 | pyridin-2-yl | 3-COOCH₂CH₃ | 4-Cl | H |
| I.1.1293 | pyridin-2-yl | 3-COOCH₂CH₃ | 5-Cl | H |
| I.1.1294 | pyridin-2-yl | 3-COOCH₂CH₃ | 6-Cl | H |
| I.1.1295 | pyridin-2-yl | 3-COOCH₂CH₃ | 4-F | H |
| I.1.1296 | pyridin-2-yl | 3-COOCH₂CH₃ | 5-F | H |
| I.1.1297 | pyridin-2-yl | 3-COOCH₂CH₃ | 6-F | H |
| I.1.1298 | pyridin-2-yl | 3-COOCH₂CH₃ | 4-Br | H |
| I.1.1299 | pyridin-2-yl | 3-COOCH₂CH₃ | 5-Br | H |
| I.1.1300 | pyridin-2-yl | 3-COOCH₂CH₃ | 6-Br | H |
| I.1.1301 | pyridin-2-yl | 3-COOCH₂CH₃ | 4-CH₃ | H |
| I.1.1302 | pyridin-2-yl | 3-COOCH₂CH₃ | 5-CH₃ | H |
| I.1.1303 | pyridin-2-yl | 3-COOCH₂CH₃ | 6-CH₃ | H |
| I.1.1304 | pyridin-2-yl | 3-COOCH₂CH₃ | 4-CF₃ | H |
| I.1.1305 | pyridin-2-yl | 3-COOCH₂CH₃ | 5-CF₃ | H |
| I.1.1306 | pyridin-2-yl | 3-COOCH₂CH₃ | 6-CF₃ | H |
| I.1.1307 | pyridin-2-yl | 3-COOCH₂CH₃ | 4-vinyl | H |
| I.1.1308 | pyridin-2-yl | 3-COOCH₂CH₃ | 5-vinyl | H |
| I.1.1309 | pyridin-2-yl | 3-COOCH₂CH₃ | 6-vinyl | H |
| I.1.1310 | pyridin-2-yl | 3-COOCH₂CH₃ | 4-Cl | 6-F |
| I.1.1311 | pyridin-2-yl | 3-COOCH₂CH₃ | 5-Cl | 6-F |
| I.1.1312 | pyridin-2-yl | 3-CONHCH₃ | 4-Cl | 5-F |
| I.1.1313 | pyridin-2-yl | 3-CONHCH₃ | 4-Cl | H |
| I.1.1314 | pyridin-2-yl | 3-CONHCH₃ | 5-Cl | H |
| I.1.1315 | pyridin-2-yl | 3-CONHCH₃ | 6-Cl | H |
| I.1.1316 | pyridin-2-yl | 3-CONHCH₃ | 4-F | H |
| I.1.1317 | pyridin-2-yl | 3-CONHCH₃ | 5-F | H |
| I.1.1318 | pyridin-2-yl | 3-CONHCH₃ | 6-F | H |
| I.1.1319 | pyridin-2-yl | 3-CONHCH₃ | 4-Br | H |
| I.1.1320 | pyridin-2-yl | 3-CONHCH₃ | 5-Br | H |
| I.1.1321 | pyridin-2-yl | 3-CONHCH₃ | 6-Br | H |
| I.1.1322 | pyridin-2-yl | 3-CONHCH₃ | 4-CH₃ | H |
| I.1.1323 | pyridin-2-yl | 3-CONHCH₃ | 5-CH₃ | H |
| I.1.1324 | pyridin-2-yl | 3-CONHCH₃ | 6-CH₃ | H |
| I.1.1325 | pyridin-2-yl | 3-CONHCH₃ | 4-CF₃ | H |
| I.1.1326 | pyridin-2-yl | 3-CONHCH₃ | 5-CF₃ | H |
| I.1.1327 | pyridin-2-yl | 3-CONHCH₃ | 6-CF₃ | H |
| I.1.1328 | pyridin-2-yl | 3-CONHCH₃ | 4-vinyl | H |
| I.1.1329 | pyridin-2-yl | 3-CONHCH₃ | 5-vinyl | H |
| I.1.1330 | pyridin-2-yl | 3-CONHCH₃ | 6-vinyl | H |
| I.1.1331 | pyridin-2-yl | 3-CONHCH₃ | 4-Cl | 6-F |
| I.1.1332 | pyridin-2-yl | 3-CONHCH₃ | 5-Cl | 6-F |
| I.1.1333 | pyridin-2-yl | 3-CON(CH₃)₂ | 4-Cl | 5-F |
| I.1.1334 | pyridin-2-yl | 3-CON(CH₃)₂ | 4-Cl | H |
| I.1.1335 | pyridin-2-yl | 3-CON(CH₃)₂ | 5-Cl | H |
| I.1.1336 | pyridin-2-yl | 3-CON(CH₃)₂ | 6-Cl | H |
| I.1.1337 | pyridin-2-yl | 3-CON(CH₃)₂ | 4-F | H |
| I.1.1338 | pyridin-2-yl | 3-CON(CH₃)₂ | 5-F | H |
| I.1.1339 | pyridin-2-yl | 3-CON(CH₃)₂ | 6-F | H |
| I.1.1340 | pyridin-2-yl | 3-CON(CH₃)₂ | 4-Br | H |
| I.1.1341 | pyridin-2-yl | 3-CON(CH₃)₂ | 5-Br | H |
| I.1.1342 | pyridin-2-yl | 3-CON(CH₃)₂ | 6-Br | H |
| I.1.1343 | pyridin-2-yl | 3-CON(CH₃)₂ | 4-CH₃ | H |
| I.1.1344 | pyridin-2-yl | 3-CON(CH₃)₂ | 5-CH₃ | H |
| I.1.1345 | pyridin-2-yl | 3-CON(CH₃)₂ | 6-CH₃ | H |
| I.1.1346 | pyridin-2-yl | 3-CON(CH₃)₂ | 4-CF₃ | H |
| I.1.1347 | pyridin-2-yl | 3-CON(CH₃)₂ | 5-CF₃ | H |
| I.1.1348 | pyridin-2-yl | 3-CON(CH₃)₂ | 6-CF₃ | H |
| I.1.1349 | pyridin-2-yl | 3-CON(CH₃)₂ | 4-vinyl | H |
| I.1.1350 | pyridin-2-yl | 3-CON(CH₃)₂ | 5-vinyl | H |
| I.1.1351 | pyridin-2-yl | 3-CON(CH₃)₂ | 6-vinyl | H |
| I.1.1352 | pyridin-2-yl | 3-CON(CH₃)₂ | 4-Cl | 6-F |
| I.1.1353 | pyridin-2-yl | 3-CON(CH₃)₂ | 5-Cl | 6-F |
| I.1.1354 | pyridin-3-yl | 2-NO₂ | 4-Cl | 5-F |
| I.1.1355 | pyridin-3-yl | 2-NO₂ | 4-Cl | H |
| I.1.1356 | pyridin-3-yl | 2-NO₂ | 5-Cl | H |
| I.1.1357 | pyridin-3-yl | 2-NO₂ | 6-Cl | H |
| I.1.1358 | pyridin-3-yl | 2-NO₂ | 4-F | H |
| I.1.1359 | pyridin-3-yl | 2-NO₂ | 5-F | H |
| I.1.1360 | pyridin-3-yl | 2-NO₂ | 6-F | H |
| I.1.1361 | pyridin-3-yl | 2-NO₂ | 4-Br | H |
| I.1.1362 | pyridin-3-yl | 2-NO₂ | 5-Br | H |
| I.1.1363 | pyridin-3-yl | 2-NO₂ | 6-Br | H |
| I.1.1364 | pyridin-3-yl | 2-NO₂ | 4-CH₃ | H |
| I.1.1365 | pyridin-3-yl | 2-NO₂ | 5-CH₃ | H |
| I.1.1366 | pyridin-3-yl | 2-NO₂ | 6-CH₃ | H |
| I.1.1367 | pyridin-3-yl | 2-NO₂ | 4-CF₃ | H |
| I.1.1368 | pyridin-3-yl | 2-NO₂ | 5-CF₃ | H |
| I.1.1369 | pyridin-3-yl | 2-NO₂ | 6-CF₃ | H |
| I.1.1370 | pyridin-3-yl | 2-NO₂ | 4-vinyl | H |
| I.1.1371 | pyridin-3-yl | 2-NO₂ | 5-vinyl | H |
| I.1.1372 | pyridin-3-yl | 2-NO₂ | 6-vinyl | H |
| I.1.1373 | pyridin-3-yl | 2-NO₂ | 4-Cl | 6-F |
| I.1.1374 | pyridin-3-yl | 2-NO₂ | 5-Cl | 6-F |
| I.1.1412 | pyridin-3-yl | 2-Br | 4-vinyl | H |
| I.1.1413 | pyridin-3-yl | 2-Br | 5-vinyl | H |
| I.1.1414 | pyridin-3-yl | 2-Br | 6-vinyl | H |
| I.1.1415 | pyridin-3-yl | 2-Br | 4-Cl | 6-F |
| I.1.1416 | pyridin-3-yl | 2-Br | 5-Cl | 6-F |
| I.1.1417 | pyridin-3-yl | 2-Cl | 4-Cl | 5-F |
| I.1.1418 | pyridin-3-yl | 2-Cl | 4-Cl | H |
| I.1.1419 | pyridin-3-yl | 2-Cl | 5-Cl | H |
| I.1.1420 | pyridin-3-yl | 2-Cl | 6-Cl | H |
| I.1.1421 | pyridin-3-yl | 2-Cl | 4-F | H |
| I.1.1422 | pyridin-3-yl | 2-Cl | 5-F | H |
| I.1.1423 | pyridin-3-yl | 2-Cl | 6-F | H |
| I.1.1424 | pyridin-3-yl | 2-Cl | 4-Br | H |
| I.1.1425 | pyridin-3-yl | 2-Cl | 5-Br | H |
| I.1.1426 | pyridin-3-yl | 2-Cl | 6-Br | H |
| I.1.1427 | pyridin-3-yl | 2-Cl | 4-CH₃ | H |
| I.1.1428 | pyridin-3-yl | 2-Cl | 5-CH₃ | H |
| I.1.1429 | pyridin-3-yl | 2-Cl | 6-CH₃ | H |
| I.1.1430 | pyridin-3-yl | 2-Cl | 4-CF₃ | H |
| I.1.1431 | pyridin-3-yl | 2-Cl | 5-CF₃ | H |
| I.1.1432 | pyridin-3-yl | 2-Cl | 6-CF₃ | H |
| I.1.1433 | pyridin-3-yl | 2-Cl | 4-vinyl | H |
| I.1.1434 | pyridin-3-yl | 2-Cl | 5-vinyl | H |
| I.1.1435 | pyridin-3-yl | 2-Cl | 6-vinyl | H |
| I.1.1436 | pyridin-3-yl | 2-Cl | 4-Cl | 6-F |
| I.1.1437 | pyridin-3-yl | 2-Cl | 5-Cl | 6-F |
| I.1.1438 | pyridin-3-yl | 2-ethynyl | 4-Cl | 5-F |
| I.1.1439 | pyridin-3-yl | 2-ethynyl | 4-Cl | H |
| I.1.1440 | pyridin-3-yl | 2-ethynyl | 5-Cl | H |
| I.1.1441 | pyridin-3-yl | 2-ethynyl | 6-Cl | H |
| I.1.1442 | pyridin-3-yl | 2-ethynyl | 4-F | H |
| I.1.1443 | pyridin-3-yl | 2-ethynyl | 5-F | H |
| I.1.1444 | pyridin-3-yl | 2-ethynyl | 6-F | H |
| I.1.1445 | pyridin-3-yl | 2-ethynyl | 4-Br | H |
| I.1.1446 | pyridin-3-yl | 2-ethynyl | 5-Br | H |
| I.1.1447 | pyridin-3-yl | 2-ethynyl | 6-Br | H |
| I.1.1448 | pyridin-3-yl | 2-ethynyl | 4-CH₃ | H |
| I.1.1449 | pyridin-3-yl | 2-ethynyl | 5-CH₃ | H |
| I.1.1450 | pyridin-3-yl | 2-ethynyl | 6-CH₃ | H |
| I.1.1451 | pyridin-3-yl | 2-ethynyl | 4-CF₃ | H |
| I.1.1452 | pyridin-3-yl | 2-ethynyl | 5-CF₃ | H |
| I.1.1453 | pyridin-3-yl | 2-ethynyl | 6-CF₃ | H |
| I.1.1454 | pyridin-3-yl | 2-ethynyl | 4-vinyl | H |
| I.1.1455 | pyridin-3-yl | 2-ethynyl | 5-vinyl | H |
| I.1.1456 | pyridin-3-yl | 2-ethynyl | 6-vinyl | H |
| I.1.1457 | pyridin-3-yl | 2-ethynyl | 4-Cl | 6-F |
| I.1.1458 | pyridin-3-yl | 2-ethynyl | 5-Cl | 6-F |
| I.1.1459 | pyridin-3-yl | 2-I | 4-Cl | 5-F |
| I.1.1460 | pyridin-3-yl | 2-I | 4-Cl | H |
| I.1.1461 | pyridin-3-yl | 2-I | 5-Cl | H |
| I.1.1462 | pyridin-3-yl | 2-I | 6-Cl | H |
| I.1.1463 | pyridin-3-yl | 2-I | 4-F | H |
| I.1.1464 | pyridin-3-yl | 2-I | 5-F | H |
| I.1.1465 | pyridin-3-yl | 2-I | 6-F | H |
| I.1.1466 | pyridin-3-yl | 2-I | 4-Br | H |
| I.1.1467 | pyridin-3-yl | 2-I | 5-Br | H |
| I.1.1468 | pyridin-3-yl | 2-I | 6-Br | H |
| I.1.1469 | pyridin-3-yl | 2-I | 4-CH₃ | H |
| I.1.1470 | pyridin-3-yl | 2-I | 5-CH₃ | H |
| I.1.1471 | pyridin-3-yl | 2-I | 6-CH₃ | H |
| I.1.1472 | pyridin-3-yl | 2-I | 4-CF₃ | H |
| I.1.1473 | pyridin-3-yl | 2-I | 5-CF₃ | H |
| I.1.1474 | pyridin-3-yl | 2-I | 6-CF₃ | H |
| I.1.1475 | pyridin-3-yl | 2-I | 4-vinyl | H |
| I.1.1476 | pyridin-3-yl | 2-I | 5-vinyl | H |
| I.1.1477 | pyridin-3-yl | 2-I | 6-vinyl | H |
| I.1.1478 | pyridin-3-yl | 2-I | 4-Cl | 6-F |
| I.1.1479 | pyridin-3-yl | 2-I | 5-Cl | 6-F |
| I.1.1480 | pyridin-3-yl | 2-COOH | 4-Cl | 5-F |
| I.1.1481 | pyridin-3-yl | 2-COOH | 4-Cl | H |
| I.1.1482 | pyridin-3-yl | 2-COOH | 5-Cl | H |
| I.1.1483 | pyridin-3-yl | 2-COOH | 6-Cl | H |
| I.1.1484 | pyridin-3-yl | 2-COOH | 4-F | H |
| I.1.1485 | pyridin-3-yl | 2-COOH | 5-F | H |
| I.1.1486 | pyridin-3-yl | 2-COOH | 6-F | H |
| I.1.1487 | pyridin-3-yl | 2-COOH | 4-Br | H |
| I.1.1488 | pyridin-3-yl | 2-COOH | 5-Br | H |
| I.1.1489 | pyridin-3-yl | 2-COOH | 6-Br | H |
| I.1.1490 | pyridin-3-yl | 2-COOH | 4-CH₃ | H |
| I.1.1491 | pyridin-3-yl | 2-COOH | 5-CH₃ | H |
| I.1.1492 | pyridin-3-yl | 2-COOH | 6-CH₃ | H |
| I.1.1493 | pyridin-3-yl | 2-COOH | 4-CF₃ | H |
| I.1.1494 | pyridin-3-yl | 2-COOH | 5-CF₃ | H |
| I.1.1495 | pyridin-3-yl | 2-COOH | 6-CF₃ | H |
| I.1.1496 | pyridin-3-yl | 2-COOH | 4-vinyl | H |
| I.1.1497 | pyridin-3-yl | 2-COOH | 5-vinyl | H |
| I.1.1498 | pyridin-3-yl | 2-COOH | 6-vinyl | H |
| I.1.1499 | pyridin-3-yl | 2-COOH | 4-Cl | 6-F |
| I.1.1500 | pyridin-3-yl | 2-COOH | 5-Cl | 6-F |
| I.1.1501 | pyridin-3-yl | 2-COOCH₃ | 4-Cl | 5-F |
| I.1.1502 | pyridin-3-yl | 2-COOCH₃ | 4-Cl | H |
| I.1.1503 | pyridin-3-yl | 2-COOCH₃ | 5-Cl | H |
| I.1.1504 | pyridin-3-yl | 2-COOCH₃ | 6-Cl | H |
| I.1.1505 | pyridin-3-yl | 2-COOCH₃ | 4-F | H |
| I.1.1506 | pyridin-3-yl | 2-COOCH₃ | 5-F | H |
| I.1.1507 | pyridin-3-yl | 2-COOCH₃ | 6-F | H |
| I.1.1508 | pyridin-3-yl | 2-COOCH₃ | 4-Br | H |
| I.1.1509 | pyridin-3-yl | 2-COOCH₃ | 5-Br | H |
| I.1.1510 | pyridin-3-yl | 2-COOCH₃ | 6-Br | H |
| I.1.1511 | pyridin-3-yl | 2-COOCH₃ | 4-CH₃ | H |
| I.1.1512 | pyridin-3-yl | 2-COOCH₃ | 5-CH₃ | H |
| I.1.1513 | pyridin-3-yl | 2-COOCH₃ | 6-CH₃ | H |
| I.1.1514 | pyridin-3-yl | 2-COOCH₃ | 4-CF₃ | H |
| I.1.1515 | pyridin-3-yl | 2-COOCH₃ | 5-CF₃ | H |
| I.1.1516 | pyridin-3-yl | 2-COOCH₃ | 6-CF₃ | H |
| I.1.1517 | pyridin-3-yl | 2-COOCH₃ | 4-vinyl | H |
| I.1.1518 | pyridin-3-yl | 2-COOCH₃ | 5-vinyl | H |
| I.1.1519 | pyridin-3-yl | 2-COOCH₃ | 6-vinyl | H |
| I.1.1520 | pyridin-3-yl | 2-COOCH₃ | 4-Cl | 6-F |
| I.1.1521 | pyridin-3-yl | 2-COOCH₃ | 5-Cl | 6-F |
| I.1.1522 | pyridin-3-yl | 2-COOCH₂CH₃ | 4-Cl | 5-F |
| I.1.1523 | pyridin-3-yl | 2-COOCH₂CH₃ | 4-Cl | H |
| I.1.1524 | pyridin-3-yl | 2-COOCH₂CH₃ | 5-Cl | H |
| I.1.1525 | pyridin-3-yl | 2-COOCH₂CH₃ | 6-Cl | H |
| I.1.1526 | pyridin-3-yl | 2-COOCH₂CH₃ | 4-F | H |
| I.1.1527 | pyridin-3-yl | 2-COOCH₂CH₃ | 5-F | H |
| I.1.1528 | pyridin-3-yl | 2-COOCH₂CH₃ | 6-F | H |
| I.1.1529 | pyridin-3-yl | 2-COOCH₂CH₃ | 4-Br | H |
| I.1.1530 | pyridin-3-yl | 2-COOCH₂CH₃ | 5-Br | H |
| I.1.1531 | pyridin-3-yl | 2-COOCH₂CH₃ | 6-Br | H |
| I.1.1532 | pyridin-3-yl | 2-COOCH₂CH₃ | 4-CH₃ | H |
| I.1.1533 | pyridin-3-yl | 2-COOCH₂CH₃ | 5-CH₃ | H |
| I.1.1534 | pyridin-3-yl | 2-COOCH₂CH₃ | 6-CH₃ | H |
| I.1.1535 | pyridin-3-yl | 2-COOCH₂CH₃ | 4-CF₃ | H |
| I.1.1536 | pyridin-3-yl | 2-COOCH₂CH₃ | 5-CF₃ | H |
| I.1.1537 | pyridin-3-yl | 2-COOCH₂CH₃ | 6-CF₃ | H |
| I.1.1538 | pyridin-3-yl | 2-COOCH₂CH₃ | 4-vinyl | H |
| I.1.1539 | pyridin-3-yl | 2-COOCH₂CH₃ | 5-vinyl | H |
| I.1.1540 | pyridin-3-yl | 2-COOCH₂CH₃ | 6-vinyl | H |
| I.1.1541 | pyridin-3-yl | 2-COOCH₂CH₃ | 4-Cl | 6-F |
| I.1.1542 | pyridin-3-yl | 2-COOCH₂CH₃ | 5-Cl | 6-F |
| I.1.1543 | pyridin-3-yl | 2-CONHCH₃ | 4-Cl | 5-F |
| I.1.1544 | pyridin-3-yl | 2-CONHCH₃ | 4-Cl | H |
| I.1.1545 | pyridin-3-yl | 2-CONHCH₃ | 5-Cl | H |
| I.1.1546 | pyridin-3-yl | 2-CONHCH₃ | 6-Cl | H |
| I.1.1547 | pyridin-3-yl | 2-CONHCH₃ | 4-F | H |
| I.1.1548 | pyridin-3-yl | 2-CONHCH₃ | 5-F | H |
| I.1.1549 | pyridin-3-yl | 2-CONHCH₃ | 6-F | H |
| I.1.1550 | pyridin-3-yl | 2-CONHCH₃ | 4-Br | H |
| I.1.1551 | pyridin-3-yl | 2-CONHCH₃ | 5-Br | H |
| I.1.1552 | pyridin-3-yl | 2-CONHCH₃ | 6-Br | H |
| I.1.1553 | pyridin-3-yl | 2-CONHCH₃ | 4-CH₃ | H |
| I.1.1554 | pyridin-3-yl | 2-CONHCH₃ | 5-CH₃ | H |
| I.1.1555 | pyridin-3-yl | 2-CONHCH₃ | 6-CH₃ | H |
| I.1.1556 | pyridin-3-yl | 2-CONHCH₃ | 4-CF₃ | H |
| I.1.1557 | pyridin-3-yl | 2-CONHCH₃ | 5-CF₃ | H |
| I.1.1558 | pyridin-3-yl | 2-CONHCH₃ | 6-CF₃ | H |
| I.1.1559 | pyridin-3-yl | 2-CONHCH₃ | 4-vinyl | H |
| I.1.1560 | pyridin-3-yl | 2-CONHCH₃ | 5-vinyl | H |
| I.1.1561 | pyridin-3-yl | 2-CONHCH₃ | 6-vinyl | H |
| I.1.1562 | pyridin-3-yl | 2-CONHCH₃ | 4-Cl | 6-F |
| I.1.1563 | pyridin-3-yl | 2-CONHCH₃ | 5-Cl | 6-F |
| I.1.1564 | pyridin-3-yl | 2-CON(CH₃)₂ | 4-Cl | 5-F |
| I.1.1565 | pyridin-3-yl | 2-CON(CH₃)₂ | 4-Cl | H |
| I.1.1566 | pyridin-3-yl | 2-CON(CH₃)₂ | 5-Cl | H |
| I.1.1567 | pyridin-3-yl | 2-CON(CH₃)₂ | 6-Cl | H |
| I.1.1568 | pyridin-3-yl | 2-CON(CH₃)₂ | 4-F | H |
| I.1.1569 | pyridin-3-yl | 2-CON(CH₃)₂ | 5-F | H |
| I.1.1570 | pyridin-3-yl | 2-CON(CH₃)₂ | 6-F | H |
| I.1.1571 | pyridin-3-yl | 2-CON(CH₃)₂ | 4-Br | H |
| I.1.1572 | pyridin-3-yl | 2-CON(CH₃)₂ | 5-Br | H |
| I.1.1573 | pyridin-3-yl | 2-CON(CH₃)₂ | 6-Br | H |
| I.1.1574 | pyridin-3-yl | 2-CON(CH₃)₂ | 4-CH₃ | H |
| I.1.1575 | pyridin-3-yl | 2-CON(CH₃)₂ | 5-CH₃ | H |
| I.1.1576 | pyridin-3-yl | 2-CON(CH₃)₂ | 6-CH₃ | H |
| I.1.1577 | pyridin-3-yl | 2-CON(CH₃)₂ | 4-CF₃ | H |
| I.1.1578 | pyridin-3-yl | 2-CON(CH₃)₂ | 5-CF₃ | H |
| I.1.1579 | pyridin-3-yl | 2-CON(CH₃)₂ | 6-CF₃ | H |
| I.1.1580 | pyridin-3-yl | 2-CON(CH₃)₂ | 4-vinyl | H |
| I.1.1581 | pyridin-3-yl | 2-CON(CH₃)₂ | 5-vinyl | H |
| I.1.1582 | pyridin-3-yl | 2-CON(CH₃)₂ | 6-vinyl | H |
| I.1.1583 | pyridin-3-yl | 2-CON(CH₃)₂ | 4-Cl | 6-F |
| I.1.1584 | pyridin-3-yl | 2-CON(CH₃)₂ | 5-Cl | 6-F |
| I.1.1585 | pyridin-3-yl | 4-NO₂ | 2-Cl | 5-F |
| I.1.1586 | pyridin-3-yl | 4-NO₂ | 2-Cl | H |
| I.1.1587 | pyridin-3-yl | 4-NO₂ | 5-Cl | H |
| I.1.1588 | pyridin-3-yl | 4-NO₂ | 6-Cl | H |
| I.1.1589 | pyridin-3-yl | 4-NO₂ | 2-F | H |
| I.1.1590 | pyridin-3-yl | 4-NO₂ | 5-F | H |
| I.1.1591 | pyridin-3-yl | 4-NO₂ | 6-F | H |
| I.1.1592 | pyridin-3-yl | 4-NO₂ | 2-Br | H |
| I.1.1593 | pyridin-3-yl | 4-NO₂ | 5-Br | H |
| I.1.1594 | pyridin-3-yl | 4-NO₂ | 6-Br | H |
| I.1.1595 | pyridin-3-yl | 4-NO₂ | 2-CH₃ | H |
| I.1.1596 | pyridin-3-yl | 4-NO₂ | 5-CH₃ | H |
| I.1.1597 | pyridin-3-yl | 4-NO₂ | 6-CH₃ | H |
| I.1.1598 | pyridin-3-yl | 4-NO₂ | 2-CF₃ | H |
| I.1.1599 | pyridin-3-yl | 4-NO₂ | 5-CF₃ | H |
| I.1.1600 | pyridin-3-yl | 4-NO₂ | 6-CF₃ | H |
| I.1.1601 | pyridin-3-yl | 4-NO₂ | 2-vinyl | H |
| I.1.1602 | pyridin-3-yl | 4-NO₂ | 5-vinyl | H |
| I.1.1603 | pyridin-3-yl | 4-NO₂ | 6-vinyl | H |
| I.1.1604 | pyridin-3-yl | 4-NO₂ | 2-Cl | 6-F |
| I.1.1605 | pyridin-3-yl | 4-NO₂ | 5-Cl | 6-F |
| I.1.1606 | pyridin-3-yl | 4-CN | 2-Cl | 5-F |
| I.1.1607 | pyridin-3-yl | 4-CN | 2-Cl | H |
| I.1.1608 | pyridin-3-yl | 4-CN | 5-Cl | H |
| I.1.1609 | pyridin-3-yl | 4-CN | 6-Cl | H |
| I.1.1610 | pyridin-3-yl | 4-CN | 2-F | H |
| I.1.1611 | pyridin-3-yl | 4-CN | 5-F | H |
| I.1.1612 | pyridin-3-yl | 4-CN | 6-F | H |
| I.1.1613 | pyridin-3-yl | 4-CN | 2-Br | H |
| I.1.1614 | pyridin-3-yl | 4-CN | 5-Br | H |
| I.1.1615 | pyridin-3-yl | 4-CN | 6-Br | H |
| I.1.1616 | pyridin-3-yl | 4-CN | 2-CH₃ | H |
| I.1.1617 | pyridin-3-yl | 4-CN | 5-CH₃ | H |
| I.1.1618 | pyridin-3-yl | 4-CN | 6-CH₃ | H |
| I.1.1619 | pyridin-3-yl | 4-CN | 2-CF₃ | H |
| I.1.1620 | pyridin-3-yl | 4-CN | 5-CF₃ | H |
| I.1.1621 | pyridin-3-yl | 4-CN | 6-CF₃ | H |
| I.1.1622 | pyridin-3-yl | 4-CN | 2-vinyl | H |
| I.1.1623 | pyridin-3-yl | 4-CN | 5-vinyl | H |
| I.1.1624 | pyridin-3-yl | 4-CN | 6-vinyl | H |
| I.1.1625 | pyridin-3-yl | 4-CN | 2-Cl | 6-F |
| I.1.1626 | pyridin-3-yl | 4-CN | 5-Cl | 6-F |
| I.1.1627 | pyridin-3-yl | 4-Br | 2-Cl | 5-F |
| I.1.1628 | pyridin-3-yl | 4-Br | 2-Cl | H |
| I.1.1629 | pyridin-3-yl | 4-Br | 5-Cl | H |
| I.1.1630 | pyridin-3-yl | 4-Br | 6-Cl | H |
| I.1.1631 | pyridin-3-yl | 4-Br | 2-F | H |
| I.1.1632 | pyridin-3-yl | 4-Br | 5-F | H |
| I.1.1633 | pyridin-3-yl | 4-Br | 6-F | H |
| I.1.1634 | pyridin-3-yl | 4-Br | 2-Br | H |
| I.1.1635 | pyridin-3-yl | 4-Br | 5-Br | H |
| I.1.1636 | pyridin-3-yl | 4-Br | 6-Br | H |
| I.1.1637 | pyridin-3-yl | 4-Br | 2-CH₃ | H |
| I.1.1638 | pyridin-3-yl | 4-Br | 5-CH₃ | H |
| I.1.1639 | pyridin-3-yl | 4-Br | 6-CH₃ | H |
| I.1.1640 | pyridin-3-yl | 4-Br | 2-CF₃ | H |
| I.1.1641 | pyridin-3-yl | 4-Br | 5-CF₃ | H |
| I.1.1642 | pyridin-3-yl | 4-Br | 6-CF₃ | H |
| I.1.1643 | pyridin-3-yl | 4-Br | 2-vinyl | H |
| I.1.1644 | pyridin-3-yl | 4-Br | 5-vinyl | H |
| I.1.1645 | pyridin-3-yl | 4-Br | 6-vinyl | H |
| I.1.1646 | pyridin-3-yl | 4-Br | 2-Cl | 6-F |
| I.1.1647 | pyridin-3-yl | 4-Br | 5-Cl | 6-F |
| I.1.1648 | pyridin-3-yl | 4-Cl | 2-Cl | 5-F |
| I.1.1649 | pyridin-3-yl | 4-Cl | 2-Cl | H |
| I.1.1650 | pyridin-3-yl | 4-Cl | 5-Cl | H |
| I.1.1651 | pyridin-3-yl | 4-Cl | 6-Cl | H |
| I.1.1652 | pyridin-3-yl | 4-Cl | 2-F | H |
| I.1.1653 | pyridin-3-yl | 4-Cl | 5-F | H |
| I.1.1654 | pyridin-3-yl | 4-Cl | 6-F | H |
| I.1.1655 | pyridin-3-yl | 4-Cl | 2-Br | H |
| I.1.1656 | pyridin-3-yl | 4-Cl | 5-Br | H |
| I.1.1657 | pyridin-3-yl | 4-Cl | 6-Br | H |
| I.1.1658 | pyridin-3-yl | 4-Cl | 2-CH₃ | H |
| I.1.1659 | pyridin-3-yl | 4-Cl | 5-CH₃ | H |
| I.1.1660 | pyridin-3-yl | 4-Cl | 6-CH₃ | H |
| I.1.1661 | pyridin-3-yl | 4-Cl | 2-CF₃ | H |
| I.1.1662 | pyridin-3-yl | 4-Cl | 5-CF₃ | H |
| I.1.1663 | pyridin-3-yl | 4-Cl | 6-CF₃ | H |
| I.1.1664 | pyridin-3-yl | 4-Cl | 2-vinyl | H |
| I.1.1665 | pyridin-3-yl | 4-Cl | 5-vinyl | H |
| I.1.1666 | pyridin-3-yl | 4-Cl | 6-vinyl | H |
| I.1.1667 | pyridin-3-yl | 4-Cl | 2-Cl | 6-F |
| I.1.1668 | pyridin-3-yl | 4-Cl | 5-Cl | 6-F |
| I.1.1669 | pyridin-3-yl | 4-ethynyl | 2-Cl | 5-F |
| I.1.1670 | pyridin-3-yl | 4-ethynyl | 2-Cl | H |
| I.1.1671 | pyridin-3-yl | 4-ethynyl | 5-Cl | H |
| I.1.1672 | pyridin-3-yl | 4-ethynyl | 6-Cl | H |
| I.1.1673 | pyridin-3-yl | 4-ethynyl | 2-F | H |
| I.1.1674 | pyridin-3-yl | 4-ethynyl | 5-F | H |
| I.1.1675 | pyridin-3-yl | 4-ethynyl | 6-F | H |
| I.1.1676 | pyridin-3-yl | 4-ethynyl | 2-Br | H |
| I.1.1677 | pyridin-3-yl | 4-ethynyl | 5-Br | H |
| I.1.1678 | pyridin-3-yl | 4-ethynyl | 6-Br | H |
| I.1.1679 | pyridin-3-yl | 4-ethynyl | 2-CH₃ | H |
| I.1.1680 | pyridin-3-yl | 4-ethynyl | 5-CH₃ | H |
| I.1.1681 | pyridin-3-yl | 4-ethynyl | 6-CH₃ | H |
| I.1.1682 | pyridin-3-yl | 4-ethynyl | 2-CF₃ | H |
| I.1.1683 | pyridin-3-yl | 4-ethynyl | 5-CF₃ | H |
| I.1.1684 | pyridin-3-yl | 4-ethynyl | 6-CF₃ | H |
| I.1.1685 | pyridin-3-yl | 4-ethynyl | 2-vinyl | H |
| I.1.1686 | pyridin-3-yl | 4-ethynyl | 5-vinyl | H |
| I.1.1687 | pyridin-3-yl | 4-ethynyl | 6-vinyl | H |
| I.1.1688 | pyridin-3-yl | 4-ethynyl | 2-Cl | 6-F |
| I.1.1689 | pyridin-3-yl | 4-ethynyl | 5-Cl | 6-F |
| I.1.1690 | pyridin-3-yl | 4-I | 2-Cl | 5-F |
| I.1.1691 | pyridin-3-yl | 4-I | 2-Cl | H |
| I.1.1692 | pyridin-3-yl | 4-I | 5-Cl | H |
| I.1.1693 | pyridin-3-yl | 4-I | 6-Cl | H |
| I.1.1694 | pyridin-3-yl | 4-I | 2-F | H |
| I.1.1695 | pyridin-3-yl | 4-I | 5-F | H |
| I.1.1696 | pyridin-3-yl | 4-I | 6-F | H |
| I.1.1697 | pyridin-3-yl | 4-I | 2-Br | H |
| I.1.1698 | pyridin-3-yl | 4-I | 5-Br | H |
| I.1.1699 | pyridin-3-yl | 4-I | 6-Br | H |
| I.1.1700 | pyridin-3-yl | 4-I | 2-CH₃ | H |
| I.1.1701 | pyridin-3-yl | 4-I | 5-CH₃ | H |
| I.1.1702 | pyridin-3-yl | 4-I | 6-CH₃ | H |
| I.1.1703 | pyridin-3-yl | 4-I | 2-CF₃ | H |
| I.1.1704 | pyridin-3-yl | 4-I | 5-CF₃ | H |
| I.1.1705 | pyridin-3-yl | 4-I | 6-CF₃ | H |
| I.1.1706 | pyridin-3-yl | 4-I | 2-vinyl | H |
| I.1.1707 | pyridin-3-yl | 4-I | 5-vinyl | H |
| I.1.1708 | pyridin-3-yl | 4-I | 6-vinyl | H |
| I.1.1709 | pyridin-3-yl | 4-I | 2-Cl | 6-F |
| I.1.1710 | pyridin-3-yl | 4-I | 5-Cl | 6-F |
| I.1.1711 | pyridin-3-yl | 4-COOH | 2-Cl | 5-F |
| I.1.1712 | pyridin-3-yl | 4-COOH | 2-Cl | H |
| I.1.1713 | pyridin-3-yl | 4-COOH | 5-Cl | H |
| I.1.1714 | pyridin-3-yl | 4-COOH | 6-Cl | H |
| I.1.1715 | pyridin-3-yl | 4-COOH | 2-F | H |
| I.1.1716 | pyridin-3-yl | 4-COOH | 5-F | H |
| I.1.1717 | pyridin-3-yl | 4-COOH | 6-F | H |
| I.1.1718 | pyridin-3-yl | 4-COOH | 2-Br | H |
| I.1.1719 | pyridin-3-yl | 4-COOH | 5-Br | H |
| I.1.1720 | pyridin-3-yl | 4-COOH | 6-Br | H |
| I.1.1721 | pyridin-3-yl | 4-COOH | 2-CH₃ | H |
| I.1.1722 | pyridin-3-yl | 4-COOH | 5-CH₃ | H |
| I.1.1723 | pyridin-3-yl | 4-COOH | 6-CH₃ | H |
| I.1.1724 | pyridin-3-yl | 4-COOH | 2-CF₃ | H |
| I.1.1725 | pyridin-3-yl | 4-COOH | 5-CF₃ | H |
| I.1.1726 | pyridin-3-yl | 4-COOH | 6-CF₃ | H |
| I.1.1727 | pyridin-3-yl | 4-COOH | 2-vinyl | H |
| I.1.1728 | pyridin-3-yl | 4-COOH | 5-vinyl | H |
| I.1.1729 | pyridin-3-yl | 4-COOH | 6-vinyl | H |
| I.1.1730 | pyridin-3-yl | 4-COOH | 2-Cl | 6-F |
| I.1.1731 | pyridin-3-yl | 4-COOH | 5-Cl | 6-F |
| I.1.1732 | pyridin-3-yl | 4-COOCH₃ | 2-Cl | 5-F |
| I.1.1733 | pyridin-3-yl | 4-COOCH₃ | 2-Cl | H |
| I.1.1734 | pyridin-3-yl | 4-COOCH₃ | 5-Cl | H |
| I.1.1735 | pyridin-3-yl | 4-COOCH₃ | 6-Cl | H |
| I.1.1736 | pyridin-3-yl | 4-COOCH₃ | 2-F | H |
| I.1.1737 | pyridin-3-yl | 4-COOCH₃ | 5-F | H |
| I.1.1738 | pyridin-3-yl | 4-COOCH₃ | 6-F | H |
| I.1.1739 | pyridin-3-yl | 4-COOCH₃ | 2-Br | H |
| I.1.1740 | pyridin-3-yl | 4-COOCH₃ | 5-Br | H |
| I.1.1741 | pyridin-3-yl | 4-COOCH₃ | 6-Br | H |
| I.1.1742 | pyridin-3-yl | 4-COOCH₃ | 2-CH₃ | H |
| I.1.1743 | pyridin-3-yl | 4-COOCH₃ | 5-CH₃ | H |
| I.1.1744 | pyridin-3-yl | 4-COOCH₃ | 6-CH₃ | H |
| I.1.1745 | pyridin-3-yl | 4-COOCH₃ | 2-CF₃ | H |
| I.1.1746 | pyridin-3-yl | 4-COOCH₃ | 5-CF₃ | H |
| I.1.1747 | pyridin-3-yl | 4-COOCH₃ | 6-CF₃ | H |
| I.1.1748 | pyridin-3-yl | 4-COOCH₃ | 2-vinyl | H |
| I.1.1749 | pyridin-3-yl | 4-COOCH₃ | 5-vinyl | H |
| I.1.1750 | pyridin-3-yl | 4-COOCH₃ | 6-vinyl | H |
| I.1.1751 | pyridin-3-yl | 4-COOCH₃ | 2-Cl | 6-F |
| I.1.1752 | pyridin-3-yl | 4-COOCH₃ | 5-Cl | 6-F |
| I.1.1753 | pyridin-3-yl | 4-COOCH₂CH₃ | 2-Cl | 5-F |
| I.1.1754 | pyridin-3-yl | 4-COOCH₂CH₃ | 2-Cl | H |
| I.1.1755 | pyridin-3-yl | 4-COOCH₂CH₃ | 5-Cl | H |
| I.1.1756 | pyridin-3-yl | 4-COOCH₂CH₃ | 6-Cl | H |
| I.1.1757 | pyridin-3-yl | 4-COOCH₂CH₃ | 2-F | H |
| I.1.1758 | pyridin-3-yl | 4-COOCH₂CH₃ | 5-F | H |
| I.1.1759 | pyridin-3-yl | 4-COOCH₂CH₃ | 6-F | H |
| I.1.1760 | pyridin-3-yl | 4-COOCH₂CH₃ | 2-Br | H |
| I.1.1761 | pyridin-3-yl | 4-COOCH₂CH₃ | 5-Br | H |
| I.1.1762 | pyridin-3-yl | 4-COOCH₂CH₃ | 6-Br | H |
| I.1.1763 | pyridin-3-yl | 4-COOCH₂CH₃ | 2-CH₃ | H |
| I.1.1764 | pyridin-3-yl | 4-COOCH₂CH₃ | 5-CH₃ | H |
| I.1.1765 | pyridin-3-yl | 4-COOCH₂CH₃ | 6-CH₃ | H |
| I.1.1766 | pyridin-3-yl | 4-COOCH₂CH₃ | 2-CF₃ | H |
| I.1.1767 | pyridin-3-yl | 4-COOCH₂CH₃ | 5-CF₃ | H |
| I.1.1768 | pyridin-3-yl | 4-COOCH₂CH₃ | 6-CF₃ | H |
| I.1.1769 | pyridin-3-yl | 4-COOCH₂CH₃ | 2-vinyl | H |
| I.1.1770 | pyridin-3-yl | 4-COOCH₂CH₃ | 5-vinyl | H |
| I.1.1771 | pyridin-3-yl | 4-COOCH₂CH₃ | 6-vinyl | H |
| I.1.1772 | pyridin-3-yl | 4-COOCH₂CH₃ | 2-Cl | 6-F |
| I.1.1773 | pyridin-3-yl | 4-COOCH₂CH₃ | 5-Cl | 6-F |
| I.1.1774 | pyridin-3-yl | 4-CONHCH₃ | 2-Cl | 5-F |
| I.1.1775 | pyridin-3-yl | 4-CONHCH₃ | 2-Cl | H |
| I.1.1776 | pyridin-3-yl | 4-CONHCH₃ | 5-Cl | H |
| I.1.1777 | pyridin-3-yl | 4-CONHCH₃ | 6-Cl | H |
| I.1.1778 | pyridin-3-yl | 4-CONHCH₃ | 2-F | H |
| I.1.1779 | pyridin-3-yl | 4-CONHCH₃ | 5-F | H |
| I.1.1780 | pyridin-3-yl | 4-CONHCH₃ | 6-F | H |
| I.1.1781 | pyridin-3-yl | 4-CONHCH₃ | 2-Br | H |
| I.1.1782 | pyridin-3-yl | 4-CONHCH₃ | 5-Br | H |
| I.1.1783 | pyridin-3-yl | 4-CONHCH₃ | 6-Br | H |
| I.1.1784 | pyridin-3-yl | 4-CONHCH₃ | 2-CH₃ | H |
| I.1.1785 | pyridin-3-yl | 4-CONHCH₃ | 5-CH₃ | H |
| I.1.1786 | pyridin-3-yl | 4-CONHCH₃ | 6-CH₃ | H |
| I.1.1787 | pyridin-3-yl | 4-CONHCH₃ | 2-CF₃ | H |
| I.1.1788 | pyridin-3-yl | 4-CONHCH₃ | 5-CF₃ | H |
| I.1.1789 | pyridin-3-yl | 4-CONHCH₃ | 6-CF₃ | H |
| I.1.1790 | pyridin-3-yl | 4-CONHCH₃ | 2-vinyl | H |
| I.1.1791 | pyridin-3-yl | 4-CONHCH₃ | 5-vinyl | H |
| I.1.1792 | pyridin-3-yl | 4-CONHCH₃ | 6-vinyl | H |
| I.1.1793 | pyridin-3-yl | 4-CONHCH₃ | 2-Cl | 6-F |
| I.1.1794 | pyridin-3-yl | 4-CONHCH₃ | 5-Cl | 6-F |
| I.1.1795 | pyridin-3-yl | 4-CON(CH₃)₂ | 2-Cl | 5-F |
| I.1.1796 | pyridin-3-yl | 4-CON(CH₃)₂ | 2-Cl | H |
| I.1.1797 | pyridin-3-yl | 4-CON(CH₃)₂ | 5-Cl | H |
| I.1.1798 | pyridin-3-yl | 4-CON(CH₃)₂ | 6-Cl | H |
| I.1.1799 | pyridin-3-yl | 4-CON(CH₃)₂ | 2-F | H |
| I.1.1800 | pyridin-3-yl | 4-CON(CH₃)₂ | 5-F | H |
| I.1.1801 | pyridin-3-yl | 4-CON(CH₃)₂ | 6-F | H |
| I.1.1802 | pyridin-3-yl | 4-CON(CH₃)₂ | 2-Br | H |
| I.1.1803 | pyridin-3-yl | 4-CON(CH₃)₂ | 5-Br | H |
| I.1.1804 | pyridin-3-yl | 4-CON(CH₃)₂ | 6-Br | H |
| I.1.1805 | pyridin-3-yl | 4-CON(CH₃)₂ | 2-CH₃ | H |
| I.1.1806 | pyridin-3-yl | 4-CON(CH₃)₂ | 5-CH₃ | H |
| I.1.1807 | pyridin-3-yl | 4-CON(CH₃)₂ | 6-CH₃ | H |
| I.1.1808 | pyridin-3-yl | 4-CON(CH₃)₂ | 2-CF₃ | H |
| I.1.1809 | pyridin-3-yl | 4-CON(CH₃)₂ | 5-CF₃ | H |
| I.1.1810 | pyridin-3-yl | 4-CON(CH₃)₂ | 6-CF₃ | H |
| I.1.1811 | pyridin-3-yl | 4-CON(CH₃)₂ | 2-vinyl | H |
| I.1.1812 | pyridin-3-yl | 4-CON(CH₃)₂ | 5-vinyl | H |
| I.1.1813 | pyridin-3-yl | 4-CON(CH₃)₂ | 6-vinyl | H |
| I.1.1814 | pyridin-3-yl | 4-CON(CH₃)₂ | 2-Cl | 6-F |
| I.1.1815 | pyridin-3-yl | 4-CON(CH₃)₂ | 5-Cl | 6-F |
| I.1.1816 | pyridin-4-yl | 3-NO₂ | 2-Cl | 5-F |
| I.1.1817 | pyridin-4-yl | 3-NO₂ | 2-Cl | H |
| I.1.1818 | pyridin-4-yl | 3-NO₂ | 5-Cl | H |
| I.1.1819 | pyridin-4-yl | 3-NO₂ | 6-Cl | H |
| I.1.1820 | pyridin-4-yl | 3-NO₂ | 2-F | H |
| I.1.1821 | pyridin-4-yl | 3-NO₂ | 5-F | H |
| I.1.1822 | pyridin-4-yl | 3-NO₂ | 6-F | H |
| I.1.1823 | pyridin-4-yl | 3-NO₂ | 2-Br | H |
| I.1.1824 | pyridin-4-yl | 3-NO₂ | 5-Br | H |
| I.1.1825 | pyridin-4-yl | 3-NO₂ | 6-Br | H |
| I.1.1826 | pyridin-4-yl | 3-NO₂ | 2-CH₃ | H |
| I.1.1827 | pyridin-4-yl | 3-NO₂ | 5-CH₃ | H |
| I.1.1828 | pyridin-4-yl | 3-NO₂ | 6-CH₃ | H |
| I.1.1829 | pyridin-4-yl | 3-NO₂ | 2-CF₃ | H |
| I.1.1830 | pyridin-4-yl | 3-NO₂ | 5-CF₃ | H |
| I.1.1831 | pyridin-4-yl | 3-NO₂ | 6-CF₃ | H |
| I.1.1832 | pyridin-4-yl | 3-NO₂ | 2-vinyl | H |
| I.1.1833 | pyridin-4-yl | 3-NO₂ | 5-vinyl | H |
| I.1.1834 | pyridin-4-yl | 3-NO₂ | 6-vinyl | H |
| I.1.1835 | pyridin-4-yl | 3-NO₂ | 2-Cl | 6-F |
| I.1.1836 | pyridin-4-yl | 3-NO₂ | 5-Cl | 6-F |
| I.1.1837 | pyridin-4-yl | 3-CN | 2-Cl | 5-F |
| I.1.1838 | pyridin-4-yl | 3-CN | 2-Cl | H |
| I.1.1839 | pyridin-4-yl | 3-CN | 5-Cl | H |
| I.1.1840 | pyridin-4-yl | 3-CN | 6-Cl | H |
| I.1.1841 | pyridin-4-yl | 3-CN | 2-F | H |
| I.1.1842 | pyridin-4-yl | 3-CN | 5-F | H |
| I.1.1843 | pyridin-4-yl | 3-CN | 6-F | H |
| I.1.1844 | pyridin-4-yl | 3-CN | 2-Br | H |
| I.1.1845 | pyridin-4-yl | 3-CN | 5-Br | H |
| I.1.1846 | pyridin-4-yl | 3-CN | 6-Br | H |
| I.1.1847 | pyridin-4-yl | 3-CN | 2-CH₃ | H |
| I.1.1848 | pyridin-4-yl | 3-CN | 5-CH₃ | H |
| I.1.1849 | pyridin-4-yl | 3-CN | 6-CH₃ | H |
| I.1.1850 | pyridin-4-yl | 3-CN | 2-CF₃ | H |
| I.1.1851 | pyridin-4-yl | 3-CN | 5-CF₃ | H |
| I.1.1852 | pyridin-4-yl | 3-CN | 6-CF₃ | H |
| I.1.1853 | pyridin-4-yl | 3-CN | 2-vinyl | H |
| I.1.1854 | pyridin-4-yl | 3-CN | 5-vinyl | H |
| I.1.1855 | pyridin-4-yl | 3-CN | 6-vinyl | H |
| I.1.1856 | pyridin-4-yl | 3-CN | 2-Cl | 6-F |
| I.1.1857 | pyridin-4-yl | 3-CN | 5-Cl | 6-F |
| I.1.1858 | pyridin-4-yl | 3-Br | 2-Cl | 5-F |
| I.1.1859 | pyridin-4-yl | 3-Br | 2-Cl | H |
| I.1.1860 | pyridin-4-yl | 3-Br | 5-Cl | H |
| I.1.1861 | pyridin-4-yl | 3-Br | 6-Cl | H |
| I.1.1862 | pyridin-4-yl | 3-Br | 2-F | H |
| I.1.1863 | pyridin-4-yl | 3-Br | 5-F | H |
| I.1.1864 | pyridin-4-yl | 3-Br | 6-F | H |
| I.1.1865 | pyridin-4-yl | 3-Br | 2-Br | H |
| I.1.1866 | pyridin-4-yl | 3-Br | 5-Br | H |
| I.1.1867 | pyridin-4-yl | 3-Br | 6-Br | H |
| I.1.1868 | pyridin-4-yl | 3-Br | 2-CH₃ | H |
| I.1.1869 | pyridin-4-yl | 3-Br | 5-CH₃ | H |
| I.1.1870 | pyridin-4-yl | 3-Br | 6-CH₃ | H |
| I.1.1871 | pyridin-4-yl | 3-Br | 2-CF₃ | H |
| I.1.1872 | pyridin-4-yl | 3-Br | 5-CF₃ | H |
| I.1.1873 | pyridin-4-yl | 3-Br | 6-CF₃ | H |
| I.1.1874 | pyridin-4-yl | 3-Br | 2-vinyl | H |
| I.1.1875 | pyridin-4-yl | 3-Br | 5-vinyl | H |
| I.1.1876 | pyridin-4-yl | 3-Br | 6-vinyl | H |
| I.1.1877 | pyridin-4-yl | 3-Br | 2-Cl | 6-F |
| I.1.1878 | pyridin-4-yl | 3-Br | 5-Cl | 6-F |
| I.1.1879 | pyridin-4-yl | 3-Cl | 2-Cl | 5-F |
| I.1.1880 | pyridin-4-yl | 3-Cl | 2-Cl | H |
| I.1.1881 | pyridin-4-yl | 3-Cl | 5-Cl | H |
| I.1.1882 | pyridin-4-yl | 3-Cl | 6-Cl | H |
| I.1.1883 | pyridin-4-yl | 3-Cl | 2-F | H |
| I.1.1884 | pyridin-4-yl | 3-Cl | 5-F | H |
| I.1.1885 | pyridin-4-yl | 3-Cl | 6-F | H |
| I.1.1886 | pyridin-4-yl | 3-Cl | 2-Br | H |
| I.1.1887 | pyridin-4-yl | 3-Cl | 5-Br | H |
| I.1.1888 | pyridin-4-yl | 3-Cl | 6-Br | H |
| I.1.1889 | pyridin-4-yl | 3-Cl | 2-CH₃ | H |
| I.1.1890 | pyridin-4-yl | 3-Cl | 5-CH₃ | H |
| I.1.1891 | pyridin-4-yl | 3-Cl | 6-CH₃ | H |
| I.1.1892 | pyridin-4-yl | 3-Cl | 2-CF₃ | H |
| I.1.1893 | pyridin-4-yl | 3-Cl | 5-CF₃ | H |
| I.1.1894 | pyridin-4-yl | 3-Cl | 6-CF₃ | H |
| I.1.1895 | pyridin-4-yl | 3-Cl | 2-vinyl | H |
| I.1.1896 | pyridin-4-yl | 3-Cl | 5-vinyl | H |
| I.1.1897 | pyridin-4-yl | 3-Cl | 6-vinyl | H |
| I.1.1898 | pyridin-4-yl | 3-Cl | 2-Cl | 6-F |
| I.1.1899 | pyridin-4-yl | 3-Cl | 5-Cl | 6-F |
| I.1.1900 | pyridin-4-yl | 3-ethynyl | 2-Cl | 5-F |
| I.1.1901 | pyridin-4-yl | 3-ethynyl | 2-Cl | H |
| I.1.1902 | pyridin-4-yl | 3-ethynyl | 5-Cl | H |
| I.1.1903 | pyridin-4-yl | 3-ethynyl | 6-Cl | H |
| I.1.1904 | pyridin-4-yl | 3-ethynyl | 2-F | H |
| I.1.1905 | pyridin-4-yl | 3-ethynyl | 5-F | H |
| I.1.1906 | pyridin-4-yl | 3-ethynyl | 6-F | H |
| I.1.1907 | pyridin-4-yl | 3-ethynyl | 2-Br | H |
| I.1.1908 | pyridin-4-yl | 3-ethynyl | 5-Br | H |
| I.1.1909 | pyridin-4-yl | 3-ethynyl | 6-Br | H |
| I.1.1910 | pyridin-4-yl | 3-ethynyl | 2-CH₃ | H |
| I.1.1911 | pyridin-4-yl | 3-ethynyl | 5-CH₃ | H |
| I.1.1912 | pyridin-4-yl | 3-ethynyl | 6-CH₃ | H |
| I.1.1913 | pyridin-4-yl | 3-ethynyl | 2-CF₃ | H |
| I.1.1914 | pyridin-4-yl | 3-ethynyl | 5-CF₃ | H |
| I.1.1915 | pyridin-4-yl | 3-ethynyl | 6-CF₃ | H |
| I.1.1916 | pyridin-4-yl | 3-ethynyl | 2-vinyl | H |
| I.1.1917 | pyridin-4-yl | 3-ethynyl | 5-vinyl | H |
| I.1.1918 | pyridin-4-yl | 3-ethynyl | 6-vinyl | H |
| I.1.1919 | pyridin-4-yl | 3-ethynyl | 2-Cl | 6-F |
| I.1.1920 | pyridin-4-yl | 3-ethynyl | 5-Cl | 6-F |
| I.1.1921 | pyridin-4-yl | 3-I | 2-Cl | 5-F |
| I.1.1922 | pyridin-4-yl | 3-I | 2-Cl | H |
| I.1.1923 | pyridin-4-yl | 3-I | 5-Cl | H |
| I.1.1924 | pyridin-4-yl | 3-I | 6-Cl | H |
| I.1.1925 | pyridin-4-yl | 3-I | 2-F | H |
| I.1.1926 | pyridin-4-yl | 3-I | 5-F | H |
| I.1.1927 | pyridin-4-yl | 3-I | 6-F | H |
| I.1.1928 | pyridin-4-yl | 3-I | 2-Br | H |
| I.1.1929 | pyridin-4-yl | 3-I | 5-Br | H |
| I.1.1930 | pyridin-4-yl | 3-I | 6-Br | H |
| I.1.1931 | pyridin-4-yl | 3-I | 2-CH₃ | H |
| I.1.1932 | pyridin-4-yl | 3-I | 5-CH₃ | H |
| I.1.1933 | pyridin-4-yl | 3-I | 6-CH₃ | H |
| I.1.1934 | pyridin-4-yl | 3-I | 2-CF₃ | H |
| I.1.1935 | pyridin-4-yl | 3-I | 5-CF₃ | H |
| I.1.1936 | pyridin-4-yl | 3-I | 6-CF₃ | H |
| I.1.1937 | pyridin-4-yl | 3-I | 2-vinyl | H |
| I.1.1938 | pyridin-4-yl | 3-I | 5-vinyl | H |
| I.1.1939 | pyridin-4-yl | 3-I | 6-vinyl | H |
| I.1.1940 | pyridin-4-yl | 3-I | 2-Cl | 6-F |
| I.1.1941 | pyridin-4-yl | 3-I | 5-Cl | 6-F |
| I.1.1942 | pyridin-4-yl | 3-COOH | 2-Cl | 5-F |
| I.1.1943 | pyridin-4-yl | 3-COOH | 2-Cl | H |
| I.1.1944 | pyridin-4-yl | 3-COOH | 5-Cl | H |
| I.1.1945 | pyridin-4-yl | 3-COOH | 6-Cl | H |
| I.1.1946 | pyridin-4-yl | 3-COOH | 2-F | H |
| I.1.1947 | pyridin-4-yl | 3-COOH | 5-F | H |
| I.1.1948 | pyridin-4-yl | 3-COOH | 6-F | H |
| I.1.1949 | pyridin-4-yl | 3-COOH | 2-Br | H |
| I.1.1950 | pyridin-4-yl | 3-COOH | 5-Br | H |
| I.1.1951 | pyridin-4-yl | 3-COOH | 6-Br | H |
| I.1.1952 | pyridin-4-yl | 3-COOH | 2-CH₃ | H |
| I.1.1953 | pyridin-4-yl | 3-COOH | 5-CH₃ | H |
| I.1.1954 | pyridin-4-yl | 3-COOH | 6-CH₃ | H |
| I.1.1955 | pyridin-4-yl | 3-COOH | 2-CF₃ | H |
| I.1.1956 | pyridin-4-yl | 3-COOH | 5-CF₃ | H |
| I.1.1957 | pyridin-4-yl | 3-COOH | 6-CF₃ | H |
| I.1.1958 | pyridin-4-yl | 3-COOH | 2-vinyl | H |
| I.1.1959 | pyridin-4-yl | 3-COOH | 5-vinyl | H |
| I.1.1960 | pyridin-4-yl | 3-COOH | 6-vinyl | H |
| I.1.1961 | pyridin-4-yl | 3-COOH | 2-Cl | 6-F |
| I.1.1962 | pyridin-4-yl | 3-COOH | 5-Cl | 6-F |
| I.1.1963 | pyridin-4-yl | 3-COOCH₃ | 2-Cl | 5-F |
| I.1.1964 | pyridin-4-yl | 3-COOCH₃ | 2-Cl | H |
| I.1.1965 | pyridin-4-yl | 3-COOCH₃ | 5-Cl | H |
| I.1.1966 | pyridin-4-yl | 3-COOCH₃ | 6-Cl | H |
| I.1.1967 | pyridin-4-yl | 3-COOCH₃ | 2-F | H |
| I.1.1968 | pyridin-4-yl | 3-COOCH₃ | 5-F | H |
| I.1.1969 | pyridin-4-yl | 3-COOCH₃ | 6-F | H |
| I.1.1970 | pyridin-4-yl | 3-COOCH₃ | 2-Br | H |
| I.1.1971 | pyridin-4-yl | 3-COOCH₃ | 5-Br | H |
| I.1.1972 | pyridin-4-yl | 3-COOCH₃ | 6-Br | H |
| I.1.1973 | pyridin-4-yl | 3-COOCH₃ | 2-CH₃ | H |
| I.1.1974 | pyridin-4-yl | 3-COOCH₃ | 5-CH₃ | H |
| I.1.1975 | pyridin-4-yl | 3-COOCH₃ | 6-CH₃ | H |
| I.1.1976 | pyridin-4-yl | 3-COOCH₃ | 2-CF₃ | H |
| I.1.1977 | pyridin-4-yl | 3-COOCH₃ | 5-CF₃ | H |
| I.1.1978 | pyridin-4-yl | 3-COOCH₃ | 6-CF₃ | H |
| I.1.1979 | pyridin-4-yl | 3-COOCH₃ | 2-vinyl | H |
| I.1.1980 | pyridin-4-yl | 3-COOCH₃ | 5-vinyl | H |
| I.1.1981 | pyridin-4-yl | 3-COOCH₃ | 6-vinyl | H |
| I.1.1982 | pyridin-4-yl | 3-COOCH₃ | 2-Cl | 6-F |
| I.1.1983 | pyridin-4-yl | 3-COOCH₃ | 5-Cl | 6-F |
| I.1.1984 | pyridin-4-yl | 3-COOCH₂CH₃ | 2-Cl | 5-F |
| I.1.1985 | pyridin-4-yl | 3-COOCH₂CH₃ | 2-Cl | H |
| I.1.1986 | pyridin-4-yl | 3-COOCH₂CH₃ | 5-Cl | H |
| I.1.1987 | pyridin-4-yl | 3-COOCH₂CH₃ | 6-Cl | H |
| I.1.1988 | pyridin-4-yl | 3-COOCH₂CH₃ | 2-F | H |
| I.1.1989 | pyridin-4-yl | 3-COOCH₂CH₃ | 5-F | H |
| I.1.1990 | pyridin-4-yl | 3-COOCH₂CH₃ | 6-F | H |
| I.1.1991 | pyridin-4-yl | 3-COOCH₂CH₃ | 2-Br | H |
| I.1.1992 | pyridin-4-yl | 3-COOCH₂CH₃ | 5-Br | H |
| I.1.1993 | pyridin-4-yl | 3-COOCH₂CH₃ | 6-Br | H |
| I.1.1994 | pyridin-4-yl | 3-COOCH₂CH₃ | 2-CH₃ | H |
| I.1.1995 | pyridin-4-yl | 3-COOCH₂CH₃ | 5-CH₃ | H |
| I.1.1996 | pyridin-4-yl | 3-COOCH₂CH₃ | 6-CH₃ | H |
| I.1.1997 | pyridin-4-yl | 3-COOCH₂CH₃ | 2-CF₃ | H |
| I.1.1998 | pyridin-4-yl | 3-COOCH₂CH₃ | 5-CF₃ | H |
| I.1.1999 | pyridin-4-yl | 3-COOCH₂CH₃ | 6-CF₃ | H |
| I.1.2000 | pyridin-4-yl | 3-COOCH₂CH₃ | 2-vinyl | H |
| I.1.2001 | pyridin-4-yl | 3-COOCH₂CH₃ | 5-vinyl | H |
| I.1.2002 | pyridin-4-yl | 3-COOCH₂CH₃ | 6-vinyl | H |
| I.1.2003 | pyridin-4-yl | 3-COOCH₂CH₃ | 2-Cl | 6-F |
| I.1.2004 | pyridin-4-yl | 3-COOCH₂CH₃ | 5-Cl | 6-F |
| I.1.2005 | pyridin-4-yl | 3-CONHCH₃ | 2-Cl | 5-F |
| I.1.2006 | pyridin-4-yl | 3-CONHCH₃ | 2-Cl | H |
| I.1.2007 | pyridin-4-yl | 3-CONHCH₃ | 5-Cl | H |
| I.1.2008 | pyridin-4-yl | 3-CONHCH₃ | 6-Cl | H |
| I.1.2009 | pyridin-4-yl | 3-CONHCH₃ | 2-F | H |
| I.1.2010 | pyridin-4-yl | 3-CONHCH₃ | 5-F | H |
| I.1.2011 | pyridin-4-yl | 3-CONHCH₃ | 6-F | H |
| I.1.2012 | pyridin-4-yl | 3-CONHCH₃ | 2-Br | H |
| I.1.2013 | pyridin-4-yl | 3-CONHCH₃ | 5-Br | H |
| I.1.2014 | pyridin-4-yl | 3-CONHCH₃ | 6-Br | H |
| I.1.2015 | pyridin-4-yl | 3-CONHCH₃ | 2-CH₃ | H |
| I.1.2016 | pyridin-4-yl | 3-CONHCH₃ | 5-CH₃ | H |
| I.1.2017 | pyridin-4-yl | 3-CONHCH₃ | 6-CH₃ | H |
| I.1.2018 | pyridin-4-yl | 3-CONHCH₃ | 2-CF₃ | H |
| I.1.2019 | pyridin-4-yl | 3-CONHCH₃ | 5-CF₃ | H |
| I.1.2020 | pyridin-4-yl | 3-CONHCH₃ | 6-CF₃ | H |
| I.1.2021 | pyridin-4-yl | 3-CONHCH₃ | 2-vinyl | H |
| I.1.2022 | pyridin-4-yl | 3-CONHCH₃ | 5-vinyl | H |
| I.1.2023 | pyridin-4-yl | 3-CONHCH₃ | 6-vinyl | H |
| I.1.2024 | pyridin-4-yl | 3-CONHCH₃ | 2-Cl | 6-F |
| I.1.2025 | pyridin-4-yl | 3-CONHCH₃ | 5-Cl | 6-F |
| I.1.2026 | pyridin-4-yl | 3-CON(CH₃)₂ | 2-Cl | 5-F |
| I.1.2027 | pyridin-4-yl | 3-CON(CH₃)₂ | 2-Cl | H |
| I.1.2028 | pyridin-4-yl | 3-CON(CH₃)₂ | 5-Cl | H |
| I.1.2029 | pyridin-4-yl | 3-CON(CH₃)₂ | 6-Cl | H |
| I.1.2030 | pyridin-4-yl | 3-CON(CH₃)₂ | 2-F | H |
| I.1.2031 | pyridin-4-yl | 3-CON(CH₃)₂ | 5-F | H |
| I.1.2032 | pyridin-4-yl | 3-CON(CH₃)₂ | 6-F | H |
| I.1.2033 | pyridin-4-yl | 3-CON(CH₃)₂ | 2-Br | H |
| I.1.2034 | pyridin-4-yl | 3-CON(CH₃)₂ | 5-Br | H |
| I.1.2035 | pyridin-4-yl | 3-CON(CH₃)₂ | 6-Br | H |
| I.1.2036 | pyridin-4-yl | 3-CON(CH₃)₂ | 2-CH₃ | H |
| I.1.2037 | pyridin-4-yl | 3-CON(CH₃)₂ | 5-CH₃ | H |
| I.1.2038 | pyridin-4-yl | 3-CON(CH₃)₂ | 6-CH₃ | H |
| I.1.2039 | pyridin-4-yl | 3-CON(CH₃)₂ | 2-CF₃ | H |
| I.1.2040 | pyridin-4-yl | 3-CON(CH₃)₂ | 5-CF₃ | H |
| I.1.2041 | pyridin-4-yl | 3-CON(CH₃)₂ | 6-CF₃ | H |
| I.1.2042 | pyridin-4-yl | 3-CON(CH₃)₂ | 2-vinyl | H |
| I.1.2043 | pyridin-4-yl | 3-CON(CH₃)₂ | 5-vinyl | H |
| I.1.2044 | pyridin-4-yl | 3-CON(CH₃)₂ | 6-vinyl | H |
| I.1.2045 | pyridin-4-yl | 3-CON(CH₃)₂ | 2-Cl | 6-F |
| I.1.2046 | pyridin-4-yl | 3-CON(CH₃)₂ | 5-Cl | 6-F |
| I.1.2047 | furan-2-yl | 3-NO₂ | 4-Cl | 5-F |
| I.1.2048 | furan-2-yl | 3-NO₂ | 4-Cl | H |
| I.1.2049 | furan-2-yl | 3-NO₂ | 5-Cl | H |
| I.1.2050 | furan-2-yl | 3-NO₂ | 4-F | H |
| I.1.2051 | furan-2-yl | 3-NO₂ | 5-F | H |
| I.1.2052 | furan-2-yl | 3-NO₂ | 4-CH₃ | H |
| I.1.2053 | furan-2-yl | 3-NO₂ | 5-CH₃ | H |
| I.1.2054 | furan-2-yl | 3-NO₂ | 4-CN | H |
| I.1.2055 | furan-2-yl | 3-NO₂ | 5-CN | H |
| I.1.2056 | furan-2-yl | 3-NO₂ | 4-CF₃ | H |
| I.1.2057 | furan-2-yl | 3-NO₂ | 5-CF₃ | H |
| I.1.2058 | furan-2-yl | 3-CN | 4-Cl | 5-F |
| I.1.2059 | furan-2-yl | 3-CN | 4-Cl | H |
| I.1.2060 | furan-2-yl | 3-CN | 5-Cl | H |
| I.1.2061 | furan-2-yl | 3-CN | 4-F | H |
| I.1.2062 | furan-2-yl | 3-CN | 5-F | H |
| I.1.2063 | furan-2-yl | 3-CN | 4-CH₃ | H |
| I.1.2064 | furan-2-yl | 3-CN | 5-CH₃ | H |
| I.1.2065 | furan-2-yl | 3-CN | 4-CN | H |
| I.1.2066 | furan-2-yl | 3-CN | 5-CN | H |
| I.1.2067 | furan-2-yl | 3-CN | 4-CF₃ | H |
| I.1.2068 | furan-2-yl | 3-CN | 5-CF₃ | H |
| I.1.2069 | furan-2-yl | 3-COOH | 4-Cl | 5-F |
| I.1.2070 | furan-2-yl | 3-COOH | 4-Cl | H |
| I.1.2071 | furan-2-yl | 3-COOH | 5-Cl | H |
| I.1.2072 | furan-2-yl | 3-COOH | 4-F | H |
| I.1.2073 | furan-2-yl | 3-COOH | 5-F | H |
| I.1.2074 | furan-2-yl | 3-COOH | 4-CH₃ | H |
| I.1.2075 | furan-2-yl | 3-COOH | 5-CH₃ | H |
| I.1.2076 | furan-2-yl | 3-COOH | 4-CN | H |
| I.1.2077 | furan-2-yl | 3-COOH | 5-CN | H |
| I.1.2078 | furan-2-yl | 3-COOH | 4-CF₃ | H |
| I.1.2079 | furan-2-yl | 3-COOH | 5-CF₃ | H |
| I.1.2080 | furan-2-yl | 3-COOCH₃ | 4-Cl | 5-F |
| I.1.2081 | furan-2-yl | 3-COOCH₃ | 4-Cl | H |
| I.1.2082 | furan-2-yl | 3-COOCH₃ | 5-Cl | H |
| I.1.2083 | furan-2-yl | 3-COOCH₃ | 4-F | H |
| I.1.2084 | furan-2-yl | 3-COOCH₃ | 5-F | H |
| I.1.2085 | furan-2-yl | 3-COOCH₃ | 4-CH₃ | H |
| I.1.2086 | furan-2-yl | 3-COOCH₃ | 5-CH₃ | H |
| I.1.2087 | furan-2-yl | 3-COOCH₃ | 4-CN | H |
| I.1.2088 | furan-2-yl | 3-COOCH₃ | 5-CN | H |
| I.1.2089 | furan-2-yl | 3-COOCH₃ | 4-CF₃ | H |
| I.1.2090 | furan-2-yl | 3-COOCH₃ | 5-CF₃ | H |
| I.1.2091 | furan-2-yl | 3-COOCH₂CH₃ | 4-Cl | 5-F |
| I.1.2092 | furan-2-yl | 3-COOCH₂CH₃ | 4-Cl | H |
| I.1.2093 | furan-2-yl | 3-COOCH₂CH₃ | 5-Cl | H |
| I.1.2094 | furan-2-yl | 3-COOCH₂CH₃ | 4-F | H |
| I.1.2095 | furan-2-yl | 3-COOCH₂CH₃ | 5-F | H |
| I.1.2096 | furan-2-yl | 3-COOCH₂CH₃ | 4-CH₃ | H |
| I.1.2097 | furan-2-yl | 3-COOCH₂CH₃ | 5-CH₃ | H |
| I.1.2098 | furan-2-yl | 3-COOCH₂CH₃ | 4-CN | H |
| I.1.2099 | furan-2-yl | 3-COOCH₂CH₃ | 5-CN | H |
| I.1.2100 | furan-2-yl | 3-COOCH₂CH₃ | 4-CF₃ | H |
| I.1.2101 | furan-2-yl | 3-COOCH₂CH₃ | 5-CF₃ | H |
| I.1.2102 | furan-2-yl | 3-ethynyl | 4-Cl | 5-F |
| I.1.2103 | furan-2-yl | 3-ethynyl | 4-Cl | H |
| I.1.2104 | furan-2-yl | 3-ethynyl | 5-Cl | H |
| I.1.2105 | furan-2-yl | 3-ethynyl | 4-F | H |
| I.1.2106 | furan-2-yl | 3-ethynyl | 5-F | H |
| I.1.2107 | furan-2-yl | 3-ethynyl | 4-CH₃ | H |
| I.1.2108 | furan-2-yl | 3-ethynyl | 5-CH₃ | H |
| I.1.2109 | furan-2-yl | 3-ethynyl | 4-CN | H |
| I.1.2110 | furan-2-yl | 3-ethynyl | 5-CN | H |
| I.1.2111 | furan-2-yl | 3-ethynyl | 4-CF₃ | H |
| I.1.2112 | furan-2-yl | 3-ethynyl | 5-CF₃ | H |
| I.1.2113 | furan-2-yl | 3-I | 4-Cl | 5-F |
| I.1.2114 | furan-2-yl | 3-I | 4-Cl | H |
| I.1.2115 | furan-2-yl | 3-I | 5-Cl | H |
| I.1.2116 | furan-2-yl | 3-I | 4-F | H |
| I.1.2117 | furan-2-yl | 3-I | 5-F | H |
| I.1.2118 | furan-2-yl | 3-I | 4-CH₃ | H |
| I.1.2119 | furan-2-yl | 3-I | 5-CH₃ | H |
| I.1.2120 | furan-2-yl | 3-I | 4-CN | H |
| I.1.2121 | furan-2-yl | 3-I | 5-CN | H |
| I.1.2122 | furan-2-yl | 3-I | 4-CF₃ | H |
| I.1.2123 | furan-2-yl | 3-I | 5-CF₃ | H |
| I.1.2124 | furan-2-yl | 3-Cl | 4-Cl | 5-F |
| I.1.2125 | furan-2-yl | 3-Cl | 4-Cl | H |
| I.1.2126 | furan-2-yl | 3-Cl | 5-Cl | H |
| I.1.2127 | furan-2-yl | 3-Cl | 4-F | H |
| I.1.2128 | furan-2-yl | 3-Cl | 5-F | H |
| I.1.2129 | furan-2-yl | 3-Cl | 4-CH₃ | H |
| I.1.2130 | furan-2-yl | 3-Cl | 5-CH₃ | H |
| I.1.2131 | furan-2-yl | 3-Cl | 4-CN | H |
| I.1.2132 | furan-2-yl | 3-Cl | 5-CN | H |
| I.1.2133 | furan-2-yl | 3-Cl | 4-CF₃ | H |
| I.1.2134 | furan-2-yl | 3-Cl | 5-CF₃ | H |
| I.1.2135 | furan-2-yl | 3-Br | 4-Cl | 5-F |
| I.1.2136 | furan-2-yl | 3-Br | 4-Cl | H |
| I.1.2137 | furan-2-yl | 3-Br | 5-Cl | H |
| I.1.2138 | furan-2-yl | 3-Br | 4-F | H |
| I.1.2139 | furan-2-yl | 3-Br | 5-F | H |
| I.1.2140 | furan-2-yl | 3-Br | 4-CH₃ | H |
| I.1.2141 | furan-2-yl | 3-Br | 5-CH₃ | H |
| I.1.2142 | furan-2-yl | 3-Br | 4-CN | H |
| I.1.2143 | furan-2-yl | 3-Br | 5-CN | H |
| I.1.2144 | furan-2-yl | 3-Br | 4-CF₃ | H |
| I.1.2145 | furan-2-yl | 3-Br | 5-CF₃ | H |
| I.1.2146 | furan-2-yl | 3-CON(CH₃)₂ | 4-Cl | 5-F |
| I.1.2147 | furan-2-yl | 3-CON(CH₃)₂ | 4-Cl | H |
| I.1.2148 | furan-2-yl | 3-CON(CH₃)₂ | 5-Cl | H |
| I.1.2149 | furan-2-yl | 3-CON(CH₃)₂ | 4-F | H |
| I.1.2150 | furan-2-yl | 3-CON(CH₃)₂ | 5-F | H |
| I.1.2151 | furan-2-yl | 3-CON(CH₃)₂ | 4-CH₃ | H |
| I.1.2152 | furan-2-yl | 3-CON(CH₃)₂ | 5-CH₃ | H |
| I.1.2153 | furan-2-yl | 3-CON(CH₃)₂ | 4-CN | H |
| I.1.2154 | furan-2-yl | 3-CON(CH₃)₂ | 5-CN | H |
| I.1.2155 | furan-2-yl | 3-CON(CH₃)₂ | 4-CF₃ | H |
| I.1.2156 | furan-2-yl | 3-CON(CH₃)₂ | 5-CF₃ | H |
| I.1.2157 | furan-2-yl | 3-CONHCH₃ | 4-Cl | 5-F |
| I.1.2158 | furan-2-yl | 3-CONHCH₃ | 4-Cl | H |
| I.1.2159 | furan-2-yl | 3-CONHCH₃ | 5-Cl | H |
| I.1.2160 | furan-2-yl | 3-CONHCH₃ | 4-F | H |
| I.1.2161 | furan-2-yl | 3-CONHCH₃ | 5-F | H |
| I.1.2162 | furan-2-yl | 3-CONHCH₃ | 4-CH₃ | H |
| I.1.2163 | furan-2-yl | 3-CONHCH₃ | 5-CH₃ | H |
| I.1.2164 | furan-2-yl | 3-CONHCH₃ | 4-CN | H |
| I.1.2165 | furan-2-yl | 3-CONHCH₃ | 5-CN | H |
| I.1.2166 | furan-2-yl | 3-CONHCH₃ | 4-CF₃ | H |
| I.1.2167 | furan-2-yl | 3-CONHCH₃ | 5-CF₃ | H |
| I.1.2168 | furan-3-yl | 2-NO₂ | 4-Cl | 5-F |
| I.1.2169 | furan-3-yl | 2-NO₂ | 4-Cl | H |
| I.1.2170 | furan-3-yl | 2-NO₂ | 5-Cl | H |
| I.1.2171 | furan-3-yl | 2-NO₂ | 4-F | H |
| I.1.2172 | furan-3-yl | 2-NO₂ | 5-F | H |
| I.1.2173 | furan-3-yl | 2-NO₂ | 4-CH₃ | H |
| I.1.2174 | furan-3-yl | 2-NO₂ | 5-CH₃ | H |
| I.1.2175 | furan-3-yl | 2-NO₂ | 4-CN | H |
| I.1.2176 | furan-3-yl | 2-NO₂ | 5-CN | H |
| I.1.2177 | furan-3-yl | 2-NO₂ | 4-CF₃ | H |
| I.1.2178 | furan-3-yl | 2-NO₂ | 5-CF₃ | H |
| I.1.2179 | furan-3-yl | 2-CN | 4-Cl | 5-F |
| I.1.2180 | furan-3-yl | 2-CN | 4-Cl | H |
| I.1.2181 | furan-3-yl | 2-CN | 5-Cl | H |
| I.1.2182 | furan-3-yl | 2-CN | 4-F | H |
| I.1.2183 | furan-3-yl | 2-CN | 5-F | H |
| I.1.2184 | furan-3-yl | 2-CN | 4-CH₃ | H |
| I.1.2185 | furan-3-yl | 2-CN | 5-CH₃ | H |
| I.1.2186 | furan-3-yl | 2-CN | 4-CN | H |
| I.1.2187 | furan-3-yl | 2-CN | 5-CN | H |
| I.1.2188 | furan-3-yl | 2-CN | 4-CF₃ | H |
| I.1.2189 | furan-3-yl | 2-CN | 5-CF₃ | H |
| I.1.2190 | furan-3-yl | 2-COOH | 4-Cl | 5-F |
| I.1.2191 | furan-3-yl | 2-COOH | 4-Cl | H |
| I.1.2192 | furan-3-yl | 2-COOH | 5-Cl | H |
| I.1.2193 | furan-3-yl | 2-COOH | 4-F | H |
| I.1.2194 | furan-3-yl | 2-COOH | 5-F | H |
| I.1.2195 | furan-3-yl | 2-COOH | 4-CH₃ | H |
| I.1.2196 | furan-3-yl | 2-COOH | 5-CH₃ | H |
| I.1.2197 | furan-3-yl | 2-COOH | 4-CN | H |
| I.1.2198 | furan-3-yl | 2-COOH | 5-CN | H |
| I.1.2199 | furan-3-yl | 2-COOH | 4-CF₃ | H |
| I.1.2200 | furan-3-yl | 2-COOH | 5-CF₃ | H |
| I.1.2201 | furan-3-yl | 2-COOCH₃ | 4-Cl | 5-F |
| I.1.2202 | furan-3-yl | 2-COOCH₃ | 4-Cl | H |
| I.1.2203 | furan-3-yl | 2-COOCH₃ | 5-Cl | H |
| I.1.2204 | furan-3-yl | 2-COOCH₃ | 4-F | H |
| I.1.2205 | furan-3-yl | 2-COOCH₃ | 5-F | H |
| I.1.2206 | furan-3-yl | 2-COOCH₃ | 4-CH₃ | H |
| I.1.2207 | furan-3-yl | 2-COOCH₃ | 5-CH₃ | H |
| I.1.2208 | furan-3-yl | 2-COOCH₃ | 4-CN | H |
| I.1.2209 | furan-3-yl | 2-COOCH₃ | 5-CN | H |
| I.1.2210 | furan-3-yl | 2-COOCH₃ | 4-CF₃ | H |
| I.1.2211 | furan-3-yl | 2-COOCH₃ | 5-CF₃ | H |
| I.1.2212 | furan-3-yl | 2-COOCH₂CH₃ | 4-Cl | 5-F |
| I.1.2213 | furan-3-yl | 2-COOCH₂CH₃ | 4-Cl | H |
| I.1.2214 | furan-3-yl | 2-COOCH₂CH₃ | 5-Cl | H |
| I.1.2215 | furan-3-yl | 2-COOCH₂CH₃ | 4-F | H |
| I.1.2216 | furan-3-yl | 2-COOCH₂CH₃ | 5-F | H |
| I.1.2217 | furan-3-yl | 2-COOCH₂CH₃ | 4-CH₃ | H |
| I.1.2218 | furan-3-yl | 2-COOCH₂CH₃ | 5-CH₃ | H |
| I.1.2219 | furan-3-yl | 2-COOCH₂CH₃ | 4-CN | H |
| I.1.2220 | furan-3-yl | 2-COOCH₂CH₃ | 5-CN | H |
| I.1.2221 | furan-3-yl | 2-COOCH₂CH₃ | 4-CF₃ | H |
| I.1.2222 | furan-3-yl | 2-COOCH₂CH₃ | 5-CF₃ | H |
| I.1.2223 | furan-3-yl | 2-ethynyl | 4-Cl | 5-F |
| I.1.2224 | furan-3-yl | 2-ethynyl | 4-Cl | H |
| I.1.2225 | furan-3-yl | 2-ethynyl | 5-Cl | H |
| I.1.2226 | furan-3-yl | 2-ethynyl | 4-F | H |
| I.1.2227 | furan-3-yl | 2-ethynyl | 5-F | H |
| I.1.2228 | furan-3-yl | 2-ethynyl | 4-CH₃ | H |
| I.1.2229 | furan-3-yl | 2-ethynyl | 5-CH₃ | H |
| I.1.2230 | furan-3-yl | 2-ethynyl | 4-CN | H |
| I.1.2231 | furan-3-yl | 2-ethynyl | 5-CN | H |
| I.1.2232 | furan-3-yl | 2-ethynyl | 4-CF₃ | H |
| I.1.2233 | furan-3-yl | 2-ethynyl | 5-CF₃ | H |
| I.1.2234 | furan-3-yl | 2-I | 4-Cl | 5-F |
| I.1.2235 | furan-3-yl | 2-I | 4-Cl | H |
| I.1.2236 | furan-3-yl | 2-I | 5-Cl | H |
| I.1.2237 | furan-3-yl | 2-I | 4-F | H |
| I.1.2238 | furan-3-yl | 2-I | 5-F | H |
| I.1.2239 | furan-3-yl | 2-I | 4-CH₃ | H |
| I.1.2240 | furan-3-yl | 2-I | 5-CH₃ | H |
| I.1.2241 | furan-3-yl | 2-I | 4-CN | H |
| I.1.2242 | furan-3-yl | 2-I | 5-CN | H |
| I.1.2243 | furan-3-yl | 2-I | 4-CF₃ | H |
| I.1.2244 | furan-3-yl | 2-I | 5-CF₃ | H |
| I.1.2245 | furan-3-yl | 2-Cl | 4-Cl | 5-F |
| I.1.2246 | furan-3-yl | 2-Cl | 4-Cl | H |
| I.1.2247 | furan-3-yl | 2-Cl | 5-Cl | H |
| I.1.2248 | furan-3-yl | 2-Cl | 4-F | H |
| I.1.2249 | furan-3-yl | 2-Cl | 5-F | H |
| I.1.2250 | furan-3-yl | 2-Cl | 4-CH₃ | H |
| I.1.2251 | furan-3-yl | 2-Cl | 5-CH₃ | H |
| I.1.2252 | furan-3-yl | 2-Cl | 4-CN | H |
| I.1.2253 | furan-3-yl | 2-Cl | 5-CN | H |
| I.1.2254 | furan-3-yl | 2-Cl | 4-CF₃ | H |
| I.1.2255 | furan-3-yl | 2-Cl | 5-CF₃ | H |
| I.1.2256 | furan-3-yl | 2-Br | 4-Cl | 5-F |
| I.1.2257 | furan-3-yl | 2-Br | 4-Cl | H |
| I.1.2258 | furan-3-yl | 2-Br | 5-Cl | H |
| I.1.2259 | furan-3-yl | 2-Br | 4-F | H |
| I.1.2260 | furan-3-yl | 2-Br | 5-F | H |
| I.1.2261 | furan-3-yl | 2-Br | 4-CH₃ | H |
| I.1.2262 | furan-3-yl | 2-Br | 5-CH₃ | H |
| I.1.2263 | furan-3-yl | 2-Br | 4-CN | H |
| I.1.2264 | furan-3-yl | 2-Br | 5-CN | H |
| I.1.2265 | furan-3-yl | 2-Br | 4-CF₃ | H |
| I.1.2266 | furan-3-yl | 2-Br | 5-CF₃ | H |
| I.1.2267 | furan-3-yl | 2-CON(CH₃)₂ | 4-Cl | 5-F |
| I.1.2268 | furan-3-yl | 2-CON(CH₃)₂ | 4-Cl | H |
| I.1.2269 | furan-3-yl | 2-CON(CH₃)₂ | 5-Cl | H |
| I.1.2270 | furan-3-yl | 2-CON(CH₃)₂ | 4-F | H |
| I.1.2271 | furan-3-yl | 2-CON(CH₃)₂ | 5-F | H |
| I.1.2272 | furan-3-yl | 2-CON(CH₃)₂ | 4-CH₃ | H |
| I.1.2273 | furan-3-yl | 2-CON(CH₃)₂ | 5-CH₃ | H |
| I.1.2274 | furan-3-yl | 2-CON(CH₃)₂ | 4-CN | H |
| I.1.2275 | furan-3-yl | 2-CON(CH₃)₂ | 5-CN | H |
| I.1.2276 | furan-3-yl | 2-CON(CH₃)₂ | 4-CF₃ | H |
| I.1.2277 | furan-3-yl | 2-CON(CH₃)₂ | 5-CF₃ | H |
| I.1.2278 | furan-3-yl | 2-CONHCH₃ | 4-Cl | 5-F |
| I.1.2279 | furan-3-yl | 2-CONHCH₃ | 4-Cl | H |
| I.1.2280 | furan-3-yl | 2-CONHCH₃ | 5-Cl | H |
| I.1.2281 | furan-3-yl | 2-CONHCH₃ | 4-F | H |
| I.1.2282 | furan-3-yl | 2-CONHCH₃ | 5-F | H |
| I.1.2283 | furan-3-yl | 2-CONHCH₃ | 4-CH₃ | H |
| I.1.2284 | furan-3-yl | 2-CONHCH₃ | 5-CH₃ | H |
| I.1.2285 | furan-3-yl | 2-CONHCH₃ | 4-CN | H |
| I.1.2286 | furan-3-yl | 2-CONHCH₃ | 5-CN | H |
| I.1.2287 | furan-3-yl | 2-CONHCH₃ | 4-CF₃ | H |
| I.1.2288 | furan-3-yl | 2-CONHCH₃ | 5-CF₃ | H |
| I.1.2289 | furan-3-yl | 4-NO₂ | 5-Cl | 2-F |
| I.1.2290 | furan-3-yl | 4-NO₂ | 5-Cl | H |
| I.1.2291 | furan-3-yl | 4-NO₂ | 2-Cl | H |
| I.1.2292 | furan-3-yl | 4-NO₂ | 5-F | H |
| I.1.2293 | furan-3-yl | 4-NO₂ | 2-F | H |
| I.1.2294 | furan-3-yl | 4-NO₂ | 5-CH₃ | H |
| I.1.2295 | furan-3-yl | 4-NO₂ | 2-CH₃ | H |
| I.1.2296 | furan-3-yl | 4-NO₂ | 5-CN | H |
| I.1.2297 | furan-3-yl | 4-NO₂ | 2-CN | H |
| I.1.2298 | furan-3-yl | 4-NO₂ | 5-CF₃ | H |
| I.1.2299 | furan-3-yl | 4-NO₂ | 2-CF₃ | H |
| I.1.2300 | furan-3-yl | 4-CN | 5-Cl | 2-F |
| I.1.2301 | furan-3-yl | 4-CN | 5-Cl | H |
| I.1.2302 | furan-3-yl | 4-CN | 2-Cl | H |
| I.1.2303 | furan-3-yl | 4-CN | 5-F | H |
| I.1.2304 | furan-3-yl | 4-CN | 2-F | H |
| I.1.2305 | furan-3-yl | 4-CN | 5-CH₃ | H |
| I.1.2306 | furan-3-yl | 4-CN | 2-CH₃ | H |
| I.1.2307 | furan-3-yl | 4-CN | 5-CN | H |
| I.1.2308 | furan-3-yl | 4-CN | 2-CN | H |
| I.1.2309 | furan-3-yl | 4-CN | 5-CF₃ | H |
| I.1.2310 | furan-3-yl | 4-CN | 2-CF₃ | H |
| I.1.2311 | furan-3-yl | 4-COOH | 5-Cl | 2-F |
| I.1.2312 | furan-3-yl | 4-COOH | 5-Cl | H |
| I.1.2313 | furan-3-yl | 4-COOH | 2-Cl | H |
| I.1.2314 | furan-3-yl | 4-COOH | 5-F | H |
| I.1.2315 | furan-3-yl | 4-COOH | 2-F | H |
| I.1.2316 | furan-3-yl | 4-COOH | 5-CH₃ | H |
| I.1.2317 | furan-3-yl | 4-COOH | 2-CH₃ | H |
| I.1.2318 | furan-3-yl | 4-COOH | 5-CN | H |
| I.1.2319 | furan-3-yl | 4-COOH | 2-CN | H |
| I.1.2320 | furan-3-yl | 4-COOH | 5-CF₃ | H |
| I.1.2321 | furan-3-yl | 4-COOH | 2-CF₃ | H |
| I.1.2322 | furan-3-yl | 4-COOCH₃ | 5-Cl | 2-F |
| I.1.2323 | furan-3-yl | 4-COOCH₃ | 5-Cl | H |
| I.1.2324 | furan-3-yl | 4-COOCH₃ | 2-Cl | H |
| I.1.2325 | furan-3-yl | 4-COOCH₃ | 5-F | H |
| I.1.2326 | furan-3-yl | 4-COOCH₃ | 2-F | H |
| I.1.2327 | furan-3-yl | 4-COOCH₃ | 5-CH₃ | H |
| I.1.2328 | furan-3-yl | 4-COOCH₃ | 2-CH₃ | H |
| I.1.2329 | furan-3-yl | 4-COOCH₃ | 5-CN | H |
| I.1.2330 | furan-3-yl | 4-COOCH₃ | 2-CN | H |
| I.1.2331 | furan-3-yl | 4-COOCH₃ | 5-CF₃ | H |
| I.1.2332 | furan-3-yl | 4-COOCH₃ | 2-CF₃ | H |
| I.1.2333 | furan-3-yl | 4-COOCH₂CH₃ | 5-Cl | 2-F |
| I.1.2334 | furan-3-yl | 4-COOCH₂CH₃ | 5-Cl | H |
| I.1.2335 | furan-3-yl | 4-COOCH₂CH₃ | 2-Cl | H |
| I.1.2336 | furan-3-yl | 4-COOCH₂CH₃ | 5-F | H |
| I.1.2337 | furan-3-yl | 4-COOCH₂CH₃ | 2-F | H |
| I.1.2338 | furan-3-yl | 4-COOCH₂CH₃ | 5-CH₃ | H |
| I.1.2339 | furan-3-yl | 4-COOCH₂CH₃ | 2-CH₃ | H |
| I.1.2340 | furan-3-yl | 4-COOCH₂CH₃ | 5-CN | H |
| I.1.2341 | furan-3-yl | 4-COOCH₂CH₃ | 2-CN | H |
| I.1.2342 | furan-3-yl | 4-COOCH₂CH₃ | 5-CF₃ | H |
| I.1.2343 | furan-3-yl | 4-COOCH₂CH₃ | 2-CF₃ | H |
| I.1.2344 | furan-3-yl | 4-ethynyl | 5-Cl | 2-F |
| I.1.2345 | furan-3-yl | 4-ethynyl | 5-Cl | H |
| I.1.2346 | furan-3-yl | 4-ethynyl | 2-Cl | H |
| I.1.2347 | furan-3-yl | 4-ethynyl | 5-F | H |
| I.1.2348 | furan-3-yl | 4-ethynyl | 2-F | H |
| I.1.2349 | furan-3-yl | 4-ethynyl | 5-CH₃ | H |
| I.1.2350 | furan-3-yl | 4-ethynyl | 2-CH₃ | H |
| I.1.2351 | furan-3-yl | 4-ethynyl | 5-CN | H |
| I.1.2352 | furan-3-yl | 4-ethynyl | 2-CN | H |
| I.1.2353 | furan-3-yl | 4-ethynyl | 5-CF₃ | H |
| I.1.2354 | furan-3-yl | 4-ethynyl | 2-CF₃ | H |
| I.1.2355 | furan-3-yl | 4-I | 5-Cl | 2-F |
| I.1.2356 | furan-3-yl | 4-I | 5-Cl | H |
| I.1.2357 | furan-3-yl | 4-I | 2-Cl | H |
| I.1.2358 | furan-3-yl | 4-I | 5-F | H |
| I.1.2359 | furan-3-yl | 4-I | 2-F | H |
| I.1.2360 | furan-3-yl | 4-I | 5-CH₃ | H |
| I.1.2361 | furan-3-yl | 4-I | 2-CH₃ | H |
| I.1.2362 | furan-3-yl | 4-I | 5-CN | H |
| I.1.2363 | furan-3-yl | 4-I | 2-CN | H |
| I.1.2364 | furan-3-yl | 4-I | 5-CF₃ | H |
| I.1.2365 | furan-3-yl | 4-I | 2-CF₃ | H |
| I.1.2366 | furan-3-yl | 4-Cl | 5-Cl | 2-F |
| I.1.2367 | furan-3-yl | 4-Cl | 5-Cl | H |
| I.1.2368 | furan-3-yl | 4-Cl | 2-Cl | H |
| I.1.2369 | furan-3-yl | 4-Cl | 5-F | H |
| I.1.2370 | furan-3-yl | 4-Cl | 2-F | H |
| I.1.2371 | furan-3-yl | 4-Cl | 5-CH₃ | H |
| I.1.2372 | furan-3-yl | 4-Cl | 2-CH₃ | H |
| I.1.2373 | furan-3-yl | 4-Cl | 5-CN | H |
| I.1.2374 | furan-3-yl | 4-Cl | 2-CN | H |
| I.1.2375 | furan-3-yl | 4-Cl | 5-CF₃ | H |
| I.1.2376 | furan-3-yl | 4-Cl | 2-CF₃ | H |
| I.1.2377 | furan-3-yl | 4-Br | 5-Cl | 2-F |
| I.1.2378 | furan-3-yl | 4-Br | 5-Cl | H |
| I.1.2379 | furan-3-yl | 4-Br | 2-Cl | H |
| I.1.2380 | furan-3-yl | 4-Br | 5-F | H |
| I.1.2381 | furan-3-yl | 4-Br | 2-F | H |
| I.1.2382 | furan-3-yl | 4-Br | 5-CH₃ | H |
| I.1.2383 | furan-3-yl | 4-Br | 2-CH₃ | H |
| I.1.2384 | furan-3-yl | 4-Br | 5-CN | H |
| I.1.2385 | furan-3-yl | 4-Br | 2-CN | H |
| I.1.2386 | furan-3-yl | 4-Br | 5-CF₃ | H |
| I.1.2387 | furan-3-yl | 4-Br | 2-CF₃ | H |
| I.1.2388 | furan-3-yl | 4-CON(CH₃)₂ | 5-Cl | 2-F |
| I.1.2389 | furan-3-yl | 4-CON(CH₃)₂ | 5-Cl | H |
| I.1.2390 | furan-3-yl | 4-CON(CH₃)₂ | 2-Cl | H |
| I.1.2391 | furan-3-yl | 4-CON(CH₃)₂ | 5-F | H |
| I.1.2392 | furan-3-yl | 4-CON(CH₃)₂ | 2-F | H |
| I.1.2393 | furan-3-yl | 4-CON(CH₃)₂ | 5-CH₃ | H |
| I.1.2394 | furan-3-yl | 4-CON(CH₃)₂ | 2-CH₃ | H |
| I.1.2395 | furan-3-yl | 4-CON(CH₃)₂ | 5-CN | H |
| I.1.2396 | furan-3-yl | 4-CON(CH₃)₂ | 2-CN | H |
| I.1.2397 | furan-3-yl | 4-CON(CH₃)₂ | 5-CF₃ | H |
| I.1.2398 | furan-3-yl | 4-CON(CH₃)₂ | 2-CF₃ | H |
| I.1.2399 | furan-3-yl | 4-CONHCH₃ | 5-Cl | 2-F |
| I.1.2400 | furan-3-yl | 4-CONHCH₃ | 5-Cl | H |
| I.1.2401 | furan-3-yl | 4-CONHCH₃ | 2-Cl | H |
| I.1.2402 | furan-3-yl | 4-CONHCH₃ | 5-F | H |
| I.1.2403 | furan-3-yl | 4-CONHCH₃ | 2-F | H |
| I.1.2404 | furan-3-yl | 4-CONHCH₃ | 5-CH₃ | H |
| I.1.2405 | furan-3-yl | 4-CONHCH₃ | 2-CH₃ | H |
| I.1.2406 | furan-3-yl | 4-CONHCH₃ | 5-CN | H |
| I.1.2407 | furan-3-yl | 4-CONHCH₃ | 2-CN | H |
| I.1.2408 | furan-3-yl | 4-CONHCH₃ | 5-CF₃ | H |
| I.1.2409 | furan-3-yl | 4-CONHCH₃ | 2-CF₃ | H |
| I.1.2410 | thiophen-2-yl | 3-NO₂ | 4-Cl | 5-F |
| I.1.2411 | thiophen-2-yl | 3-NO₂ | 4-Cl | H |
| I.1.2412 | thiophen-2-yl | 3-NO₂ | 5-Cl | H |
| I.1.2413 | thiophen-2-yl | 3-NO₂ | 4-F | H |
| I.1.2414 | thiophen-2-yl | 3-NO₂ | 5-F | H |
| I.1.2415 | thiophen-2-yl | 3-NO₂ | 4-CH₃ | H |
| I.1.2416 | thiophen-2-yl | 3-NO₂ | 5-CH₃ | H |
| I.1.2417 | thiophen-2-yl | 3-NO₂ | 4-CN | H |
| I.1.2418 | thiophen-2-yl | 3-NO₂ | 5-CN | H |
| I.1.2419 | thiophen-2-yl | 3-NO₂ | 4-CF₃ | H |
| I.1.2420 | thiophen-2-yl | 3-NO₂ | 5-CF₃ | H |
| I.1.2421 | thiophen-2-yl | 3-CN | 4-Cl | 5-F |
| I.1.2422 | thiophen-2-yl | 3-CN | 4-Cl | H |
| I.1.2423 | thiophen-2-yl | 3-CN | 5-Cl | H |
| I.1.2424 | thiophen-2-yl | 3-CN | 4-F | H |
| I.1.2425 | thiophen-2-yl | 3-CN | 5-F | H |
| I.1.2426 | thiophen-2-yl | 3-CN | 4-CH₃ | H |
| I.1.2427 | thiophen-2-yl | 3-CN | 5-CH₃ | H |
| I.1.2428 | thiophen-2-yl | 3-CN | 4-CN | H |
| I.1.2429 | thiophen-2-yl | 3-CN | 5-CN | H |
| I.1.2430 | thiophen-2-yl | 3-CN | 4-CF₃ | H |
| I.1.2431 | thiophen-2-yl | 3-CN | 5-CF₃ | H |
| I.1.2432 | thiophen-2-yl | 3-COOH | 4-Cl | 5-F |
| I.1.2433 | thiophen-2-yl | 3-COOH | 4-Cl | H |
| I.1.2434 | thiophen-2-yl | 3-COOH | 5-Cl | H |
| I.1.2435 | thiophen-2-yl | 3-COOH | 4-F | H |
| I.1.2436 | thiophen-2-yl | 3-COOH | 5-F | H |
| I.1.2437 | thiophen-2-yl | 3-COOH | 4-CH₃ | H |
| I.1.2438 | thiophen-2-yl | 3-COOH | 5-CH₃ | H |
| I.1.2439 | thiophen-2-yl | 3-COOH | 4-CN | H |
| I.1.2440 | thiophen-2-yl | 3-COOH | 5-CN | H |
| I.1.2441 | thiophen-2-yl | 3-COOH | 4-CF₃ | H |
| I.1.2442 | thiophen-2-yl | 3-COOH | 5-CF₃ | H |
| I.1.2443 | thiophen-2-yl | 3-COOCH₃ | 4-Cl | 5-F |
| I.1.2444 | thiophen-2-yl | 3-COOCH₃ | 4-Cl | H |
| I.1.2445 | thiophen-2-yl | 3-COOCH₃ | 5-Cl | H |
| I.1.2446 | thiophen-2-yl | 3-COOCH₃ | 4-F | H |
| I.1.2447 | thiophen-2-yl | 3-COOCH₃ | 5-F | H |
| I.1.2448 | thiophen-2-yl | 3-COOCH₃ | 4-CH₃ | H |
| I.1.2449 | thiophen-2-yl | 3-COOCH₃ | 5-CH₃ | H |
| I.1.2450 | thiophen-2-yl | 3-COOCH₃ | 4-CN | H |
| I.1.2451 | thiophen-2-yl | 3-COOCH₃ | 5-CN | H |
| I.1.2452 | thiophen-2-yl | 3-COOCH₃ | 4-CF₃ | H |
| I.1.2453 | thiophen-2-yl | 3-COOCH₃ | 5-CF₃ | H |
| I.1.2454 | thiophen-2-yl | 3-COOCH₂CH₃ | 4-Cl | 5-F |
| I.1.2455 | thiophen-2-yl | 3-COOCH₂CH₃ | 4-Cl | H |
| I.1.2456 | thiophen-2-yl | 3-COOCH₂CH₃ | 5-Cl | H |
| I.1.2457 | thiophen-2-yl | 3-COOCH₂CH₃ | 4-F | H |
| I.1.2458 | thiophen-2-yl | 3-COOCH₂CH₃ | 5-F | H |
| I.1.2459 | thiophen-2-yl | 3-COOCH₂CH₃ | 4-CH₃ | H |
| I.1.2460 | thiophen-2-yl | 3-COOCH₂CH₃ | 5-CH₃ | H |
| I.1.2461 | thiophen-2-yl | 3-COOCH₂CH₃ | 4-CN | H |
| I.1.2462 | thiophen-2-yl | 3-COOCH₂CH₃ | 5-CN | H |
| I.1.2463 | thiophen-2-yl | 3-COOCH₂CH₃ | 4-CF₃ | H |
| I.1.2464 | thiophen-2-yl | 3-COOCH₂CH₃ | 5-CF₃ | H |
| I.1.2465 | thiophen-2-yl | 3-ethynyl | 4-Cl | 5-F |
| I.1.2466 | thiophen-2-yl | 3-ethynyl | 4-Cl | H |
| I.1.2467 | thiophen-2-yl | 3-ethynyl | 5-Cl | H |
| I.1.2468 | thiophen-2-yl | 3-ethynyl | 4-F | H |
| I.1.2469 | thiophen-2-yl | 3-ethynyl | 5-F | H |
| I.1.2470 | thiophen-2-yl | 3-ethynyl | 4-CH₃ | H |
| I.1.2471 | thiophen-2-yl | 3-ethynyl | 5-CH₃ | H |
| I.1.2472 | thiophen-2-yl | 3-ethynyl | 4-CN | H |
| I.1.2473 | thiophen-2-yl | 3-ethynyl | 5-CN | H |
| I.1.2474 | thiophen-2-yl | 3-ethynyl | 4-CF₃ | H |
| I.1.2475 | thiophen-2-yl | 3-ethynyl | 5-CF₃ | H |
| I.1.2476 | thiophen-2-yl | 3-I | 4-Cl | 5-F |
| I.1.2477 | thiophen-2-yl | 3-I | 4-Cl | H |
| I.1.2478 | thiophen-2-yl | 3-I | 5-Cl | H |
| I.1.2479 | thiophen-2-yl | 3-I | 4-F | H |
| I.1.2480 | thiophen-2-yl | 3-I | 5-F | H |
| I.1.2481 | thiophen-2-yl | 3-I | 4-CH₃ | H |
| I.1.2482 | thiophen-2-yl | 3-I | 5-CH₃ | H |
| I.1.2483 | thiophen-2-yl | 3-I | 4-CN | H |
| I.1.2484 | thiophen-2-yl | 3-I | 5-CN | H |
| I.1.2485 | thiophen-2-yl | 3-I | 4-CF₃ | H |
| I.1.2486 | thiophen-2-yl | 3-I | 5-CF₃ | H |
| I.1.2487 | thiophen-2-yl | 3-Cl | 4-Cl | 5-F |
| I.1.2488 | thiophen-2-yl | 3-Cl | 4-Cl | H |
| I.1.2489 | thiophen-2-yl | 3-Cl | 5-Cl | H |
| I.1.2490 | thiophen-2-yl | 3-Cl | 4-F | H |
| I.1.2491 | thiophen-2-yl | 3-Cl | 5-F | H |
| I.1.2492 | thiophen-2-yl | 3-Cl | 4-CH₃ | H |
| I.1.2493 | thiophen-2-yl | 3-Cl | 5-CH₃ | H |
| I.1.2494 | thiophen-2-yl | 3-Cl | 4-CN | H |
| I.1.2495 | thiophen-2-yl | 3-Cl | 5-CN | H |
| I.1.2496 | thiophen-2-yl | 3-Cl | 4-CF₃ | H |
| I.1.2497 | thiophen-2-yl | 3-Cl | 5-CF₃ | H |
| I.1.2498 | thiophen-2-yl | 3-Br | 4-Cl | 5-F |
| I.1.2499 | thiophen-2-yl | 3-Br | 4-Cl | H |
| I.1.2500 | thiophen-2-yl | 3-Br | 5-Cl | H |
| I.1.2501 | thiophen-2-yl | 3-Br | 4-F | H |
| I.1.2502 | thiophen-2-yl | 3-Br | 5-F | H |
| I.1.2503 | thiophen-2-yl | 3-Br | 4-CH₃ | H |
| I.1.2504 | thiophen-2-yl | 3-Br | 5-CH₃ | H |
| I.1.2505 | thiophen-2-yl | 3-Br | 4-CN | H |
| I.1.2506 | thiophen-2-yl | 3-Br | 5-CN | H |
| I.1.2507 | thiophen-2-yl | 3-Br | 4-CF₃ | H |
| I.1.2508 | thiophen-2-yl | 3-Br | 5-CF₃ | H |
| I.1.2509 | thiophen-2-yl | 3-CON(CH₃)₂ | 4-Cl | 5-F |
| I.1.2510 | thiophen-2-yl | 3-CON(CH₃)₂ | 4-Cl | H |
| I.1.2511 | thiophen-2-yl | 3-CON(CH₃)₂ | 5-Cl | H |
| I.1.2512 | thiophen-2-yl | 3-CON(CH₃)₂ | 4-F | H |
| I.1.2513 | thiophen-2-yl | 3-CON(CH₃)₂ | 5-F | H |
| I.1.2514 | thiophen-2-yl | 3-CON(CH₃)₂ | 4-CH₃ | H |
| I.1.2515 | thiophen-2-yl | 3-CON(CH₃)₂ | 5-CH₃ | H |
| I.1.2516 | thiophen-2-yl | 3-CON(CH₃)₂ | 4-CN | H |
| I.1.2517 | thiophen-2-yl | 3-CON(CH₃)₂ | 5-CN | H |
| I.1.2518 | thiophen-2-yl | 3-CON(CH₃)₂ | 4-CF₃ | H |
| I.1.2519 | thiophen-2-yl | 3-CON(CH₃)₂ | 5-CF₃ | H |
| I.1.2520 | thiophen-2-yl | 3-CONHCH₃ | 4-Cl | 5-F |
| I.1.2521 | thiophen-2-yl | 3-CONHCH₃ | 4-Cl | H |
| I.1.2522 | thiophen-2-yl | 3-CONHCH₃ | 5-Cl | H |
| I.1.2523 | thiophen-2-yl | 3-CONHCH₃ | 4-F | H |
| I.1.2524 | thiophen-2-yl | 3-CONHCH₃ | 5-F | H |
| I.1.2525 | thiophen-2-yl | 3-CONHCH₃ | 4-CH₃ | H |
| I.1.2526 | thiophen-2-yl | 3-CONHCH₃ | 5-CH₃ | H |
| I.1.2527 | thiophen-2-yl | 3-CONHCH₃ | 4-CN | H |
| I.1.2528 | thiophen-2-yl | 3-CONHCH₃ | 5-CN | H |
| I.1.2529 | thiophen-2-yl | 3-CONHCH₃ | 4-CF₃ | H |
| I.1.2530 | thiophen-2-yl | 3-CONHCH₃ | 5-CF₃ | H |
| I.1.2531 | thiophen-3-yl | 2-NO₂ | 4-Cl | 5-F |
| I.1.2532 | thiophen-3-yl | 2-NO₂ | 4-Cl | H |
| I.1.2533 | thiophen-3-yl | 2-NO₂ | 5-Cl | H |
| I.1.2534 | thiophen-3-yl | 2-NO₂ | 4-F | H |
| I.1.2535 | thiophen-3-yl | 2-NO₂ | 5-F | H |
| I.1.2536 | thiophen-3-yl | 2-NO₂ | 4-CH₃ | H |
| I.1.2537 | thiophen-3-yl | 2-NO₂ | 5-CH₃ | H |
| I.1.2538 | thiophen-3-yl | 2-NO₂ | 4-CN | H |
| I.1.2539 | thiophen-3-yl | 2-NO₂ | 5-CN | H |
| I.1.2540 | thiophen-3-yl | 2-NO₂ | 4-CF₃ | H |
| I.1.2541 | thiophen-3-yl | 2-NO₂ | 5-CF₃ | H |
| I.1.2542 | thiophen-3-yl | 2-CN | 4-Cl | 5-F |
| I.1.2543 | thiophen-3-yl | 2-CN | 4-Cl | H |
| I.1.2544 | thiophen-3-yl | 2-CN | 5-Cl | H |
| I.1.2545 | thiophen-3-yl | 2-CN | 4-F | H |
| I.1.2546 | thiophen-3-yl | 2-CN | 5-F | H |
| I.1.2547 | thiophen-3-yl | 2-CN | 4-CH₃ | H |
| I.1.2548 | thiophen-3-yl | 2-CN | 5-CH₃ | H |
| I.1.2549 | thiophen-3-yl | 2-CN | 4-CN | H |
| I.1.2550 | thiophen-3-yl | 2-CN | 5-CN | H |
| I.1.2551 | thiophen-3-yl | 2-CN | 4-CF₃ | H |
| I.1.2552 | thiophen-3-yl | 2-CN | 5-CF₃ | H |
| I.1.2553 | thiophen-3-yl | 2-COOH | 4-Cl | 5-F |
| I.1.2554 | thiophen-3-yl | 2-COOH | 4-Cl | H |
| I.1.2555 | thiophen-3-yl | 2-COOH | 5-Cl | H |
| I.1.2556 | thiophen-3-yl | 2-COOH | 4-F | H |
| I.1.2557 | thiophen-3-yl | 2-COOH | 5-F | H |
| I.1.2558 | thiophen-3-yl | 2-COOH | 4-CH₃ | H |
| I.1.2559 | thiophen-3-yl | 2-COOH | 5-CH₃ | H |
| I.1.2560 | thiophen-3-yl | 2-COOH | 4-CN | H |
| I.1.2561 | thiophen-3-yl | 2-COOH | 5-CN | H |
| I.1.2562 | thiophen-3-yl | 2-COOH | 4-CF₃ | H |
| I.1.2563 | thiophen-3-yl | 2-COOH | 5-CF₃ | H |
| I.1.2564 | thiophen-3-yl | 2-COOCH₃ | 4-Cl | 5-F |
| I.1.2565 | thiophen-3-yl | 2-COOCH₃ | 4-Cl | H |
| I.1.2566 | thiophen-3-yl | 2-COOCH₃ | 5-Cl | H |
| I.1.2567 | thiophen-3-yl | 2-COOCH₃ | 4-F | H |
| I.1.2568 | thiophen-3-yl | 2-COOCH₃ | 5-F | H |
| I.1.2569 | thiophen-3-yl | 2-COOCH₃ | 4-CH₃ | H |
| I.1.2570 | thiophen-3-yl | 2-COOCH₃ | 5-CH₃ | H |
| I.1.2571 | thiophen-3-yl | 2-COOCH₃ | 4-CN | H |
| I.1.2572 | thiophen-3-yl | 2-COOCH₃ | 5-CN | H |
| I.1.2573 | thiophen-3-yl | 2-COOCH₃ | 4-CF₃ | H |
| I.1.2574 | thiophen-3-yl | 2-COOCH₃ | 5-CF₃ | H |
| I.1.2575 | thiophen-3-yl | 2-COOCH₂CH₃ | 4-Cl | 5-F |
| I.1.2576 | thiophen-3-yl | 2-COOCH₂CH₃ | 4-Cl | H |
| I.1.2577 | thiophen-3-yl | 2-COOCH₂CH₃ | 5-Cl | H |
| I.1.2578 | thiophen-3-yl | 2-COOCH₂CH₃ | 4-F | H |
| I.1.2579 | thiophen-3-yl | 2-COOCH₂CH₃ | 5-F | H |
| I.1.2580 | thiophen-3-yl | 2-COOCH₂CH₃ | 4-CH₃ | H |
| I.1.2581 | thiophen-3-yl | 2-COOCH₂CH₃ | 5-CH₃ | H |
| I.1.2582 | thiophen-3-yl | 2-COOCH₂CH₃ | 4-CN | H |
| I.1.2583 | thiophen-3-yl | 2-COOCH₂CH₃ | 5-CN | H |
| I.1.2584 | thiophen-3-yl | 2-COOCH₂CH₃ | 4-CF₃ | H |
| I.1.2585 | thiophen-3-yl | 2-COOCH₂CH₃ | 5-CF₃ | H |
| I.1.2586 | thiophen-3-yl | 2-ethynyl | 4-Cl | 5-F |
| I.1.2587 | thiophen-3-yl | 2-ethynyl | 4-Cl | H |
| I.1.2588 | thiophen-3-yl | 2-ethynyl | 5-Cl | H |
| I.1.2589 | thiophen-3-yl | 2-ethynyl | 4-F | H |
| I.1.2590 | thiophen-3-yl | 2-ethynyl | 5-F | H |
| I.1.2591 | thiophen-3-yl | 2-ethynyl | 4-CH₃ | H |
| I.1.2592 | thiophen-3-yl | 2-ethynyl | 5-CH₃ | H |
| I.1.2593 | thiophen-3-yl | 2-ethynyl | 4-CN | H |
| I.1.2594 | thiophen-3-yl | 2-ethynyl | 5-CN | H |
| I.1.2595 | thiophen-3-yl | 2-ethynyl | 4-CF₃ | H |
| I.1.2596 | thiophen-3-yl | 2-ethynyl | 5-CF₃ | H |
| I.1.2597 | thiophen-3-yl | 2-I | 4-Cl | 5-F |
| I.1.2598 | thiophen-3-yl | 2-I | 4-Cl | H |
| I.1.2599 | thiophen-3-yl | 2-I | 5-Cl | H |
| I.1.2600 | thiophen-3-yl | 2-I | 4-F | H |
| I.1.2601 | thiophen-3-yl | 2-I | 5-F | H |
| I.1.2602 | thiophen-3-yl | 2-I | 4-CH₃ | H |
| I.1.2603 | thiophen-3-yl | 2-I | 5-CH₃ | H |
| I.1.2604 | thiophen-3-yl | 2-I | 4-CN | H |
| I.1.2605 | thiophen-3-yl | 2-I | 5-CN | H |
| I.1.2606 | thiophen-3-yl | 2-I | 4-CF₃ | H |
| I.1.2607 | thiophen-3-yl | 2-I | 5-CF₃ | H |
| I.1.2608 | thiophen-3-yl | 2-Cl | 4-Cl | 5-F |
| I.1.2609 | thiophen-3-yl | 2-Cl | 4-Cl | H |
| I.1.2610 | thiophen-3-yl | 2-Cl | 5-Cl | H |
| I.1.2611 | thiophen-3-yl | 2-Cl | 4-F | H |
| I.1.2612 | thiophen-3-yl | 2-Cl | 5-F | H |
| I.1.2613 | thiophen-3-yl | 2-Cl | 4-CH₃ | H |
| I.1.2614 | thiophen-3-yl | 2-Cl | 5-CH₃ | H |
| I.1.2615 | thiophen-3-yl | 2-Cl | 4-CN | H |
| I.1.2616 | thiophen-3-yl | 2-Cl | 5-CN | H |
| I.1.2617 | thiophen-3-yl | 2-Cl | 4-CF₃ | H |
| I.1.2618 | thiophen-3-yl | 2-Cl | 5-CF₃ | H |
| I.1.2619 | thiophen-3-yl | 2-Br | 4-Cl | 5-F |
| I.1.2620 | thiophen-3-yl | 2-Br | 4-Cl | H |
| I.1.2621 | thiophen-3-yl | 2-Br | 5-Cl | H |
| I.1.2622 | thiophen-3-yl | 2-Br | 4-F | H |
| I.1.2623 | thiophen-3-yl | 2-Br | 5-F | H |
| I.1.2624 | thiophen-3-yl | 2-Br | 4-CH₃ | H |
| I.1.2625 | thiophen-3-yl | 2-Br | 5-CH₃ | H |
| I.1.2626 | thiophen-3-yl | 2-Br | 4-CN | H |
| I.1.2627 | thiophen-3-yl | 2-Br | 5-CN | H |
| I.1.2628 | thiophen-3-yl | 2-Br | 4-CF₃ | H |
| I.1.2629 | thiophen-3-yl | 2-Br | 5-CF₃ | H |
| I.1.2630 | thiophen-3-yl | 2-CON(CH₃)₂ | 4-Cl | 5-F |
| I.1.2631 | thiophen-3-yl | 2-CON(CH₃)₂ | 4-Cl | H |
| I.1.2632 | thiophen-3-yl | 2-CON(CH₃)₂ | 5-Cl | H |
| I.1.2633 | thiophen-3-yl | 2-CON(CH₃)₂ | 4-F | H |
| I.1.2634 | thiophen-3-yl | 2-CON(CH₃)₂ | 5-F | H |
| I.1.2635 | thiophen-3-yl | 2-CON(CH₃)₂ | 4-CH₃ | H |
| I.1.2636 | thiophen-3-yl | 2-CON(CH₃)₂ | 5-CH₃ | H |
| I.1.2637 | thiophen-3-yl | 2-CON(CH₃)₂ | 4-CN | H |
| I.1.2638 | thiophen-3-yl | 2-CON(CH₃)₂ | 5-CN | H |
| I.1.2639 | thiophen-3-yl | 2-CON(CH₃)₂ | 4-CF₃ | H |
| I.1.2640 | thiophen-3-yl | 2-CON(CH₃)₂ | 5-CF₃ | H |
| I.1.2641 | thiophen-3-yl | 2-CONHCH₃ | 4-Cl | 5-F |
| I.1.2642 | thiophen-3-yl | 2-CONHCH₃ | 4-Cl | H |
| I.1.2643 | thiophen-3-yl | 2-CONHCH₃ | 5-Cl | H |
| I.1.2644 | thiophen-3-yl | 2-CONHCH₃ | 4-F | H |
| I.1.2645 | thiophen-3-yl | 2-CONHCH₃ | 5-F | H |
| I.1.2646 | thiophen-3-yl | 2-CONHCH₃ | 4-CH₃ | H |
| I.1.2647 | thiophen-3-yl | 2-CONHCH₃ | 5-CH₃ | H |
| I.1.2648 | thiophen-3-yl | 2-CONHCH₃ | 4-CN | H |
| I.1.2649 | thiophen-3-yl | 2-CONHCH₃ | 5-CN | H |
| I.1.2650 | thiophen-3-yl | 2-CONHCH₃ | 4-CF₃ | H |
| I.1.2651 | thiophen-3-yl | 2-CONHCH₃ | 5-CF₃ | H |
| I.1.2652 | thiophen-3-yl | 4-NO₂ | 5-Cl | 2-F |
| I.1.2653 | thiophen-3-yl | 4-NO₂ | 5-Cl | H |
| I.1.2654 | thiophen-3-yl | 4-NO₂ | 2-Cl | H |
| I.1.2655 | thiophen-3-yl | 4-NO₂ | 5-F | H |
| I.1.2656 | thiophen-3-yl | 4-NO₂ | 2-F | H |
| I.1.2657 | thiophen-3-yl | 4-NO₂ | 5-CH₃ | H |
| I.1.2658 | thiophen-3-yl | 4-NO₂ | 2-CH₃ | H |
| I.1.2659 | thiophen-3-yl | 4-NO₂ | 5-CN | H |
| I.1.2660 | thiophen-3-yl | 4-NO₂ | 2-CN | H |
| I.1.2661 | thiophen-3-yl | 4-NO₂ | 5-CF₃ | H |
| I.1.2662 | thiophen-3-yl | 4-NO₂ | 2-CF₃ | H |
| I.1.2663 | thiophen-3-yl | 4-CN | 5-Cl | 2-F |
| I.1.2664 | thiophen-3-yl | 4-CN | 5-Cl | H |
| I.1.2665 | thiophen-3-yl | 4-CN | 2-Cl | H |
| I.1.2666 | thiophen-3-yl | 4-CN | 5-F | H |
| I.1.2667 | thiophen-3-yl | 4-CN | 2-F | H |
| I.1.2668 | thiophen-3-yl | 4-CN | 5-CH₃ | H |
| I.1.2669 | thiophen-3-yl | 4-CN | 2-CH₃ | H |
| I.1.2670 | thiophen-3-yl | 4-CN | 5-CN | H |
| I.1.2671 | thiophen-3-yl | 4-CN | 2-CN | H |
| I.1.2672 | thiophen-3-yl | 4-CN | 5-CF₃ | H |
| I.1.2673 | thiophen-3-yl | 4-CN | 2-CF₃ | H |
| I.1.2674 | thiophen-3-yl | 4-COOH | 5-Cl | 2-F |
| I.1.2675 | thiophen-3-yl | 4-COOH | 5-Cl | H |
| I.1.2676 | thiophen-3-yl | 4-COOH | 2-Cl | H |
| I.1.2677 | thiophen-3-yl | 4-COOH | 5-F | H |
| I.1.2678 | thiophen-3-yl | 4-COOH | 2-F | H |
| I.1.2679 | thiophen-3-yl | 4-COOH | 5-CH₃ | H |
| I.1.2680 | thiophen-3-yl | 4-COOH | 2-CH₃ | H |
| I.1.2681 | thiophen-3-yl | 4-COOH | 5-CN | H |
| I.1.2682 | thiophen-3-yl | 4-COOH | 2-CN | H |
| I.1.2683 | thiophen-3-yl | 4-COOH | 5-CF₃ | H |
| I.1.2684 | thiophen-3-yl | 4-COOH | 2-CF₃ | H |
| I.1.2685 | thiophen-3-yl | 4-COOCH₃ | 5-Cl | 2-F |
| I.1.2686 | thiophen-3-yl | 4-COOCH₃ | 5-Cl | H |
| I.1.2687 | thiophen-3-yl | 4-COOCH₃ | 2-Cl | H |
| I.1.2688 | thiophen-3-yl | 4-COOCH₃ | 5-F | H |
| I.1.2689 | thiophen-3-yl | 4-COOCH₃ | 2-F | H |
| I.1.2690 | thiophen-3-yl | 4-COOCH₃ | 5-CH₃ | H |
| I.1.2691 | thiophen-3-yl | 4-COOCH₃ | 2-CH₃ | H |
| I.1.2692 | thiophen-3-yl | 4-COOCH₃ | 5-CN | H |
| I.1.2693 | thiophen-3-yl | 4-COOCH₃ | 2-CN | H |
| I.1.2694 | thiophen-3-yl | 4-COOCH₃ | 5-CF₃ | H |
| I.1.2695 | thiophen-3-yl | 4-COOCH₃ | 2-CF₃ | H |
| I.1.2696 | thiophen-3-yl | 4-COOCH₂CH₃ | 5-Cl | 2-F |
| I.1.2697 | thiophen-3-yl | 4-COOCH₂CH₃ | 5-Cl | H |
| I.1.2698 | thiophen-3-yl | 4-COOCH₂CH₃ | 2-Cl | H |
| I.1.2699 | thiophen-3-yl | 4-COOCH₂CH₃ | 5-F | H |
| I.1.2700 | thiophen-3-yl | 4-COOCH₂CH₃ | 2-F | H |
| I.1.2701 | thiophen-3-yl | 4-COOCH₂CH₃ | 5-CH₃ | H |
| I.1.2702 | thiophen-3-yl | 4-COOCH₂CH₃ | 2-CH₃ | H |
| I.1.2703 | thiophen-3-yl | 4-COOCH₂CH₃ | 5-CN | H |
| I.1.2704 | thiophen-3-yl | 4-COOCH₂CH₃ | 2-CN | H |
| I.1.2705 | thiophen-3-yl | 4-COOCH₂CH₃ | 5-CF₃ | H |
| I.1.2706 | thiophen-3-yl | 4-COOCH₂CH₃ | 2-CF₃ | H |
| I.1.2707 | thiophen-3-yl | 4-ethynyl | 5-Cl | 2-F |
| I.1.2708 | thiophen-3-yl | 4-ethynyl | 5-Cl | H |
| I.1.2709 | thiophen-3-yl | 4-ethynyl | 2-Cl | H |
| I.1.2710 | thiophen-3-yl | 4-ethynyl | 5-F | H |
| I.1.2711 | thiophen-3-yl | 4-ethynyl | 2-F | H |
| I.1.2712 | thiophen-3-yl | 4-ethynyl | 5-CH₃ | H |
| I.1.2713 | thiophen-3-yl | 4-ethynyl | 2-CH₃ | H |
| I.1.2714 | thiophen-3-yl | 4-ethynyl | 5-CN | H |
| I.1.2715 | thiophen-3-yl | 4-ethynyl | 2-CN | H |
| I.1.2716 | thiophen-3-yl | 4-ethynyl | 5-CF₃ | H |
| I.1.2717 | thiophen-3-yl | 4-ethynyl | 2-CF₃ | H |
| I.1.2718 | thiophen-3-yl | 4-I | 5-Cl | 2-F |
| I.1.2719 | thiophen-3-yl | 4-I | 5-Cl | H |
| I.1.2720 | thiophen-3-yl | 4-I | 2-Cl | H |
| I.1.2721 | thiophen-3-yl | 4-I | 5-F | H |
| I.1.2722 | thiophen-3-yl | 4-I | 2-F | H |
| I.1.2723 | thiophen-3-yl | 4-I | 5-CH₃ | H |
| I.1.2724 | thiophen-3-yl | 4-I | 2-CH₃ | H |
| I.1.2725 | thiophen-3-yl | 4-I | 5-CN | H |
| I.1.2726 | thiophen-3-yl | 4-I | 2-CN | H |
| I.1.2727 | thiophen-3-yl | 4-I | 5-CF₃ | H |
| I.1.2728 | thiophen-3-yl | 4-I | 2-CF₃ | H |
| I.1.2729 | thiophen-3-yl | 4-Cl | 5-Cl | 2-F |
| I.1.2730 | thiophen-3-yl | 4-Cl | 5-Cl | H |
| I.1.2731 | thiophen-3-yl | 4-Cl | 2-Cl | H |
| I.1.2732 | thiophen-3-yl | 4-Cl | 5-F | H |
| I.1.2733 | thiophen-3-yl | 4-Cl | 2-F | H |
| I.1.2734 | thiophen-3-yl | 4-Cl | 5-CH₃ | H |
| I.1.2735 | thiophen-3-yl | 4-Cl | 2-CH₃ | H |
| I.1.2736 | thiophen-3-yl | 4-Cl | 5-CN | H |
| I.1.2737 | thiophen-3-yl | 4-Cl | 2-CN | H |
| I.1.2738 | thiophen-3-yl | 4-Cl | 5-CF₃ | H |
| I.1.2739 | thiophen-3-yl | 4-Cl | 2-CF₃ | H |
| I.1.2740 | thiophen-3-yl | 4-Br | 5-Cl | 2-F |
| I.1.2741 | thiophen-3-yl | 4-Br | 5-Cl | H |
| I.1.2742 | thiophen-3-yl | 4-Br | 2-Cl | H |
| I.1.2743 | thiophen-3-yl | 4-Br | 5-F | H |
| I.1.2744 | thiophen-3-yl | 4-Br | 2-F | H |
| I.1.2745 | thiophen-3-yl | 4-Br | 5-CH₃ | H |
| I.1.2746 | thiophen-3-yl | 4-Br | 2-CH₃ | H |
| I.1.2747 | thiophen-3-yl | 4-Br | 5-CN | H |
| I.1.2748 | thiophen-3-yl | 4-Br | 2-CN | H |
| I.1.2749 | thiophen-3-yl | 4-Br | 5-CF₃ | H |
| I.1.2750 | thiophen-3-yl | 4-Br | 2-CF₃ | H |
| I.1.2751 | thiophen-3-yl | 4-CON(CH₃)₂ | 5-Cl | 2-F |
| I.1.2752 | thiophen-3-yl | 4-CON(CH₃)₂ | 5-Cl | H |
| I.1.2753 | thiophen-3-yl | 4-CON(CH₃)₂ | 2-Cl | H |
| I.1.2754 | thiophen-3-yl | 4-CON(CH₃)₂ | 5-F | H |
| I.1.2755 | thiophen-3-yl | 4-CON(CH₃)₂ | 2-F | H |
| I.1.2756 | thiophen-3-yl | 4-CON(CH₃)₂ | 5-CH₃ | H |
| I.1.2757 | thiophen-3-yl | 4-CON(CH₃)₂ | 2-CH₃ | H |
| I.1.2758 | thiophen-3-yl | 4-CON(CH₃)₂ | 5-CN | H |
| I.1.2759 | thiophen-3-yl | 4-CON(CH₃)₂ | 2-CN | H |
| I.1.2760 | thiophen-3-yl | 4-CON(CH₃)₂ | 5-CF₃ | H |
| I.1.2761 | thiophen-3-yl | 4-CON(CH₃)₂ | 2-CF₃ | H |
| I.1.2762 | thiophen-3-yl | 4-CONHCH₃ | 5-Cl | 2-F |
| I.1.2763 | thiophen-3-yl | 4-CONHCH₃ | 5-Cl | H |
| I.1.2764 | thiophen-3-yl | 4-CONHCH₃ | 2-Cl | H |
| I.1.2765 | thiophen-3-yl | 4-CONHCH₃ | 5-F | H |
| I.1.2766 | thiophen-3-yl | 4-CONHCH₃ | 2-F | H |
| I.1.2767 | thiophen-3-yl | 4-CONHCH₃ | 5-CH₃ | H |
| I.1.2768 | thiophen-3-yl | 4-CONHCH₃ | 2-CH₃ | H |
| I.1.2769 | thiophen-3-yl | 4-CONHCH₃ | 5-CN | H |
| I.1.2770 | thiophen-3-yl | 4-CONHCH₃ | 2-CN | H |
| I.1.2771 | thiophen-3-yl | 4-CONHCH₃ | 5-CF₃ | H |
| I.1.2772 | thiophen-3-yl | 4-CONHCH₃ | 2-CF₃ | H |
| I.1.2773 | phenyl | 2-CF₃ | H | H |
| I.1.2774 | phenyl | 2-CF₃ | 3-Cl | H |
| I.1.2775 | phenyl | 2-CF₃ | 4-Cl | H |
| I.1.2776 | phenyl | 2-CF₃ | 5-Cl | H |
| I.1.2777 | phenyl | 2-CF₃ | 6-Cl | H |
| I.1.2778 | phenyl | 2-CF₃ | 3-CF₃ | H |
| I.1.2779 | phenyl | 2-CF₃ | 4-CF₃ | H |
| I.1.2780 | phenyl | 2-CF₃ | 5-CF₃ | H |
| I.1.2781 | phenyl | 2-CF₃ | 6-CF₃ | H |
| I.1.2782 | phenyl | 2-CF₃ | 3-F | H |
| I.1.2783 | phenyl | 2-CF₃ | 4-F | H |
| I.1.2784 | phenyl | 2-CF₃ | 5-F | H |
| I.1.2785 | phenyl | 2-CF₃ | 6-F | H |
| I.1.2786 | phenyl | 2-CF₃ | 3-CH₃ | H |
| I.1.2787 | phenyl | 2-CF₃ | 4-CH₃ | H |
| I.1.2788 | phenyl | 2-CF₃ | 5-CH₃ | H |
| I.1.2789 | phenyl | 2-CF₃ | 6-CH₃ | H |
| I.1.2790 | phenyl | 2-CF₃ | 5-vinyl | H |
| I.1.2791 | phenyl | 2-CF₃ | 6-vinyl | H |
| I.1.2792 | phenyl | 2-CF₃ | 5-CF₃ | 3-Cl |
| I.1.2793 | phenyl | 2-CF₃ | 5-CF₃ | 4-Cl |
| I.1.2794 | phenyl | 2-CF₃ | 4-CF₃ | 5-Cl |
| I.1.2795 | phenyl | 2-CF₃ | 5-CF₃ | 6-Cl |
| I.1.2796 | phenyl | 2-CF₃ | 5-CF₃ | 3-F |
| I.1.2797 | phenyl | 2-CF₃ | 5-CF₃ | 4-F |
| I.1.2798 | phenyl | 2-CF₃ | 4-CF₃ | 5-F |
| I.1.2799 | phenyl | 2-CF₃ | 5-CF₃ | 6-F |
| I.1.2800 | phenyl | 2-CF₃ | 5-CF₃ | 3-CH₃ |
| I.1.2801 | phenyl | 2-CF₃ | 5-CF₃ | 4-CH₃ |
| I.1.2802 | phenyl | 2-CF₃ | 4-CF₃ | 5-CH₃ |
| I.1.2803 | phenyl | 2-CF₃ | 5-CF₃ | 6-CH₃ |
| I.1.2804 | phenyl | 2-CF₃ | 4-CF₃ | 5-vinyl |
| I.1.2805 | phenyl | 2-CF₃ | 4-CF₃ | 6-vinyl |
| I.1.2806 | phenyl | 2-CF₃ | 3-Cl | 4-F |
| I.1.2807 | phenyl | 2-CF₃ | 3-Cl | 5-F |
| I.1.2808 | phenyl | 2-CF₃ | 3-Cl | 6-F |
| I.1.2809 | phenyl | 2-CF₃ | 3-Cl | 4-Cl |
| I.1.2810 | phenyl | 2-CF₃ | 3-Cl | 5-Cl |
| I.1.2811 | phenyl | 2-CF₃ | 3-Cl | 6-Cl |
| I.1.2812 | phenyl | 2-CF₃ | 3-Cl | 4-CH₃ |
| I.1.2813 | phenyl | 2-CF₃ | 3-Cl | 5-CH₃ |
| I.1.2814 | phenyl | 2-CF₃ | 3-Cl | 6-CH₃ |
| I.1.2815 | phenyl | 2-CF₃ | 3-Cl | 5-vinyl |
| I.1.2816 | phenyl | 2-CF₃ | 3-Cl | 6-vinyl |
| I.1.2817 | phenyl | 2-CF₃ | 3-F | 4-F |
| I.1.2818 | phenyl | 2-CF₃ | 3-F | 5-F |
| I.1.2819 | phenyl | 2-CF₃ | 3-F | 6-F |
| I.1.2820 | phenyl | 2-CF₃ | 3-F | 4-Cl |
| I.1.2821 | phenyl | 2-CF₃ | 3-F | 5-Cl |
| I.1.2822 | phenyl | 2-CF₃ | 3-F | 6-Cl |
| I.1.2823 | phenyl | 2-CF₃ | 3-F | 4-CH₃ |
| I.1.2824 | phenyl | 2-CF₃ | 3-F | 5-CH₃ |
| I.1.2825 | phenyl | 2-CF₃ | 3-F | 6-CH₃ |
| I.1.2826 | phenyl | 2-CF₃ | 3-F | 5-vinyl |
| I.1.2827 | phenyl | 2-CF₃ | 3-F | 6-vinyl |
| I.1.2828 | phenyl | 2-CF₃ | 3-CH₃ | 4-F |
| I.1.2829 | phenyl | 2-CF₃ | 3-CH₃ | 5-F |
| I.1.2830 | phenyl | 2-CF₃ | 3-CH₃ | 6-F |
| I.1.2831 | phenyl | 2-CF₃ | 3-CH₃ | 4-Cl |
| I.1.2832 | phenyl | 2-CF₃ | 3-CH₃ | 5-Cl |
| I.1.2833 | phenyl | 2-CF₃ | 3-CH₃ | 6-Cl |
| I.1.2834 | phenyl | 2-CF₃ | 3-CH₃ | 4-CH₃ |
| I.1.2835 | phenyl | 2-CF₃ | 3-CH₃ | 5-CH₃ |
| I.1.2836 | phenyl | 2-CF₃ | 3-CH₃ | 6-CH₃ |
| I.1.2837 | phenyl | 2-CF₃ | 3-CH₃ | 5-vinyl |
| I.1.2838 | phenyl | 2-CF₃ | 3-CH₃ | 6-vinyl |
| I.1.2839 | phenyl | 2-CF₃ | 4-Cl | 5-F |
| I.1.2840 | phenyl | 2-CF₃ | 4-Cl | 6-F |
| I.1.2841 | phenyl | 2-CF₃ | 4-Cl | 5-Cl |
| I.1.2842 | phenyl | 2-CF₃ | 4-Cl | 6-Cl |
| I.1.2843 | phenyl | 2-CF₃ | 4-Cl | 5-CH₃ |
| I.1.2844 | phenyl | 2-CF₃ | 4-Cl | 6-CH₃ |
| I.1.2845 | phenyl | 2-CF₃ | 4-Cl | 5-vinyl |
| I.1.2846 | phenyl | 2-CF₃ | 4-Cl | 6-vinyl |
| I.1.2847 | phenyl | 2-CF₃ | 4-F | 5-F |
| I.1.2848 | phenyl | 2-CF₃ | 4-F | 6-F |
| I.1.2849 | phenyl | 2-CF₃ | 4-F | 5-Cl |
| I.1.2850 | phenyl | 2-CF₃ | 4-F | 6-Cl |
| I.1.2851 | phenyl | 2-CF₃ | 4-F | 5-CH₃ |
| I.1.2852 | phenyl | 2-CF₃ | 4-F | 6-CH₃ |
| I.1.2853 | phenyl | 2-CF₃ | 4-F | 5-vinyl |
| I.1.2854 | phenyl | 2-CF₃ | 4-F | 6-vinyl |
| I.1.2855 | phenyl | 2-CF₃ | 4-CH₃ | 5-F |
| I.1.2856 | phenyl | 2-CF₃ | 4-CH₃ | 6-F |
| I.1.2857 | phenyl | 2-CF₃ | 4-CH₃ | 5-Cl |
| I.1.2858 | phenyl | 2-CF₃ | 4-CH₃ | 6-Cl |
| I.1.2859 | phenyl | 2-CF₃ | 4-CH₃ | 5-CH₃ |
| I.1.2860 | phenyl | 2-CF₃ | 4-CH₃ | 6-CH₃ |
| I.1.2861 | phenyl | 2-CF₃ | 4-CH₃ | 5-vinyl |
| I.1.2862 | phenyl | 2-CF₃ | 4-CH₃ | 6-vinyl |
| I.1.2863 | phenyl | 2-CF₃ | 5-Cl | 6-Cl |
| I.1.2864 | phenyl | 2-CF₃ | 5-Cl | 6-F |
| I.1.2865 | phenyl | 2-CF₃ | 5-Cl | 6-CH₃ |
| I.1.2866 | phenyl | 2-CF₃ | 5-Cl | 6-vinyl |
| I.1.2867 | phenyl | 2-CF₃ | 5-F | 6-Cl |
| I.1.2868 | phenyl | 2-CF₃ | 5-F | 6-F |
| I.1.2869 | phenyl | 2-CF₃ | 5-F | 6-CH₃ |
| I.1.2870 | phenyl | 2-CF₃ | 5-F | 6-vinyl |
| I.1.2871 | phenyl | 2-CF₃ | 5-CH₃ | 6-Cl |
| I.1.2872 | phenyl | 2-CF₃ | 5-CH₃ | 6-F |
| I.1.2873 | phenyl | 2-CF₃ | 5-CH₃ | 6-CH₃ |
| I.1.2874 | phenyl | 2-CF₃ | 5-CH₃ | 6-vinyl |
| I.1.2875 | phenyl | 2-CF₃ | 5-vinyl | 6-Cl |
| I.1.2876 | phenyl | 2-CF₃ | 5-vinyl | 6-F |
| I.1.2877 | phenyl | 2-CF₃ | 5-vinyl | 6-CH₃ |
| I.1.2878 | phenyl | 2-CF₃ | 5-vinyl | 6-vinyl |
| I.1.2879 | phenyl | 2-CH₃ | H | H |
| I.1.2880 | phenyl | 2-CH₃ | 3-Cl | H |
| I.1.2881 | phenyl | 2-CH₃ | 4-Cl | H |
| I.1.2882 | phenyl | 2-CH₃ | 5-Cl | H |
| I.1.2883 | phenyl | 2-CH₃ | 6-Cl | H |
| I.1.2884 | phenyl | 2-CH₃ | 3-CF₃ | H |
| I.1.2885 | phenyl | 2-CH₃ | 4-CF₃ | H |
| I.1.2886 | phenyl | 2-CH₃ | 5-CF₃ | H |
| I.1.2887 | phenyl | 2-CH₃ | 6-CF₃ | H |
| I.1.2888 | phenyl | 2-CH₃ | 3-F | H |
| I.1.2889 | phenyl | 2-CH₃ | 4-F | H |
| I.1.2890 | phenyl | 2-CH₃ | 5-F | H |
| I.1.2891 | phenyl | 2-CH₃ | 6-F | H |
| I.1.2892 | phenyl | 2-CH₃ | 3-CH₃ | H |
| I.1.2893 | phenyl | 2-CH₃ | 4-CH₃ | H |
| I.1.2894 | phenyl | 2-CH₃ | 5-CH₃ | H |
| I.1.2895 | phenyl | 2-CH₃ | 6-CH₃ | H |
| I.1.2896 | phenyl | 2-CH₃ | 5-vinyl | H |
| I.1.2897 | phenyl | 2-CH₃ | 6-vinyl | H |
| I.1.2898 | phenyl | 2-CH₃ | 5-CF₃ | 3-Cl |
| I.1.2899 | phenyl | 2-CH₃ | 5-CF₃ | 4-Cl |
| I.1.2900 | phenyl | 2-CH₃ | 4-CF₃ | 5-Cl |
| I.1.2901 | phenyl | 2-CH₃ | 5-CF₃ | 6-Cl |
| I.1.2902 | phenyl | 2-CH₃ | 5-CF₃ | 3-F |
| I.1.2903 | phenyl | 2-CH₃ | 5-CF₃ | 4-F |
| I.1.2904 | phenyl | 2-CH₃ | 4-CF₃ | 5-F |
| I.1.2905 | phenyl | 2-CH₃ | 5-CF₃ | 6-F |
| I.1.2906 | phenyl | 2-CH₃ | 5-CF₃ | 3-CH₃ |
| I.1.2907 | phenyl | 2-CH₃ | 5-CF₃ | 4-CH₃ |
| I.1.2908 | phenyl | 2-CH₃ | 4-CF₃ | 5-CH₃ |
| I.1.2909 | phenyl | 2-CH₃ | 5-CF₃ | 6-CH₃ |
| I.1.2910 | phenyl | 2-CH₃ | 4-CF₃ | 5-vinyl |
| I.1.2911 | phenyl | 2-CH₃ | 4-CF₃ | 6-vinyl |
| I.1.2912 | phenyl | 2-CH₃ | 3-Cl | 4-F |
| I.1.2913 | phenyl | 2-CH₃ | 3-Cl | 5-F |
| I.1.2914 | phenyl | 2-CH₃ | 3-Cl | 6-F |
| I.1.2915 | phenyl | 2-CH₃ | 3-Cl | 4-Cl |
| I.1.2916 | phenyl | 2-CH₃ | 3-Cl | 5-Cl |
| I.1.2917 | phenyl | 2-CH₃ | 3-Cl | 6-Cl |
| I.1.2918 | phenyl | 2-CH₃ | 3-Cl | 4-CH₃ |
| I.1.2919 | phenyl | 2-CH₃ | 3-Cl | 5-CH₃ |
| I.1.2920 | phenyl | 2-CH₃ | 3-Cl | 6-CH₃ |
| I.1.2921 | phenyl | 2-CH₃ | 3-Cl | 5-vinyl |
| I.1.2922 | phenyl | 2-CH₃ | 3-Cl | 6-vinyl |
| I.1.2923 | phenyl | 2-CH₃ | 3-F | 4-F |
| I.1.2924 | phenyl | 2-CH₃ | 3-F | 5-F |
| I.1.2925 | phenyl | 2-CH₃ | 3-F | 6-F |
| I.1.2926 | phenyl | 2-CH₃ | 3-F | 4-Cl |
| I.1.2927 | phenyl | 2-CH₃ | 3-F | 5-Cl |
| I.1.2928 | phenyl | 2-CH₃ | 3-F | 6-Cl |
| I.1.2929 | phenyl | 2-CH₃ | 3-F | 4-CH₃ |
| I.1.2930 | phenyl | 2-CH₃ | 3-F | 5-CH₃ |
| I.1.2931 | phenyl | 2-CH₃ | 3-F | 6-CH₃ |
| I.1.2932 | phenyl | 2-CH₃ | 3-F | 5-vinyl |
| I.1.2933 | phenyl | 2-CH₃ | 3-F | 6-vinyl |
| I.1.2934 | phenyl | 2-CH₃ | 3-CH₃ | 4-F |
| I.1.2935 | phenyl | 2-CH₃ | 3-CH₃ | 5-F |
| I.1.2936 | phenyl | 2-CH₃ | 3-CH₃ | 6-F |
| I.1.2937 | phenyl | 2-CH₃ | 3-CH₃ | 4-Cl |
| I.1.2938 | phenyl | 2-CH₃ | 3-CH₃ | 5-Cl |
| I.1.2939 | phenyl | 2-CH₃ | 3-CH₃ | 6-Cl |
| I.1.2940 | phenyl | 2-CH₃ | 3-CH₃ | 4-CH₃ |
| I.1.2941 | phenyl | 2-CH₃ | 3-CH₃ | 5-CH₃ |
| I.1.2942 | phenyl | 2-CH₃ | 3-CH₃ | 6-CH₃ |
| I.1.2943 | phenyl | 2-CH₃ | 3-CH₃ | 5-vinyl |
| I.1.2944 | phenyl | 2-CH₃ | 3-CH₃ | 6-vinyl |
| I.1.2945 | phenyl | 2-CH₃ | 4-Cl | 5-F |
| I.1.2946 | phenyl | 2-CH₃ | 4-Cl | 6-F |
| I.1.2947 | phenyl | 2-CH₃ | 4-Cl | 5-Cl |
| I.1.2948 | phenyl | 2-CH₃ | 4-Cl | 6-Cl |
| I.1.2949 | phenyl | 2-CH₃ | 4-Cl | 5-CH₃ |
| I.1.2950 | phenyl | 2-CH₃ | 4-Cl | 6-CH₃ |
| I.1.2951 | phenyl | 2-CH₃ | 4-Cl | 5-vinyl |
| I.1.2952 | phenyl | 2-CH₃ | 4-Cl | 6-vinyl |
| I.1.2953 | phenyl | 2-CH₃ | 4-F | 5-F |
| I.1.2954 | phenyl | 2-CH₃ | 4-F | 6-F |
| I.1.2955 | phenyl | 2-CH₃ | 4-F | 5-Cl |
| I.1.2956 | phenyl | 2-CH₃ | 4-F | 6-Cl |
| I.1.2957 | phenyl | 2-CH₃ | 4-F | 5-CH₃ |
| I.1.2958 | phenyl | 2-CH₃ | 4-F | 6-CH₃ |
| I.1.2959 | phenyl | 2-CH₃ | 4-F | 5-vinyl |
| I.1.2960 | phenyl | 2-CH₃ | 4-F | 6-vinyl |
| I.1.2961 | phenyl | 2-CH₃ | 4-CH₃ | 5-F |
| I.1.2962 | phenyl | 2-CH₃ | 4-CH₃ | 6-F |
| I.1.2963 | phenyl | 2-CH₃ | 4-CH₃ | 5-Cl |
| I.1.2964 | phenyl | 2-CH₃ | 4-CH₃ | 6-Cl |
| I.1.2965 | phenyl | 2-CH₃ | 4-CH₃ | 5-CH₃ |
| I.1.2966 | phenyl | 2-CH₃ | 4-CH₃ | 6-CH₃ |
| I.1.2967 | phenyl | 2-CH₃ | 4-CH₃ | 5-vinyl |
| I.1.2968 | phenyl | 2-CH₃ | 4-CH₃ | 6-vinyl |
| I.1.2969 | phenyl | 2-CH₃ | 5-Cl | 6-Cl |
| I.1.2970 | phenyl | 2-CH₃ | 5-Cl | 6-F |
| I.1.2971 | phenyl | 2-CH₃ | 5-Cl | 6-CH₃ |
| I.1.2972 | phenyl | 2-CH₃ | 5-Cl | 6-vinyl |
| I.1.2973 | phenyl | 2-CH₃ | 5-F | 6-Cl |
| I.1.2974 | phenyl | 2-CH₃ | 5-F | 6-F |
| I.1.2975 | phenyl | 2-CH₃ | 5-F | 6-CH₃ |
| I.1.2976 | phenyl | 2-CH₃ | 5-F | 6-vinyl |
| I.1.2977 | phenyl | 2-CH₃ | 5-CH₃ | 6-Cl |
| I.1.2978 | phenyl | 2-CH₃ | 5-CH₃ | 6-F |
| I.1.2979 | phenyl | 2-CH₃ | 5-CH₃ | 6-CH₃ |
| I.1.2980 | phenyl | 2-CH₃ | 5-CH₃ | 6-vinyl |
| I.1.2981 | phenyl | 2-CH₃ | 5-vinyl | 6-Cl |
| I.1.2982 | phenyl | 2-CH₃ | 5-vinyl | 6-F |
| I.1.2983 | phenyl | 2-CH₃ | 5-vinyl | 6-CH₃ |
| I.1.2984 | phenyl | 2-CH₃ | 5-vinyl | 6-vinyl |
| I.1.2985 | pyridin-2-yl | 3-CF₃ | 4-Cl | 5-F |
| I.1.2986 | pyridin-2-yl | 3-CF₃ | 4-Cl | H |
| I.1.2987 | pyridin-2-yl | 3-CF₃ | 5-Cl | H |
| I.1.2988 | pyridin-2-yl | 3-CF₃ | 6-Cl | H |
| I.1.2989 | pyridin-2-yl | 3-CF₃ | 4-F | H |
| I.1.2990 | pyridin-2-yl | 3-CF₃ | 5-F | H |
| I.1.2991 | pyridin-2-yl | 3-CF₃ | 6-F | H |
| I.1.2992 | pyridin-2-yl | 3-CF₃ | 4-Br | H |
| I.1.2993 | pyridin-2-yl | 3-CF₃ | 5-Br | H |
| I.1.2994 | pyridin-2-yl | 3-CF₃ | 6-Br | H |
| I.1.2995 | pyridin-2-yl | 3-CF₃ | 4-CH₃ | H |
| I.1.2996 | pyridin-2-yl | 3-CF₃ | 5-CH₃ | H |
| I.1.2997 | pyridin-2-yl | 3-CF₃ | 6-CH₃ | H |
| I.1.2998 | pyridin-2-yl | 3-CF₃ | 4-CF₃ | H |
| I.1.2999 | pyridin-2-yl | 3-CF₃ | 5-CF₃ | H |
| I.1.3000 | pyridin-2-yl | 3-CF₃ | 6-CF₃ | H |
| I.1.3001 | pyridin-2-yl | 3-CF₃ | 4-vinyl | H |
| I.1.3002 | pyridin-2-yl | 3-CF₃ | 5-vinyl | H |
| I.1.3003 | pyridin-2-yl | 3-CF₃ | 6-vinyl | H |
| I.1.3004 | pyridin-2-yl | 3-CF₃ | 4-Cl | 6-F |
| I.1.3005 | pyridin-2-yl | 3-CF₃ | 5-Cl | 6-F |
| I.1.3006 | pyridin-2-yl | 3-CH₃ | 4-Cl | 5-F |
| I.1.3007 | pyridin-2-yl | 3-CH₃ | 4-Cl | H |
| I.1.3008 | pyridin-2-yl | 3-CH₃ | 5-Cl | H |
| I.1.3009 | pyridin-2-yl | 3-CH₃ | 6-Cl | H |
| I.1.3010 | pyridin-2-yl | 3-CH₃ | 4-F | H |
| I.1.3011 | pyridin-2-yl | 3-CH₃ | 5-F | H |
| I.1.3012 | pyridin-2-yl | 3-CH₃ | 6-F | H |
| I.1.3013 | pyridin-2-yl | 3-CH₃ | 4-Br | H |
| I.1.3014 | pyridin-2-yl | 3-CH₃ | 5-Br | H |
| I.1.3015 | pyridin-2-yl | 3-CH₃ | 6-Br | H |
| I.1.3016 | pyridin-2-yl | 3-CH₃ | 4-CH₃ | H |
| I.1.3017 | pyridin-2-yl | 3-CH₃ | 5-CH₃ | H |
| I.1.3018 | pyridin-2-yl | 3-CH₃ | 6-CH₃ | H |
| I.1.3019 | pyridin-2-yl | 3-CH₃ | 4-CF₃ | H |
| I.1.3020 | pyridin-2-yl | 3-CH₃ | 5-CF₃ | H |
| I.1.3021 | pyridin-2-yl | 3-CH₃ | 6-CF₃ | H |
| I.1.3022 | pyridin-2-yl | 3-CH₃ | 4-vinyl | H |
| I.1.3023 | pyridin-2-yl | 3-CH₃ | 5-vinyl | H |
| I.1.3024 | pyridin-2-yl | 3-CH₃ | 6-vinyl | H |
| I.1.3025 | pyridin-2-yl | 3-CH₃ | 4-Cl | 6-F |
| I.1.3026 | pyridin-2-yl | 3-CH₃ | 5-Cl | 6-F |
| I.1.3027 | pyridin-3-yl | 2-CF₃ | 4-Cl | 5-F |
| I.1.3028 | pyridin-3-yl | 2-CF₃ | 4-Cl | H |
| I.1.3029 | pyridin-3-yl | 2-CF₃ | 5-Cl | H |
| I.1.3030 | pyridin-3-yl | 2-CF₃ | 6-Cl | H |
| I.1.3031 | pyridin-3-yl | 2-CF₃ | 4-F | H |
| I.1.3032 | pyridin-3-yl | 2-CF₃ | 5-F | H |
| I.1.3033 | pyridin-3-yl | 2-CF₃ | 6-F | H |
| I.1.3034 | pyridin-3-yl | 2-CF₃ | 4-Br | H |
| I.1.3035 | pyridin-3-yl | 2-CF₃ | 5-Br | H |
| I.1.3036 | pyridin-3-yl | 2-CF₃ | 6-Br | H |
| I.1.3037 | pyridin-3-yl | 2-CF₃ | 4-CH₃ | H |
| I.1.3038 | pyridin-3-yl | 2-CF₃ | 5-CH₃ | H |
| I.1.3039 | pyridin-3-yl | 2-CF₃ | 6-CH₃ | H |
| I.1.3040 | pyridin-3-yl | 2-CF₃ | 4-CF₃ | H |
| I.1.3041 | pyridin-3-yl | 2-CF₃ | 5-CF₃ | H |
| I.1.3042 | pyridin-3-yl | 2-CF₃ | 6-CF₃ | H |
| I.1.3043 | pyridin-3-yl | 2-CF₃ | 4-vinyl | H |
| I.1.3044 | pyridin-3-yl | 2-CF₃ | 5-vinyl | H |
| I.1.3045 | pyridin-3-yl | 2-CF₃ | 6-vinyl | H |
| I.1.3046 | pyridin-3-yl | 2-CF₃ | 4-Cl | 6-F |
| I.1.3047 | pyridin-3-yl | 2-CF₃ | 5-Cl | 6-F |
| I.1.3048 | pyridin-3-yl | 2-CH₃ | 4-Cl | 5-F |
| I.1.3049 | pyridin-3-yl | 2-CH₃ | 4-Cl | H |
| I.1.3050 | pyridin-3-yl | 2-CH₃ | 5-Cl | H |
| I.1.3051 | pyridin-3-yl | 2-CH₃ | 6-Cl | H |
| I.1.3052 | pyridin-3-yl | 2-CH₃ | 4-F | H |
| I.1.3053 | pyridin-3-yl | 2-CH₃ | 5-F | H |
| I.1.3054 | pyridin-3-yl | 2-CH₃ | 6-F | H |
| I.1.3055 | pyridin-3-yl | 2-CH₃ | 4-Br | H |
| I.1.3056 | pyridin-3-yl | 2-CH₃ | 5-Br | H |
| I.1.3057 | pyridin-3-yl | 2-CH₃ | 6-Br | H |
| I.1.3058 | pyridin-3-yl | 2-CH₃ | 4-CH₃ | H |
| I.1.3059 | pyridin-3-yl | 2-CH₃ | 5-CH₃ | H |
| I.1.3060 | pyridin-3-yl | 2-CH₃ | 6-CH₃ | H |
| I.1.3061 | pyridin-3-yl | 2-CH₃ | 4-CF₃ | H |
| I.1.3062 | pyridin-3-yl | 2-CH₃ | 5-CF₃ | H |
| I.1.3063 | pyridin-3-yl | 2-CH₃ | 6-CF₃ | H |
| I.1.3064 | pyridin-3-yl | 2-CH₃ | 4-vinyl | H |
| I.1.3065 | pyridin-3-yl | 2-CH₃ | 5-vinyl | H |
| I.1.3066 | pyridin-3-yl | 2-CH₃ | 6-vinyl | H |
| I.1.3067 | pyridin-3-yl | 2-CH₃ | 4-Cl | 6-F |
| I.1.3068 | pyridin-3-yl | 2-CH₃ | 5-Cl | 6-F |
| I.1.3069 | pyridin-3-yl | 4-CF₃ | 2-Cl | 5-F |
| I.1.3070 | pyridin-3-yl | 4-CF₃ | 2-Cl | H |
| I.1.3071 | pyridin-3-yl | 4-CF₃ | 5-Cl | H |
| I.1.3072 | pyridin-3-yl | 4-CF₃ | 6-Cl | H |
| I.1.3073 | pyridin-3-yl | 4-CF₃ | 2-F | H |
| I.1.3074 | pyridin-3-yl | 4-CF₃ | 5-F | H |
| I.1.3075 | pyridin-3-yl | 4-CF₃ | 6-F | H |
| I.1.3076 | pyridin-3-yl | 4-CF₃ | 2-Br | H |
| I.1.3077 | pyridin-3-yl | 4-CF₃ | 5-Br | H |
| I.1.3078 | pyridin-3-yl | 4-CF₃ | 6-Br | H |
| I.1.3079 | pyridin-3-yl | 4-CF₃ | 2-CH₃ | H |
| I.1.3080 | pyridin-3-yl | 4-CF₃ | 5-CH₃ | H |
| I.1.3081 | pyridin-3-yl | 4-CF₃ | 6-CH₃ | H |
| I.1.3082 | pyridin-3-yl | 4-CF₃ | 2-CF₃ | H |
| I.1.3083 | pyridin-3-yl | 4-CF₃ | 5-CF₃ | H |
| I.1.3084 | pyridin-3-yl | 4-CF₃ | 6-CF₃ | H |
| I.1.3085 | pyridin-3-yl | 4-CF₃ | 2-vinyl | H |
| I.1.3086 | pyridin-3-yl | 4-CF₃ | 5-vinyl | H |
| I.1.3087 | pyridin-3-yl | 4-CF₃ | 6-vinyl | H |
| I.1.3088 | pyridin-3-yl | 4-CF₃ | 2-Cl | 6-F |
| I.1.3089 | pyridin-3-yl | 4-CF₃ | 5-Cl | 6-F |
| I.1.3090 | pyridin-3-yl | 4-CH₃ | 2-Cl | 5-F |
| I.1.3091 | pyridin-3-yl | 4-CH₃ | 2-Cl | H |
| I.1.3092 | pyridin-3-yl | 4-CH₃ | 5-Cl | H |
| I.1.3093 | pyridin-3-yl | 4-CH₃ | 6-Cl | H |
| I.1.3094 | pyridin-3-yl | 4-CH₃ | 2-F | H |
| I.1.3095 | pyridin-3-yl | 4-CH₃ | 5-F | H |
| I.1.3096 | pyridin-3-yl | 4-CH₃ | 6-F | H |
| I.1.3097 | pyridin-3-yl | 4-CH₃ | 2-Br | H |
| I.1.3098 | pyridin-3-yl | 4-CH₃ | 5-Br | H |
| I.1.3099 | pyridin-3-yl | 4-CH₃ | 6-Br | H |
| I.1.3100 | pyridin-3-yl | 4-CH₃ | 2-CH₃ | H |
| I.1.3101 | pyridin-3-yl | 4-CH₃ | 5-CH₃ | H |
| I.1.3102 | pyridin-3-yl | 4-CH₃ | 6-CH₃ | H |
| I.1.3103 | pyridin-3-yl | 4-CH₃ | 2-CF₃ | H |
| I.1.3104 | pyridin-3-yl | 4-CH₃ | 5-CF₃ | H |
| I.1.3105 | pyridin-3-yl | 4-CH₃ | 6-CF₃ | H |
| I.1.3106 | pyridin-3-yl | 4-CH₃ | 2-vinyl | H |
| I.1.3107 | pyridin-3-yl | 4-CH₃ | 5-vinyl | H |
| I.1.3108 | pyridin-3-yl | 4-CH₃ | 6-vinyl | H |
| I.1.3109 | pyridin-3-yl | 4-CH₃ | 2-Cl | 6-F |
| I.1.3110 | pyridin-3-yl | 4-CH₃ | 5-Cl | 6-F |
| I.1.3111 | pyridin-4-yl | 3-CF₃ | 2-Cl | 5-F |
| I.1.3112 | pyridin-4-yl | 3-CF₃ | 2-Cl | H |
| I.1.3113 | pyridin-4-yl | 3-CF₃ | 5-Cl | H |
| I.1.3114 | pyridin-4-yl | 3-CF₃ | 6-Cl | H |
| I.1.3115 | pyridin-4-yl | 3-CF₃ | 2-F | H |
| I.1.3116 | pyridin-4-yl | 3-CF₃ | 5-F | H |
| I.1.3117 | pyridin-4-yl | 3-CF₃ | 6-F | H |
| I.1.3118 | pyridin-4-yl | 3-CF₃ | 2-Br | H |
| I.1.3119 | pyridin-4-yl | 3-CF₃ | 5-Br | H |
| I.1.3120 | pyridin-4-yl | 3-CF₃ | 6-Br | H |
| I.1.3121 | pyridin-4-yl | 3-CF₃ | 2-CH₃ | H |
| I.1.3122 | pyridin-4-yl | 3-CF₃ | 5-CH₃ | H |
| I.1.3123 | pyridin-4-yl | 3-CF₃ | 6-CH₃ | H |
| I.1.3124 | pyridin-4-yl | 3-CF₃ | 2-CF₃ | H |
| I.1.3125 | pyridin-4-yl | 3-CF₃ | 5-CF₃ | H |
| I.1.3126 | pyridin-4-yl | 3-CF₃ | 6-CF₃ | H |
| I.1.3127 | pyridin-4-yl | 3-CF₃ | 2-vinyl | H |
| I.1.3128 | pyridin-4-yl | 3-CF₃ | 5-vinyl | H |
| I.1.3129 | pyridin-4-yl | 3-CF₃ | 6-vinyl | H |
| I.1.3130 | pyridin-4-yl | 3-CF₃ | 2-Cl | 6-F |
| I.1.3131 | pyridin-4-yl | 3-CF₃ | 5-Cl | 6-F |
| I.1.3132 | pyridin-4-yl | 3-CH₃ | 2-Cl | 5-F |
| I.1.3133 | pyridin-4-yl | 3-CH₃ | 2-Cl | H |
| I.1.3134 | pyridin-4-yl | 3-CH₃ | 5-Cl | H |
| I.1.3135 | pyridin-4-yl | 3-CH₃ | 6-Cl | H |
| I.1.3136 | pyridin-4-yl | 3-CH₃ | 2-F | H |
| I.1.3137 | pyridin-4-yl | 3-CH₃ | 5-F | H |
| I.1.3138 | pyridin-4-yl | 3-CH₃ | 6-F | H |
| I.1.3139 | pyridin-4-yl | 3-CH₃ | 2-Br | H |
| I.1.3140 | pyridin-4-yl | 3-CH₃ | 5-Br | H |
| I.1.3141 | pyridin-4-yl | 3-CH₃ | 6-Br | H |
| I.1.3142 | pyridin-4-yl | 3-CH₃ | 2-CH₃ | H |
| I.1.3143 | pyridin-4-yl | 3-CH₃ | 5-CH₃ | H |
| I.1.3144 | pyridin-4-yl | 3-CH₃ | 6-CH₃ | H |
| I.1.3145 | pyridin-4-yl | 3-CH₃ | 2-CF₃ | H |
| I.1.3146 | pyridin-4-yl | 3-CH₃ | 5-CF₃ | H |
| I.1.3147 | pyridin-4-yl | 3-CH₃ | 6-CF₃ | H |
| I.1.3148 | pyridin-4-yl | 3-CH₃ | 2-vinyl | H |
| I.1.3149 | pyridin-4-yl | 3-CH₃ | 5-vinyl | H |
| I.1.3150 | pyridin-4-yl | 3-CH₃ | 6-vinyl | H |
| I.1.3151 | pyridin-4-yl | 3-CH₃ | 2-Cl | 6-F |
| I.1.3152 | pyridin-4-yl | 3-CH₃ | 5-Cl | 6-F |
| I.1.3153 | furan-2-yl | 3-CF₃ | 4-Cl | 5-F |
| I.1.3154 | furan-2-yl | 3-CF₃ | 4-Cl | H |
| I.1.3155 | furan-2-yl | 3-CF₃ | 5-Cl | H |
| I.1.3156 | furan-2-yl | 3-CF₃ | 4-F | H |
| I.1.3157 | furan-2-yl | 3-CF₃ | 5-F | H |
| I.1.3158 | furan-2-yl | 3-CF₃ | 4-CH₃ | H |
| I.1.3159 | furan-2-yl | 3-CF₃ | 5-CH₃ | H |
| I.1.3160 | furan-2-yl | 3-CF₃ | 4-CN | H |
| I.1.3161 | furan-2-yl | 3-CF₃ | 5-CN | H |
| I.1.3162 | furan-2-yl | 3-CF₃ | 4-CF₃ | H |
| I.1.3163 | furan-2-yl | 3-CF₃ | 5-CF₃ | H |
| I.1.3164 | furan-2-yl | 3-CH₃ | 4-Cl | 5-F |
| I.1.3165 | furan-2-yl | 3-CH₃ | 4-Cl | H |
| I.1.3166 | furan-2-yl | 3-CH₃ | 5-Cl | H |
| I.1.3167 | furan-2-yl | 3-CH₃ | 4-F | H |
| I.1.3168 | furan-2-yl | 3-CH₃ | 5-F | H |
| I.1.3169 | furan-2-yl | 3-CH₃ | 4-CH₃ | H |
| I.1.3170 | furan-2-yl | 3-CH₃ | 5-CH₃ | H |
| I.1.3171 | furan-2-yl | 3-CH₃ | 4-CN | H |
| I.1.3172 | furan-2-yl | 3-CH₃ | 5-CN | H |
| I.1.3173 | furan-2-yl | 3-CH₃ | 4-CF₃ | H |
| I.1.3174 | furan-2-yl | 3-CH₃ | 5-CF₃ | H |
| I.1.3175 | furan-3-yl | 2-CF₃ | 4-Cl | 5-F |
| I.1.3176 | furan-3-yl | 2-CF₃ | 4-Cl | H |
| I.1.3177 | furan-3-yl | 2-CF₃ | 5-Cl | H |
| I.1.3178 | furan-3-yl | 2-CF₃ | 4-F | H |
| I.1.3179 | furan-3-yl | 2-CF₃ | 5-F | H |
| I.1.3180 | furan-3-yl | 2-CF₃ | 4-CH₃ | H |
| I.1.3181 | furan-3-yl | 2-CF₃ | 5-CH₃ | H |
| I.1.3182 | furan-3-yl | 2-CF₃ | 4-CN | H |
| I.1.3183 | furan-3-yl | 2-CF₃ | 5-CN | H |
| I.1.3184 | furan-3-yl | 2-CF₃ | 4-CF₃ | H |
| I.1.3185 | furan-3-yl | 2-CF₃ | 5-CF₃ | H |
| I.1.3186 | furan-3-yl | 2-CH₃ | 4-Cl | 5-F |
| I.1.3187 | furan-3-yl | 2-CH₃ | 4-Cl | H |
| I.1.3188 | furan-3-yl | 2-CH₃ | 5-Cl | H |
| I.1.3189 | furan-3-yl | 2-CH₃ | 4-F | H |
| I.1.3190 | furan-3-yl | 2-CH₃ | 5-F | H |
| I.1.3191 | furan-3-yl | 2-CH₃ | 4-CH₃ | H |
| I.1.3192 | furan-3-yl | 2-CH₃ | 5-CH₃ | H |
| I.1.3193 | furan-3-yl | 2-CH₃ | 4-CN | H |
| I.1.3194 | furan-3-yl | 2-CH₃ | 5-CN | H |
| I.1.3195 | furan-3-yl | 2-CH₃ | 4-CF₃ | H |
| I.1.3196 | furan-3-yl | 2-CH₃ | 5-CF₃ | H |
| I.1.3197 | furan-3-yl | 4-CF₃ | 5-Cl | 2-F |
| I.1.3198 | furan-3-yl | 4-CF₃ | 5-Cl | H |
| I.1.3199 | furan-3-yl | 4-CF₃ | 2-Cl | H |
| I.1.3200 | furan-3-yl | 4-CF₃ | 5-F | H |
| I.1.3201 | furan-3-yl | 4-CF₃ | 2-F | H |
| I.1.3202 | furan-3-yl | 4-CF₃ | 5-CH₃ | H |
| I.1.3203 | furan-3-yl | 4-CF₃ | 2-CH₃ | H |
| I.1.3204 | furan-3-yl | 4-CF₃ | 5-CN | H |
| I.1.3205 | furan-3-yl | 4-CF₃ | 2-CN | H |
| I.1.3206 | furan-3-yl | 4-CF₃ | 5-CF₃ | H |
| I.1.3207 | furan-3-yl | 4-CF₃ | 2-CF₃ | H |
| I.1.3208 | furan-3-yl | 4-CH₃ | 5-Cl | 2-F |
| I.1.3209 | furan-3-yl | 4-CH₃ | 5-Cl | H |
| I.1.3210 | furan-3-yl | 4-CH₃ | 2-Cl | H |
| I.1.3211 | furan-3-yl | 4-CH₃ | 5-F | H |
| I.1.3212 | furan-3-yl | 4-CH₃ | 2-F | H |
| I.1.3213 | furan-3-yl | 4-CH₃ | 5-CH₃ | H |
| I.1.3214 | furan-3-yl | 4-CH₃ | 2-CH₃ | H |
| I.1.3215 | furan-3-yl | 4-CH₃ | 5-CN | H |
| I.1.3216 | furan-3-yl | 4-CH₃ | 2-CN | H |
| I.1.3217 | furan-3-yl | 4-CH₃ | 5-CF₃ | H |
| I.1.3218 | furan-3-yl | 4-CH₃ | 2-CF₃ | H |
| I.1.3219 | thiophen-2-yl | 3-CF₃ | 4-Cl | 5-F |
| I.1.3220 | thiophen-2-yl | 3-CF₃ | 4-Cl | H |
| I.1.3221 | thiophen-2-yl | 3-CF₃ | 5-Cl | H |
| I.1.3222 | thiophen-2-yl | 3-CF₃ | 4-F | H |
| I.1.3223 | thiophen-2-yl | 3-CF₃ | 5-F | H |
| I.1.3224 | thiophen-2-yl | 3-CF₃ | 4-CH₃ | H |
| I.1.3225 | thiophen-2-yl | 3-CF₃ | 5-CH₃ | H |
| I.1.3226 | thiophen-2-yl | 3-CF₃ | 4-CN | H |
| I.1.3227 | thiophen-2-yl | 3-CF₃ | 5-CN | H |
| I.1.3228 | thiophen-2-yl | 3-CF₃ | 4-CF₃ | H |
| I.1.3229 | thiophen-2-yl | 3-CF₃ | 5-CF₃ | H |
| I.1.3230 | thiophen-2-yl | 3-CH₃ | 4-Cl | 5-F |
| I.1.3231 | thiophen-2-yl | 3-CH₃ | 4-Cl | H |
| I.1.3232 | thiophen-2-yl | 3-CH₃ | 5-Cl | H |
| I.1.3233 | thiophen-2-yl | 3-CH₃ | 4-F | H |
| I.1.3234 | thiophen-2-yl | 3-CH₃ | 5-F | H |
| I.1.3235 | thiophen-2-yl | 3-CH₃ | 4-CH₃ | H |
| I.1.3236 | thiophen-2-yl | 3-CH₃ | 5-CH₃ | H |
| I.1.3237 | thiophen-2-yl | 3-CH₃ | 4-CN | H |
| I.1.3238 | thiophen-2-yl | 3-CH₃ | 5-CN | H |
| I.1.3239 | thiophen-2-yl | 3-CH₃ | 4-CF₃ | H |
| I.1.3240 | thiophen-2-yl | 3-CH₃ | 5-CF₃ | H |
| I.1.3241 | thiophen-3-yl | 2-CF₃ | 4-Cl | 5-F |
| I.1.3242 | thiophen-3-yl | 2-CF₃ | 4-Cl | H |
| I.1.3243 | thiophen-3-yl | 2-CF₃ | 5-Cl | H |
| I.1.3244 | thiophen-3-yl | 2-CF₃ | 4-F | H |
| I.1.3245 | thiophen-3-yl | 2-CF₃ | 5-F | H |
| I.1.3246 | thiophen-3-yl | 2-CF₃ | 4-CH₃ | H |
| I.1.3247 | thiophen-3-yl | 2-CF₃ | 5-CH₃ | H |
| I.1.3248 | thiophen-3-yl | 2-CF₃ | 4-CN | H |
| I.1.3249 | thiophen-3-yl | 2-CF₃ | 5-CN | H |
| I.1.3250 | thiophen-3-yl | 2-CF₃ | 4-CF₃ | H |
| I.1.3251 | thiophen-3-yl | 2-CF₃ | 5-CF₃ | H |
| I.1.3252 | thiophen-3-yl | 2-CH₃ | 4-Cl | 5-F |
| I.1.3253 | thiophen-3-yl | 2-CH₃ | 4-Cl | H |
| I.1.3254 | thiophen-3-yl | 2-CH₃ | 5-Cl | H |
| I.1.3255 | thiophen-3-yl | 2-CH₃ | 4-F | H |
| I.1.3256 | thiophen-3-yl | 2-CH₃ | 5-F | H |
| I.1.3257 | thiophen-3-yl | 2-CH₃ | 4-CH₃ | H |
| I.1.3258 | thiophen-3-yl | 2-CH₃ | 5-CH₃ | H |
| I.1.3259 | thiophen-3-yl | 2-CH₃ | 4-CN | H |
| I.1.3260 | thiophen-3-yl | 2-CH₃ | 5-CN | H |
| I.1.3261 | thiophen-3-yl | 2-CH₃ | 4-CF₃ | H |
| I.1.3262 | thiophen-3-yl | 2-CH₃ | 5-CF₃ | H |
| I.1.3263 | thiophen-3-yl | 4-CF₃ | 5-Cl | 2-F |
| I.1.3264 | thiophen-3-yl | 4-CF₃ | 5-Cl | H |
| I.1.3265 | thiophen-3-yl | 4-CF₃ | 2-Cl | H |
| I.1.3266 | thiophen-3-yl | 4-CF₃ | 5-F | H |
| I.1.3267 | thiophen-3-yl | 4-CF₃ | 2-F | H |
| I.1.3268 | thiophen-3-yl | 4-CF₃ | 5-CH₃ | H |
| I.1.3269 | thiophen-3-yl | 4-CF₃ | 2-CH₃ | H |
| I.1.3270 | thiophen-3-yl | 4-CF₃ | 5-CN | H |
| I.1.3271 | thiophen-3-yl | 4-CF₃ | 2-CN | H |
| I.1.3272 | thiophen-3-yl | 4-CF₃ | 5-CF₃ | H |
| I.1.3273 | thiophen-3-yl | 4-CF₃ | 2-CF₃ | H |
| I.1.3274 | thiophen-3-yl | 4-CH₃ | 5-Cl | 2-F |
| I.1.3275 | thiophen-3-yl | 4-CH₃ | 5-Cl | H |
| I.1.3276 | thiophen-3-yl | 4-CH₃ | 2-Cl | H |
| I.1.3277 | thiophen-3-yl | 4-CH₃ | 5-F | H |
| I.1.3278 | thiophen-3-yl | 4-CH₃ | 2-F | H |
| I.1.3279 | thiophen-3-yl | 4-CH₃ | 5-CH₃ | H |
| I.1.3280 | thiophen-3-yl | 4-CH₃ | 2-CH₃ | H |
| I.1.3281 | thiophen-3-yl | 4-CH₃ | 5-CN | H |
| I.1.3282 | thiophen-3-yl | 4-CH₃ | 2-CN | H |
| I.1.3283 | thiophen-3-yl | 4-CH₃ | 5-CF₃ | H |
| I.1.3284 | thiophen-3-yl | 4-CH₃ | 2-CF₃ | H |
| I.1.3285 | phenyl | 3-methylisoxazol-5-yl | H | H |
| I.1.3286 | phenyl | 3-methylisoxazol-5-yl | 3-Cl | H |
| I.1.3287 | phenyl | 3-methylisoxazol-5-yl | 4-Cl | H |
| I.1.3288 | phenyl | 3-methylisoxazol-5-yl | 5-Cl | H |
| I.1.3289 | phenyl | 3-methylisoxazol-5-yl | 6-Cl | H |
| I.1.3290 | phenyl | 3-methylisoxazol-5-yl | 3-F | H |
| I.1.3291 | phenyl | 3-methylisoxazol-5-yl | 4-F | H |
| I.1.3292 | phenyl | 3-methylisoxazol-5-yl | 5-F | H |
| I.1.3293 | phenyl | 3-methylisoxazol-5-yl | 6-F | H |
| I.1.3294 | phenyl | 3-methylisoxazol-5-yl | 3-CH₃ | H |
| I.1.3295 | phenyl | 3-methylisoxazol-5-yl | 4-CH₃ | H |
| I.1.3296 | phenyl | 3-methylisoxazol-5-yl | 5-CH₃ | H |
| I.1.3297 | phenyl | 3-methylisoxazol-5-yl | 6-CH₃ | H |
| I.1.3298 | phenyl | 3-methylisoxazol-5-yl | 5-vinyl | H |
| I.1.3299 | phenyl | 3-methylisoxazol-5-yl | 6-vinyl | H |
| I.1.3300 | phenyl | 2-furyl | H | H |
| I.1.3301 | phenyl | 2-furyl | 3-Cl | H |
| I.1.3302 | phenyl | 2-furyl | 4-Cl | H |
| I.1.3303 | phenyl | 2-furyl | 5-Cl | H |
| I.1.3304 | phenyl | 2-furyl | 6-Cl | H |
| I.1.3305 | phenyl | 2-furyl | 3-F | H |
| I.1.3306 | phenyl | 2-furyl | 4-F | H |
| I.1.3307 | phenyl | 2-furyl | 5-F | H |
| I.1.3308 | phenyl | 2-furyl | 6-F | H |
| I.1.3309 | phenyl | 2-furyl | 3-CH₃ | H |
| I.1.3310 | phenyl | 2-furyl | 4-CH₃ | H |
| I.1.3311 | phenyl | 2-furyl | 5-CH₃ | H |
| I.1.3312 | phenyl | 2-furyl | 6-CH₃ | H |
| I.1.3313 | phenyl | 2-furyl | 5-vinyl | H |
| I.1.3314 | phenyl | 2-furyl | 6-vinyl | H |
| I.1.3315 | phenyl | 2-thienyl | H | H |
| I.1.3316 | phenyl | 2-thienyl | 3-Cl | H |
| I.1.3317 | phenyl | 2-thienyl | 4-Cl | H |
| I.1.3318 | phenyl | 2-thienyl | 5-Cl | H |
| I.1.3319 | phenyl | 2-thienyl | 6-Cl | H |
| I.1.3320 | phenyl | 2-thienyl | 3-F | H |
| I.1.3321 | phenyl | 2-thienyl | 4-F | H |
| I.1.3322 | phenyl | 2-thienyl | 5-F | H |
| I.1.3323 | phenyl | 2-thienyl | 6-F | H |
| I.1.3324 | phenyl | 2-thienyl | 3-CH₃ | H |
| I.1.3325 | phenyl | 2-thienyl | 4-CH₃ | H |
| I.1.3326 | phenyl | 2-thienyl | 5-CH₃ | H |
| I.1.3327 | phenyl | 2-thienyl | 6-CH₃ | H |
| I.1.3328 | phenyl | 2-thienyl | 5-vinyl | H |
| I.1.3329 | phenyl | 2-thienyl | 6-vinyl | H |
| I.1.3330 | phenyl | 2-oxazolyl | H | H |
| I.1.3331 | phenyl | 2-oxazolyl | 3-Cl | H |
| I.1.3332 | phenyl | 2-oxazolyl | 4-Cl | H |
| I.1.3333 | phenyl | 2-oxazolyl | 5-Cl | H |
| I.1.3334 | phenyl | 2-oxazolyl | 6-Cl | H |
| I.1.3335 | phenyl | 2-oxazolyl | 3-F | H |
| I.1.3336 | phenyl | 2-oxazolyl | 4-F | H |
| I.1.3337 | phenyl | 2-oxazolyl | 5-F | H |
| I.1.3338 | phenyl | 2-oxazolyl | 6-F | H |
| I.1.3339 | phenyl | 2-oxazolyl | 3-CH₃ | H |
| I.1.3340 | phenyl | 2-oxazolyl | 4-CH₃ | H |
| I.1.3341 | phenyl | 2-oxazolyl | 5-CH₃ | H |
| I.1.3342 | phenyl | 2-oxazolyl | 6-CH₃ | H |
| I.1.3343 | phenyl | 2-oxazolyl | 5-vinyl | H |
| I.1.3344 | phenyl | 2-oxazolyl | 6-vinyl | H |
| I.1.3345 | phenyl | 4-methyloxazol-2-yl | H | H |
| I.1.3346 | phenyl | 4-methyloxazol-2-yl | 3-Cl | H |
| I.1.3347 | phenyl | 4-methyloxazol-2-yl | 4-Cl | H |
| I.1.3348 | phenyl | 4-methyloxazol-2-yl | 5-Cl | H |
| I.1.3349 | phenyl | 4-methyloxazol-2-yl | 6-Cl | H |
| I.1.3350 | phenyl | 4-methyloxazol-2-yl | 3-F | H |
| I.1.3351 | phenyl | 4-methyloxazol-2-yl | 4-F | H |
| I.1.3352 | phenyl | 4-methyloxazol-2-yl | 5-F | H |
| I.1.3353 | phenyl | 4-methyloxazol-2-yl | 6-F | H |
| I.1.3354 | phenyl | 4-methyloxazol-2-yl | 3-CH₃ | H |
| I.1.3355 | phenyl | 4-methyloxazol-2-yl | 4-CH₃ | H |
| I.1.3356 | phenyl | 4-methyloxazol-2-yl | 5-CH₃ | H |
| I.1.3357 | phenyl | 4-methyloxazol-2-yl | 6-CH₃ | H |
| I.1.3358 | phenyl | 4-methyloxazol-2-yl | 5-vinyl | H |
| I.1.3359 | phenyl | 4-methyloxazol-2-yl | 6-vinyl | H |
| I.1.3360 | phenyl | 2-thiazolyl | H | H |
| I.1.3361 | phenyl | 2-thiazolyl | 3-Cl | H |
| I.1.3362 | phenyl | 2-thiazolyl | 4-Cl | H |
| I.1.3363 | phenyl | 2-thiazolyl | 5-Cl | H |
| I.1.3364 | phenyl | 2-thiazolyl | 6-Cl | H |
| I.1.3365 | phenyl | 2-thiazolyl | 3-F | H |
| I.1.3366 | phenyl | 2-thiazolyl | 4-F | H |
| I.1.3367 | phenyl | 2-thiazolyl | 5-F | H |
| I.1.3368 | phenyl | 2-thiazolyl | 6-F | H |
| I.1.3369 | phenyl | 2-thiazolyl | 3-CH₃ | H |
| I.1.3370 | phenyl | 2-thiazolyl | 4-CH₃ | H |
| I.1.3371 | phenyl | 2-thiazolyl | 5-CH₃ | H |
| I.1.3372 | phenyl | 2-thiazolyl | 6-CH₃ | H |
| I.1.3373 | phenyl | 2-thiazolyl | 5-vinyl | H |
| I.1.3374 | phenyl | 2-thiazolyl | 6-vinyl | H |
| I.1.3375 | phenyl | 2-pyridinyl | H | H |
| I.1.3376 | phenyl | 2-pyridinyl | 3-Cl | H |
| I.1.3377 | phenyl | 2-pyridinyl | 4-Cl | H |
| I.1.3378 | phenyl | 2-pyridinyl | 5-Cl | H |
| I.1.3379 | phenyl | 2-pyridinyl | 6-Cl | H |
| I.1.3380 | phenyl | 2-pyridinyl | 3-F | H |
| I.1.3381 | phenyl | 2-pyridinyl | 4-F | H |
| I.1.3382 | phenyl | 2-pyridinyl | 5-F | H |
| I.1.3383 | phenyl | 2-pyridinyl | 6-F | H |
| I.1.3384 | phenyl | 2-pyridinyl | 3-CH₃ | H |
| I.1.3385 | phenyl | 2-pyridinyl | 4-CH₃ | H |
| I.1.3386 | phenyl | 2-pyridinyl | 5-CH₃ | H |
| I.1.3387 | phenyl | 2-pyridinyl | 6-CH₃ | H |
| I.1.3388 | phenyl | 2-pyridinyl | 5-vinyl | H |
| I.1.3389 | phenyl | 2-pyridinyl | 6-vinyl | H |
| I.1.3390 | phenyl | 3-pyridinyl | H | H |
| I.1.3391 | phenyl | 3-pyridinyl | 3-Cl | H |
| I.1.3392 | phenyl | 3-pyridinyl | 4-Cl | H |
| I.1.3393 | phenyl | 3-pyridinyl | 5-Cl | H |
| I.1.3394 | phenyl | 3-pyridinyl | 6-Cl | H |
| I.1.3395 | phenyl | 3-pyridinyl | 3-F | H |
| I.1.3396 | phenyl | 3-pyridinyl | 4-F | H |
| I.1.3397 | phenyl | 3-pyridinyl | 5-F | H |
| I.1.3398 | phenyl | 3-pyridinyl | 6-F | H |
| I.1.3399 | phenyl | 3-pyridinyl | 3-CH₃ | H |
| I.1.3400 | phenyl | 3-pyridinyl | 4-CH₃ | H |
| I.1.3401 | phenyl | 3-pyridinyl | 5-CH₃ | H |
| I.1.3402 | phenyl | 3-pyridinyl | 6-CH₃ | H |
| I.1.3403 | phenyl | 3-pyridinyl | 5-vinyl | H |
| I.1.3404 | phenyl | 3-pyridinyl | 6-vinyl | H |
| I.1.3405 | phenyl | 2-pyrimidinyl | H | H |
| I.1.3406 | phenyl | 2-pyrimidinyl | 3-Cl | H |
| I.1.3407 | phenyl | 2-pyrimidinyl | 4-Cl | H |
| I.1.3408 | phenyl | 2-pyrimidinyl | 5-Cl | H |
| I.1.3409 | phenyl | 2-pyrimidinyl | 6-Cl | H |
| I.1.3410 | phenyl | 2-pyrimidinyl | 3-F | H |
| I.1.3411 | phenyl | 2-pyrimidinyl | 4-F | H |
| I.1.3412 | phenyl | 2-pyrimidinyl | 5-F | H |
| I.1.3413 | phenyl | 2-pyrimidinyl | 6-F | H |
| I.1.3414 | phenyl | 2-pyrimidinyl | 3-CH₃ | H |
| I.1.3415 | phenyl | 2-pyrimidinyl | 4-CH₃ | H |
| I.1.3416 | phenyl | 2-pyrimidinyl | 5-CH₃ | H |
| I.1.3417 | phenyl | 2-pyrimidinyl | 6-CH₃ | H |
| I.1.3418 | phenyl | 2-pyrimidinyl | 5-vinyl | H |
| I.1.3419 | phenyl | 2-pyrimidinyl | 6-vinyl | H |
| I.1.3420 | phenyl | 2-pyrazinyl | H | H |
| I.1.3421 | phenyl | 2-pyrazinyl | 3-Cl | H |
| I.1.3422 | phenyl | 2-pyrazinyl | 4-Cl | H |
| I.1.3423 | phenyl | 2-pyrazinyl | 5-Cl | H |
| I.1.3424 | phenyl | 2-pyrazinyl | 6-Cl | H |
| I.1.3425 | phenyl | 2-pyrazinyl | 3-F | H |
| I.1.3426 | phenyl | 2-pyrazinyl | 4-F | H |
| I.1.3427 | phenyl | 2-pyrazinyl | 5-F | H |
| I.1.3428 | phenyl | 2-pyrazinyl | 6-F | H |
| I.1.3429 | phenyl | 2-pyrazinyl | 3-CH₃ | H |
| I.1.3430 | phenyl | 2-pyrazinyl | 4-CH₃ | H |
| I.1.3431 | phenyl | 2-pyrazinyl | 5-CH₃ | H |
| I.1.3432 | phenyl | 2-pyrazinyl | 6-CH₃ | H |
| I.1.3433 | phenyl | 2-pyrazinyl | 5-vinyl | H |
| I.1.3434 | phenyl | 2-pyrazinyl | 6-vinyl | H |

Preference is likewise given to the compounds of the formula I.2, particularly preferably the compounds I.2.1-I.2.3434, in particular the compounds (Z,S)-I.2.1-I.2.3434, which differ from the corresponding compounds I.1.1-I.1.3434 or (Z,S)-I.1.1-I.1.3434 in that A²R^{d}R^{e}R^{f} is 2-chlorophenyl.

Preference is likewise given to the compounds of the formula I.3, particularly preferably the compounds I.3.1-I.3.3434, in particular the compounds (Z,S)-I.3.1-I.3.3434, which differ from the corresponding compounds I.1.1-I.1.3434 or (Z,S)-I.1.1-I.1.3434 in that A²R^{d}R^{e}R^{f} is 3-chlorophenyl.

Preference is likewise given to the compounds of the formula I.4, particularly preferably the compounds I.4.1-I.4.3434, in particular the compounds (Z,S)-I.4.1-I.4.3434, which differ from the corresponding compounds I.1.1-I.1.3434 or (Z,S)-I.1.1-I.1.3434 in that A²R^{d}R^{e}R^{f} is 4-chlorophenyl.

Preference is likewise given to the compounds of the formula I.5, particularly preferably the compounds I.5.1-I.5.3434, in particular the compounds (Z,S)-I.5.1-I.5.3434, which differ from the corresponding compounds I.1.1-I.1.3434 or (Z,S)-I.1.1-I.1.3434 in that A²R^{d}R^{e}R^{f} is 2-trifluoromethylphenyl.

Preference is likewise given to the compounds of the formula I.6, particularly preferably the compounds I.6.1-I.6.3434, in particular the compounds (Z,S)-I.6.1-I.6.3434, which differ from the corresponding compounds I.1.1-I.1.3434 or (Z,S)-I.1.1-I.1.3434 in that A²R^{d}R^{e}R^{f} is 3-trifluoromethylphenyl.

Preference is likewise given to the compounds of the formula I.7, particularly preferably the compounds I.7.1-I.7.3434, in particular the compounds (Z,S)-I.7.1-I.7.3434, which differ from the corresponding compounds I.1.1-I.1.3434 or (Z,S)-I.1.1-I.1.3434 in that A²R^{d}R^{e}R^{f} is 4-trifluoromethylphenyl.

Preference is likewise given to the compounds of the formula I.8, particularly preferably the compounds I.8.1-I.8.3434, in particular the compounds (Z,S)-1.8.1-I.8.3434, which differ from the corresponding compounds I.1.1-I.1.3434 or (Z,S)-I.1.1-I.1.3434 in that A²R^{d}R^{e}R^{f} is 2-methoxyphenyl.

Preference is likewise given to the compounds of the formula I.9, particularly preferably the compounds I.9.1-I.9.3434, in particular the compounds (Z,S)-I.9.1-I.9.3434, which differ from the corresponding compounds I.1.1-I.1.3434 or (Z,S)-I.1.1-I.1.3434 in that A²R^{d}R^{e}R^{f} is 3-methoxyphenyl.

Preference is likewise given to the compounds of the formula I.10, particularly preferably the compounds I.10.1-I.10.3434, in particular the compounds (Z,S)-I.10.1-I.10.3434, which differ from the corresponding compounds I.1.1-I.1.3434 or (Z,S)-I.1.1-I.1.3434 in that A²R^{d}R^{e}R^{f} is 4-methoxyphenyl.

Preference is likewise given to the compounds of the formula I.11, particularly preferably the compounds I.11.1-I.11.3434, in particular the compounds (Z,S)-I.11.1-I.11.3434, which differ from the corresponding compounds I.1.1-I.1.3434 or (Z,S)-1.1.1-I.1.3434 in that A²R^{d}R^{e}R^{f} is 2-fluorophenyl.

Preference is likewise given to the compounds of the formula I.12, particularly preferably the compounds I.12.1-I.12.3434, in particular the compounds (Z,S)-I.12.1-I.12.3434, which differ from the corresponding compounds I.1.1-I.1.3434 or (Z,S)-I.1.1-I.1.3434 in that A²R^{d}R^{e}R^{f} is 3-fluorophenyl.

Preference is likewise given to the compounds of the formula I.13, particularly preferably the compounds I.13.1-I.13.3434, in particular the compounds (Z,S)-I.13.1-I.13.3434, which differ from the corresponding compounds I.1.1-I.1.3434 or (Z,S)-I.1.1-I.1.3434 in that A²R^{d}R^{e}R^{f} is 4-fluorophenyl.

Preference is likewise given to the compounds of the formula I.14, particularly preferably the compounds I.14.1-I.14.3434, in particular the compounds (Z,S)-I.14.1-I.14.3434, which differ from the corresponding compounds I.1.1-I.1.3434 or (Z,S)-I.1.1-I.1.3434 in that A²R^{d}R^{e}R^{f} is 2-methylphenyl.

Preference is likewise given to the compounds of the formula I.15, particularly preferably the compounds I.15.1-I.15.3434, in particular the compounds (Z,S)-I.15.1-I.15.3434, which differ from the corresponding compounds I.1.1-I.1.3434 or (Z,S)-I.1.1-I.1.3434 in that A²R^{d}R^{e}R^{f} is 3-methylphenyl.

Preference is likewise given to the compounds of the formula I.16, particularly preferably the compounds I.16.1-I.16.3434, in particular the compounds (Z,S)-I.16.1-I.16.3434, which differ from the corresponding compounds I.1.1-I.1.3434 or (Z,S)-I.1.1-I.1.3434 in that A²R^{d}R^{e}R^{f} is 4-methylphenyl.

Preference is likewise given to the compounds of the formula I.17, particularly preferably the compounds I.17.1-I.17.3434, in particular the compounds (Z,S)-I.17.1-I.17.3434, which differ from the corresponding compounds I.1.1-I.1.3434 or (Z,S)-I.1.1-I.1.3434 in that A²R^{d}R^{e}R^{f} is 2-chloro-3-methylphenyl.

Preference is likewise given to the compounds of the formula I.18, particularly preferably the compounds I.18.1-I.18.3434, in particular the compounds (Z,S)-I.18.1-I.18.3434, which differ from the corresponding compounds I.1.1-I.1.3434 or (Z,S)-I.1.1-I.1.3434 in that A²R^{d}R^{e}R^{f} is 2-chloro-4-methylphenyl.

Preference is likewise given to the compounds of the formula I.19, particularly preferably the compounds I.19.1-I.19.3434, in particular the compounds (Z,S)-I.19.1-I.19.3434, which differ from the corresponding compounds I.1.1-I.1.3434 or (Z,S)-I.1.1-I.1.3434 in that A²R^{d}R^{e}R^{f} is 2-chloro-5-methylphenyl.

Preference is likewise given to the compounds of the formula I.20, particularly preferably the compounds I.20.1-I.20.3434, in particular the compounds (Z,S)-I.20.1-I.20.3434, which differ from the corresponding compounds I.1.1-I.1.3434 or (Z,S)-I.1.1-I.1.3434 in that A²R^{d}R^{e}R^{f} is 2-chloro-6-methylphenyl.

Preference is likewise given to the compounds of the formula I.21, particularly preferably the compounds I.21.1-I.21.3434, in particular the compounds (Z,S)-I.21.1-I.21.3434, which differ from the corresponding compounds I.1.1-I.1.3434 or (Z,S)-I.1.1-I.1.3434 in that A²R^{d}R^{e}R^{f} is 2,3-dichlorophenyl.

Preference is likewise given to the compounds of the formula I.22, particularly preferably the compounds I.22.1-I.22.3434, in particular the compounds (Z,S)-I.22.1-I.22.3434, which differ from the corresponding compounds I.1.1-I.1.3434 or (Z,S)-I.1.1-I.1.3434 in that A²R^{d}R^{e}R^{f} is 2,4-dichlorophenyl.

Preference is likewise given to the compounds of the formula I.23, particularly preferably the compounds I.23.1-I.23.3434, in particular the compounds (Z,S)-I.23.1-I.23.3434, which differ from the corresponding compounds I.1.1-I.1.3434 or (Z,S)-I.1.1-I.1.3434 in that A²R^{d}R^{e}R^{f} is 2,5-dichlorophenyl.

Preference is likewise given to the compounds of the formula I.24, particularly preferably the compounds I.24.1-I.24.3434, in particular the compounds (Z,S)-I.24.1-I.24.3434, which differ from the corresponding compounds I.1.1-I.1.3434 or (Z,S)-I.1.1-I.1.3434 in that A²R^{d}R^{e}R^{f} is 2,6-dichlorophenyl.

Preference is likewise given to the compounds of the formula I.25, particularly preferably the compounds I.25.1-I.25.3434, in particular the compounds (Z,S)-I.25.1-I.25.3434, which differ from the corresponding compounds I.1.1-I.1.3434 or (Z,S)-I.1.1-I.1.3434 in that A²R^{d}R^{e}R^{f} is 2-chloro-3-fluorophenyl.

Preference is likewise given to the compounds of the formula I.26, particularly preferably the compounds I.26.1-I.26.3434, in particular the compounds (Z,S)-I.26.1-I.26.3434, which differ from the corresponding compounds I.1.1-I.1.3434 or (Z,S)-I.1.1-I.1.3434 in that A²R^{d}R^{e}R^{f} is 2-chloro-4-fluorophenyl.

Preference is likewise given to the compounds of the formula I.27, particularly preferably the compounds I.27.1-I.27.3434, in particular the compounds (Z,S)-I.27.1-I.27.3434, which differ from the corresponding compounds I.1.1-I.1.3434 or (Z,S)-I.1.1-I.1.3434 in that A²R^{d}R^{e}R^{f} is 2-chloro-5-fluorophenyl.

Preference is likewise given to the compounds of the formula I.28, particularly preferably the compounds I.28.1-I.28.3434, in particular the compounds (Z,S)-I.28.1-I.28.3434, which differ from the corresponding compounds I.1.1-I.1.3434 or (Z,S)-I.1.1-I.1.3434 in that A²R^{d}R^{e}R^{f} is 2-chloro-6-fluorophenyl.

Preference is likewise given to the compounds of the formula I.29, particularly preferably the compounds I.29.1-I.29.3434, in particular the compounds (Z,S)-I.29.1-I.29.3434, which differ from the corresponding compounds I.1.1-I.1.3434 or (Z,S)-I.1.1-I.1.3434 in that A²R^{d}R^{e}R^{f} is 2,3-difluorophenyl.

Preference is likewise given to the compounds of the formula I.30, particularly preferably the compounds I.30.1-I.30.3434, in particular the compounds (Z,S)-I.30.1-I.30.3434, which differ from the corresponding compounds I.1.1-I.1.3434 or (Z,S)-I.1.1-I.1.3434 in that A² is 2,4-difluorophenyl.

Preference is likewise given to the compounds of the formula I.31, particularly preferably the compounds I.31.1-I.31.3434, in particular the compounds (Z,S)-I.31.1-I.31.3434, which differ from the corresponding compounds I.1.1-I.1.3434 or (Z,S)-I.1.1-I.1.3434 in that A²R^{d}R^{e}R^{f} is 2,5-difluorophenyl.

Preference is likewise given to the compounds of the formula I.32, particularly preferably the compounds I.32.1-I.32.3434, in particular the compounds (Z,S)-I.32.1-I.32.3434, which differ from the corresponding compounds I.1.1-I.1.3434 or (Z,S)-I.1.1-I.1.3434 in that A²R^{d}R^{e}R^{f} is 2,6-difluorophenyl.

Preference is likewise given to the compounds of the formula I.33, particularly preferably the compounds I.33.1-I.33.3434, in particular the compounds (Z,S)-I.33.1-I.33.3434, which differ from the corresponding compounds I.1.1-I.1.3434 or (Z,S)-I.1.1-I.1.3434 in that A²R^{d}R^{e}R^{f} is 2-fluoro-3-chlorophenyl.

Preference is likewise given to the compounds of the formula I.34, particularly preferably the compounds I.34.1-I.34.3434, in particular the compounds (Z,S)-I.34.1-I.34.3434, which differ from the corresponding compounds I.1.1-I.1.3434 or (Z,S)-I.1.1-I.1.3434 in that A²R^{d}R^{e}R^{f} is 2-fluoro-4-chlorophenyl.

Preference is likewise given to the compounds of the formula I.35, particularly preferably the compounds I.35.1-I.35.3434, in particular the compounds (Z,S)-I.35.1-I.35.3434, which differ from the corresponding compounds I.1.1-I.1.3434 or (Z,S)-I.1.1-I.1.3434 in that A²R^{d}R^{e}R^{f} is 2-fluoro-5-chlorophenyl.

Preference is likewise given to the compounds of the formula I.36, particularly preferably the compounds I.36.1-I.36.3434, in particular the compounds (Z,S)-I.36.1-I.36.3434, which differ from the corresponding compounds I.1.1-I.1.3434 or (Z,S)-I.1.1-I.1.3434 in that A²R^{d}R^{e}R^{f} is 3,4-difluorophenyl.

Preference is likewise given to the compounds of the formula I.37, particularly preferably the compounds I.37.1-I.37.3434, in particular the compounds (Z,S)-I.37.1-I.37.3434, which differ from the corresponding compounds I.1.1-I.1.3434 or (Z,S)-I.1.1-I.1.3434 in that A²R^{d}R^{e}R^{f} is 3,5-difluorophenyl.

Preference is likewise given to the compounds of the formula I.38, particularly preferably the compounds I.38.1-I.38.3434, in particular the compounds (Z,S)-I.38.1-I.38.3434, which differ from the corresponding compounds I.1.1-I.1.3434 or (Z,S)-I.1.1-I.1.3434 in that A²R^{d}R^{e}R^{f} is 3,4-dichlorophenyl.

Preference is likewise given to the compounds of the formula I.39, particularly preferably the compounds I.39.1-I.39.3434, in particular the compounds (Z,S)-I.39.1-I.39.3434, which differ from the corresponding compounds I.1.1-I.1.3434 or (Z,S)-I.1.1-I.1.3434 in that A²R^{d}R^{e}R^{f} is 3,5-dichlorophenyl.

Preference is likewise given to the compounds of the formula I.40, particularly preferably the compounds I.40.1-I.40.3434, in particular the compounds (Z,S)-I.40.1-I.40.3434, which differ from the corresponding compounds I.1.1-I.1.3434 or (Z,S)-I.1.1-I.1.3434 in that A²R^{d}R^{e}R^{f} is 3-fluoro,4-chlorophenyl.

Preference is likewise given to the compounds of the formula I.41, particularly preferably the compounds I.41.1-I.41.3434, in particular the compounds (Z,S)-I.41.1-I.41.3434, which differ from the corresponding compounds I.1.1-I.1.3434 or (Z,S)-I.1.1-I.1.3434 in that A²R^{d}R^{e}R^{f} is 4-fluoro,3-chlorophenyl.

Preference is likewise given to the compounds of the formula I.42, particularly preferably the compounds I.42.1-I.42.3434, in particular the compounds (Z,S)-I.42.1-I.42.3434, which differ from the corresponding compounds I.1.1-I.1.3434 or (Z,S)-I.1.1-I.1.3434 in that A²R^{d}R^{e}R^{f} is 3-fluoro,5-chlorophenyl.

Preference is likewise given to the compounds of the formula I.43, particularly preferably the compounds I.43.1-I.43.3434, in particular the compounds (Z,S)-I.43.1-I.43.3434, which differ from the corresponding compounds I.1.1-I.1.3434 or (Z,S)-I.1.1-I.1.3434 in that A²R^{d}R^{e}R^{f} is pyridin-2-yl.

Preference is likewise given to the compounds of the formula I.44, particularly preferably the compounds I.44.1-I.44.3434, in particular the compounds (Z,S)-I.44.1-I.44.3434, which differ from the corresponding compounds I.1.1-I.1.3434 or (Z,S)-I.1.1-I.1.3434 in that A²R^{d}R^{e}R^{f} is 3-chloropyridin-2-yl.

Preference is likewise given to the compounds of the formula I.45, particularly preferably the compounds I.45.1-I.45.3434, in particular the compounds (Z,S)-I.45.1-I.45.3434, which differ from the corresponding compounds I.1.1-I.1.3434 or (Z,S)-I.1.1-I.1.3434 in that A²R^{d}R^{e}R^{f} is 4-chloropyridin-2-yl.

Preference is likewise given to the compounds of the formula I.46, particularly preferably the compounds I.46.1-I.46.3434, in particular the compounds (Z,S)-I.46.1-I.46.3434, which differ from the corresponding compounds I.1.1-I.1.3434 or (Z,S)-I.1.1-I.1.3434 in that A²R^{d}R^{e}R^{f} is 5-chloropyridin-2-yl.

Preference is likewise given to the compounds of the formula I.47, particularly preferably the compounds I.47.1-I.47.3434, in particular the compounds (Z,S)-I.47.1-I.47.3434, which differ from the corresponding compounds I.1.1-I.1.3434 or (Z,S)-I.1.1-I.1.3434 in that A²R^{d}R^{e}R^{f} is 6-chloropyridin-2-yl.

Preference is likewise given to the compounds of the formula I.48, particularly preferably the compounds I.48.1-I.48.3434, in particular the compounds (Z,S)-I.48.1-I.48.3434, which differ from the corresponding compounds I.1.1-I.1.3434 or (Z,S)-I.1.1-I.1.3434 in that A²R^{d}R^{e}R^{f} is 3-trifluoromethylpyridin-2-yl.

Preference is likewise given to the compounds of the formula I.49, particularly preferably the compounds I.49.1-I.49.3434, in particular the compounds (Z,S)-I.49.1-I.49.3434, which differ from the corresponding compounds I.1.1-I.1.3434 or (Z,S)-I.1.1-I.1.3434 in that A²R^{d}R^{e}R^{f} is 4-trifluoromethylpyridin-2-yl.

Preference is likewise given to the compounds of the formula I.50, particularly preferably the compounds I.50.1-I.50.3434, in particular the compounds (Z,S)-I.50.1-I.50.3434, which differ from the corresponding compounds I.1.1-I.1.3434 or (Z,S)-I.1.1-I.1.3434 in that A²R^{d}R^{e}R^{f} is 5-trifluoromethylpyridin-2-yl.

Preference is likewise given to the compounds of the formula I.51, particularly preferably the compounds I.51.1-I.51.3434, in particular the compounds (Z,S)-I.51.1-I.51.3434, which differ from the corresponding compounds I.1.1-I.1.3434 or (Z,S)-I.1.1-I.1.3434 in that A²R^{d}R^{e}R^{f} is 6-trifluoromethylpyridin-2-yl.

Preference is likewise given to the compounds of the formula I.52, particularly preferably the compounds I.52.1-I.52.3434, in particular the compounds (Z,S)-I.52.1-I.52.3434, which differ from the corresponding compounds I.1.1-I.1.3434 or (Z,S)-I.1.1-I.1.3434 in that A²R^{d}R^{e}R^{f} is 3-methoxypyridin-2-yl.

Preference is likewise given to the compounds of the formula I.53, particularly preferably the compounds I.53.1-I.53.3434, in particular the compounds (Z,S)-I.53.1-I.53.3434, which differ from the corresponding compounds I.1.1-I.1.3434 or (Z,S)-I.1.1-I.1.3434 in that A²R^{d}R^{e}R^{f} is 4-methoxypyridin-2-yl.

Preference is likewise given to the compounds of the formula I.54, particularly preferably the compounds I.54.1-I.54.3434, in particular the compounds (Z,S)-I.54.1-I.54.3434, which differ from the corresponding compounds I.1.1-I.1.3434 or (Z,S)-I.1.1-I.1.3434 in that A²R^{d}R^{e}R^{f} is 5-methoxypyridin-2-yl.

Preference is likewise given to the compounds of the formula I.55, particularly preferably the compounds I.55.1-I.55.3434, in particular the compounds (Z,S)-I.55.1-I.55.3434, which differ from the corresponding compounds I.1.1-I.1.3434 or (Z,S)-I.1.1-I.1.3434 in that A²R^{d}R^{e}R^{f} is 6-methoxypyridin-2-yl.

Preference is likewise given to the compounds of the formula I.56, particularly preferably the compounds I.56.1-I.56.3434, in particular the compounds (Z,S)-I.56.1-I.56.3434, which differ from the corresponding compounds I.1.1-I.1.3434 or (Z,S)-I.1.1-I.1.3434 in that A²R^{d}R^{e}R^{f} is 3-fluoropyridin-2-yl.

Preference is likewise given to the compounds of the formula I.57, particularly preferably the compounds I.57.1-I.57.3434, in particular the compounds (Z,S)-I.57.1-I.57.3434, which differ from the corresponding compounds I.1.1-I.1.3434 or (Z,S)-I.1.1-I.1.3434 in that A²R^{d}R^{e}R^{f} is 4-fluoropyridin-2-yl.

Preference is likewise given to the compounds of the formula I.58, particularly preferably the compounds I.58.1-I.58.3434, in particular the compounds (Z,S)-I.58.1-I.58.3434, which differ from the corresponding compounds I.1.1-I.1.3434 or (Z,S)-I.1.1-I.1.3434 in that A²R^{d}R^{e}R^{f} is 5-fluoropyridin-2-yl.

Preference is likewise given to the compounds of the formula I.59, particularly preferably the compounds I.59.1-I.59.3434, in particular the compounds (Z,S)-I.59.1-I.59.3434, which differ from the corresponding compounds I.1.1-I.1.3434 or (Z,S)-I.1.1-I.1.3434 in that A²R^{d}R^{e}R^{f} is 6-fluoropyridin-2-yl.

Preference is likewise given to the compounds of the formula I.60, particularly preferably the compounds I.60.1-I.60.3434, in particular the compounds (Z,S)-I.60.1-I.60.3434, which differ from the corresponding compounds I.1.1-I.1.3434 or (Z,S)-I.1.1-I.1.3434 in that A²R^{d}R^{e}R^{f} is pyridin-3-yl.

Preference is likewise given to the compounds of the formula I.61, particularly preferably the compounds I.61.1-I.61.3434, in particular the compounds (Z,S)-I.61.1-I.61.3434, which differ from the corresponding compounds I.1.1-I.1.3434 or (Z,S)-I.1.1-I.1.3434 in that A²R^{d}R^{e}R^{f} is 2-chloropyridin-3-yl.

Preference is likewise given to the compounds of the formula I.62, particularly preferably the compounds I.62.1-I.62.3434, in particular the compounds (Z,S)-I.62.1-I.62.3434, which differ from the corresponding compounds I.1.1-I.1.3434 or (Z,S)-I.1.1-I.1.3434 in that A²R^{d}R^{e}R^{f} is 4-chloropyridin-3-yl.

Preference is likewise given to the compounds of the formula I.63, particularly preferably the compounds I.63.1-I.63.3434, in particular the compounds (Z,S)-I.63.1-I.63.3434, which differ from the corresponding compounds I.1.1-I.1.3434 or (Z,S)-I.1.1-I.1.3434 in that A²R^{d}R^{e}R^{f} is 5-chloropyridin-3-yl.

Preference is likewise given to the compounds of the formula I.64, particularly preferably the compounds I.64.1-I.64.3434, in particular the compounds (Z,S)-I.64.1-I.64.3434, which differ from the corresponding compounds I.1.1-I.1.3434 or (Z,S)-I.1.1-I.1.3434 in that A²R^{d}R^{e}R^{f} is 6-chloropyridin-3-yl.

Preference is likewise given to the compounds of the formula I.65, particularly preferably the compounds I.65.1-I.65.3434, in particular the compounds (Z,S)-I.65.1-I.65.3434, which differ from the corresponding compounds I.1.1-I.1.3434 or (Z,S)-I.1.1-I.1.3434 in that A²R^{d}R^{e}R^{f} is 2-trifluoromethylpyridin-3-yl.

Preference is likewise given to the compounds of the formula I.66, particularly preferably the compounds I.66.1-I.66.3434, in particular the compounds (Z,S)-I.66.1-I.66.3434, which differ from the corresponding compounds I.1.1-I.1.3434 or (Z,S)-I.1.1-I.1.3434 in that A²R^{d}R^{e}R^{f} is 4-trifluoromethylpyridin-3-yl.

Preference is likewise given to the compounds of the formula I.67, particularly preferably the compounds I.67.1-I.67.3434, in particular the compounds (Z,S)-I.67.1-I.67.3434, which differ from the corresponding compounds I.1.1-I.1.3434 or (Z,S)-I.1.1-I.1.3434 in that A²R^{d}R^{e}R^{f} is 5-trifluoromethylpyridin-3-yl.

Preference is likewise given to the compounds of the formula I.68, particularly preferably the compounds I.68.1-I.68.3434, in particular the compounds (Z,S)-I.68.1-I.68.3434, which differ from the corresponding compounds I.1.1-I.1.3434 or (Z,S)-I.1.1-I.1.3434 in that A²R^{d}R^{e}R^{f} is 6-trifluoromethylpyridin-3-yl.

Preference is likewise given to the compounds of the formula I.69, particularly preferably the compounds I.69.1-I.69.3434, in particular the compounds (Z,S)-I.69.1-I.69.3434, which differ from the corresponding compounds I.1.1-I.1.3434 or (Z,S)-I.1.1-I.1.3434 in that A²R^{d}R^{e}R^{f} is 2-methoxypyridin-3-yl.

Preference is likewise given to the compounds of the formula I.70, particularly preferably the compounds I.70.1-I.70.3434, in particular the compounds (Z,S)-I.70.1-I.70.3434, which differ from the corresponding compounds I.1.1-I.1.3434 or (Z,S)-I.1.1-I.1.3434 in that A²R^{d}R^{e}R^{f} is 4-methoxypyridin-3-yl.

Preference is likewise given to the compounds of the formula I.71, particularly preferably the compounds I.71.1-I.71.3434, in particular the compounds (Z,S)-I.71.1-I.71.3434, which differ from the corresponding compounds I.1.1-I.1.3434 or (Z,S)-I.1.1-I.1.3434 in that A²R^{d}R^{e}R^{f} is 5-methoxypyridin-3-yl.

Preference is likewise given to the compounds of the formula I.72, particularly preferably the compounds I.72.1-I.72.3434, in particular the compounds (Z,S)-I.72.1-I.72.3434, which differ from the corresponding compounds I.1.1-I.1.3434 or (Z,S)-I.1.1-I.1.3434 in that A²R^{d}R^{e}R^{f} is 6-methoxypyridin-3-yl.

Preference is likewise given to the compounds of the formula I.73, particularly preferably the compounds I.73.1-I.73.3434, in particular the compounds (Z,S)-I.73.1-I.3.3434, which differ from the corresponding compounds I.1.1-I.1.3434 or (Z,S)-I.1.1-I.1.3434 in that A²R^{d}R^{e}R^{f} is 2-fluoropyridin-3-yl.

Preference is likewise given to the compounds of the formula I.74, particularly preferably the compounds I.74.1-I.74.3434, in particular the compounds (Z,S)-I.74.1-I.74.3434, which differ from the corresponding compounds I.1.1-I.1.3434 or (Z,S)-I.1.1-I.1.3434 in that A²R^{d}R^{e}R^{f} is 4-fluoropyridin-3-yl.

Preference is likewise given to the compounds of the formula I.75, particularly preferably the compounds I.75.1-I.75.3434, in particular the compounds (Z,S)-I.75.1-I.5.3434, which differ from the corresponding compounds I.1.1-I.1.3434 or (Z,S)-I.1.1-I.1.3434 in that A²R^{d}R^{e}R^{f} is 5-fluoropyridin-3-yl.

Preference is likewise given to the compounds of the formula I.76, particularly preferably the compounds I.76.1-I.76.3434, in particular the compounds (Z,S)-I.76.1-I.76.3434, which differ from the corresponding compounds I.1.1-I.1.3434 or (Z,S)-I.1.1-I.1.3434 in that A²R^{d}R^{e}R^{f} is 6-fluoropyridin-3-yl.

Preference is likewise given to the compounds of the formula I.77, particularly preferably the compounds I.77.1-I.77.3434, in particular the compounds (Z,S)-I.77.1-I.77.3434, which differ from the corresponding compounds I.1.1-I.1.3434 or (Z,S)-I.1.1-I.1.3434 in that A²R^{d}R^{e}R^{f} is pyridin-4-yl.

Preference is likewise given to the compounds of the formula I.78, particularly preferably the compounds I.78.1-I.78.3434, in particular the compounds (Z,S)-I.78.1-I.78.3434, which differ from the corresponding compounds I.1.1-I.1.3434 or (Z,S)-I.1.1-I.1.3434 in that A²R^{d}R^{e}R^{f} is 2-fluoropyridin-4-yl.

Preference is likewise given to the compounds of the formula I.79, particularly preferably the compounds I.79.1-I.79.3434, in particular the compounds (Z,S)-I.79.1-I.79.3434, which differ from the corresponding compounds I.1.1-I.1.3434 or (Z,S)-I.1.1-I.1.3434 in that A²R^{d}R^{e}R^{f} is 3-fluoropyridin-4-yl.

Preference is likewise given to the compounds of the formula I.80, particularly preferably the compounds I.80.1-I.80.3434, in particular the compounds (Z,S)-I.80.1-I.80.3434, which differ from the corresponding compounds I.1.1-I.1.3434 or (Z,S)-I.1.1-I.1.3434 in that A²R^{d}R^{e}R^{f} is 2-methoxypyridin-4-yl.

Preference is likewise given to the compounds of the formula I.81, particularly preferably the compounds I.81.1-I.81.3434, in particular the compounds (Z,S)-I.81.1-I.81.3434, which differ from the corresponding compounds I.1.1-I.1.3434 or (Z,S)-I.1.1-I.1.3434 in that A²R^{d}R^{e}R^{f} is 3-methoxypyridin-4-yl.

Preference is likewise given to the compounds of the formula I.82, particularly preferably the compounds I.82.1-I.82.3434, in particular the compounds (Z,S)-I.82.1-I.82.3434, which differ from the corresponding compounds I.1.1-I.1.3434 or (Z,S)-I.1.1-I.1.3434 in that A²R^{d}R^{e}R^{f} is 2-trifluoromethylpyridin-4-yl.

Preference is likewise given to the compounds of the formula I.83, particularly preferably the compounds I.83.1-I.83.3434, in particular the compounds (Z,S)-I.83.1-I.83.3434, which differ from the corresponding compounds I.1.1-I.1.3434 or (Z,S)-I.1.1-I.1.3434 in that A²R^{d}R^{e}R^{f} is 3-trifluoromethylpyridin-4-yl.

Preference is likewise given to the compounds of the formula I.84, particularly preferably the compounds I.84.1-I.84.3434, in particular the compounds (Z,S)-I.84.1-I.84.3434, which differ from the corresponding compounds I.1.1-I.1.3434 or (Z,S)-I.1.1-I.1.3434 in that A²R^{d}R^{e}R^{f} is 2-chloropyridin-4-yl.

Preference is likewise given to the compounds of the formula I.85, particularly preferably the compounds I.85.1-I.85.3434, in particular the compounds (Z,S)-I.85.1-I.85.3434, which differ from the corresponding compounds I.1.1-I.1.3434 or (Z,S)-I.1.1-I.1.3434 in that A²R^{d}R^{e}R^{f} is 3-chloropyridin-4-yl.

Preference is likewise given to the compounds of the formula I.86, particularly preferably the compounds I.86.1-I.86.3434, in particular the compounds (Z,S)-I.86.1-I.86.3434, which differ from the corresponding compounds I.1.1-I.1.3434 or (Z,S)-I.1.1-I.1.3434 in that A²R^{d}R^{e}R^{f} is thiophen-2-yl.

Preference is likewise given to the compounds of the formula I.87, particularly preferably the compounds I.87.1-I.87.3434, in particular the compounds (Z,S)-I.87.1-I.87.3434, which differ from the corresponding compounds I.1.1-I.1.3434 or (Z,S)-I.1.1-I.1.3434 in that A²R^{d}R^{e}R^{f} is 3-chlorothiophen-2-yl.

Preference is likewise given to the compounds of the formula I.88, particularly preferably the compounds I.88.1-I.88.3434, in particular the compounds (Z,S)-I.88.1-I.88.3434, which differ from the corresponding compounds I.1.1-I.1.3434 or (Z,S)-I.1.1-I.1.3434 in that A²R^{d}R^{e}R^{f} is 4-chlorothiophen-2-yl.

Preference is likewise given to the compounds of the formula I.89, particularly preferably the compounds I.89.1-I.89.3434, in particular the compounds (Z,S)-I.89.1-I.89.3434, which differ from the corresponding compounds I.1.1-I.1.3434 or (Z,S)-I.1.1-I.1.3434 in that A²R^{d}R^{e}R^{f} is 5-chlorothiophen-2-yl.

Preference is likewise given to the compounds of the formula I.90, particularly preferably the compounds I.90.1-I.90.3434, in particular the compounds (Z,S)-I.90.1-I.90.3434, which differ from the corresponding compounds I.1.1-I.1.3434 or (Z,S)-I.1.1-I.1.3434 in that A²R^{d}R^{e}R^{f} is 3-trifluoromethylthiophen-2-yl.

Preference is likewise given to the compounds of the formula I.91, particularly preferably the compounds I.91.1-I.91.3434, in particular the compounds (Z,S)-I.91.1-I.91.3434, which differ from the corresponding compounds I.1.1-I.1.3434 or (Z,S)-I.1.1-I.1.3434 in that A²R^{d}R^{e}R^{f} is 4-trifluoromethylthiophen-2-yl.

Preference is likewise given to the compounds of the formula I.92, particularly preferably the compounds I.92.1-I.92.3434, in particular the compounds (Z,S)-I.92.1-I.92.3434, which differ from the corresponding compounds I.1.1-I.1.3434 or (Z,S)-I.1.1-I.1.3434 in that A²R^{d}R^{e}R^{f} is 5-trifluoromethylthiophen-2-yl.

Preference is likewise given to the compounds of the formula I.93, particularly preferably the compounds I.93.1-I.93.3434, in particular the compounds (Z,S)-I.93.1-I.93.3434, which differ from the corresponding compounds I.1.1-I.1.3434 or (Z,S)-I.1.1-I.1.3434 in that A²R^{d}R^{e}R^{f} is 3-methoxythiophen-2-yl.

Preference is likewise given to the compounds of the formula I.94, particularly preferably the compounds I.94.1-I.94.3434, in particular the compounds (Z,S)-I.94.1-I.94.3434, which differ from the corresponding compounds I.1.1-I.1.3434 or (Z,S)-I.1.1-I.1.3434 in that A²R^{d}R^{e}R^{f} is 4-methoxythiophen-2-yl.

Preference is likewise given to the compounds of the formula I.95, particularly preferably the compounds I.95.1-I.95.3434, in particular the compounds (Z,S)-I.95.1-I.95.3434, which differ from the corresponding compounds I.1.1-I.1.3434 or (Z,S)-I.1.1-I.1.3434 in that A²R^{d}R^{e}R^{f} is 5-methoxythiophen-2-yl.

Preference is likewise given to the compounds of the formula I.96, particularly preferably the compounds I.96.1-I.96.3434, in particular the compounds (Z,S)-I.96.1-I.96.3434, which differ from the corresponding compounds I.1.1-I.1.3434 or (Z,S)-I.1.1-I.1.3434 in that A²R^{d}R^{e}R^{f} is 3-fluorothiophen-2-yl.

Preference is likewise given to the compounds of the formula I.97, particularly preferably the compounds I.97.1-I.97.3434, in particular the compounds (Z,S)-I.97.1-I.97.3434, which differ from the corresponding compounds I.1.1-I.1.3434 or (Z,S)-I.1.1-I.1.3434 in that A²R^{d}R^{e}R^{f} is 4-fluorothiophen-2-yl.

Preference is likewise given to the compounds of the formula I.98, particularly preferably the compounds I.98.1-I.98.3434, in particular the compounds (Z,S)-I.98.1-I.98.3434, which differ from the corresponding compounds I.1.1-I.1.3434 or (Z,S)-I.1.1-I.1.3434 in that A²R^{d}R^{e}R^{f} is 5-fluorothiophen-2-yl.

Preference is likewise given to the compounds of the formula I.99, particularly preferably the compounds I.99.1-I.99.3434, in particular the compounds (Z,S)-I.99.1-I.99.3434, which differ from the corresponding compounds I.1.1-I.1.3434 or (Z,S)-I.1.1-I.1.3434 in that A²R^{d}R^{e}R^{f} is thiophen-3-yl.

Preference is likewise given to the compounds of the formula I.100, particularly preferably the compounds I.100.1-I.100.3434, in particular the compounds (Z,S)-I.100.1-I.100.3434, which differ from the corresponding compounds I.1.1-I.1.3434 or (Z,S)-I.1.1-I.1.3434 in that A²R^{d}R^{e}R^{f} is 2-chlorothiophen-3-yl.

Preference is likewise given to the compounds of the formula I.101, particularly preferably the compounds I.101.1-I.101.3434, in particular the compounds (Z,S)-I.101.1-I.101.3434, which differ from the corresponding compounds I.1.1-I.1.3434 or (Z,S)-I.1.1-I.1.3434 in that A²R^{d}R^{e}R^{f} is 4-chlorothiophen-3-yl.

Preference is likewise given to the compounds of the formula I.102, particularly preferably the compounds I.102.1-I.102.3434, in particular the compounds (Z,S)-I.102.1-I.102.3434, which differ from the corresponding compounds I.1.1-I.1.3434 or (Z,S)-I.1.1-I.1.3434 in that A²R^{d}R^{e}R^{f} is 5-chlorothiophen-3-yl.

Preference is likewise given to the compounds of the formula I.103, particularly preferably the compounds I.103.1-I.103.3434, in particular the compounds (Z,S)-I.103.1-I.103.3434, which differ from the corresponding compounds I.1.1-I.1.3434 or (Z,S)-I.1.1-I.1.3434 in that A²R^{d}R^{e}R^{f} is 2-fluorothiophen-3-yl.

Preference is likewise given to the compounds of the formula I.104, particularly preferably the compounds I.104.1-I.104.3434, in particular the compounds (Z,S)-I.104.1-I.104.3434, which differ from the corresponding compounds I.1.1-I.1.3434 or (Z,S)-I.1.1-I.1.3434 in that A²R^{d}R^{e}R^{f} is 4-fluorothiophen-3-yl.

Preference is likewise given to the compounds of the formula I.105, particularly preferably the compounds I.105.1-I.105.3434, in particular the compounds (Z,S)-I.105.1-I.105.3434, which differ from the corresponding compounds I.1.1-I.1.3434 or (Z,S)-I.1.1-I.1.3434 in that A²R^{d}R^{e}R^{f} is 5-fluorothiophen-3-yl.

Preference is likewise given to the compounds of the formula I.106, particularly preferably the compounds I.106.1-I.106.3434, in particular the compounds (Z,S)-I.106.1-I.106.3434, which differ from the corresponding compounds I.1.1-I.1.3434 or (Z,S)-I.1.1-I.1.3434 in that A²R^{d}R^{e}R^{f} is 2-methoxythiophen-3-yl.

Preference is likewise given to the compounds of the formula I.107, particularly preferably the compounds I.107.1-I.107.3434, in particular the compounds (Z,S)-I.107.1-I.107.3434, which differ from the corresponding compounds I.1.1-I.1.3434 or (Z,S)-I.1.1-I.1.3434 in that A²R^{d}R^{e}R^{f} is 4-methoxythiophen-3-yl.

Preference is likewise given to the compounds of the formula I.108, particularly preferably the compounds I.108.1-I.108.3434, in particular the compounds (Z,S)-I.108.1-I.108.3434, which differ from the corresponding compounds I.1.1-I.1.3434 or (Z,S)-I.1.1-I.1.3434 in that A²R^{d}R^{e}R^{f} is 5-methoxythiophen-3-yl.

Preference is likewise given to the compounds of the formula I.109, particularly preferably the compounds I.109.1-I.109.3434, in particular the compounds (Z,S)-I.109.1-I.109.3434, which differ from the corresponding compounds I.1.1-I.1.3434 or (Z,S)-I.1.1-I.1.3434 in that A²R^{d}R^{e}R^{f} is 2-trifluoromethylthiophen-3-yl.

Preference is likewise given to the compounds of the formula I.110, particularly preferably the compounds I.110.1-I.110.3434, in particular the compounds (Z,S)-I.110.1-I.110.3434, which differ from the corresponding compounds I.1.1-I.1.3434 or (Z,S)-I.1.1-I.1.3434 in that A²R^{d}R^{e}R^{f} is 4-trifluoromethylthiophen-3-yl.

Preference is likewise given to the compounds of the formula I.111, particularly preferably the compounds I.111.1-I.111.3434, in particular the compounds (Z,S)-I.111.1-I.111.3434, which differ from the corresponding compounds I.1.1-I.1.3434 or (Z,S)-I.1.1-I.1.3434 in that A²R^{d}R^{e}R^{f} is 5-trifluoromethylthiophen-3-yl.

Preference is likewise given to the compounds of the formula I.112, particularly preferably the compounds I.112.1-I.112.3434, in particular the compounds (Z,S)-I.112.1-I.112.3434, which differ from the corresponding compounds I.1.1-I.1.3434 or (Z,S)-I.1.1-I.1.3434 in that A²R^{d}R^{e}R^{f} is furan-2-yl.

Preference is likewise given to the compounds of the formula I.113, particularly preferably the compounds I.113.1-I.113.3434, in particular the compounds (Z,S)-I.113.1-I.113.3434, which differ from the corresponding compounds I.1.1-I.1.3434 or (Z,S)-1.1.1-I.1.3434 in that A²R^{d}R^{e}R^{f} is 3-chlorofuran-2-yl.

Preference is likewise given to the compounds of the formula I.114, particularly preferably the compounds I.114.1-I.114.3434, in particular the compounds (Z,S)-I.114.1-I.114.3434, which differ from the corresponding compounds I.1.1-I.1.3434 or (Z,S)-I.1.1-I.1.3434 in that A²R^{d}R^{e}R^{f} is 4-chlorofuran-2-yl.

Preference is likewise given to the compounds of the formula I.115, particularly preferably the compounds I.115.1-I.115.3434, in particular the compounds (Z,S)-I.115.1-I.115.3434, which differ from the corresponding compounds I.1.1-I.1.3434 or (Z,S)-I.1.1-I.1.3434 in that A²R^{d}R^{e}R^{f} is 5-chlorofuran-2-yl.

Preference is likewise given to the compounds of the formula I.116, particularly preferably the compounds I.116.1-I.116.3434, in particular the compounds (Z,S)-I.116.1-I.116.3434, which differ from the corresponding compounds I.1.1-I.1.3434 or (Z,S)-I.1.1-I.1.3434 in that A²R^{d}R^{e}R^{f} is 3-fluorofuran-2-yl.

Preference is likewise given to the compounds of the formula I.117, particularly preferably the compounds I.117.1-I.117.3434, in particular the compounds (Z,S)-I.117.1-I.117.3434, which differ from the corresponding compounds I.1.1-I.1.3434 or (Z,S)-I.1.1-I.1.3434 in that A²R^{d}R^{e}R^{f} is 4-fluorofuran-2-yl.

Preference is likewise given to the compounds of the formula I.118, particularly preferably the compounds I.118.1-I.118.3434, in particular the compounds (Z,S)-I.118.1-I.118.3434, which differ from the corresponding compounds I.1.1-I.1.3434 or (Z,S)-I.1.1-I.1.3434 in that A²R^{d}R^{e}R^{f} is 5-fluorofuran-2-yl.

Preference is likewise given to the compounds of the formula I.119, particularly preferably the compounds I.119.1-I.119.3434, in particular the compounds (Z,S)-I.119.1-I.119.3434, which differ from the corresponding compounds I.1.1-I.1.3434 or (Z,S)-I.1.1-I.1.3434 in that A²R^{d}R^{e}R^{f} is furan-3-yl.

Preference is likewise given to the compounds of the formula I.120, particularly preferably the compounds I.120.1-I.120.3434, in particular the compounds (Z,S)-I.120.1-I.120.3434, which differ from the corresponding compounds I.1.1-I.1.3434 or (Z,S)-I.1.1-I.1.3434 in that A²R^{d}R^{e}R^{f} is 2-chlorofuran-3-yl.

Preference is likewise given to the compounds of the formula I.121, particularly preferably the compounds I.121.1-I.121.3434, in particular the compounds (Z,S)-I.121.1-I.121.3434, which differ from the corresponding compounds I.1.1-I.1.3434 or (Z,S)-I.1.1-I.1.3434 in that A²R^{d}R^{e}R^{f} is 4-chlorofuran-3-yl.

Preference is likewise given to the compounds of the formula I.122, particularly preferably the compounds I.122.1-I.122.3434, in particular the compounds (Z,S)-I.122.1-I.122.3434, which differ from the corresponding compounds I.1.1-I.1.3434 or (Z,S)-I.1.1-I.1.3434 in that A²R^{d}R^{e}R^{f} is 5-chlorofuran-3-yl.

Preference is likewise given to the compounds of the formula I.123, particularly preferably the compounds I.123.1-I.123.3434, in particular the compounds (Z,S)-I.123.1-I.123.3434, which differ from the corresponding compounds I.1.1-I.1.3434 or (Z,S)-I.1.1-I.1.3434 in that A²R^{d}R^{e}R^{f} is 2-fluorofuran-3-yl.

Preference is likewise given to the compounds of the formula I.124, particularly preferably the compounds I.124.1-I.124.3434, in particular the compounds (Z,S)-I.24.1-I.124.3434, which differ from the corresponding compounds I.1.1-I.1.3434 or (Z,S)-I.1.1-I.1.3434 in that A²R^{d}R^{e}R^{f} is 4-fluorofuran-3-yl.

Preference is likewise given to the compounds of the formula I.125, particularly preferably the compounds I.125.1-I.125.3434, in particular the compounds (Z,S)-I.125.1-I.125.3434, which differ from the corresponding compounds I.1.1-I.1.3434 or (Z,S)-I.1.1-I.1.3434 in that A²R^{d}R^{e}R^{f} is 5-fluorofuran-3-yl.

Preference is likewise given to the compounds of the formula I.126, particularly preferably the compounds I.126.1-I.126.3434, in particular the compounds (Z,S)-I.126.1-I.126.3434, which differ from the corresponding compounds I.1.1-I.1.3434 or (Z,S)-I.1.1-I.1.3434 in that A²R^{d}R^{e}R^{f} is 5-fluorofuran-3-yl.

Preference is likewise given to the compounds of the formula I.127, particularly preferably the compounds I.127.1-I.127.3434, in particular the compounds (Z,S)-I.127.1-I.127.3434, which differ from the corresponding compounds I.1.1-I.1.3434 or (Z,S)-I.1.1-I.1.3434 in that R¹ is CH₃.

Preference is likewise given to the compounds of the formula I.128, particularly preferably the compounds I.128.1-I.128.3434, in particular the compounds (Z,S)-I.128.1-I.128.3434, which differ from the corresponding compounds I.1.1-I.1.3434 or (Z,S)-I.1.1-I.1.3434 in that A²R^{d}R^{e}R^{f} is 2-chlorophenyl and R¹ is CH₃.

Preference is likewise given to the compounds of the formula I.129, particularly preferably the compounds I.129.1-I.129.3434, in particular the compounds (Z,S)-I.129.1-I.129.3434, which differ from the corresponding compounds I.1.1-I.1.3434 or (Z,S)-I.1.1-I.1.3434 in that A²R^{d}R^{e}R^{f} is 3-chlorophenyl and R¹ is CH₃.

Preference is likewise given to the compounds of the formula I.130, particularly preferably the compounds I.130.1-I.130.3434, in particular the compounds (Z,S)-I.130.1-I.130.3434, which differ from the corresponding compounds I.1.1-I.1.3434 or (Z,S)-I.1.1-I.1.3434 in that A²R^{d}R^{e}R^{f} is 4-chlorophenyl and R¹ is CH₃.

Preference is likewise given to the compounds of the formula I.131, particularly preferably the compounds I.131.1-I.131.3434, in particular the compounds (Z,S)-I.131.1-I.131.3434, which differ from the corresponding compounds I.1.1-I.1.3434 or (Z,S)-I.1.1-I.1.3434 in that A²R^{d}R^{e}R^{f} is 2-trifluoromethylphenyl and R¹ is CH₃.

Preference is likewise given to the compounds of the formula I.132, particularly preferably the compounds I.132.1-I.132.3434, in particular the compounds (Z,S)-I.132.1-I.132.3434, which differ from the corresponding compounds I.1.1-I.1.3434 or (Z,S)-I.1.1-I.1.3434 in that A²R^{d}R^{e}R^{f} is 3-trifluoromethylphenyl and R¹ is CH₃.

Preference is likewise given to the compounds of the formula I.133, particularly preferably the compounds I.133.1-I.133.3434, in particular the compounds (Z,S)-I.133.1-I.133.3434, which differ from the corresponding compounds I.1.1-I.1.3434 or (Z,S)-I.1.1-I.1.3434 in that A²R^{d}R^{e}R^{f} is 4-trifluoromethylphenyl and R¹ is CH₃.

Preference is likewise given to the compounds of the formula I.134, particularly preferably the compounds I.134.1-I.134.3434, in particular the compounds (Z,S)-I.134.1-I.134.3434, which differ from the corresponding compounds I.1.1-I.1.3434 or (Z,S)-I.1.1-I.1.3434 in that A²R^{d}R^{e}R^{f} is 2-methoxyphenyl and R¹ is CH₃.

Preference is likewise given to the compounds of the formula I.135, particularly preferably the compounds I.135.1-I.135.3434, in particular the compounds (Z,S)-I.135.1-I.135.3434, which differ from the corresponding compounds I.1.1-I.1.3434 or (Z,S)-I.1.1-I.1.3434 in that A²R^{d}R^{e}R^{f} is 3-methoxyphenyl and R¹ is CH₃.

Preference is likewise given to the compounds of the formula I.136, particularly preferably the compounds I.136.1-I.136.3434, in particular the compounds (Z,S)-I.136.1-I.136.3434, which differ from the corresponding compounds I.1.1-I.1.3434 or (Z,S)-I.1.1-I.1.3434 in that A²R^{d}R^{e}R^{f} is 4-methoxyphenyl and R¹ is CH₃.

Preference is likewise given to the compounds of the formula I.137, particularly preferably the compounds I.137.1-I.137.3434, in particular the compounds (Z,S)-I.37.1-I.137.3434, which differ from the corresponding compounds I.1.1-I.1.3434 or (Z,S)-I.1.1-I.1.3434 in that A²R^{d}R^{e}R^{f} is 2-fluorophenyl and R¹ is CH₃.

Preference is likewise given to the compounds of the formula I.138, particularly preferably the compounds I.138.1-I.138.3434, in particular the compounds (Z,S)-I.138.1-I.138.3434, which differ from the corresponding compounds I.1.1-I.1.3434 or (Z,S)-I.1.1-I.1.3434 in that A²R^{d}R^{e}R^{f} is 3-fluorophenyl and R¹ is CH₃.

Preference is likewise given to the compounds of the formula I.139, particularly preferably the compounds I.139.1-I.139.3434, in particular the compounds (Z,S)-I.139.1-I.139.3434, which differ from the corresponding compounds I.1.1-I.1.3434 or (Z,S)-I.1.1-I.1.3434 in that A²R^{d}R^{e}R^{f} is 4-fluorophenyl and R¹ is CH₃.

Preference is likewise given to the compounds of the formula I.140, particularly preferably the compounds I.140.1-I.140.3434, in particular the compounds (Z,S)-I.140.1-I.140.3434, which differ from the corresponding compounds I.1.1-I.1.3434 or (Z,S)-I.1.1-I.1.3434 in that A²R^{d}R^{e}R^{f} is 2-methylphenyl and R¹ is CH₃.

Preference is likewise given to the compounds of the formula I.141, particularly preferably the compounds I.141.1-I.141.3434, in particular the compounds (Z,S)-I.141.1-I.141.3434, which differ from the corresponding compounds I.1.1-I.1.3434 or (Z,S)-I.1.1-I.1.3434 in that A²R^{d}R^{e}R^{f} is 3-methylphenyl and R¹ is CH₃.

Preference is likewise given to the compounds of the formula I.142, particularly preferably the compounds I.142.1-I.142.3434, in particular the compounds (Z,S)-I.142.1-I.142.3434, which differ from the corresponding compounds I.1.1-I.1.3434 or (Z,S)-I.1.1-I.1.3434 in that A²R^{d}R^{e}R^{f} is 4-methylphenyl and R¹ is CH₃.

Preference is likewise given to the compounds of the formula I.143, particularly preferably the compounds I.143.1-I.143.3434, in particular the compounds (Z,S)-I.143.1-I.143.3434, which differ from the corresponding compounds I.1.1-I.1.3434 or (Z,S)-I.1.1-I.1.3434 in that A²R^{d}R^{e}R^{f} is 2-chloro-3-methylphenyl and R¹ is CH₃.

Preference is likewise given to the compounds of the formula I.144, particularly preferably the compounds I.144.1-I.144.3434, in particular the compounds (Z,S)-I.144.1-I.144.3434, which differ from the corresponding compounds I.1.1-I.1.3434 or (Z,S)-I.1.1-I.1.3434 in that A²R^{d}R^{e}R^{f} is 2-chloro-4-methylphenyl and R¹ is CH₃.

Preference is likewise given to the compounds of the formula I.145, particularly preferably the compounds I.145.1-I.145.3434, in particular the compounds (Z,S)-I.145.1-I.145.3434, which differ from the corresponding compounds I.1.1-I.1.3434 or (Z,S)-I.1.1-I.1.3434 in that A²R^{d}R^{e}R^{f} is 2-chloro-5-methylphenyl and R¹ is CH₃.

Preference is likewise given to the compounds of the formula I.146, particularly preferably the compounds I.146.1-I.146.3434, in particular the compounds (Z,S)-I.146.1-I.146.3434, which differ from the corresponding compounds I.1.1-I.1.3434 or (Z,S)-I.1.1-I.1.3434 in that A²R^{d}R^{e}R^{f} is 2-chloro-6-methylphenyl and R¹ is CH₃.

Preference is likewise given to the compounds of the formula I.147, particularly preferably the compounds I.147.1-I.147.3434, in particular the compounds (Z,S)-I.147.1-I.147.3434, which differ from the corresponding compounds I.1.1-I.1.3434 or (Z,S)-I.1.1-I.1.3434 in that A²R^{d}R^{e}R^{f} is 2,3-dichlorophenyl and R¹ is CH₃.

Preference is likewise given to the compounds of the formula I.148, particularly preferably the compounds I.148.1-I.148.3434, in particular the compounds (Z,S)-I.148.1-I.148.3434, which differ from the corresponding compounds I.1.1-I.1.3434 or (Z,S)-I.1.1-I.1.3434 in that A²R^{d}R^{e}R^{f} is 2,4-dichlorophenyl and R¹ is CH₃.

Preference is likewise given to the compounds of the formula I.149, particularly preferably the compounds I.149.1-I.149.3434, in particular the compounds (Z,S)-I.149.1-I.149.3434, which differ from the corresponding compounds I.1.1-I.1.3434 or (Z,S)-I.1.1-I.1.3434 in that A²R^{d}R^{e}R^{f} is 2,5-dichlorophenyl and R¹ is CH₃.

Preference is likewise given to the compounds of the formula I.150, particularly preferably the compounds I.150.1-I.150.3434, in particular the compounds (Z,S)-I.150.1-I.150.3434, which differ from the corresponding compounds I.1.1-I.1.3434 or (Z,S)-I.1.1-I.1.3434 in that A²R^{d}R^{e}R^{f} is 2,6-dichlorophenyl and R¹ is CH₃.

Preference is likewise given to the compounds of the formula I.151, particularly preferably the compounds I.151.1-I.151.3434, in particular the compounds (Z,S)-I.151.1-I.151.3434, which differ from the corresponding compounds I.1.1-I.1.3434 or (Z,S)-I.1.1-I.1.3434 in that A²R^{d}R^{e}R^{f} is 2-chloro-3-fluorophenyl and R¹ is CH₃.

Preference is likewise given to the compounds of the formula I.152, particularly preferably the compounds I.152.1-I.152.3434, in particular the compounds (Z,S)-I.152.1-I.152.3434, which differ from the corresponding compounds I.1.1-I.1.3434 or (Z,S)-I.1.1-I.1.3434 in that A²R^{d}R^{e}R^{f} is 2-chloro-4-fluorophenyl and R¹ is CH₃.

Preference is likewise given to the compounds of the formula I.153, particularly preferably the compounds I.153.1-I.153.3434, in particular the compounds (Z,S)-I.153.1-I.153.3434, which differ from the corresponding compounds I.1.1-I.1.3434 or (Z,S)-I.1.1-I.1.3434 in that A²R^{d}R^{e}R^{f} is 2-chloro-5-fluorophenyl and R¹ is CH₃.

Preference is likewise given to the compounds of the formula I.154, particularly preferably the compounds I.154.1-I.154.3434, in particular the compounds (Z,S)-I.154.1-I.154.3434, which differ from the corresponding compounds I.1.1-I.1.3434 or (Z,S)-I.1.1-I.1.3434 in that A²R^{d}R^{e}R^{f} is 2-chloro-6-fluorophenyl and R¹ is CH₃.

Preference is likewise given to the compounds of the formula I.155, particularly preferably the compounds I.155.1-I.155.3434, in particular the compounds (Z,S)-I.155.1-I.155.3434, which differ from the corresponding compounds I.1.1-I.1.3434 or (Z,S)-I.1.1-I.1.3434 in that A²R^{d}R^{e}R^{f} is 2,3-difluorophenyl and R¹ is CH₃.

Preference is likewise given to the compounds of the formula I.156, particularly preferably the compounds I.156.1-I.156.3434, in particular the compounds (Z,S)-I.156.1-I.156.3434, which differ from the corresponding compounds I.1.1-I.1.3434 or (Z,S)-I.1.1-I.1.3434 in that A²R^{d}R^{e}R^{f} is 2,4-difluorophenyl and R¹ is CH₃.

Preference is likewise given to the compounds of the formula I.157, particularly preferably the compounds I.157.1-I.157.3434, in particular the compounds (Z,S)-I.157.1-I.157.3434, which differ from the corresponding compounds I.1.1-I.1.3434 or (Z,S)-I.1.1-I.1.3434 in that A²R^{d}R^{e}R^{f} is 2,5-difluorophenyl and R¹ is CH₃.

Preference is likewise given to the compounds of the formula I.158, particularly preferably the compounds I.158.1-I.158.3434, in particular the compounds (Z,S)-I.158.1-I.158.3434, which differ from the corresponding compounds I.1.1-I.1.3434 or (Z,S)-I.1.1-I.1.3434 in that A²R^{d}R^{e}R^{f} is 2,6-difluorophenyl and R¹ is CH₃.

Preference is likewise given to the compounds of the formula I.159, particularly preferably the compounds I.159.1-I.159.3434, in particular the compounds (Z,S)-I.159.1-I.159.3434, which differ from the corresponding compounds I.1.1-I.1.3434 or (Z,S)-I.1.1-I.1.3434 in that A²R^{d}R^{e}R^{f} is 2-fluoro-3-chlorophenyl and R¹ is CH₃.

Preference is likewise given to the compounds of the formula I.160, particularly preferably the compounds I.160.1-I.160.3434, in particular the compounds (Z,S)-I.160.1-I.160.3434, which differ from the corresponding compounds I.1.1-I.1.3434 or (Z,S)-I.1.1-I.1.3434 in that A²R^{d}R^{e}R^{f} is 2-fluoro-4-chlorophenyl and R¹ is CH₃.

Preference is likewise given to the compounds of the formula I.161, particularly preferably the compounds I.161.1-I.161.3434, in particular the compounds (Z,S)-I.161.1-I.161.3434, which differ from the corresponding compounds I.1.1-I.1.3434 or (Z,S)-I.1.1-I.1.3434 in that A²R^{d}R^{e}R^{f} is 2-fluoro-5-chlorophenyl and R¹ is CH₃.

Preference is likewise given to the compounds of the formula I.162, particularly preferably the compounds I.162.1-I.162.3434, in particular the compounds (Z,S)-I.162.1-I.162.3434, which differ from the corresponding compounds I.1.1-I.1.3434 or (Z,S)-I.1.1-I.1.3434 in that A²R^{d}R^{e}R^{f} is 3,4-difluorophenyl and R¹ is CH₃.

Preference is likewise given to the compounds of the formula I.163, particularly preferably the compounds I.163.1-I.163.3434, in particular the compounds (Z,S)-I.163.1-I.163.3434, which differ from the corresponding compounds I.1.1-I.1.3434 or (Z,S)-I.1.1-I.1.3434 in that A²R^{d}R^{e}R^{f} is 3,5-difluorophenyl and R¹ is CH₃.

Preference is likewise given to the compounds of the formula I.164, particularly preferably the compounds I.164.1-I.164.3434, in particular the compounds (Z,S)-I.164.1-I.164.3434, which differ from the corresponding compounds I.1.1-I.1.3434 or (Z,S)-I.1.1-I.1.3434 in that A²R^{d}R^{e}R^{f} is 3,4-dichlorophenyl and R¹ is CH₃.

Preference is likewise given to the compounds of the formula I.165, particularly preferably the compounds I.165.1-I.165.3434, in particular the compounds (Z,S)-I.165.1-I.165.3434, which differ from the corresponding compounds I.1.1-I.1.3434 or (Z,S)-I.1.1-I.1.3434 in that A²R^{d}R^{e}R^{f} is 3,5-dichlorophenyl and R¹ is CH₃.

Preference is likewise given to the compounds of the formula I.166, particularly preferably the compounds I.166.1-I.166.3434, in particular the compounds (Z,S)-I.166.1-I.166.3434, which differ from the corresponding compounds I.1.1-I.1.3434 or (Z,S)-I.1.1-I.1.3434 in that A²R^{d}R^{e}R^{f} is 3-fluoro-4-chlorophenyl and R¹ is CH₃.

Preference is likewise given to the compounds of the formula I.167, particularly preferably the compounds I.167.1-I.167.3434, in particular the compounds (Z,S)-I.167.1-I.167.3434, which differ from the corresponding compounds I.1.1-I.1.3434 or (Z,S)-I.1.1-I.1.3434 in that A²R^{d}R^{e}R^{f} is 4-fluoro-3-chlorophenyl and R¹ is CH₃.

Preference is likewise given to the compounds of the formula I.168, particularly preferably the compounds I.168.1-I.168.3434, in particular the compounds (Z,S)-I.168.1-I.168.3434, which differ from the corresponding compounds I.1.1-I.1.3434 or (Z,S)-I.1.1-I.1.3434 in that A²R^{d}R^{e}R^{f} is 3-fluoro-5-chlorophenyl and R¹ is CH₃.

Preference is likewise given to the compounds of the formula I.169, particularly preferably the compounds I.169.1-I.169.3434, in particular the compounds (Z,S)-I.169.1-I.169.3434, which differ from the corresponding compounds I.1.1-I.1.3434 or (Z,S)-I.1.1-I.1.3434 in that A²R^{d}R^{e}R^{f} is pyridin-2-yl and R¹ is CH₃.

Preference is likewise given to the compounds of the formula I.170, particularly preferably the compounds I.170.1-I.170.3434, in particular the compounds (Z,S)-I.170.1-I.170.3434, which differ from the corresponding compounds I.1.1-I.1.3434 or (Z,S)-I.1.1-I.1.3434 in that A²R^{d}R^{e}R^{f} is 3-chloropyridin-2-yl and R¹ is CH₃.

Preference is likewise given to the compounds of the formula I.171, particularly preferably the compounds I.171.1-I.171.3434, in particular the compounds (Z,S)-I.171.1-I.171.3434, which differ from the corresponding compounds I.1.1-I.1.3434 or (Z,S)-I.1.1-I.1.3434 in that A²R^{d}R^{e}R^{f} is 4-chloropyridin-2-yl and R¹ is CH₃.

Preference is likewise given to the compounds of the formula I.172, particularly preferably the compounds I.172.1-I.172.3434, in particular the compounds (Z,S)-I.172.1-I.172.3434, which differ from the corresponding compounds I.1.1-I.1.3434 or (Z,S)-I.1.1-I.1.3434 in that A²R^{d}R^{e}R^{f} is 5-chloropyridin-2-yl and R¹ is CH₃.

Preference is likewise given to the compounds of the formula I.173, particularly preferably the compounds I.173.1-I.173.3434, in particular the compounds (Z,S)-I.173.1-I.173.3434, which differ from the corresponding compounds I.1.1-I.1.3434 or (Z,S)-I.1.1-I.1.3434 in that A²R^{d}R^{e}R^{f} is 6-chloropyridin-2-yl and R¹ is CH₃.

Preference is likewise given to the compounds of the formula I.174, particularly preferably the compounds I.174.1-I.174.3434, in particular the compounds (Z,S)-I.174.1-I.174.3434, which differ from the corresponding compounds I.1.1-I.1.3434 or (Z,S)-I.1.1-I.1.3434 in that A²R^{d}R^{e}R^{f} is 3-trifluoromethylpyridin-2-yl and R¹ is CH₃.

Preference is likewise given to the compounds of the formula I.175, particularly preferably the compounds I.175.1-I.175.3434, in particular the compounds (Z,S)-I.175.1-I.175.3434, which differ from the corresponding compounds I.1.1-I.1.3434 or (Z,S)-I.1.1-I.1.3434 in that A²R^{d}R^{e}R^{f} is 4-trifluoromethylpyridin-2-yl and R¹ is CH₃.

Preference is likewise given to the compounds of the formula I.176, particularly preferably the compounds I.176.1-I.176.3434, in particular the compounds (Z,S)-I.176.1-I.176.3434, which differ from the corresponding compounds I.1.1-I.1.3434 or (Z,S)-I.1.1-I.1.3434 in that A²R^{d}R^{e}R^{f} is 5-trifluoromethylpyridin-2-yl and R¹ is CH₃.

Preference is likewise given to the compounds of the formula I.177, particularly preferably the compounds I.177.1-I.177.3434, in particular the compounds (Z,S)-I.177.1-I.177.3434, which differ from the corresponding compounds I.1.1-I.1.3434 or (Z,S)-I.1.1-I.1.3434 in that A²R^{d}R^{e}R^{f} is 6-trifluoromethylpyridin-2-yl and R¹ is CH₃.

Preference is likewise given to the compounds of the formula I.178, particularly preferably the compounds I.178.1-I.178.3434, in particular the compounds (Z,S)-I.178.1-I.178.3434, which differ from the corresponding compounds I.1.1-I.1.3434 or (Z,S)-I.1.1-I.1.3434 in that A²R^{d}R^{e}R^{f} is 3-methoxypyridin-2-yl and R¹ is CH₃.

Preference is likewise given to the compounds of the formula I.179, particularly preferably the compounds I.179.1-I.179.3434, in particular the compounds (Z,S)-I.179.1-I.179.3434, which differ from the corresponding compounds I.1.1-I.1.3434 or (Z,S)-I.1.1-I.1.3434 in that A²R^{d}R^{e}R^{f} is 4-methoxypyridin-2-yl and R¹ is CH₃.

Preference is likewise given to the compounds of the formula I.180, particularly preferably the compounds I.180.1-I.180.3434, in particular the compounds (Z,S)-I.180.1-I.180.3434, which differ from the corresponding compounds I.1.1-I.1.3434 or (Z,S)-I.1.1-I.1.3434 in that A²R^{d}R^{e}R^{f} is 5-methoxypyridin-2-yl and R¹ is CH₃.

Preference is likewise given to the compounds of the formula I.181, particularly preferably the compounds I.181.1-I.181.3434, in particular the compounds (Z,S)-I.181.1-I.181.3434, which differ from the corresponding compounds I.1.1-I.1.3434 or (Z,S)-I.1.1-I.1.3434 in that A²R^{d}R^{e}R^{f} is 6-methoxypyridin-2-yl and R¹ is CH₃.

Preference is likewise given to the compounds of the formula I.182, particularly preferably the compounds I.182.1-I.182.3434, in particular the compounds (Z,S)-I.182.1-I.182.3434, which differ from the corresponding compounds I.1.1-I.1.3434 or (Z,S)-I.1.1-I.1.3434 in that A²R^{d}R^{e}R^{f} is 3-fluoropyridin-2-yl and R¹ is CH₃.

Preference is likewise given to the compounds of the formula I.183, particularly preferably the compounds I.183.1-I.183.3434, in particular the compounds (Z,S)-I.83.1-I.183.3434, which differ from the corresponding compounds I.1.1-I.1.3434 or (Z,S)-I.1.1-I.1.3434 in that A²R^{d}R^{e}R^{f} is 4-fluoropyridin-2-yl and R¹ is CH₃.

Preference is likewise given to the compounds of the formula I.184, particularly preferably the compounds I.184.1-I.184.3434, in particular the compounds (Z,S)-I.184.1-I.184.3434, which differ from the corresponding compounds I.1.1-I.1.3434 or (Z,S)-I.1.1-I.1.3434 in that A²R^{d}R^{e}R^{f} is 5-fluoropyridin-2-yl and R¹ is CH₃.

Preference is likewise given to the compounds of the formula I.185, particularly preferably the compounds I.185.1-I.185.3434, in particular the compounds (Z,S)-I.185.1-I.185.3434, which differ from the corresponding compounds I.1.1-I.1.3434 or (Z,S)-I.1.1-I.1.3434 in that A²R^{d}R^{e}R^{f} is 6-fluoropyridin-2-yl and R¹ is CH₃.

Preference is likewise given to the compounds of the formula I.186, particularly preferably the compounds I.186.1-I.186.3434, in particular the compounds (Z,S)-I.186.1-I.186.3434, which differ from the corresponding compounds I.1.1-I.1.3434 or (Z,S)-I.1.1-I.1.3434 in that A²R^{d}R^{e}R^{f} is pyridin-3-yl and R¹ is CH₃.

Preference is likewise given to the compounds of the formula I.187, particularly preferably the compounds I.187.1-I.187.3434, in particular the compounds (Z,S)-I.187.1-I.187.3434, which differ from the corresponding compounds I.1.1-I.1.3434 or (Z,S)-I.1.1-I.1.3434 in that A²R^{d}R^{e}R^{f} is 2-chloropyridin-3-yl and R¹ is CH₃.

Preference is likewise given to the compounds of the formula I.188, particularly preferably the compounds I.188.1-I.188.3434, in particular the compounds (Z,S)-I.188.1-I.188.3434, which differ from the corresponding compounds I.1.1-I.1.3434 or (Z,S)-I.1.1-I.1.3434 in that A²R^{d}R^{e}R^{f} is 4-chloropyridin-3-yl and R¹ is CH₃.

Preference is likewise given to the compounds of the formula I.189, particularly preferably the compounds I.189.1-I.189.3434, in particular the compounds (Z,S)-I.189.1-I.189.3434, which differ from the corresponding compounds I.1.1-I.1.3434 or (Z,S)-I.1.1-I.1.3434 in that A²R^{d}R^{e}R^{f} is 5-chloropyridin-3-yl and R¹ is CH₃.

Preference is likewise given to the compounds of the formula I.190, particularly preferably the compounds I.190.1-I.190.3434, in particular the compounds (Z,S)-I.190.1-I.190.3434, which differ from the corresponding compounds I.1.1-I.1.3434 or (Z,S)-I.1.1-I.1.3434 in that A²R^{d}R^{e}R^{f} is 6-chloropyridin-3-yl and R¹ is CH₃.

Preference is likewise given to the compounds of the formula I.191, particularly preferably the compounds I.191.1-I.191.3434, in particular the compounds (Z,S)-I.191.1-I.191.3434, which differ from the corresponding compounds I.1.1-I.1.3434 or (Z,S)-I.1.1-I.1.3434 in that A²R^{d}R^{e}R^{f} is 2-trifluoromethylpyridin-3-yl and R¹ is CH₃.

Preference is likewise given to the compounds of the formula I.192, particularly preferably the compounds I.192.1-I.192.3434, in particular the compounds (Z,S)-I.192.1-I.192.3434, which differ from the corresponding compounds I.1.1-I.1.3434 or (Z,S)-I.1.1-I.1.3434 in that A²R^{d}R^{e}R^{f} is 4-trifluoromethylpyridin-3-yl and R¹ is CH₃.

Preference is likewise given to the compounds of the formula I.193, particularly preferably the compounds I.193.1-I.193.3434, in particular the compounds (Z,S)-I.193.1-I.193.3434, which differ from the corresponding compounds I.1.1-I.1.3434 or (Z,S)-I.1.1-I.1.3434 in that A²R^{d}R^{e}R^{f} is 5-trifluoromethylpyridin-3-yl and R¹ is CH₃.

Preference is likewise given to the compounds of the formula I.194, particularly preferably the compounds I.194.1-I.194.3434, in particular the compounds (Z,S)-I.194.1-I.194.3434, which differ from the corresponding compounds I.1.1-I.1.3434 or (Z,S)-I.1.1-I.1.3434 in that A²R^{d}R^{e}R^{f} is 6-trifluoromethylpyridin-3-yl and R¹ is CH₃.

Preference is likewise given to the compounds of the formula I.195, particularly preferably the compounds I.195.1-I.195.3434, in particular the compounds (Z,S)-I.195.1-I.195.3434, which differ from the corresponding compounds I.1.1-I.1.3434 or (Z,S)-I.1.1-I.1.3434 in that A²R^{d}R^{e}R^{f} is 2-methoxypyridin-3-yl and R¹ is CH₃.

Preference is likewise given to the compounds of the formula I.196, particularly preferably the compounds I.196.1-I.196.3434, in particular the compounds (Z,S)-I.196.1-I.196.3434, which differ from the corresponding compounds I.1.1-I.1.3434 or (Z,S)-I.1.1-I.1.3434 in that A²R^{d}R^{e}R^{f} is 4-methoxypyridin-3-yl and R¹ is CH₃.

Preference is likewise given to the compounds of the formula I.197, particularly preferably the compounds I.197.1-I.197.3434, in particular the compounds (Z,S)-I.197.1-I.197.3434, which differ from the corresponding compounds I.1.1-I.1.3434 or (Z,S)-I.1.1-I.1.3434 in that A²R^{d}R^{e}R^{f} is 5-methoxypyridin-3-yl and R¹ is CH₃.

Preference is likewise given to the compounds of the formula I.198, particularly preferably the compounds I.198.1-I.198.3434, in particular the compounds (Z,S)-I.198.1-I.198.3434, which differ from the corresponding compounds I.1.1-I.1.3434 or (Z,S)-I.1.1-I.1.3434 in that A²R^{d}R^{e}R^{f} is 6-methoxypyridin-3-yl and R¹ is CH₃.

Preference is likewise given to the compounds of the formula I.199, particularly preferably the compounds I.199.1-I.199.3434, in particular the compounds (Z,S)-I.199.1-I.199.3434, which differ from the corresponding compounds I.1.1-I.1.3434 or (Z,S)-I.1.1-I.1.3434 in that A²R^{d}R^{e}R^{f} is 2-fluoropyridin-3-yl and R¹ is CH₃.

Preference is likewise given to the compounds of the formula I.200, particularly preferably the compounds I.200.1-I.200.3434, in particular the compounds (Z,S)-I.200.1-I.200.3434, which differ from the corresponding compounds I.1.1-I.1.3434 or (Z,S)-I.1.1-I.1.3434 in that A²R^{d}R^{e}R^{f} is 4-fluoropyridin-3-yl and R¹ is CH₃.

Preference is likewise given to the compounds of the formula I.201, particularly preferably the compounds I.201.1-I.201.3434, in particular the compounds (Z,S)-I.201.1-I.201.3434, which differ from the corresponding compounds I.1.1-I.1.3434 or (Z,S)-I.1.1-I.1.3434 in that A²R^{d}R^{e}R^{f} is 5-fluoropyridin-3-yl and R¹ is CH₃.

Preference is likewise given to the compounds of the formula I.202, particularly preferably the compounds I.202.1-I.202.3434, in particular the compounds (Z,S)-I.202.1-I.202.3434, which differ from the corresponding compounds I.1.1-I.1.3434 or (Z,S)-I.1.1-I.1.3434 in that A²R^{d}R^{e}R^{f} is 6-fluoropyridin-3-yl and R¹ is CH₃.

Preference is likewise given to the compounds of the formula I.203, particularly preferably the compounds I.203.1-I.203.3434, in particular the compounds (Z,S)-I.203.1-I.203.3434, which differ from the corresponding compounds I.1.1-I.1.3434 or (Z,S)-I.1.1-I.1.3434 in that A²R^{d}R^{e}R^{f} is pyridin-4-yl and R¹ is CH₃.

Preference is likewise given to the compounds of the formula I.204, particularly preferably the compounds I.204.1-I.204.3434, in particular the compounds (Z,S)-I.204.1-I.204.3434, which differ from the corresponding compounds I.1.1-I.1.3434 or (Z,S)-I.1.1-I.1.3434 in that A²R^{d}R^{e}R^{f} is 2-fluoropyridin-4-yl and R¹ is CH₃.

Preference is likewise given to the compounds of the formula I.205, particularly preferably the compounds I.205.1-I.205.3434, in particular the compounds (Z,S)-I.205.1-I.205.3434, which differ from the corresponding compounds I.1.1-I.1.3434 or (Z,S)-I.1.1-I.1.3434 in that A²R^{d}R^{e}R^{f} is 3-fluoropyridin-4-yl and R¹ is CH₃.

Preference is likewise given to the compounds of the formula I.206, particularly preferably the compounds I.206.1-I.206.3434, in particular the compounds (Z,S)-I.206.1-I.206.3434, which differ from the corresponding compounds I.1.1-I.1.3434 or (Z,S)-I.1.1-I.1.3434 in that A²R^{d}R^{e}R^{f} is 2-methoxypyridin-4-yl and R¹ is CH₃.

Preference is likewise given to the compounds of the formula I.207, particularly preferably the compounds I.207.1-I.207.3434, in particular the compounds (Z,S)-I.207.1-I.207.3434, which differ from the corresponding compounds I.1.1-I.1.3434 or (Z,S)-I.1.1-I.1.3434 in that A²R^{d}R^{e}R^{f} is 3-methoxypyridin-4-yl and R¹ is CH₃.

Preference is likewise given to the compounds of the formula I.208, particularly preferably the compounds I.208.1-I.208.3434, in particular the compounds (Z,S)-I.208.1-I.208.3434, which differ from the corresponding compounds I.1.1-I.1.3434 or (Z,S)-I.1.1-I.1.3434 in that A²R^{d}R^{e}R^{f} is 2-trifluoromethylpyridin-4-yl and R¹ is CH₃.

Preference is likewise given to the compounds of the formula I.209, particularly preferably the compounds I.209.1-I.209.3434, in particular the compounds (Z,S)-I.209.1-I.209.3434, which differ from the corresponding compounds I.1.1-I.1.3434 or (Z,S)-I.1.1-I.1.3434 in that A²R^{d}R^{e}R^{f} is 3-trifluoromethylpyridin-4-yl and R¹ is CH₃.

Preference is likewise given to the compounds of the formula I.210, particularly preferably the compounds I.210.1-I.210.3434, in particular the compounds (Z,S)-I.210.1-I.210.3434, which differ from the corresponding compounds I.1.1-I.1.3434 or (Z,S)-I.1.1-I.1.3434 in that A²R^{d}R^{e}R^{f} is 2-chloropyridin-4-yl and R¹ is CH₃.

Preference is likewise given to the compounds of the formula I.211, particularly preferably the compounds I.211.1-I.211.3434, in particular the compounds (Z,S)-I.211.1-I.211.3434, which differ from the corresponding compounds I.1.1-I.1.3434 or (Z,S)-I.1.1-I.1.3434 in that A²R^{d}R^{e}R^{f} is 3-chloropyridin-4-yl and R¹ is CH₃.

Preference is likewise given to the compounds of the formula I.212, particularly preferably the compounds I.212.1-I.212.3434, in particular the compounds (Z,S)-I.212.1-I.212.3434, which differ from the corresponding compounds I.1.1-I.1.3434 or (Z,S)-I.1.1-I.1.3434 in that A²R^{d}R^{e}R^{f} is thiophen-2-yl and R¹ is CH₃.

Preference is likewise given to the compounds of the formula I.213, particularly preferably the compounds I.213.1-I.213.3434, in particular the compounds (Z,S)-I.213.1-I.213.3434, which differ from the corresponding compounds I.1.1-I.1.3434 or (Z,S)-I.1.1-I.1.3434 in that A²R^{d}R^{e}R^{f} is 3-chlorothiophen-2-yl and R¹ is CH₃.

Preference is likewise given to the compounds of the formula I.214, particularly preferably the compounds I.214.1-I.214.3434, in particular the compounds (Z,S)-I.214.1-I.214.3434, which differ from the corresponding compounds I.1.1-I.1.3434 or (Z,S)-I.1.1-I.1.3434 in that A²R^{d}R^{e}R^{f} is 4-chlorothiophen-2-yl and R¹ is CH₃.

Preference is likewise given to the compounds of the formula I.215, particularly preferably the compounds I.215.1-I.215.3434, in particular the compounds (Z,S)-I.215.1-I.215.3434, which differ from the corresponding compounds I.1.1-I.1.3434 or (Z,S)-I.1.1-I.1.3434 in that A²R^{d}R^{e}R^{f} is 5-chlorothiophen-2-yl and R¹ is CH₃.

Preference is likewise given to the compounds of the formula I.216, particularly preferably the compounds I.216.1-I.216.3434, in particular the compounds (Z,S)-I.216.1-I.216.3434, which differ from the corresponding compounds I.1.1-I.1.3434 or (Z,S)-I.1.1-I.1.3434 in that A²R^{d}R^{e}R^{f} is 3-trifluoromethylthiophen-2-yl and R¹ is CH₃.

Preference is likewise given to the compounds of the formula I.217, particularly preferably the compounds I.217.1-I.217.3434, in particular the compounds (Z,S)-I.217.1-I.217.3434, which differ from the corresponding compounds I.1.1-I.1.3434 or (Z,S)-I.1.1-I.1.3434 in that A²R^{d}R^{e}R^{f} is 4-trifluoromethylthiophen-2-yl and R¹ is CH₃.

Preference is likewise given to the compounds of the formula I.218, particularly preferably the compounds I.218.1-I.218.3434, in particular the compounds (Z,S)-I.218.1-I.218.3434, which differ from the corresponding compounds I.1.1-I.1.3434 or (Z,S)-I.1.1-I.1.3434 in that A²R^{d}R^{e}R^{f} is 5-trifluoromethylthiophen-2-yl and R¹ is CH₃.

Preference is likewise given to the compounds of the formula I.219, particularly preferably the compounds I.219.1-I.219.3434, in particular the compounds (Z,S)-I.219.1-I.219.3434, which differ from the corresponding compounds I.1.1-I.1.3434 or (Z,S)-I.1.1-I.1.3434 in that A²R^{d}R^{e}R^{f} is 3-methoxythiophen-2-yl and R¹ is CH₃.

Preference is likewise given to the compounds of the formula I.220, particularly preferably the compounds I.220.1-I.220.3434, in particular the compounds (Z,S)-I.220.1-I.220.3434, which differ from the corresponding compounds I.1.1-I.1.3434 or (Z,S)-I.1.1-I.1.3434 in that A²R^{d}R^{e}R^{f} is 4-methoxythiophen-2-yl and R¹ is CH₃.

Preference is likewise given to the compounds of the formula I.221, particularly preferably the compounds I.221.1-I.221.3434, in particular the compounds (Z,S)-I.221.1-I.221.3434, which differ from the corresponding compounds I.1.1-I.1.3434 or (Z,S)-I.1.1-I.1.3434 in that A²R^{d}R^{e}R^{f} is 5-methoxythiophen-2-yl and R¹ is CH₃.

Preference is likewise given to the compounds of the formula I.222, particularly preferably the compounds I.222.1-I.222.3434, in particular the compounds (Z,S)-I.222.1-I.222.3434, which differ from the corresponding compounds I.1.1-I.1.3434 or (Z,S)-I.1.1-I.1.3434 in that A²R^{d}R^{e}R^{f} is 3-fluorothiophen-2-yl and R¹ is CH₃.

Preference is likewise given to the compounds of the formula I.223, particularly preferably the compounds I.223.1-I.223.3434, in particular the compounds (Z,S)-I.223.1-I.223.3434, which differ from the corresponding compounds I.1.1-I.1.3434 or (Z,S)-I.1.1-I.1.3434 in that A²R^{d}R^{e}R^{f} is 4-fluorothiophen-2-yl and R¹ is CH₃.

Preference is likewise given to the compounds of the formula I.224, particularly preferably the compounds I.224.1-I.224.3434, in particular the compounds (Z,S)-I.224.1-I.224.3434, which differ from the corresponding compounds I.1.1-I.1.3434 or (Z,S)-I.1.1-I.1.3434 in that A²R^{d}R^{e}R^{f} is 5-fluorothiophen-2-yl and R¹ is CH₃.

Preference is likewise given to the compounds of the formula I.225, particularly preferably the compounds I.225.1-I.225.3434, in particular the compounds (Z,S)-I.225.1-I.225.3434, which differ from the corresponding compounds I.1.1-I.1.3434 or (Z,S)-I.1.1-I.13434 in that A²R^{d}R^{e}R^{f} is thiophen-3-yl and R¹ is CH₃.

Preference is likewise given to the compounds of the formula I.226, particularly preferably the compounds I.226.1-I.226.3434, in particular the compounds (Z,S)-I.226.1-I.226.3434, which differ from the corresponding compounds I.1.1-I.1.3434 or (Z,S)-I.1.1-I.1.3434 in that A²R^{d}R^{e}R^{f} is 2-chlorothiophen-3-yl and R¹ is CH₃.

Preference is likewise given to the compounds of the formula I.227, particularly preferably the compounds I.227.1-I.227.3434, in particular the compounds (Z,S)-I.227.1-I.227.3434, which differ from the corresponding compounds I.1.1-I.1.3434 or (Z,S)-I.1.1-I.1.3434 in that A²R^{d}R^{e}R^{f} is 4-chlorothiophen-3-yl and R¹ is CH₃.

Preference is likewise given to the compounds of the formula I.228, particularly preferably the compounds I.228.1-I.228.3434, in particular the compounds (Z,S)-I.228.1-I.228.3434, which differ from the corresponding compounds I.1.1-I.1.3434 or (Z,S)-I.1.1-I.1.3434 in that A²R^{d}R^{e}R^{f} is 5-chlorothiophen-3-yl and R¹ is CH₃.

Preference is likewise given to the compounds of the formula I.229, particularly preferably the compounds I.229.1-I.229.3434, in particular the compounds (Z,S)-I.229.1-I.229.3434, which differ from the corresponding compounds I.1.1-I.1.3434 or (Z,S)-I.1.1-I.1.3434 in that A²R^{d}R^{e}R^{f} is 2-fluorothiophen-3-yl and R¹ is CH₃.

Preference is likewise given to the compounds of the formula I.230, particularly preferably the compounds I.230.1-I.230.3434, in particular the compounds (Z,S)-I.230.1-I.230.3434, which differ from the corresponding compounds I.1.1-I.1.3434 or (Z,S)-I.1.1-I.1.13434 in that A²R^{d}R^{e}R^{f} is 4-fluorothiophen-3-yl and R¹ is CH₃.

Preference is likewise given to the compounds of the formula I.231, particularly preferably the compounds I.231.1-I.231.3434, in particular the compounds (Z,S)-I.231.1-I.231.3434, which differ from the corresponding compounds I.1.1-I.1.3434 or (Z,S)-I.1.1-I.1.3434 in that A²R^{d}R^{e}R^{f} is 5-fluorothiophen-3-yl and R¹ is CH₃.

Preference is likewise given to the compounds of the formula I.232, particularly preferably the compounds I.232.1-I.232.3434, in particular the compounds (Z,S)-I.232.1-I.232.3434, which differ from the corresponding compounds I.1.1-I.1.3434 or (Z,S)-I.1.1-I.1.3434 in that A²R^{d}R^{e}R^{f} is 2-methoxythiophen-3-yl and R¹ is CH₃.

Preference is likewise given to the compounds of the formula I.233, particularly preferably the compounds I.233.1-I.233.3434, in particular the compounds (Z,S)-I.233.1-I.233.3434, which differ from the corresponding compounds I.1.1-I.1.3434 or (Z,S)-I.1.1-I.1.3434 in that A²R^{d}R^{e}R^{f} is 4-methoxythiophen-3-yl and R¹ is CH₃.

Preference is likewise given to the compounds of the formula I.234, particularly preferably the compounds I.234.1-I.234.3434, in particular the compounds (Z,S)-I.234.1-I.234.3434, which differ from the corresponding compounds I.1.1-I.1.3434 or (Z,S)-I.1.1-I.1.3434 in that A²R^{d}R^{e}R^{f} is 5-methoxythiophen-3-yl and R¹ is CH₃.

Preference is likewise given to the compounds of the formula I.235, particularly preferably the compounds I.235.1-I.235.3434, in particular the compounds (Z,S)-I.235.1-I.235.3434, which differ from the corresponding compounds I.1.1-I.1.3434 or (Z,S)-I.1.1-I.1.3434 in that A²R^{d}R^{e}R^{f} is 2-trifluoromethylthiophen-3-yl and R¹ is CH₃.

Preference is likewise given to the compounds of the formula I.236, particularly preferably the compounds I.236.1-I.236.3434, in particular the compounds (Z,S)-I.236.1-I.236.3434, which differ from the corresponding compounds I.1.1-I.1.3434 or (Z,S)-I.1.1-I.1.3434 in that A²R^{d}R^{e}R^{f} is 4-trifluoromethylthiophen-3-yl and R¹ is CH₃.

Preference is likewise given to the compounds of the formula I.237, particularly preferably the compounds I.237.1-I.237.3434, in particular the compounds (Z,S)-I.237.1-I.237.3434, which differ from the corresponding compounds I.1.1-I.1.3434 or (Z,S)-I.1.1-I.1.3434 in that A²R^{d}R^{e}R^{f} is 5-trifluoromethylthiophen-3-yl and R¹ is CH₃.

Preference is likewise given to the compounds of the formula I.238, particularly preferably the compounds I.238.1-I.238.3434, in particular the compounds (Z,S)-I.238.1-I.238.3434, which differ from the corresponding compounds I.1.1-I.1.3434 or (Z,S)-I.1.1-I.1.3434 in that A²R^{d}R^{e}R^{f} is furan-2-yl and R¹ is CH₃.

Preference is likewise given to the compounds of the formula I.239, particularly preferably the compounds I.239.1-I.239.3434, in particular the compounds (Z,S)-I.239.1-I.239.3434, which differ from the corresponding compounds I.1.1-I.1.3434 or (Z,S)-I.1.1-I.1.3434 in that A²R^{d}R^{e}R^{f} is 3-chlorofuran-2-yl and R¹ is CH₃.

Preference is likewise given to the compounds of the formula I.240, particularly preferably the compounds I.240.1-I.240.3434, in particular the compounds (Z,S)-I.240.1-I.240.3434, which differ from the corresponding compounds I.1.1-I.1.3434 or (Z,S)-I.1.1-I.1.3434 in that A²R^{d}R^{e}R^{f} is 4-chlorofuran-2-yl and R¹ is CH₃.

Preference is likewise given to the compounds of the formula I.241, particularly preferably the compounds I.241.1-I.241.3434, in particular the compounds (Z,S)-I.241.1-I.241.3434, which differ from the corresponding compounds I.1.1-I.1.3434 or (Z,S)-I.1.1-I.1.3434 in that A²R^{d}R^{e}R^{f} is 5-chlorofuran-2-yl and R¹ is CH₃.

Preference is likewise given to the compounds of the formula I.242, particularly preferably the compounds I.242.1-I.242.3434, in particular the compounds (Z,S)-I.242.1-I.242.3434, which differ from the corresponding compounds I.1.1-I.1.3434 or (Z,S)-I.1.1-I.1.3434 in that A²R^{d}R^{e}R^{f} is 3-fluorofuran-2-yl and R¹ is CH₃.

Preference is likewise given to the compounds of the formula I.243, particularly preferably the compounds I.243.1-I.243.3434, in particular the compounds (Z,S)-I.243.1-I.243.3434, which differ from the corresponding compounds I.1.1-I.1.3434 or (Z,S)-I.1.1-I.1.3434 in that A²R^{d}R^{e}R^{f} is 4-fluorofuran-2-yl and R¹ is CH₃.

Preference is likewise given to the compounds of the formula I.244, particularly preferably the compounds I.244.1-I.244.3434, in particular the compounds (Z,S)-I.244.1-I.244.3434, which differ from the corresponding compounds I.1.1-I.1.3434 or (Z,S)-I.1.1-I.1.3434 in that A²R^{d}R^{e}R^{f} is 5-fluorofuran-2-yl and R¹ is CH₃.

Preference is likewise given to the compounds of the formula I.245, particularly preferably the compounds I.245.1-I.245.3434, in particular the compounds (Z,S)-I.245.1-I.245.3434, which differ from the corresponding compounds I.1.1-I.1.3434 or (Z,S)-I.1.1-I.1.3434 in that A²R^{d}R^{e}R^{f} is furan-3-yl and R¹ is CH₃.

Preference is likewise given to the compounds of the formula I.246, particularly preferably the compounds I.246.1-I.246.3434, in particular the compounds (Z,S)-I.246.1-I.246.3434, which differ from the corresponding compounds I.1.1-I.1.3434 or (Z,S)-I.1.1-I.1.3434 in that A²R^{d}R^{e}R^{f} is 2-chlorofuran-3-yl and R¹ is CH₃.

Preference is likewise given to the compounds of the formula I.247, particularly preferably the compounds I.247.1-I.247.3434, in particular the compounds (Z,S)-I.247.1-I.247.3434, which differ from the corresponding compounds I.1.1-I.1.3434 or (Z,S)-I.1.1-I.1.3434 in that A²R^{d}R^{e}R^{f} is 4-chlorofuran-3-yl and R¹ is CH₃.

Preference is likewise given to the compounds of the formula I.248, particularly preferably the compounds I.248.1-I.248.3434, in particular the compounds (Z,S)-I.248.1-I.248.3434, which differ from the corresponding compounds I.1.1-I.1.3434 or (Z,S)-I.1.1-I.1.3434 in that A²R^{d}R^{e}R^{f} is 5-chlorofuran-3-yl and R¹ is CH₃.

Preference is likewise given to the compounds of the formula I.249, particularly preferably the compounds I.249.1-I.249.3434, in particular the compounds (Z,S)-I.249.1-I.249.3434, which differ from the corresponding compounds I.1.1-I.1.3434 or (Z,S)-I.1.1-I.1.3434 in that A²R^{d}R^{e}R^{f} is 2-fluorofuran-3-yl and R¹ is CH₃.

Preference is likewise given to the compounds of the formula I.250, particularly preferably the compounds I.250.1-I.250.3434, in particular the compounds (Z,S)-I.250.1-I.250.3434, which differ from the corresponding compounds I.1.1-I.1.3434 or (Z,S)-I.1.1-I.1.3434 in that A²R^{d}R^{e}R^{f} is 4-fluorofuran-3-yl and R¹ is CH₃.

Preference is likewise given to the compounds of the formula I.251, particularly preferably the compounds I.251.1-I.251.3434, in particular the compounds (Z,S)-I.251.1-I.251.3434, which differ from the corresponding compounds I.1.1-I.1.3434 or (Z,S)-I.1.1-I.1.3434 in that A²R^{d}R^{e}R^{f} is 5-fluorofuran-3-yl and R¹ is CH₃.

Preference is likewise given to the compounds of the formula I.252, particularly preferably the compounds I.252.1-I.252.3434, in particular the compounds (Z,S)-I.252.1-I.252.3434, which differ from the corresponding compounds I.1.1-I.1.3434 or (Z,S)-I.1.1-I.1.3434 in that A² is 5-fluorofuran-3-yl and R¹ is CH₃.

The piperazine compounds of the formula I can be prepared by standard methods for synthesizing organic compounds by various routes, for example by the processes illustrated in more detail below:

### Process A

The compounds of the formula I can be prepared, for example, analogously to processes known from the literature by dehydrating the corresponding alcohol precursor of the formula II.

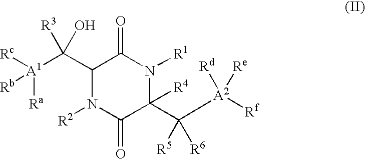

Accordingly, the invention furthermore provides a process for preparing piperazine compounds of the general formula I according to the invention which comprises reacting a compound of the formula II

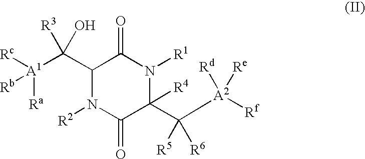

in which the variables A¹, A², R¹, R², R³, R⁴, R⁵, R⁶, R^{a}, R^{b}, R^{c}, R^{d}, R^{e} and R^{f} are as defined above for the compound of the formula I, either
(A.1) to give a compound of the formula III

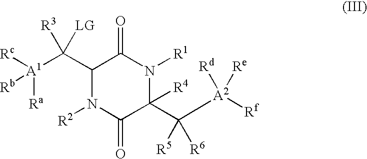

in which LG is a leaving group, and formally eliminating the compound H-LG from the compound III, giving the corresponding compound I;
or
(A.2) dehydrating corresponding compound I if LG is OH, wherein the dehydration is, if appropriate, carried out in the presence of a dehydrating agent.

For the preferred meanings of the variables A¹, A², R¹, R², R³, R⁴, R⁵, R⁶, R^{a}, R^{b}, R^{c}, R^{d}, R^{e} and R^{f}, reference is made to what was stated above. Preferred embodiments of the process according to the invention are described below.

### Variant A.1

In a first variant (variant A.1), the alcohol function of the compound II can initially be converted into a suitable leaving group, and this can then be eliminated formally as the compound H-LG. The elimination reaction is preferably carried out in the presence of a suitable base. This reaction is illustrated in the scheme below.

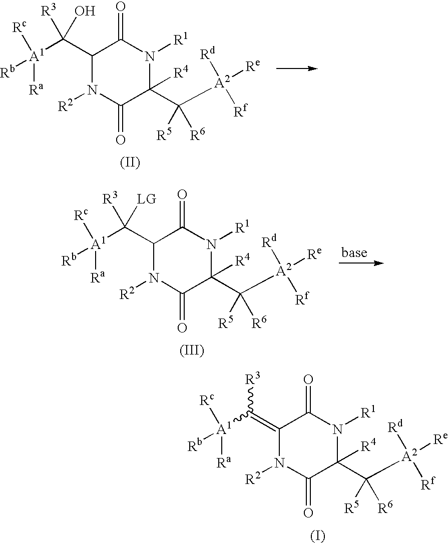

In formula II, the variables A¹, A², R¹-R⁶, R^{a}, R^{b}, R^{c}, R^{d}, R^{e} and R^{f} are as defined for formula I. LG is a leaving group.

The leaving group LG is a customary leaving group which is easy to prepare from a hydroxyl group. Examples of these are 4-toluenesulfonyloxy (LG=—O—SO₂C₆H₄CH₃), trifluoromethanesulfonyloxy (LG=—O—SO₂CF₃) and methanesulfonyloxy (LG=—O—SO₂CH₃), the latter being particularly suitable. Such a leaving group is introduced in accordance with customary processes, for example by reacting the alcohol II with a base and then with the appropriate sulfonyl chloride, for example using methanesulfonyl chloride or trifluoromethanesulfonyl chloride. Suitable bases are the bases listed below for the elimination. However, preference is given to using bases soluble in organic solvents, for example the amines or nitrogen heterocycles mentioned below. Use is made in particular of pyridine or substituted pyridines, such as dimethylaminopyridine, lutidine or collidine, or mixtures thereof. It is expedient to chose the organic bases such that they can also act as solvent.

Bases suitable for the elimination are, in general, inorganic compounds such as alkali metal and alkaline earth metal hydroxides, such as lithium hydroxide, sodium hydroxide, potassium hydroxide or calcium hydroxide, an aqueous ammonia solution, alkali metal or alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, for example lithium diisopropylamide, sodium amide and potassium amide, alkali metal and alkaline earth metal carbonates, such as lithium carbonate, potassium carbonate, cesium carbonate and calcium carbonate, and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide, potassium tert-pentoxide and dimethoxymagnesium, moreover organic bases, for example tertiary amines, such as trimethylamine, triethylamine, diisopropylethylamine, 2-hydroxypyridine and N-methylpiperidine, pyridine, substituted pyridines, such as collidine, lutidine and 4-dimethylaminopyridine, and also bicyclic amines. It is, of course, also possible to use a mixture of different bases.

Particularly suitable, however, are bases which are sufficiently basic but substantially not nucleophilic, for example sterically hindered alkali metal alkoxides, for example alkali metal tert-butoxides, such as potassium tert-butoxide, and in particular cyclic amidines, such as DBU (1,8-diazabicyclo[5.4.0]undec-7-ene) and DBN (1,5-diazabicyclo[3.4.0]non-5-ene). Preference is given to using the last-mentioned amidines.

The elimination is generally carried out in a solvent, preferably in an inert organic solvent. Suitable inert organic solvents include aromatic hydrocarbons, such as toluene, o-, m- and p-xylene, halogenated hydrocarbons, such as dichloromethane, dichloroethane, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol, tert-butanol, water and also dimethyl sulfoxide, dimethylformamide and dimethylacetamide, and also morpholine and N-methylmorpholine. It is also possible to use mixtures of the solvents mentioned. Preference is given to using tetrahydrofuran.

The dehydration of alcohols II by converting the alcohol function into a good leaving group and the subsequent elimination can be carried out analogously to known processes of the prior art, for example analogously to the processes described in Helv. Chim. Acta 1947, 30, 1454; Liebigs Ann. Chem. 1992, (7), 687-692, Carbanions. 24. Rearrangements of (E)- and (Z)-2,2-diphenyl-3-pentenylalkali metal compounds; Sch. Chem., Georgia inst. Technol., Atlanta, Ga., USA; J. Org. Chem. 1989, 54(7), 1671-1679; Chemical & Pharmaceutical Bulletin 1986, 34(7), 2786-2798, which are expressly incorporated herein in their entirety.

### Variant A.2

In a second variant (variant A.2), the compound I is prepared by dehydrating the compound II in the presence of a suitable dehydrating agent, as illustrated by the scheme below.

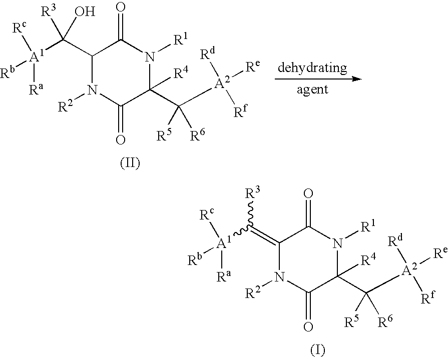

Suitable dehydrating agents are, for example, the system triphenylphosphine/DEAD (DEAD=diethyl azodicarboxylate) and the Burgess reagent. The combination of triphenylphosphine and DEAD is generally employed for the targeted inversion at a hydroxyl-substituted center of chirality (Mitsunobu reaction); however, in the absence of nucleophiles it acts as a mild dehydrating agent. Based on the compound II, the system is preferably employed in excess, where the two components triphenylphosphine and DEAD are suitably present in an approximately equimolar ratio.

The Burgess reagent is the zwitterion methyl N-(triethylammoniumsulfonylcarbamate ((C₂H₅)₃N⁺—SO₂—N⁻—COOCH₃), a mild dehydrating agent. This can be employed in an equimolar amount or in a molar excess, based on the alcohol II. The reaction with the Burgess reagent is generally carried out in an inert organic solvent. Suitable inert organic solvents include aromatic hydrocarbons, such as toluene, o-, m- and p-xylene, halogenated hydrocarbons, such as dichloromethane, dichloroethane, chloroform and chlorobenzene, ethers, such as diethyl ether, diisopropyl ether, tert-butyl methyl ether, dioxane, anisole and tetrahydrofuran, nitriles, such as acetonitrile and propionitrile, and ketones, such as acetone, methyl ethyl ketone, diethyl ketone and tert-butyl methyl ketone. Preference is given to using aromatic hydrocarbons or mixtures thereof and especially toluene.

The dehydration of alcohols II using dehydrating agents can be carried out analogously to known processes of the prior art, for example analogously to the processes described in Synthesis 2003, 201 and J. Indian Sci. 2001, 81, 461, which are expressly incorporated herein in their entirety.

### Process B

The alcohols of the formula II used in the two variants (A.1 and A.2) of process A can be prepared, for example, analogously to processes known from the literature by cyclizing corresponding dipeptide precursors of the formula IV, for example analogously to the method described by T. Kawasaki et al., Org. Lett. 2(19) (2000), 3027-3029, Igor L. Rodionov et al., Tetrahedron 58(42) (2002), 8515-8523 or A. L. Johnson et al., Tetrahedron 60 (2004), 961-965. Hereinbelow, the cyclization of dipeptides of the formula IV to the alcohols II is also referred to below as process B and is illustrated in the scheme below.

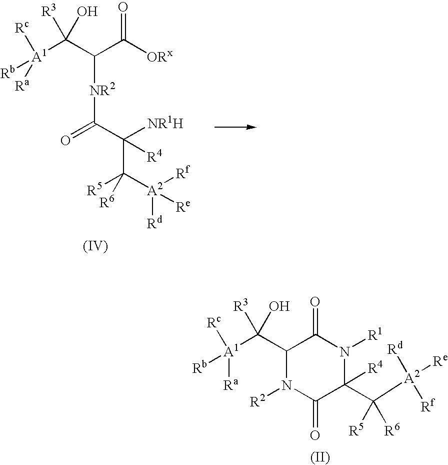

In formula IV, the variables A¹, A², R¹-R⁶, R^{a}, R^{b}, R^{c}, R^{d}, R^{e} and R^{f} are as defined for formula I. The group OR^{x} is a suitable leaving group attached via oxygen. Here, R^{x} is, for example, C₁-C₆-alkyl, in particular methyl or ethyl, or phenyl-C₁-C₆-alkyl, for example benzyl.

The cyclization can be carried out, for example, by reacting a dipeptide of the formula IV either in the presence of acid or base (acidic or basic cyclization) or by heating of the reaction mixture (thermal cyclization).

The bases or acids are added to the dipeptide IV either in equimolar amounts or in excess. In a particular embodiment of the process according to the invention, the bases or acids are employed in excess, based on the dipeptide.

The reaction of the dipeptide IV in the presence of a base is generally carried out at temperatures in the range from 0° C. to the boiling point of the reaction mixture, preferably from 10° C. to 50° C., particularly preferably from 15° C. to 35° C. In general, the reaction is carried out in a solvent, preferably in an inert organic solvent.

Suitable inert organic solvents include aliphatic hydrocarbons, such as pentane, hexane, cyclohexane and mixtures of C₅-C₈-alkanes, aromatic hydrocarbons, such as toluene, o-, m- and p-xylene, halogenated hydrocarbons, such as dichloromethane, dichloroethane, chloroform and chlorobenzene, ethers, such as diethyl ether, diisopropyl ether, tert-butyl methyl ether, dioxane, anisole and tetrahydrofuran, nitrites, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol, tert-butanol, water and also dimethyl sulfoxide, dimethylformamide and dimethylacetamide and also morpholine and N-methylmorpholine. It is also possible to use mixtures of the solvents mentioned.

In a preferred embodiment of the invention, the reaction is carried out in a tetrahydrofuran-water mixture using, for example, a mixing ratio of 1:10 to 10:1 (parts by volume).

Suitable bases are, in general, inorganic compounds, such as alkali metal and alkaline earth metal hydroxides, such as lithium hydroxide, sodium hydroxide, potassium hydroxide or calcium hydroxide, an aqueous solution of ammonia, alkali metal or alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, for example lithium diisopropylamide, sodium amide and potassium amide, alkali metal and alkaline earth metal carbonates, such as lithium carbonate, potassium carbonate, cesium carbonate and calcium carbonate and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide, potassium tert-pentoxide and dimethoxymagnesium, moreover organic bases, for example tertiary amines, such as trimethylamine, triethylamine, diisopropylethylamine, 2-hydroxypyridine and N-methylpiperidine, pyridine, substituted pyridines, such as collidine, lutidine and 4-dimethylaminopyridine, and also bicyclic amines. It is, of course, also possible to use a mixture of different bases.

In one embodiment of the process according to the invention, the reaction of IV is carried out in the presence of bases, preferably in the presence of the bases potassium tert-butoxide, 2-hydroxypyridine or an aqueous solution of ammonia or a mixture of these bases. Preference is given to using only one of these bases. In a particularly preferred embodiment, the reaction is carried out in the presence of an aqueous solution of ammonia which, for example, may be from 10 to 50% strength (w/v).

The reaction of IV in the presence of an acid is usually carried out at temperatures in the range from 10° C. to the boiling point of the reaction mixture, preferably from 50° C. to the boiling point, particularly preferably at the boiling point under reflux. In general, the reaction is carried out in a solvent, preferably in an inert organic solvent.

In principle, suitable solvents are those solvents which can also be used for the basic cyclization, in particular alcohols. In a preferred embodiment, the reaction is carried out in n-butanol.

In principle, suitable acids for the cyclization of IV are both Brönstedt and Lewis acids. In particular, it is possible to employ inorganic acids, for example hydrohalic acids, such as hydrofluoric acid, hydrochloric acid, hydrobromic acid, inorganic oxo acids, such as sulfuric acid and perchloric acid, furthermore inorganic Lewis acids, such as boron trifluoride, aluminum trichloride, iron(III) chloride, tin(IV) chloride, titanium(IV) chloride and zinc(II) chloride, and also organic acids, for example carboxylic acids and hydroxycarboxylic acids, such as formic acid, acetic acid, propionic acid, oxalic acid, citric acid and trifluoroacetic acid, and also organic sulfonic acids, such as toluenesulfonic acid, benzenesulfonic acid, camphorsulfonic acid and the like. It is, of course, also possible to use a mixture of different acids.

In one embodiment of the process according to the invention, the reaction is carried out in the presence of organic acids, for example in the presence of carboxylic acids, such as formic acid, acetic acid or trifluoroacetic acid or a mixture of these acids. Preferably, only one of these acids is used. In a preferred embodiment, the reaction is carried out in acetic acid.

A particularly preferred embodiment of the acidic cyclization is carried out in the presence of n-butanol, N-methylmorpholine and acetic acid under reflux conditions.

In a further embodiment of the invention, the reaction is carried out just by heating the reaction mixture (thermal cyclization). Here, the reaction is usually carried out at temperatures in the range from 10° C. to the boiling point of the reaction mixture, preferably from 50° C. to the boiling point of the reaction mixture, particularly preferably at the boiling point of the reaction mixture under reflux. In general, the reaction is carried out in a solvent, preferably in an inert organic solvent.

In principle, suitable solvents are those solvents which can be used for the basic cyclization. Preference is given to polar aprotic solvents, for example dimethyl sulfoxide or dimethylformamide or mixtures thereof. In a preferred embodiment, the reaction is carried out in dimethyl sulfoxide.

The reaction mixtures obtained in process B, but also the reaction mixtures obtained in processes A1 and A2 and the reaction mixtures of the processes described hereinbelow, can be worked-up in a customary manner. This may take place, for example, by mixing with water, separating the phases and, if appropriate, chromatographic purification of the crude products. Some of the intermediates and end products are obtained in the form of viscous oils which can generally be purified or freed from volatile components under reduced pressure and at moderately elevated temperature. If the intermediates and end products can be obtained as solids, the purification can also be carried out by recrystallisation or digestion.

Some of the precursors and intermediates required for preparing the compounds of the formula II are commercially available, known from the literature or can be prepared by processes known from the literature.

The dipeptide compounds of the formula IV can be prepared, for example, from N-protected dipeptides of the general formula V analogously to processes known from the literature, for example according to Glenn L. Stahl et al., J. Org. Chem. 43(11), (1978), 2285-6 or A. K. Ghosh et al., Org. Lett. 3(4), (2001), 635-638.

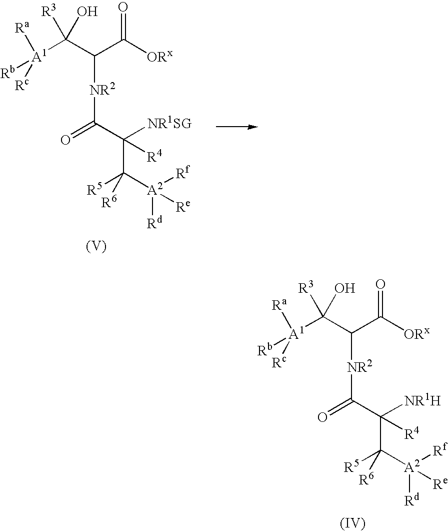

In the formulae IV and V, the variables A¹, A², R¹-R⁶, R^{a}, R^{b}, R^{c}, R^{d}, R^{e} and R^{f} are as defined for formula I, SG is a nitrogen protective group, such as Boc (=tert-butoxycarbonyl), and OR^{x} is a leaving group attached via an oxygen atom. Of course, in each case the preferred meanings for the compounds of the formula II apply correspondingly to the compounds of the formula IV or V. With respect to the leaving group OR^{x}, what was stated above for the dipeptides of the formula IV applies.

Thus, for example, a dipeptide of the formula V in which SG is Boc and OR^{x} is a suitable leaving group, where R^{x} is, for example, C₁-C₆-alkyl, in particular methyl, ethyl or benzyl, can be converted in the presence of an acid into a compound of the formula IV.

The reaction is usually carried out at temperatures in the range from −30° C. to the boiling point of the reaction mixture, preferably from 0° C. to 50° C., particularly preferably from 20° C. to 35° C.

The reaction can take place in a solvent, in particular in an inert organic solvent. Suitable solvents are, in principle, the compounds cited for the basic cyclization, in particular tetrahydrofuran or dichloromethane or mixtures thereof. In a preferred embodiment, the reaction is carried out in dichloromethane.

The acids used are the acids cited for the cyclization of dipeptide IV to piperazine II.

In one embodiment of the process according to the invention, the reaction is carried out in the presence of organic acids, for example in the presence of strong organic acids, such as formic acid, acetic acid or trifluoroacetic acid or mixtures thereof. In a preferred embodiment, the reaction is carried out in the presence of trifluoroacetic acid.

The work-up can be carried out analogously to the procedure described for the cyclization of dipeptide IV to piperazine II.

The protected dipeptides of the formula V can be prepared analogously to processes known from the literature, for example according to Wilford L. Mendelson et al., Int. J. Peptide & Protein Research 35(3), (1990), 249-57. A typical route is the amidation of a Boc-protected amino acid VI with an amino acid ester of the formula VII, as shown in the scheme below:

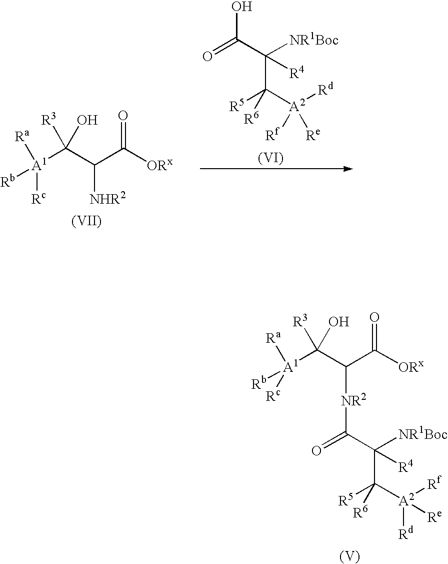

In this scheme, the variables are as defined above. Instead of Boc, it is also possible to use other amino protective groups.

In general, the reaction of VII with VI is carried out at temperatures in a range from −30° C. to the boiling point of the reaction mixture, preferably from 0° C. to 50° C., particularly preferably from 20° C. to 35° C. The reaction can be carried out in a solvent, preferable in an inert organic solvent. Suitable solvents are the solvents mentioned in the context of the basic cyclization of IV to II.

In general, the reaction requires the presence of an activating agent. Suitable activating agents are condensing agents, such as, for example, polystyrene- or non-polystyrene-supported dicyclohexylcarbodiimide (DCC), diisopropylcarbodiimide, 1-ethyl-3-(dimethylaminopropyl)carbodiimide (EDAC), carbonyldiimidazole, chlorocarbonic esters, such as methyl chloroformate, ethyl chloroformate, isopropyl chloroformate, isobutyl chloroformate, sec-butyl chloroformate or allyl chloroformate, pivaloyl chloride, polyphosphoric acid, propanephosphonic anhydride, bis(2-oxo-3-oxazolidinyl)-phosphoryl chloride (BOPCl) or sulfonyl chlorides, such as methanesulfonyl chloride, toluenesulfonyl chloride or benzenesulfonyl chloride. According to one embodiment, a preferred activating agent is EDAC or DCC.

The reaction of VII with VI is preferably carried out in the presence of a base. Suitable bases are the compounds cited for the cyclization of dipeptide IV to piperazine II. In one embodiment, the base used is triethylamine or N-ethyldiisopropylamine or mixtures thereof, particularly preferably N-ethyldiisopropylamine.

The work-up can be carried out analogously to the procedure described for the cyclization of dipeptide IV to piperazine II.

For their part, the compounds of the formula VII can be prepared by deprotecting corresponding protected amino acid compounds VIII analogously to processes known from the literature, for example according to Glenn L. Stahl et al., J. Org. Chem. 43(11), (1978), 2285-6 or A. K. Ghosh et al., Org. Lett. 3(4), (2001), 635-638. The preparation of VII from a Boc-protected amino acid compound VIII is shown in the scheme below.

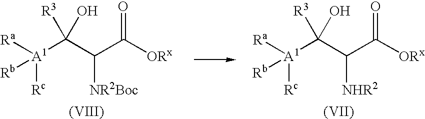

In this scheme, the variables are as defined above. Instead of Boc, it is also possible to use other amino protective groups.

The conversion of a compound of the formula VIII into the compound VII is typically carried out in the presence of an acid at temperatures in a range from −30° C. to the boiling point of the reaction mixture, preferably from 0° C. to 50° C., particularly preferably from 20° C. to 35° C. The reaction can be carried out in a solvent, preferably in an inert organic solvent.

Suitable solvents are, in principle, the compounds mentioned under the basic cyclization, in particular tetrahydrofuran or dichloromethane or mixtures thereof. In a preferred embodiment, the reaction is carried out in dichloromethane.

The acids and acidic catalysts used are the substances cited for the cyclization of dipeptide IV to piperazine II.

In one embodiment of the process according to the invention, the reaction is carried out in the presence of organic acids, for example in the presence of strong organic acids, such as formic acid, acetic acid or trifluoroacetic acid or mixtures thereof. In a preferred embodiment, the reaction is carried out in the presence of trifluoroacetic acid.

The work-up can be carried out analogously to the procedure described for the cyclization of dipeptide IV to piperazine II.

The compounds of the formula VIII can be prepared according to the reaction shown in the scheme below. The reaction of compound XI with the protected amino acid compound X can be carried out analogously to processes known from the literature, for example according to I. Ojima et al., J. Am. Chem. Soc., 109(21), (1987), 6537-6538 or J. M. McIntosh et al., Tetrahedron 48(30), (1992), 6219-6224.

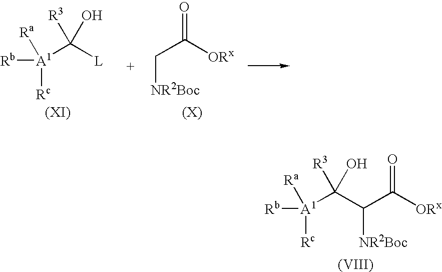

In this scheme, the variables are as defined above. L is a leaving group. Instead of Boc, it is also possible to use other amino protective groups.

The reaction of XI with X is generally carried out in the presence of base. Suitable bases are the compounds cited for the cyclization of dipeptide IV to piperazine II. In a further preferred embodiment, the base used is lithium diisopropylamide, particularly preferably in a substantially equimolar amount, in particular in an equimolar amount. Usually, the reaction is carried out at temperatures in the range from −78° C. to the boiling point of the reaction mixture, preferably from −78° C. to the boiling point, particularly preferably from 78° C. to 30° C.

The reaction can be carried out in a solvent, preferably in an inert organic solvent. Suitable solvents are, in principle, the solvents mentioned under the basic cyclization, in particular dichloromethane or tetrahydrofuran or mixtures thereof. In a preferred embodiment, the reaction is carried out in tetrahydrofuran.

The work-up can be carried out analogously to the procedure described for the cyclization of dipeptide IV to piperazine II.

Some of the compounds of the formula XI are commercially available or can be prepared by transformations, described in the literature, of the corresponding commercially available precursors.

### Process C

Compounds of the formula II.1 in which R³ is H can also be prepared by coupling, in an aldol reaction, the aldehyde XII with the piperazine XIII, as illustrated in the scheme below:

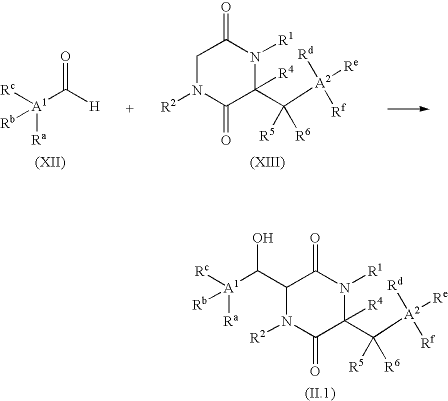

In the formulae II.1, XII and XIII, the variables A¹, A², R¹-R⁶, R^{a}, R^{b}, R^{c}, R^{d}, R^{e} and R^{f} are as defined for formula I.

The reaction is generally carried out in the presence of suitable bases. Suitable bases are bases usually employed for aldol reactions. Examples of these are the compounds mentioned for the cyclization of IV to II. Preference is given to using alkali metal amides, such as lithium diisopropylamide. Suitable reaction conditions are known from the prior art and are described, for example, in J. Org. Chem. 2000, 65 (24), 8402-8405, which are expressly incorporated herein in their entirety.

The aldol reaction may also yield directly the corresponding aldol condensation product, i.e. compounds of the formula I.1 in which R³ is H. This is the case in particular when the reaction proceeds at elevated temperatures and with relatively long reaction times.

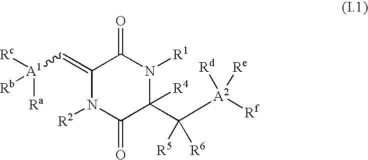

The aldehyde XII is either commercially available or can be synthesized according to known processes for the preparation of aldehydes. Such aldol condensations can be carried out analogously to the processes described in J. Org. Chem. 2000, 65 (24), 8402-8405, which is expressly incorporated herein in its entirety.

In principle, the aldol reaction or condensation can also be employed for preparing compounds I in which R³ is not necessarily hydrogen but can also be C₁-C₆-alkyl, C₃-C₆-cycloalkyl, C₂-C₆-alkenyl, C₃-C₆-cycloalkenyl, C₂-C₆-alkynyl, C₃-C₈-cycloalkynyl, phenyl, phenyl-(C₁-C₆)-alkyl, heterocyclyl, heterocyclyl-(C₁-C₆)-alkyl; phenyl-[C₁-C₆-alkoxycarbonyl]-(C₁-C₆)-alkyl or phenylheterocyclyl-(C₁-C₆)-alkyl and especially C₁-C₆-alkyl. In this case, instead of the aldehyde XII, the ketone IX is used

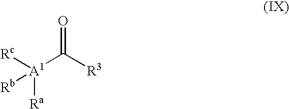

in which R³ is C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, phenyl, phenyl-(C₁-C₆)-alkyl, heterocyclyl, heterocyclyl-(C₁-C₆)-alkyl; phenyl-[C₁-C₆-alkoxycarbonyl]-(C₁-C₆)-alkyl or phenylheterocyclyl-(C₁-C₆)-alkyl and especially C₁-C₆-alkyl.

However, it is possible here that complex reaction mixtures are formed, in particular when R³ is a group in which the carbon atom attached in the α-position to the point of attachment carries a hydrogen atom. Moreover, in most cases more drastic reaction conditions are required, so that the aldolization is preferably only used for preparing compounds I.1.

The compounds of the formula XIII can be prepared by intramolecular cyclization of compounds of the general formula XIV analogously to further processes known from the literature, for example according to T. Kawasaki et al., Org. Lett. 2(19) (2000), 3027-3029, Igor L. Rodionov et al., Tetrahedron 58(42) (2002), 8515-8523 or A. L. Johnson et al., Tetrahedron 60 (2004), 961-965.

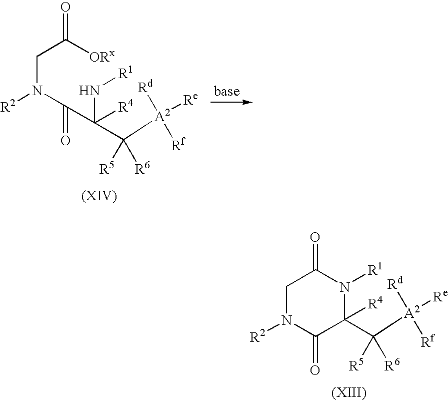

In formula XIV, the variables R^{x}, A², R¹, R², R⁴, R⁵, R⁶, R^{d}, R^{e} and R^{f} are as defined for formula IV. The group OR^{x} is a suitable leaving group attached via oxygen. Here, R^{x} is for example C₁-C₆-alkyl, in particular methyl or ethyl, or phenyl-C₁-C₈-alkyl, for example benzyl.

The cyclization of the compounds of the formula XIV can be carried out in the presence of a base. In this case, the reaction is generally carried out at temperatures in the range from 0° C. to the boiling point of the reaction mixture, preferably from 10° C. to 50° C., particularly preferably from 15° C. to 35° C. The reaction can be carried out in a solvent, preferably in an inert organic solvent.

Suitable solvents are, in principle, the compounds cited under the thermal cyclization, in particular a tetrahydrofuran-water mixture having a mixing ratio of from 1:10 to 10:1.

Suitable bases are the bases mentioned for the basic cyclization of IV to II (process B), in particular potassium tert-butoxide, 2-hydroxypyridine or an aqueous solution of ammonia or a mixture of these bases. Preferably, only one of these bases is used. In a particularly preferred embodiment, the reaction is carried out in the presence of an aqueous solution of ammonia which, for example, may be from 10 to 50% strength (w/v).

For their part, the compounds of the formula XIV can be prepared by the synthesis illustrated in the scheme below, analogously to processes known from the literature, for example according to Wilford L. Mendelson et al., Int. J. Peptide & Protein Research 35(3), (1990), 249-57, Glenn L. Stahl et al., J. Org. Chem. 43(11), (1978), 2285-6 or A. K. Ghosh et al., Org. Lett. 3(4), (2001), 635-638.

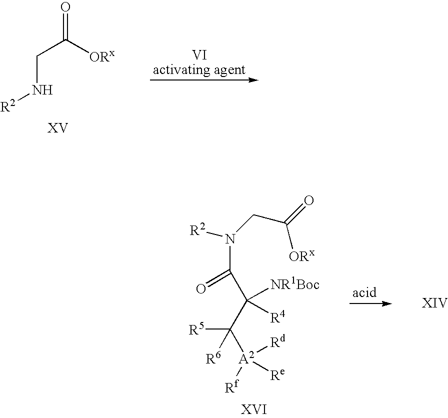

In the scheme, the variables R^{x}, A², R¹, R², R⁴, R⁵, R^{e}, R^{d}, R^{e} and R^{f} are as defined for formula IV or XIV. In a first step, the synthesis comprises the coupling of amino acid compounds XV with Boc-protected amino acids VI in the presence of an activating agent. Instead of Boc, it is also possible to use another amino protective group.

The reaction of a compound of the formula XV with a compound of the formula VI is usually carried out at temperatures in the range from −30° C. to the boiling point of the reaction mixture, preferably from 0° C. to 50° C., particularly preferably from 20° C. to 35° C. The reaction can be carried out in a solvent, preferably in an inert organic solvent. For further details, reference is made to the preparation of compound V by amidation of the amino acid compound VI with the compound VII.

In general, the reaction requires the presence of an activating agent. Suitable activating agents are condensing agents, such as, for example, polystyrene- or non-polystyrene-supported dicyclohexylcarbodiimide (DCC), diisopropylcarbodiimide, 1-ethyl-3-(dimethylaminopropyl)carbodiimide (EDAC), carbonyldiimidazole, chlorocarbonic esters, such as methyl chloroformate, ethyl chloroformate, isopropyl chloroformate, isobutyl chloroformate, sec-butyl chloroformate or allyl chloroformate, pivaloyl chloride, polyphosphoric acid, propanephosphonic anhydride, bis(2-oxo-3-oxazolidinyl)-phosphoryl chloride (BOPCl) or sulfonyl chlorides, such as methanesulfonyl chloride, toluenesulfonyl chloride or benzenesulfonyl chloride. According to one embodiment, a preferred activating agent is EDAC or DCC.

The reaction of XV with VI is preferably carried out in the presence of a base. Suitable bases are the compounds cited under process B (cyclization of dipeptide IV to piperazine II). In one embodiment, the base used is triethylamine or N-ethyldiisopropylamine or mixtures thereof, particularly preferably N-ethyldiisopropylamine.

The work-up can be carried out analogously to the procedure described for process B (cyclization of dipeptide IV to piperazine II).

The deprotection of the compound XVI to give the compound XIV is typically carried out by treatment with an acid. The reaction is usually carried out at temperatures in the range from −30° C. to the boiling point of the reaction mixture, preferably from 0° C. to 50° C., particularly preferably from 20° C. to 35° C. The reaction can be carried out in a solvent, preferably in an inert organic solvent.

Suitable solvents are, in principle, the solvents mentioned under process B in connection with the basic cyclization, in particular tetrahydrofuran or dichloromethane or mixtures thereof. In a preferred embodiment, the reaction is carried out in dichloromethane.

The acids used are the acids mentioned for process B (cyclization of dipeptide IV to piperazine II). For further details, reference is also made to the deprotection of V to compound IV. The reaction conditions mentioned there are also suitable for deprotecting compound XVI. In one embodiment of the process according to the invention, the reaction is carried out in the presence of organic acids, in particular strong organic acids, for example in the presence of formic acid, acetic acid or trifluoroacetic acid or mixtures thereof. In a preferred embodiment, the reaction is carried out in the presence of trifluoroacetic acid.

If, instead of Boc, a different protective group is used, the deprotection process employed is of course suitable for the protective group in question.

The work-up can be carried out analogously to the procedure described for process B (cyclization of dipeptide IV to piperazine II).

### Process D

The invention furthermore provides a process for preparing piperazine compounds of the general formula I which comprises reacting, in the presence of a base, a compound of the formula IX

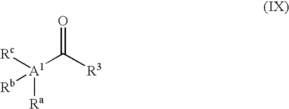

in which the variables A¹, R^{a}, R^{b} and R^{c} are as defined above and R³ is hydrogen, C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, phenyl, phenyl-(C₁-C₆)-alkyl, heterocyclyl, heterocyclyl-(C₁-C₆)-alkyl, phenyl-[C₁-C₆-alkoxycarbonyl]-(C₁-C₆)-alkyl or phenylheterocyclyl-(C₁-C₆)-alkyl, with a phosphonic ester of the formula XVII

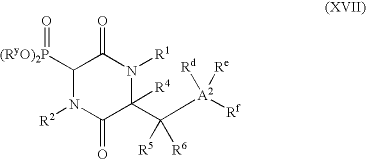

in which the variables A², R¹, R², R⁴, R⁵, R⁶, R^{d}, R^{e} and R^{f} are as defined above and R^{y} is C₁-C₄-alkyl, for example methyl, ethyl, propyl, isopropyl, butyl or isobutyl, aryl, for example phenyl, or aryl-C₁-C₄-alkyl, for example benzyl.

The reaction mentioned above is also referred to as Wittig-Horner-Emmons reaction.

R^{y} is preferably C₁-C₄-alkyl and in particular methyl or ethyl.

This process is preferably employed to prepare compounds of the formula I.1 in which R³ is H. To this end, the procedure described above is adopted, the compound IX used being the aldehyde XII (corresponds to the compound IX in which R³ is H), which is reacted with the phosphonic ester XVII. This is illustrated in the scheme below:

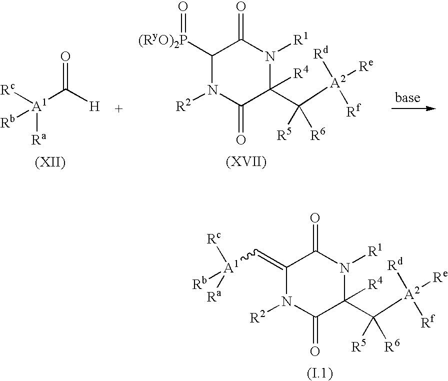

In the scheme, the variables A¹, A², R¹, R², R⁴, R⁵, R⁶, R^{a}, R^{b}, R^{c}, R^{d}, R^{e} and R^{f} are as defined above. R^{y} is C₁-C₄-alkyl, for example methyl, ethyl, propyl, isopropyl, n-butyl, or isobutyl, aryl, for example phenyl, or aryl-C₁-C₄-alkyl, for example benzyl. R^{y} is preferably C₁-C₄-alkyl and in particular methyl or ethyl.

Suitable reaction conditions are known from the prior art and described, for example, in J. Org. Chem. 2001, 66(11), 3984-3997 and in Tetrahedron Lett. 1987, 28, 4275, which are expressly incorporated herein in their entirety.

Thus, suitable bases are all bases customary for Wittig-Horner-Emmons reactions, for example alkali metal hydrides, such as sodium hydride, and alkali metal amides, such as lithium diisopropylamide. However, preference is given to relatively weak bases, such as, for example, the non-nucleophilic bases mentioned above, for example the amidines DBU and DBN or sterically hindered alkali metal alkoxides, such as potassium tert-butoxide. Suitable solvents are, of course, aprotic and are selected, for example, from halogenated hydrocarbons, such as methylene chloride, chloroform and chlorobenzene, and aromatic hydrocarbons, such as benzene and toluene.

In principle, the Wittig-Horner-Emmons reaction can also be employed for preparing compounds I in which R³ is not necessarily hydrogen but may also be C₁-C₆-alkyl, C₁-C₆-cycloalkyl, C₂-C₆-alkenyl, C₃-C₆-cycloalkenyl, C₂-C₆-alkynyl, C₃-C₆-cycloalkynyl, phenyl, phenyl-(C₁-C₆)-alkyl, heterocyclyl, heterocyclyl-(C₁-C₆)-alkyl; phenyl-[C₁-C₆-alkoxycarbonyl]-(C₁-C₆)-alkyl or phenylheterocyclyl-(C₁-C₆)-alkyl and especially C₁-C₆-alkyl. In this case, instead of the aldehyde XII, the ketone IX.1 (corresponds to compound IX in which R³ is not hydrogen) is used

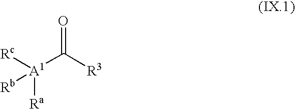

in which R³ is C₁-C₆-alkyl, C₃-C₆-cycloalkyl, C₂-C₆ alkenyl, C₃-C₆-cycloalkenyl, C₂-C₈ alkynyl, C₃-C₆-cycloalkynyl, phenyl, phenyl-(C₁-C₆)-alkyl, heterocyclyl, heterocyclyl-(C₁-C₆)-alkyl; phenyl-[C₁-C₆-alkoxycarbonyl]-(C₁-C₆)-alkyl or phenylheterocyclyl-(C₁-C₆)-alkyl and especially C₁-C₆-alkyl.

Here, what was said above with respect to the preparation of the piperazine compound of the formula I.1 applies correspondingly. However, here more drastic reaction conditions are required in most cases, for example stronger bases, higher temperatures, longer reaction times, etc., so that the above-described reaction according to Wittig, Horner and Emmons is preferably employed for the preparation of compounds I.1.

The phosphonic ester XVII can be obtained, for example, by cyclization of the phosphonate XVIII:

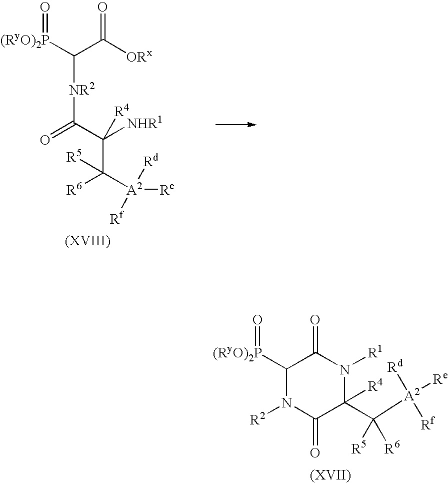

In the scheme, the variables R^{y}, A², R¹, R², R⁴, R⁵, R⁶, R^{d}, R^{e} and R^{f} are as defined above. OR^{x} is a leaving group attached via oxygen, where R^{x} is preferably C₁-C₆-alkyl, such as methyl, ethyl, propyl, isopropyl, n-butyl, isobutyl, pentyl or hexyl, or phenyl-C₁-C₆-alkyl, such as benzyl.

The cyclization can be carried out analogously to the cyclization, described above, of compound IV to compound II.

The phosphonate XVIII can be prepared by coupling of the previously N-deprotected phosphonate XIX with the N-protected amino acid XX and subsequent removal of the protective group:

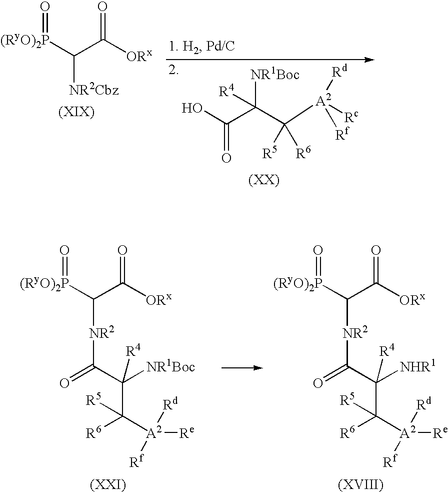

In the scheme, the variables R^{x}, R^{y}, A², R¹, R², R⁴, R⁵, R⁶, R^{d}, R^{e} and R^{f} are as defined above. Cbz is the benzyloxycarbonyl protective group. Instead of Cbz and Boc, it is also possible to use other amino protective groups.

The Cbz protective group is generally removed reductively using hydrogen in the presence of palladium-on-carbon. Other customary processes for removing Cbz groups may also be employed. If other protective groups are used, the removal is generally carried out according to the processes suitable for the group in question.

The above reaction can be carried out according to the process described in Tetrahedron 2004, 60, 961-965. Thus, the coupling in Step 2 with the amino acid XX can be carried out analogously to the preparation of compound V from compounds VI and VII. Deprotection of compound XXI to compound XVIII can be carried out analogously to the deprotection of compound V to compound IV.

The phosphonates XIX are either commercially available or they can be prepared according to the processes described in Synthesis 1986, 53-60.

### Process E

The invention furthermore provides a process for preparing piperazine compounds of the general formula I which comprises reacting, in the presence of a base, a compound of the formula IX

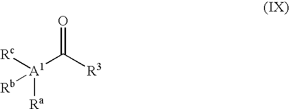

in which the variables A¹, R^{a}, R^{b} and R^{c} are as defined above and R³ is hydrogen, C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, phenyl, phenyl-(C₁-C₆)-alkyl, heterocyclyl, heterocyclyl-(C₁-C₆)-alkyl, phenyl-[C₁-C₆-alkoxycarbonyl]-(C₁-C₆)-alkyl or phenylheterocyclyl-(C₁-C₆)-alkyl,
with a phosphonic ester of the formula XXI

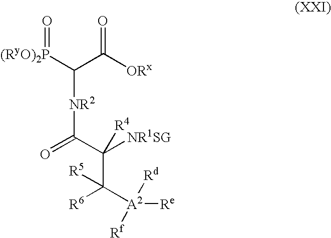

in which the variables A², R¹, R², R⁴, R⁵, R⁶, R^{d}, R^{e} and R^{f} are as defined above, OR^{x} is a leaving group attached via oxygen, where R^{x} is preferably C₁-C₆-alkyl, such as methyl, ethyl, propyl, isopropyl, n-butyl, isobutyl, pentyl or hexyl, or phenyl-C₁-C₆-alkyl, such as benzyl, R^{y} is C₁-C₄-alkyl, for example methyl, ethyl, propyl, isopropyl, n-butyl, or isobutyl, aryl, for example phenyl, or aryl-C₁-C₄-alkyl, for example benzyl, and SG is an amino protective group, for example Boc,
to give the compound of the formula XXII

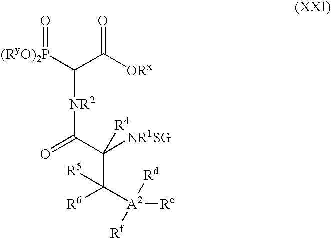

removing the amino protective group giving the compound of the formula XXIII

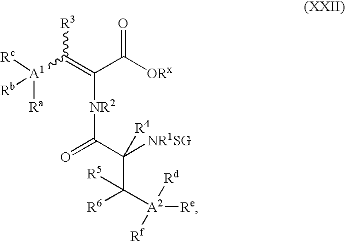

and cyclizing the compound of the formula XXIII to the compound of the formula I.

The first step of the process according to the invention is also referred to as Wittig-Horner-Emmons reaction.

R^{y} is preferably C₁-C₄-alkyl, in particular methyl or ethyl.

The process according to the invention is preferably used to prepare compounds of the formula I.1 in which R³ is H. To this end, the procedure described above is adopted, where the aldehyde XII (corresponds to the compound IX in which R³ is H) is used for the Wittig-Horner-Emmons reaction. Using the open-chain phosphonic ester XXI.1, the latter is converted into the olefin of the formula XXII.1 which, after removal of the protective group, is cyclized to piperazine I.1, as illustrated in the scheme below:

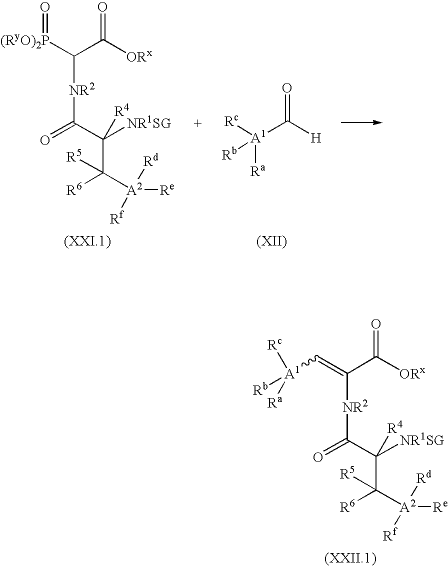

In the formulae I.1, XII, XXI, XXII.1 and XXIII.1, the variables A¹, A², R¹-R⁶, R^{a}, R^{b}, R^{c}, R^{d}, R^{e} and R^{f} are as defined for formula I. OR^{x} is a leaving group attached via oxygen, where R^{x} is preferably C₁-C₆-alkyl, such as methyl, ethyl, propyl, isopropyl, n-butyl, isobutyl, pentyl or hexyl, or phenyl-C₁-C₆-alkyl, such as benzyl. R^{y} is C₁-C₄-alkyl, for example methyl, ethyl, propyl, isopropyl, n-butyl, or isobutyl, aryl, for example phenyl, or aryl-C₁-C₄-alkyl, for example benzyl. R^{y} is preferably C₁-C₄-alkyl, in particular methyl or ethyl. SG is a suitable amino protective group, in particular Boc.

The reaction of the phosphonate XXI with the aldehyde XII can take place analogously to the reaction, described above, of the phosphonate XVII with the aldehyde XII. The reaction can also be carried out analogously to the process described in Synthesis 1992, 487-490, Synthesis 1992, 482-486 and Synthesis 1984, 53-60, which are expressly incorporated herein in their entirety.

The removal of the protective group is generally carried out by processes known form the literature. If, for example, SG is Boc, the protective group can be removed as described above for compound V, for example by reacting the compound XXII.1 with an acid. The deprotection reaction can also be carried out analogously to the process described in Tetrahedron 2004, 60, 961-965, which is expressly incorporated herein in its entirety.

The cyclization of dipeptide XXIII.1 to piperazine I.1 can be carried out analogously to the conversion of compound IV into compound II. In this case, however, only the cyclization in the presence of an acid is suitable, owing to the presence of the double bond. Here, what was stated for the cyclization of the compound IV with respect to suitable and preferred acids and solvents applies correspondingly. The acidic cyclization can also be carried out according to the process described in Tetrahedron 2004, 60, 961-965, which is expressly incorporated herein in its entirety.

Instead of the N-protected, for example the Boc-protected, compound XXI.1 it is also possible to use the corresponding deprotected compound for the Wittig-Horner-Emmons reaction, so that compound XXIII.1 is formed directly. The protective group is generally removed by processes known from the literature. If, for example, SG is Boc, the Boc protective group can be removed from compound XXI as described above for compound V, for example by reacting the compound XXI with an acid. The deprotection reaction may also be carried out analogously to the process described in Tetrahedron 2004, 60, 961-965, which is expressly incorporated herein in its entirety.

In principle, the process according to the invention can also be employed for preparing compounds I in which R³ is not necessarily hydrogen but can also be C₁-C₆-alkyl, C₃-C₆-cycloalkyl, C₂-C₆-alkenyl, C₃-C₆-cycloalkenyl, C₂-C₆-alkynyl, C₃-C₈-cycloalkynyl, phenyl, phenyl-(C₁-C₆)-alkyl, heterocyclyl, heterocyclyl-(C₁-C₆)-alkyl; phenyl-[C₁-C₆-alkoxycarbonyl]-(C₁-C₆)-alkyl or phenylheterocyclyl-(C₁-C₆)-alkyl and especially C₁-C₆-alkyl. In this case, instead of the aldehyde XII, the ketone IX.1 is used

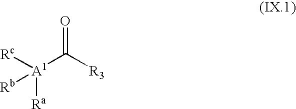

in which R³ is C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, phenyl, phenyl-(C₁-C₆)-alkyl, heterocyclyl, heterocyclyl-(C₁-C₆)-alkyl; phenyl-[C₁-C₆-alkoxycarbonyl]-(C₁-C₆)-alkyl or phenylheterocyclyl-(C₁-C₈)-alkyl and especially C₁-C₆-alkyl.

Here, what was said above with respect to the preparation of the piperazine compound of the formula I.1 applies correspondingly. However, here more drastic reaction conditions are required in most cases, for example stronger bases, higher temperatures, longer reaction times, etc., so that the above-described preparation of the compound I via phosphonate XXI is preferably employed for the preparation of compounds I.1.

### Process F

The compounds of the formula I where R¹≠hydrogen can also be prepared by reacting a piperazine compound of the formula I in which R¹ is hydrogen with an alkylating agent or acylating agent which contains the radical R¹ different from hydrogen. Such reactions can be carried out analogously to processes known from the literature, for example according to the methods described by I. O. Donkor et al., Bioorg. Med. Chem. Lett. 11 (19) (2001), 2647-2649, B. B. Snider et al., Tetrahedron 57 (16) (2001), 3301-3307, I. Yasuhiro et al., J. Am. Chem. Soc. 124(47) (2002), 14017-14019, or M. Falorni et al., Europ. J. Org. Chem. (8) (2000), 1669-1675.

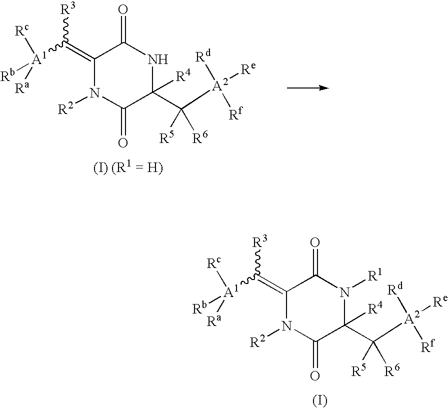

To this end, the piperazine compound of the formula I where R¹=hydrogen is reacted with a suitable alkylating agent, hereinbelow compound X¹—R¹, or acylating agent, hereinbelow compound X²—R¹, which gives a piperazine compound of the formula I where R¹≠hydrogen.

In the alkylating agents X¹—R¹, X¹ can be halogen or O—SO₂—R^{m}, where R^{m} is C₁-C₄-alkyl or aryl, which are optionally substituted by halogen, C₁-C₄-alkyl or halo-C₁-C₄-alkyl. In the acylating agents X²—R¹, X² can be halogen, in particular Cl. Here, R¹≠hydrogen and is as defined above and is in particular C₁-C₆-alkyl, C₃-C₆-cycloalkyl, C₃-C₆-alkenyl, C₃-C₆-cycloalkenyl, C₃-C₆-alkynyl, C₃-C₆-cycloalkynyl, phenyl-(C₁-C₈)-alkyl, heterocyclyl, heterocyclyl-(C₁-C₈)-alkyl; phenyl-[C₁-C₆-alkoxycarbonyl]-(C₁-C₆)-alkyl or phenylheterocyclyl-(C₁-C₆)-alkyl; or COR²¹ or SO₂R²⁵, where the abovementioned aliphatic, cyclic or aromatic moieties of R¹ may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl or C₁-C₄-alkylcarbonyloxy.

The reaction is usually carried out at temperatures in the range from −78° C. to the boiling point of the reaction mixture, preferably from −50° C. bis 65° C., in particular preferably from −30° C. to 65° C. In general, the reaction is carried out in a solvent, preferably in an inert organic solvent.

Suitable solvents are the compounds cited under process B (cyclization of dipeptide IV to piperazine II), inter alia toluene, dichloromethane, tetrahydrofuran or dimethyl formamide or mixtures thereof.

In a preferred embodiment of the invention, the reaction is carried out in tetrahydrofuran.

In a preferred embodiment, the compound I where R¹═H is reacted with the alkylating or acylating agent in the presence of a base. Suitable bases are the compounds cited under process B (cyclization of dipeptide IV to piperazine II). The bases are generally employed in equimolar amounts. They can also be used in excess or even as solvent. In a preferred embodiment, the base is added in an equimolar amount or in a substantially equimolar amount. In a further preferred embodiment, the base used is sodium hydride.

Work-up is generally carried out analogously to the procedure described under process B (cyclization of dipeptide IV to piperazine II).

Alternatively, the alkylation or acylation of the group NR¹ in which R¹ is H, can also be carried out in the precursors. Thus, for example, compounds II, IV, V, XIII, XIV, XVI, XXII or XXIII in which R¹ is H can be N-alkylated or N-acylated as described above.

### Process G

Analogously to the manner described above, compounds I or precursors thereof in which R² is hydrogen can be reacted with alkylating agents R²—X¹ or acylating agents R²—X², which gives compounds of the formula I or precursors thereof where R²≠hydrogen. The reaction conditions correspond to the conditions described above.

### Process H

Moreover, the compounds of the formula I can be modified on group A¹. Thus, they can be prepared, for example, according to the process illustrated in the scheme below by converting the substituent R^{a}, for example analogously to the methods described by J. Tsuji, Top. Organomet. Chem. (14) (2005), 332 pp., or J. Tsuji, Organic Synthesis with Palladium Compounds, (1980), 207 pp.

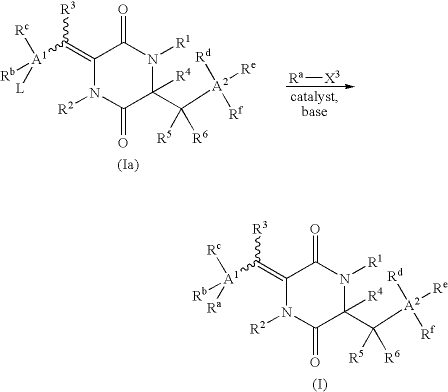

To this end, a piperazine compound of the formula Ia which, instead of the substituent R^{a}, has a suitable leaving group L is converted by reaction with a coupling reagent which contains a group R^{a} (compound R^{a}—X³) into another piperazine derivative of the formula I.

The reaction is usually carried out in the presence of a catalyst, preferably in the presence of a transition metal catalyst. In general, the reaction is carried out in the presence of a base.

This reaction sequence is illustrated below using the example of the substituent R^{a} and can of course be employed in an analogous manner for converting the substituents R^{b} and R^{c}.

Suitable leaving groups L are, for example, halogen or S(O)ₙR^{k}, where n=0, 1, 2 and R^{k} is C₁-C₆-alkyl, halo-(C₁-C₆)-alkyl or optionally halogenated or C₁-C₄-alkyl-substituted aryl.

Suitable coupling reagents X³—R^{a} are in particular those compounds in which X³, if R^{a} is C₁-C₆-alkyl, C₂-C₆-alkenyl, aryl or heteroaryl, denotes one of the following groups:
Zn—R^{I} where R^{I} is halogen, C₁-C₆-alkyl, C₂-C₆-alkenyl, aryl or heteroaryl;
B(OR^{m})₂ where R^{m} is H or C₁-C₆-alkyl, where two alkyl substituents together may form a C₂-C₄-alkylene chain; or
SnRⁿ₃ where Rⁿ is C₁-C₆-alkyl or aryl.

If R^{a} is C₂-C₆-alkynyl, X³ may also be hydrogen.

To prepare the compound I in which R^{a} is CN, the compound Ia in which L is bromine or iodine can also be reacted with copper cyanide analogously to known processes (see, for example, Organikum, 21st Edition, 2001, Wiley, p. 404 and literature cited therein).

Here, according to a preferred embodiment, L or R^{a} in the compounds of the formula I are attached in the ortho-position to the point of attachment of A¹ to a carbon atom of A¹.

This reaction is usually carried out at temperatures in the range from −78° C. to the boiling point of the reaction mixture, preferably from −30° C. to 65° C., particularly preferably at temperatures from 30° C. to 65° C. In general, the reaction is carried out in an inert organic solvent in the presence of a base.

Suitable solvents are the compounds cited under process B (cyclization of dipeptide IV to piperazine II). In one embodiment of the process according to the invention, use is made of tetrahydrofuran with a catalytic amount of water; in another embodiment, only tetrahydrofuran is used.

Suitable bases are the compounds cited under process B (cyclization of dipeptide IV to piperazine II).

The bases are generally employed in equimolar amounts. They can also be employed in excess or even as solvent.

In a preferred embodiment of the process according to the invention, the base is added in an equimolar amount. In a further preferred embodiment, the base used is triethylamine or cesium carbonate, particularly preferably cesium carbonate.

Suitable catalysts for the process according to the invention are, in principle, compounds of the transition metals Ni, Fe, Pd, or Cu. It is possible to use organic or inorganic compounds. Pd(PPh₃)₂Cl₂, Pd(OAc)₂, PdCl₂ or Na₂PdCl₄ may be mentioned by way of example. Here, Ph is phenyl.

The different catalysts can be employed either individually or else as mixtures. In a preferred embodiment of the invention, Pd(PPh₃)₂Cl₂ is used.

The work-up can be carried out analogously to the procedure described for process B (cyclization of dipeptide IV to piperazine II).

Alternatively, the conversion of group A¹ can also be carried out with the precursors of the compound Ia. Thus, for example, compounds II, IV, V, VII, VIII, IX, XI and XII in which, instead of the radical R^{a}, there is a group L attached to A¹, can be subjected to the reaction described above.

### Process I

piperazine compounds of the formula I in which one of the groups R^{a}, R^{b} or R^{c} is COOH can furthermore be prepared from piperazine compounds of the formula I in which R^{a}, R^{b} or R^{c} is COOR^{z}, where R^{z} is alkyl, for example CH₃, by hydrolysis of the ester group. The hydrolysis can be performed, for example, by reaction with (H₃C)₃SnOH, for example according to K. C. Nicolaou et al., Angew. Chem. Int. Ed. Engl. (44) (2005), 1378. The carboxylic acid obtained in this manner can then be converted by standard methods of organic synthesis, if appropriate after conversion into the acid chloride, by reaction with an amine HNR^{u}R^{v} or an alcohol HOR^{w}, into the corresponding ester or the amide (cf. e.g. Organikum, Autorenkollektiv, Leipzig 1993, 19. edition, pp. 424, 429). This reaction sequence is illustrated hereinbelow using the example of the substituent R^{a}, but it is, of course, also possible to employ this sequence in an analogous manner for converting the substituents R^{b} and R^{c}.

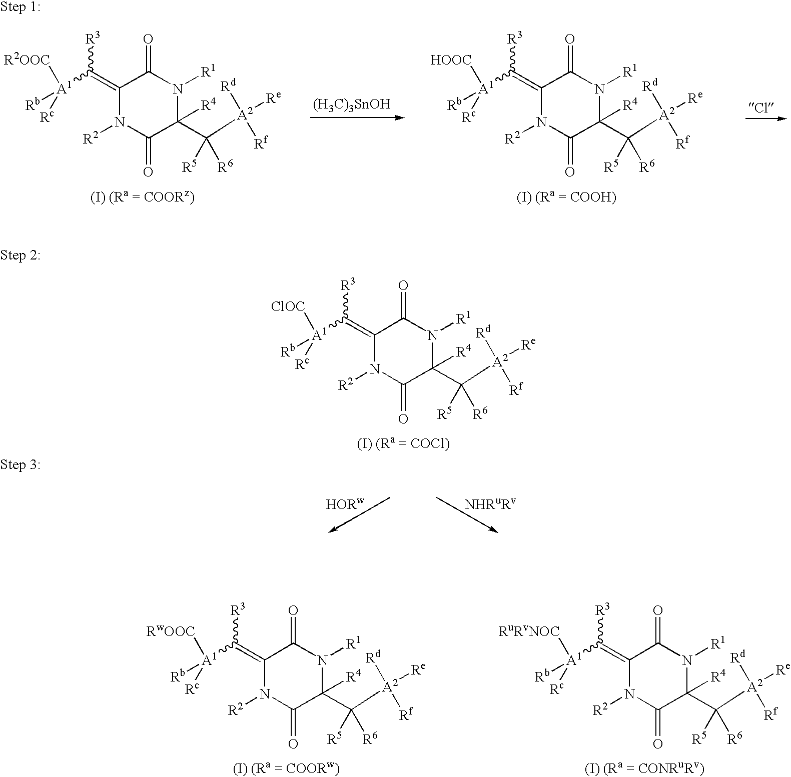

In this scheme, the variables A¹, A², R¹-R⁶, R^{b}, R^{c}, R^{d}, R^{e} and R^{f} have the meanings given above. R^{u} and R^{v} independently of one another are hydrogen, C₁-C₆-alkyl, C₂-C₆-alkenyl, C₂-C₆-alkynyl, C₁-C₆-alkoxy, C₁-C₆-alkylsulfonyl, C₁-C₆-alkylaminosulfonyl, [di-(C₁-C₆)-alkylamino]sulfonyl or optionally substituted phenyl. R^{w} is C₁-C₆-alkyl, C₃-C₆-alkenyl or C₃-C₆-alkynyl. R^{z} is C₁-C₆-alkyl, C₃-C₆-alkenyl, C₃-C₆-alkynyl, benzyl, in particular C₁-C₄-alkyl.

In a first step, the ester group in the piperazine compound I {R^{a}═COOR^{z}} is hydrolyzed. The hydrolysis can be performed, for example, by reaction with (H₃C)₃SnOH, which gives the free acid of I {R^{a}═COOH}. The conversion into the free acid is usually carried out using an excess of (H₃C)₃SnOH. In general, the reaction is carried out in an inert organic solvent. Suitable solvents include in particular dichloroethane. In general, the reaction is carried out at elevated temperature, for example at about 80° C.

In a second step, the acid I {R^{a}═COOH} is converted into its acid chloride (R^{a}═COCl). The conversion into the acid chloride is usually carried out at temperatures of from 10° C. to 50° C., preferably at room temperature, for example 25° C. In general, the reaction is carried out in an inert organic solvent. The suitable solvents include in particular dichloromethane. In a preferred embodiment, the reaction is carried out in dichloromethane and catalytic amounts of dimethylformamide. A large number of reagents are suitable for the chlorination, for example oxalyl chloride or thionyl chloride. Preference is given to using substantially equimolar amounts of the chlorinating reagent, in particular oxalyl chloride.

The reaction with an amine NHR^{u}R^{v} in the subsequent reaction is usually carried out by adding an excess of the amine in question. The reaction can be carried out in a temperature range of from 0° C. to 40° C., preferably at room temperature, for example 25° C.

The reaction with an alcohol HOR^{w} in the subsequent reaction is usually carried out by adding an excess both of the alcohol in question and of triethylamine.

The reaction can be carried out in a temperature range of from 0° C. to 40° C., preferably at room temperature, for example at 25° C.

The work-up can be carried out analogously to the procedure described for process B (cyclization of dipeptide IV to piperazine II).

The compounds I and their agriculturally useful salts are suitable, both in the form of isomer mixtures and in the form of the pure isomers, as herbicides. They are suitable as such or as an appropriately formulated composition. The herbicidal compositions comprising the compound I or Ia control vegetation on non-crop areas very efficiently, especially at high rates of application. They act against broad-leaved weeds and grass weeds in crops such as wheat, rice, maize, soya and cotton without causing any significant damage to the crop plants. This effect is mainly observed at low rates of application.

Depending on the application method in question, the compounds I or Ia, or herbicidal compositions comprising them, can additionally be employed in a further number of crop plants for eliminating undesirable plants. Examples of suitable crops are the following:

Allium cepa, Ananas comosus, Arachis hypogaea, Asparagus officinalis, Avena sativa, Beta vulgaris spec. altissima, Beta vulgaris spec. rapa, Brassica napus var. napus, Brassica napus var. napobrassica, Brassica rapa var. silvestris, Brassica oleracea, Brassica nigra, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Clycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pistacia vera, Pisum sativum, Prunus avium, Prunus persica, Pyrus communis, Prunus armeniaca, Prunus cerasus, Prunus dulcis and prunus domestica, Ribes sylvestre, Ricinus communis, Saccharum officinarum, Secale cereale, Sinapis alba, Solanum tuberosum, Sorghum bicolor (s. vulgare), Theobroma cacao, Trifolium pratense, Triticum aestivum, Triticale, Triticum durum, Vicia faba, Vitis vinifera and Zea mays.

In addition, the compounds of the formula I may also be used in crops which tolerate the action of herbicides owing to breeding, including genetic engineering methods.

In addition, the compounds I can also be used in crops which tolerate insects or fungal attack as the result of breeding, including genetic engineering methods.

Furthermore, it has been found that the compounds of the formula I are also suitable for the defoliation and/or desiccation of plant parts, for which crop plants such as cotton, potato, oilseed rape, sunflower, soybean or field beans, in particular cotton, are suitable. In this regard, there have been found compositions for the desiccation and/or defoliation of plants, processes for preparing these compositions and methods for desiccating and/or defoliating plants using the compounds of the formula I.

As desiccants, the compounds of the formula I are particularly suitable for desiccating the above-ground parts of crop plants such as potato, oilseed rape, sunflower and soybean, but also cereals. This makes possible the fully mechanical harvesting of these important crop plants.

Also of economic interest is to facilitate harvesting, which is made possible by concentrating within a certain period of time the dehiscence, or reduction of adhesion to the tree, in citrus fruit, olives and other species and varieties of pernicious fruit, stone fruit and nuts. The same mechanism, i.e. the promotion of the development of abscission tissue between fruit part or leaf part and shoot part of the plants is also essential for the controlled defoliation of useful plants, in particular cotton.

Moreover, a shortening of the time interval in which the individual cotton plants mature leads to an increased fiber quality after harvesting.

The compounds I, or the herbicidal compositions comprising them, can be used, for example, in the form of ready-to-spray aqueous solutions, powders, suspensions, also highly concentrated aqueous, oily or other suspensions or dispersions, emulsions, oil dispersions, pastes, dusts, materials for broadcasting, or granules, by means of spraying, atomizing, dusting, spreading or watering or treatment of the seed or mixing with the seed. The use forms depend on the intended purpose; in any case, they should ensure the finest possible distribution of the active compounds according to the invention.

The herbicidal compositions comprise a herbicidally effective amount of at least one compound of the formula I or an agriculturally useful salt of I, and auxiliaries which are customary for the formulation of crop protection agents.

Examples of auxiliaries customary for the formulation of crop protection agents are inert auxiliaries, solid carriers, surfactants (such as dispersants, protective colloids, emulsifiers, wetting agents and tackifiers), organic and inorganic thickeners, bactericides, antifreeze agents, antifoams, optionally colorants and, for seed formulations, adhesives.

Examples of thickeners (i.e. compounds which impart to the formulation modified flow properties, i.e. high viscosity in the state of rest and low viscosity in motion) are polysaccharides, such as xanthan gum (Keizan® from Kelco), Rhodopol® 23 (Rhone Poulenc) or Veegum® (from R.T. Vanderbilt), and also organic and inorganic sheet minerals, such as Attaclay® (from Engelhardt).

Examples of antifoams are silicone emulsions (such as, for example, Silikon® SRE, Wacker or Rhodorsil® from Rhodia), long-chain alcohols, fatty acids, salts of fatty acids, organofluorine compounds and mixtures thereof.

Bactericides can be added for stabilizing the aqueous herbicidal formulation. Examples of bactericides are bactericides based on dichlorophen and benzyl alcohol hemiformal (Proxel® from ICI or Acticide® RS from Thor Chemie and Kathon® MK from Rohm & Haas), and also isothiazolinone derivates, such as alkylisothiazolinones and benzisothiazolinones (Acticide MBS from Thor Chemie).

Examples of antifreeze agents are ethylene glycol, propylene glycol, urea or glycerol.

Examples of colorants are both sparingly water-soluble pigments and water-soluble dyes. Examples which may be mentioned are the dyes known under the names Rhodamin B, C.I. Pigment Red 112 and C.I. Solvent Red 1, and also pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of adhesives are polyvinylpyrrolidone, polyvinyl acetate, polyvinyl alcohol and tylose.

Suitable inert additives are, for example, the following:

mineral oil fractions of medium to high boiling point, such as kerosene or diesel oil, furthermore coal tar oils and oils of vegetable or animal origin, aliphatic, cyclic and aromatic hydrocarbons, for example paraffin, tetrahydronaphthalene, alkylated naphthalenes or their derivatives, alkylated benzenes or their derivatives, alcohols such as methanol, ethanol, propanol, butanol and cyclohexanol, ketones such as cyclohexanone, strongly polar solvents, for example amines such as N-methylpyrrolidone, or water.

Solid carriers are mineral earths such as silicas, silica gels, silicates, talc, kaolin, limestone, lime, chalk, bole, loess, clay, dolomite, diatomaceous earth, calcium sulfate, magnesium sulfate and magnesium oxide, ground synthetic materials, fertilizers such as ammonium sulfate, ammonium phosphate, ammonium nitrate, ureas, and products of vegetable origin, such as cereal meal, tree bark meal, wood meal and nutshell meal, cellulose powders, or other solid carriers.

Suitable surfactants (adjuvants, wetting agents, tackifiers, dispersants and also emulsifiers) are the alkali metal salts, alkaline earth metal salts and ammonium salts of aromatic sulfonic acids, for example lignosulfonic acids (e.g. Borrespers-types, Borregaard), phenolsulfonic acids, naphthalenesulfonic acids (Morwet types, Akzo Nobel) and dibutylnaphthalenesulfonic acid (Nekal types, BASF AG), and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers, condensates of sulfonated naphthalene and its derivatives with formaldehyde, condensates of naphthalene or of the naphthalenesulfonic acids with phenol and formaldehyde, polyoxyethylene octylphenol ether, ethoxylated isooctyl-, octyl- or nonylphenol, alkylphenyl or tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors and proteins, denaturated proteins, polysaccharides (e.g. methylcellulose), hydrophobically modified starches, polyvinyl alcohol (Mowiol types Clariant), polycarboxylates (BASF AG, Sokalan types), polyalkoxylates, polyvinylamine (BASF AG, Lupamine types), polyethyleneimine (BASF AG, Lupasol types), polyvinylpyrrolidone and copolymers thereof.

Powders, materials for broadcasting and dusts can be prepared by mixing or grinding the active ingredients together with a solid carrier.

Granules, for example coated granules, impregnated granules and homogeneous granules, can be prepared by binding the active ingredients to solid carriers.

Aqueous use forms can be prepared from emulsion concentrates, suspensions, pastes, wettable powders or water-dispersible granules by adding water. To prepare emulsions, pastes or oil dispersions, the compounds of the formulae I or Ia, either as such or dissolved in an oil or solvent, can be homogenized in water by means of a wetting agent, tackifier, dispersant or emulsifier. Alternatively, it is also possible to prepare concentrates comprising active substance, wetting agent, tackifier, dispersant or emulsifier and, if desired, solvent or oil, which are suitable for dilution with water.

The concentrations of the compounds of the formula I in the ready-to-use preparations can be varied within wide ranges. In general, the formulations comprise from 0.001 to 98% by weight, preferably 0.01 to 95% by weight of at least one active ingredient. The active ingredients are employed in a purity of from 90% to 100%, preferably 95% to 100% (according to NMR spectrum).

The compounds I according to the invention can, for example, be formulated as follows:

1. Products for Dilution with Water

### A Water-Soluble Concentrates

10 parts by weight of active compound are dissolved in 90 parts by weight of water or a water-soluble solvent. As an alternative, wetters or other adjuvants are added. The active compound dissolves upon dilution with water. This gives a formulation with an active compound content of 10% by weight.

### B Dispersible Concentrates

20 parts by weight of active compound are dissolved in 70 parts by weight of cyclohexanone with addition of 10 parts by weight of a dispersant, for example polyvinylpyrrolidone. Dilution with water gives a dispersion. The active compound content is 20% by weight

### C Emulsifiable Concentrates

15 parts by weight of active compound are dissolved in 75 parts by weight of an organic solvent (eg. alkylaromatics) with addition of calcium dodecylbenzenesulfonate and castor oil ethoxylate (in each case 5 parts by weight). Dilution with water gives an emulsion. The formulation has an active compound content of 15% by weight.

### D Emulsions

25 parts by weight of active compound are dissolved in 35 parts by weight of an organic solvent (eg. alkylaromatics) with addition of calcium dodecylbenzenesulfonate and castor oil ethoxylate (in each case 5 parts by weight). This mixture is introduced into 30 parts by weight of water by means of an emulsifier (e.g. Ultraturrax) and made into a homogeneous emulsion. Dilution with water gives an emulsion. The formulation has an active compound content of 25% by weight.

### E Suspensions

In an agitated ball mill, 20 parts by weight of active compound are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules

50 parts by weight of active compound are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders

75 parts by weight of active compound are ground in a rotor-stator mill with addition of 25 parts by weight of dispersants, wetters and silica gel. Dilution with water gives a stable dispersion or solution of the active compound. The active compound content of the formulation is 75% by weight.

### H Gel Formulations

In a ball mill, 20 parts by weight of active compound, 10 parts by weight of dispersant, 1 part by weight of gelling agent and 70 parts by weight of water or of an organic solvent are ground to give a fine suspension. Dilution with water gives a stable suspension with active compound content of 20% by weight.

### 2. Products to be Applied Undiluted

### I Dusts

5 parts by weight of active compound are ground finely and mixed intimately with 95 parts by weight of finely divided kaolin. This gives a tracking powder with an active compound content of 5% by weight.

### J Granules (GR, FG, GG, MG)

0.5 parts by weight of active compound are ground finely and associated with 99.5 parts by weight of carriers. Current methods here are extrusion, spray-drying or the fluidized bed. This gives granules to be applied undiluted with an active compound content of 0.5% by weight.

### K ULV Solutions (UL)

10 parts by weight of active compound are dissolved in 90 parts by weight of an organic solvent, for example xylene. This gives a product to be applied undiluted with an active compound content of 10% by weight.

Specific formulations are given below:
I 20 parts by weight of the compound of the formula I are dissolved in a mixture composed of 80 parts by weight of alkylated benzene, 10 parts by weight of the adduct of from 8 to 10 mol of ethylene oxide to 1 mol of oleic acid N-monoethanolamide, 5 parts by weight of calcium salt of dodecylbenzenesulfonic acid and 5 parts by weight of the adduct of 40 mol of ethylene oxide to 1 mol of castor oil. Pouring the solution into 100 000 parts by weight of water and finely distributing it therein gives an aqueous dispersion which comprises 0.02% by weight of the active compound.
II 20 parts by weight of the compound of the formula I are dissolved in a mixture composed of 40 parts by weight of cyclohexanone, 30 parts by weight of isobutanol, 20 parts by weight of the adduct of 7 mol of ethylene oxide to 1 mol of isooctylphenol and 10 parts by weight of the adduct of 40 mol of ethylene oxide to 1 mol of castor oil. Pouring the solution into 100 000 parts by weight of water and finely distributing it therein gives an aqueous dispersion which comprises 0.02% by weight of the active compound.
III 20 parts by weight of the compound of the formula I are dissolved in a mixture composed of 25 parts by weight of cyclohexanone, 65 parts by weight of a mineral oil fraction of boiling point 210 to 280° C. and 10 parts by weight of the adduct of 40 mol of ethylene oxide to 1 mol of castor oil. Pouring the solution into 100 000 parts by weight of water and finely distributing it therein gives an aqueous dispersion which comprises 0.02% by weight of the active compound.
IV 20 parts by weight of the compound of the formula I are mixed thoroughly with 3 parts by weight of the sodium salt of diisobutylnaphthalenesulfonic acid, 17 parts by weight of the sodium salt of a lignosulfonic acid from a sulfite waste liquor and 60 parts by weight of pulverulent silica gel, and the mixture is ground in a hammer mill. Finely distributing the mixture in 20 000 parts by weight of water gives a spray mixture which comprises 0.1% by weight of the active compound.
V 3 parts by weight of the compound of the formula I are mixed with 97 parts by weight of finely divided kaolin. This gives a dust which comprises 3% by weight of the active compound.
VI 20 parts by weight of the compound of the formula I are mixed intimately with 2 parts by weight of calcium dodecylbenzenesulfonate, 8 parts by weight of fatty alcohol polyglycol ether, 2 parts by weight of the sodium salt of a phenol/urea/formaldehyde condensate and 68 parts by weight of a paraffinic mineral oil. This gives a stable oily dispersion.
VII 1 part by weight of the compound of the formula I is dissolved in a mixture composed of 70 parts by weight of cyclohexanone, 20 parts by weight of ethoxylated isooctylphenol and 10 parts by weight of ethoxylated castor oil. This gives a stable emulsion concentrate.
VIII 1 part by weight of the compound of the formula I is dissolved in a mixture composed of 80 parts by weight of cyclohexanone and 20 parts by weight of Wettol® EM 31 (=nonionic emulsifier based on ethoxylated castor oil). This gives a stable emulsion concentrate.

The compounds I or the herbicidal compositions comprising them can be applied pre- or post-emergence, or together with the seed of a crop plant. It is also possible to apply the herbicidal compositions or active compounds by applying seed, pretreated with the herbicidal compositions or active compounds, of a crop plant. If the active ingredients are less well tolerated by certain crop plants, application techniques may be used in which the herbicidal compositions are sprayed, with the aid of the spraying equipment, in such a way that as far as possible they do not come into contact with the leaves of the sensitive crop plants, while the active ingredients reach the leaves of undesirable plants growing underneath, or the bare soil surface (post-directed, lay-by).

In a further embodiment, the compounds of the formula I or the herbicidal compositions can be applied by treating seed.

The treatment of seeds comprises essentially all procedures familiar to the person skilled in the art (seed dressing, seed coating, seed dusting, seed soaking, seed film coating, seed multilayer coating, seed encrusting, seed dripping and seed pelleting) based on the compounds of the formula I according to the invention or the compositions prepared therefrom. Here, the herbicidal compositions can be applied diluted or undiluted.

The term seed comprises seed of all types, such as, for example, corns, seeds, fruits, tubers, seedlings and similar forms. Here, preferably, the term seed describes corns and seeds.

The seed used can be seed of the useful plants mentioned above, but also the seed of transgenic plants or plants obtained by customary breeding methods.

The rates of application of the active compound are from 0.001 to 3.0, preferably 0.01 to 1.0, kg/ha of active substance (a.s.), depending on the control target, the season, the target plants and the growth stage. To treat the seed, the compounds I are generally employed in amounts of from 0.001 to 10 kg per 100 kg of seed.

To widen the spectrum of action and to achieve synergistic effects, the compounds of the formula I may be mixed with a large number of representatives of other herbicidal or growth-regulating active ingredient groups and then applied concomitantly. Suitable components for mixture partners are, for example, 1,2,4-thiadiazoles, 1,3,4-thiadiazoles, amides, aminophosphoric acid and its derivatives, aminotriazoles, anilides, (het)aryloxyalkanoic acids and their derivatives, benzoic acid and its derivatives, benzothiadiazinones, 2-aroyl-1,3-cyclohexanediones, 2-hetaroyl-1,3-cyclohexanediones, hetaryl aryl ketones, benzylisoxazolidinones, meta-CF₃-phenyl derivatives, carbamates, quinolinecarboxylic acid and its derivatives, chloroacetanilides, cyclohexenone oxime ether derivatives, diazines, dichloropropionic acid and its derivatives, dihydrobenzofurans, dihydrofuran-3-ones, dinitroanilines, dinitrophenols, diphenyl ethers, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- and hetaryloxyphenoxypropionic esters, phenylacetic acid and its derivatives, phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides and uracils, phenylpyrazolines, isoxazolines and derivatives thereof.

It may furthermore be beneficial to apply the compounds I alone or in combination with other herbicides, or in the form of a mixture with other crop protection agents, for example together with agents for controlling pests or phytopathogenic fungi or bacteria. Also of interest is the miscibility with mineral salt solutions, which are employed for treating nutritional and trace element deficiencies. Further additives such as non-phytotoxic oils and oil concentrates may also be added.

The examples below serve to illustrate the invention.

### A PREPARATION EXAMPLES

The products were characterized by their retention time RT (in min) in an HPLC/MS (High Performance Liquid Chromatography combined with Mass Spectrometry), by NMR or by their melting point (m.p.).

HPLC column: RP-18 column (Chromolith Speed ROD from Merck KgaA, Germany)

Eluent: acetonitrile+0.1% trifluoroacetic acid (TFA)/water+0.1% TFA in a gradient from 5:95 to 95:5 in 5 minutes at 40° C.

MS: Quadrupol electrospray ionization, 80 V (positive mode)

### Example 1

### 3-Benzyl-1,4-dimethyl-6-(2-nitrobenzylidene)piperazine-2,5-dione

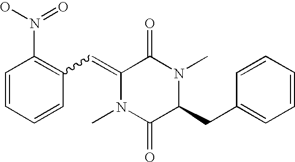

### 1.1 Ethyl 2-(tert-butoxycarbonylmethylamino)-3-(2-nitrophenyl)-3-trimethylsilanyloxy-propionate

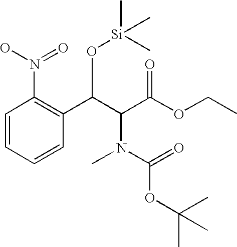 [0612] At −78° C., lithium diisopropylamide solution (2 M in tetrahydrofuran/n-heptane, 46 ml, 92 mmol) was slowly added dropwise to ethyl (tert-butoxycarbonylmethylamino)acetate (20 g, 92 mmol) in THF (abs., 50 ml). The mixture was stirred at this temperature for 3 h. 2-Nitrobenzaldehyde (13.6 g, 90 mmol) in THF (tetrahydrofuran, absolute, 30 ml) was then slowly added dropwise. The mixture was stirred at −78° C. for 1.5 h, and trimethylsilyl chloride (10 g, 92 mmol) was then added dropwise. The reaction solution was slowly (12 h) warmed to room temperature and then concentrated on a rotary evaporator. The residue was taken up in ethyl acetate, washed, dried and concentrated. The residue obtained in this manner was then purified by column chromatography (SiO₂, hexane/ethyl acetate). This gave 7.1 g (18%) of a non-polar isomer which were reacted further in the next step.

M+Na (m/z): 463.

### 1.2 Ethyl 3-hydroxy-2-methylamino-3-(2-nitrophenyl)propionate

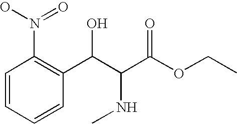 [0615] Trifluoroacetic acid (20 ml) was added to ethyl 2-(tert-butoxycarbonylmethylamino)-3-(2-nitrophenyl)-3-trimethylsilanyloxypropionate (8.6 g, 19.5 mmol) in CH₂Cl₂ (100 ml), and the mixture was stirred at room temperature for 12 h. The mixture was then neutralized with NaHCO₃ solution (saturated), the phases were separated and concentrated. The residue obtained in this manner was then purified by column chromatography (SiO₂, hexane/ethyl acetate). This gave 1.7 g (32%) of the target compound as a light-yellow solid.

M+1 (m/z): 269.

### 1.3 Ethyl 2-{[2-(tert-butoxycarbonylmethylamino)-3-phenylpropionyl]methylamino}-3-hydroxy-3-(2-nitrophenyl)propionate

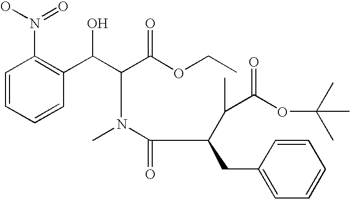 [0618] Ethyl 3-hydroxy-2-methylamino-3-(2-nitrophenyl)propionate (1.7 g, 6.3 mmol), 2-(tert-butoxycarbonylmethylamino)-3-phenylpropionic acid (2 g, 7 mmol), N-ethyldiisopropylamine (4.5 g, 35 mmol) and EDAC (3 g, 15.6 mmol) were stirred in THF (abs., 50 ml) for 3 days. The reaction solution was concentrated on a rotary evaporator. The residue was taken up in ethyl acetate and the resulting solution was washed, dried and concentrated. This gave 2.1 g (63%) of the target compound as a light-yellow oil.

M+1 (m/z): 530.

### 1.4 Ethyl 3-hydroxy-2-[methyl-(2-methylamino-3-phenylpropionyl)amino]-3-(2-nitro-phenyl)propionate

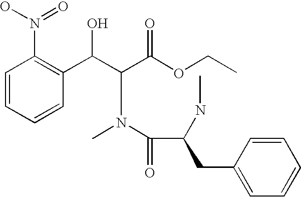 [0621] Trifluoroacetic acid (10 ml) was added to ethyl 2-{[2-(tert-butylcarbonylmethylamino)-3-phenylpropionyl]methylamino}-3-hydroxy-3-(2-nitrophenyl)propionate (2.1 g, 3.9 mmol) in CH₂Cl₂ (20 ml), and the mixture was stirred at room temperature for 2 h and then concentrated on a rotary evaporator. The residue obtained in this manner was reacted as crude product in the next step.

### 1.5 3-Benzyl-6-[hydroxy-(2-nitrophenyl)methyl]-1,4-dimethylpiperazine-2,5-dione

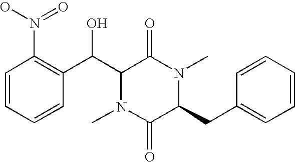 [0623] The residue obtained in 1.4 is taken up in THF (50 ml), and NH₄OH (25% in H₂O, 10 ml) is added. The mixture is stirred at room temperature for 12 h. After addition of H₂O (100 ml), the mixture is extracted with methyl tert-butyl ether and the organic phase is dried and concentrated. The residue obtained in this manner is purified by column chromatography (SiO₂, hexane/ethyl acetate). This gives 0.57 g (38%) of a polar isomer which is reacted further in the next step

M+1 (m/z): 384.

### 1.6 (5-Benzyl-1,4-dimethyl-3,6-dioxopiperazin-2-yl)-(2-nitrophenyl)methyl methanesulfonate

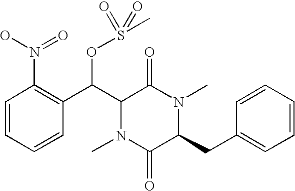 [0626] DMAP (1.8 g, 14.7 mmol) and methanesulfonyl chloride (30 ml) were added to 3-benzyl-6-[hydroxy-(2-nitrophenyl)methyl]-1,4-dimethylpiperazine-2,5-dione (5.5 g, 14.3 mmol) in pyridine (100 ml), and the mixture was stirred at room temperature for 12 h and then concentrated on a rotary evaporator. After addition of H₂O and CH₂Cl₂, the insoluble black resins were filtered off using a nutsch filter, the phases were separated and the organic phase was concentrated. The residue obtained in this manner was purified by column chromatography (SiO₂, hexane/ethyl acetate). This gave 5.1 g (77%) of the target compound as a light-yellow foam.

M+1 (m/z): 462.

### 1.7 3-Benzyl-1,4-dimethyl-6-(2-nitrobenzylidene)piperazine-2,5-dione

At 0° C., DBU (1.4 g, 9 mmol) was slowly added dropwise to (5-benzyl-1,4-dimethyl-3,6-dioxopiperazin-2-yl)-(2-nitrophenyl)methyl methanesulfonate (4.25 g, 9 mmol) in THF (100 ml), and the mixture was stirred at 0° C. for 4 h. At this temperature, the pH was then adjusted to 7 using citric acid (10%) and the mixture was then allowed to warm slowly to room temperature. After addition of H₂O and ethyl acetate, then phases were separated and the organic phase was concentrated. The residue obtained in this manner was purified by column chromatography (SiO₂, methyl tert-butyl ether/ethyl acetate). This gave 2.5 g (76%) of the target compound as a yellow foam.

The Z:E isomer mixture obtained in this manner was separated by preparative MPLC (silica gel: Merck Lichroprep RP-18 (40-63 μm), MeOH:H₂O=60:40). ¹H-NMR (CDCl₃) of the separated isomers:

a) δ=2.62 (s, 3H), 3.09 (s, 3H), 3.23 (m, 2H), 4.39 (m, 1H), 6.39 (d, 1H), 7.13 (s, 1H), 7.17 (m, 1H), 7.24 (m, 1H), 7.32 (m, 2H), 7.44 (m, 1H), 7.49 (m, 1H), 8.05 (d, 2H).

b) δ=2.91 (s, 3H), 3.15 (dm 1H), 3.33 (s, 3H), 3.29 (dm, 1H), 4.32 (m, 1H), 6.28 (s, 1H), 6.75 (m, 1H), 7.08 (m, 2H), 7.32 (m, 3H), 7.39 (m, 1H), 7.47 (m, 1H), 8.04 (d, 1H).

### Example 105/106

### 3-Benzyl-1,4-dimethyl-6-(2-thiazol-2-ylbenzylidene)piperazine-2,5-dione

In a reaction vessel, 600 mg of 3-benzyl-1,4-dimethyl-6-(2-iodobenzylidene)piperazine-2,5-dione (Example 4, prepared analogously to Example 1), 250 mg of bis(dibenzylideneacetone)palladium and 500 mg of triphenylarsan were initially charged in 30 ml of dioxane under an atmosphere of argon. 1 g of 2-(tributylstannyl)thiazole was added, and the mixture was heated at 60° C. for 16 h. The reaction mixture was concentrated under reduced pressure and the residue was chromatographed on silica gel using hexane/methyl tert-butyl ether (2:1 v/v).

In this manner, a total of 404 mg of the title compound were obtained as a wax-like solid.

### Example 115

### 3-Benzyl-1,4-dimethyl-6-(2-cyano-5,6-difluorobenzylidene)piperazine-2,5-dione

In a reaction vessel, 2.0 g of 3-benzyl-1,4-dimethyl-6-(2-bromo-5,6-difluorobenzylidene)piperazine-2,5-dione (Example 119, prepared analogously to Example 1) were reacted with 1.7 g of copper(1) cyanide in 50 ml of N-methylpyrrolidone under an atmosphere of argon at 155° C. for 18 h. The reaction mixture was concentrated under reduced pressure and the residue was taken up in ethyl acetate, and the resulting solution was washed 3 times with water, dried and again concentrated under reduced pressure. The residue was chromatographed on silica gel using hexane/ethyl acetate (1:1 v/v). This gave 331 mg of the Z isomer as a light-yellow solid of melting point 175° C. and 310 mg of the E isomer as a beige solid of melting point 205° C.

The compounds listed in Tables 2, 3 and 4 below and the compounds listed in Table 5 below were prepared in an analogous manner (Examples 2 to 214).

TABLE 2

| | | | | | | I |
|---|---|---|---|---|---|---|
| 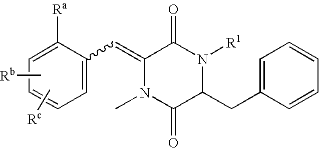 | | | | | | |
| Example | | | | | RT HPLC/MS | |
| No. | R^{a} | R^{b} | R^{c} | R¹ | or m.p. | Isomer |
| 1 | 2-NO₂ | H | H | CH₃ | 2.831 min | |
| | | | | | m/z = 366.0 | |
| | | | | | [M + H]⁺ | |
| 2 | 2-NO₂ | H | H | H | 2.724 min | isomer |
| | | | | | m/z = 352.4 | 1 |
| | | | | | [M + H]⁺ | |
| 3 | 2-NO₂ | H | H | H | 2.773 min | isomer |
| | | | | | m/z = 352.4 | 2 |
| | | | | | [M + H]⁺ | |
| 4 | 2-I | H | H | CH₃ | 3.286 min | |
| | | | | | m/z = 446.9 | |
| | | | | | [M + H]⁺ | |
| 5 | 2-(CH₃)₃Si—C≡C— | H | H | CH₃ | 4.039 min | isomer |
| | | | | | m/z = 417.5 | 1 |
| | | | | | [M + H]⁺ | |
| 6 | 2-(CH₃)₃Si—C≡C— | H | H | CH₃ | 4.064 min | isomer |
| | | | | | m/z = 417.5 | 2 |
| | | | | | [M + H]⁺ | |
| 7 | 2-HC≡C— | H | H | CH₃ | 3.053 min | |
| | | | | | m/z = 345.0 | |
| | | | | | [M + H]⁺ | |
| 8 | 2-NO₂ | 5-Cl | H | CH₃ | 3.128 min | isomer |
| | | | | | m/z = 399.9 | 1 |
| | | | | | [M + H]⁺ | |
| 9 | 2-NO₂ | 5-Cl | H | CH₃ | 3.215 min | isomer |
| | | | | | m/z = 399.9 | 2 |
| | | | | | [M + H]⁺ | |
| 10 | 2-NO₂ | 4-OCH₃ | 5-OCH₃ | CH₃ | 2.801 min | isomer |
| | | | | | m/z = 426.0 | 1 |
| | | | | | [M + H]⁺ | |
| 11 | 2-NO₂ | 4-OCH₃ | 5-OCH₃ | CH₃ | 2.828 min | isomer |
| | | | | | m/z = 426.0 | 2 |
| | | | | | [M + H]⁺ | |
| 12 | 2-NO₂ | 6-CH₃ | H | CH₃ | 3.088 min | isomer |
| | | | | | m/z = 380.0 | 1 |
| | | | | | [M + H]⁺ | |
| 13 | 2-NO₂ | 6-CH₃ | H | CH₃ | 3.091 min | isomer |
| | | | | | m/z = 380.0 | 2 |
| | | | | | [M + H]⁺ | |
| 14 | 2-CN | H | H | CH₃ | 2.721 min | |
| | | | | | m/z = 346.1 | |
| | | | | | [M + H]⁺ | |
| | | | | | 143° C. | |
| 15 | 2-NO₂ | 4-N(CH₃)₂ | H | CH₃ | 3.038 min | |
| | | | | | m/z = 409.0 | |
| | | | | | [M + H]⁺ | |
| 16 | 2-NO₂ | 4,5-(—O—CH₂—O—) | | CH₃ | 2.778 min | isomer |
| | | | | | m/z = 410.0 | 1 |
| | | | | | [M + H]⁺ | |
| 17 | 2-NO₂ | 4,5-(—O—CH₂—O—) | | CH₃ | 2.856 min | isomer |
| | | | | | m/z = 410.0 | 2 |
| | | | | | [M + H]⁺ | |
| 18 | 2-NO₂ | 6-Cl | H | CH₃ | 3.105 min | |
| | | | | | m/z = 400.1 | |
| | | | | | [M + H]⁺ | |
| 19 | 2-NO₂ | 5-F | H | CH₃ | 2.973 min | isomer |
| | | | | | m/z = 384.4 | 1 |
| | | | | | [M + H]⁺ | |
| 20 | 2-NO₂ | 5-F | H | CH₃ | 3.037 min | isomer |
| | | | | | m/z = 384.4 | 2 |
| | | | | | [M + H]⁺ | |
| 21 | 2-NO₂ | 3-OCH₃ | H | CH₃ | 2.926 min | |
| | | | | | m/z = 396.1 | |
| | | | | | [M + H]⁺ | |
| 22 | 2-NO₂ | 5-OCH₃ | 6- | CH₃ | 3.327 min | |
| | | | O—SO₂- | | m/z = 552.1 | |
| | | | Ph | | [M + H]⁺ | |
| 23 | 2-NH₂ | H | H | CH₃ | 2.249 min | isomer |
| | | | | | m/z = 336.4 | 1 |
| | | | | | [M + H]⁺ | |
| 24 | 2-NH₂ | H | H | CH₃ | 2.079 min | isomer |
| | | | | | m/z = 336.1 | 2 |
| | | | | | [M + H]⁺ | |
| 25 | 2-NH—C(O)—CH₃ | H | H | CH₃ | 2.372 min | |
| | | | | | m/z = 378.1 | |
| | | | | | [M + H]⁺ | |
| 26 | 2-NH—C(O)-Ph | H | H | CH₃ | 2.837 min | |
| | | | | | m/z = 440.2 | |
| | | | | | [M + H]⁺ | |
| 27 | 2-NH—C(O)—NH—SO₂-Ph | H | H | CH₃ | 3.095 min | |
| | | | | | m/z = 533.2 | |
| | | | | | [M + H]⁺ | |
| 28 | 2-NH—SO₂—NHCH₃ | H | H | CH₃ | 2.505 min | |
| | | | | | m/z = 429.1 | |
| | | | | | [M + H]⁺ | |
| 29 | 2-NH—C(O)—NH-Ph | H | H | CH₃ | 3.140 min | |
| | | | | | m/z = 455.2 | |
| | | | | | [M + H]⁺ | |
| 30 | 2-NH—C(O)—OCH₃ | H | H | CH₃ | 2.667 min | isomer |
| | | | | | m/z = 394.4 | 1 |
| | | | | | [M + H]⁺ | |
| 31 | 2-NH—C(O)—OCH₃ | H | H | CH₃ | 2.590 min | isomer |
| | | | | | m/z = 394.2 | 2 |
| | | | | | [M + H]⁺ | |
| 32 | 2-NH—SO₂—NHCH₂CH₃ | H | H | CH₃ | 2.642 min | |
| | | | | | m/z = 443.1 | |
| | | | | | [M + H]⁺ | |
| 33 | 2-NH—SO₂—NHCH(CH₃)₂ | H | H | CH₃ | 2.848 min | |
| | | | | | m/z = 457.1 | |
| | | | | | [M + H]⁺ | |
| 34 | 2-NH—CN | H | H | CH₃ | 2.579 min | isomer |
| | | | | | m/z = 361.1 | 1 |
| | | | | | [M + H]⁺ | |
| 35 | 2-NH—CN | H | H | CH₃ | 2.637 min | isomer |
| | | | | | m/z = 361.2 | 2 |
| | | | | | [M + H]⁺ | |
| 36 | 2-F | H | H | CH₃ | 3.009 min | isomer |
| | | | | | m/z = 339.4 | 1 |
| | | | | | [M + H]⁺ | |
| 37 | 2-F | H | H | CH₃ | 2.762 min | isomer |
| | | | | | m/z = 339.4 | 2 |
| | | | | | [M + H]⁺ | |
| 38 | 2-I | 6-F | H | CH₃ | 3.259 min | |
| | | | | | m/z = 465.0 | |
| | | | | | [M + H]⁺ | |
| 39 | 2-CN | 6-F | H | CH₃ | 3.033 min | |
| | | | | | m/z = 364.1 | |
| | | | | | [M + H]⁺ | |
| | | | | | 138° C. | |
| 40 | 2-I | 6-Cl | H | CH₃ | 3.625 min | |
| | | | | | m/z = 481.0 | |
| | | | | | [M + H]⁺ | |
| 41 | 2-Br | 6-Cl | H | CH₃ | 3.437 min | |
| | | | | | m/z = 434.9 | |
| | | | | | [M + H]⁺ | |
| 42 | 2-CN | H | H | H | 2.656 min | |
| | | | | | m/z = 332.1 | |
| | | | | | [M + H]⁺ | |
| | | | | | 158° C. | |
| 43 | 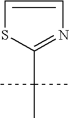 | H | H | CH₃ | 2.936 min m/z = 404.1 [M + H]⁺ | isomer 1 |
| 44 | 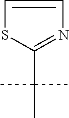 | H | H | CH₃ | 2.806 min m/z = 404.1 [M + H]⁺ | isomer 2 |
| 45 | 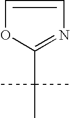 | H | H | CH₃ | 2.878 min m/z = 388.1 [M + H]⁺ | isomer 1 |
| 46 | 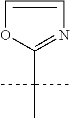 | H | H | CH₃ | 2.867 min m/z = 388.1 [M + H]⁺ 153° C. | isomer 2 |
| 47 | 2-(furan-2-yl) | H | H | CH₃ | 3.173 min | isomer |
| | | | | | m/z = 387.1 | 1 |
| | | | | | [M + H]⁺ | |
| 48 | 2-(furan-2-yl) | H | H | CH₃ | 3.284 min | isomer |
| | | | | | m/z = 387.1 | 2 |
| | | | | | [M + H]⁺ | |
| 49 | 2-(thiophen-2-yl) | H | H | CH₃ | 3.378 min | |
| | | | | | m/z = 403.1 | |
| | | | | | [M + H]⁺ | |
| 50 | 2-(pyridin-2-yl) | H | H | CH₃ | 2.131 min | |
| | | | | | m/z = 398.2 | |
| | | | | | [M + H]⁺ | |
| 51 | 2-(pyridin-3-yl) | H | H | CH₃ | 2.214 min | |
| | | | | | m/z = 398.2 | |
| | | | | | [M + H]⁺ | |
| 52 | 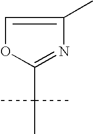 | H | H | CH₃ | 2.963 min m/z = 402.2 [M + H]⁺ | isomer 1 |
| 53 | 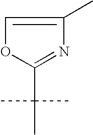 | H | H | CH₃ | 2.989 min m/z = 402.2 [M + H]⁺ 128° C. | isomer 2 |
| 54 | 2-CF₃ | H | H | CH₃ | 3.164 min m/z = 389.1 [M + H]⁺ | |
| 55 | 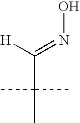 | H | H | CH₃ | 2.601 min m/z = 387.4 [M + Na]⁺ | |
| 56 | 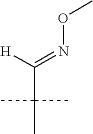 | H | H | CH₃ | 2.985 min m/z = 378.2 [M + H]⁺ | |
| 57 | 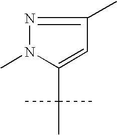 | H | H | CH₃ | 2.803 min m/z = 415.5 [M + H]⁺ | |
| 58 | 2-Br | 5-CF₃ | H | CH₃ | 3.435 min | isomer |
| | | | | | m/z = 469.0 | 1 |
| | | | | | [M + H]⁺ | |
| 59 | 2-Br | 5-CF₃ | H | CH₃ | 3.490 min | isomer |
| | | | | | m/z = 469.0 | 2 |
| | | | | | [M + H]⁺ | |
| | | | | | 113° C. | |
| 60 | 2-Br | 4-OCH₃ | 5-OCH₃ | CH₃ | 3.074 min | isomer |
| | | | | | m/z = 461.0 | 1 |
| | | | | | [M + H]⁺ | |
| | | | | | 121° C. | |
| 61 | 2-Br | 4-OCH₃ | 5-OCH₃ | CH₃ | 3.131 min | isomer |
| | | | | | m/z = 461.1 | 2 |
| | | | | | [M + H]⁺ | |
| 62 | 2-Br | 5-F | H | CH₃ | 3.380 min | |
| | | | | | m/z = 417.0 | |
| | | | | | [M + H]⁺ | |
| | | | | | 106° C. | |
| 63 | 2-CN | 5-CF₃ | H | CH₃ | 3.338 min | isomer |
| | | | | | m/z = 414.1 | 1 |
| | | | | | [M + H]⁺ | |
| 64 | 2-CN | 5-CF₃ | H | CH₃ | 3.376 min | isomer |
| | | | | | m/z = 414.1 | 2 |
| | | | | | [M + H]⁺ | |
| 65 | 2-CN | 4-OCH₃ | 5-OCH₃ | CH₃ | 2.805 min | isomer |
| | | | | | m/z = 406.1 | 1 |
| | | | | | [M + H]⁺ | |
| | | | | | 163° C. | |
| 66 | 2-CN | 4-OCH₃ | 5-OCH₃ | CH₃ | 2.764 min | isomer |
| | | | | | m/z = 406.1 | 2 |
| | | | | | [M + H]⁺ | |
| 67 | 2-CN | 5-F | H | CH₃ | 2.939 min | isomer |
| | | | | | m/z = 364.4 | 1 |
| | | | | | [M + H]⁺ | |
| 68 | 2-CN | 5-F | H | CH₃ | 2.950 min | isomer |
| | | | | | m/z = 364.1 | 2 |
| | | | | | [M + H]⁺ | |
| | | | | | 128° C. | |
| 69 | 2-COOH | H | H | CH₃ | 2.539 min | |
| | | | | | m/z = 365.1 | |
| | | | | | [M + H]⁺ | |
| 70 | 2- | H | H | CH₃ | 2.599 min | |
| | C(O)NHCH₂CH₂CH₃ | | | | m/z = 406.2 | |
| | | | | | [M + H]⁺ | |
| 71 | 2-C(O)OCH(CH₃)₂ | H | H | CH₃ | 3.271 min | |
| | | | | | m/z = 407.2 | |
| | | | | | [M + H]⁺ | |
| 72 | 2-C(O)NH—CH₂CH═CH₂ | H | H | CH₃ | 2.537 min | |
| | | | | | m/z = 404.1 | |
| | | | | | [M + H]⁺ | |
| 73 | 2-C(O)OCH₃ | H | H | CH₃ | 2.878 min | |
| | | | | | m/z = 379.1 | |
| | | | | | [M + H]⁺ | |
| 74 | 2-COOCH₂CF₃ | H | H | CH₃ | 3.291 min | |
| | | | | | m/z = 447.1 | |
| | | | | | [M + H]⁺ | |
| 75 | 2-C(O)N(CH₂CH₃)₂ | H | H | CH₃ | 2.752 min | |
| | | | | | m/z = 420.2 | |
| | | | | | [M + H]⁺ | |
| 76 | 2-C(O)OCH₂-(o-F- | H | H | CH₃ | 3.516 min | |
| | Ph) | | | | m/z = 473.2 | |
| | | | | | [M + H]⁺ | |
| 77 | 2-C(O)NHCH₂-(o, | H | H | CH₃ | 2.937 min | |
| | o′-difluoro-Ph) | | | | m/z = 490.2 | |
| | | | | | [M + H]⁺ | |
| 78 | 2-C(O)NHOCH₃ | H | H | CH₃ | 2.305 min | |
| | | | | | m/z = 394.1 | |
| | | | | | [M + H]⁺ | |
| 79 | 2-C(O)NH—SO₂—N(CH₃)(i-Pr) | H | H | CH₃ | 2.796 min | |
| | | | | | m/z = 499.2 | |
| | | | | | [M + H]⁺ | |
| 80 | 2-C(O)O-(o-NO₂- | H | H | CH₃ | 3.335 min | |
| | Ph) | | | | m/z = 486.2 | |
| | | | | | [M + H]⁺ | |
| 81 | 2-C(O)NH—SO₂—N(CH₃)(i-Pr) | H | H | CH₃ | 2.796 min | |
| | | | | | m/z = 499.2 | |
| | | | | | [M + H]⁺ | |
| 82 | 2-C(O)NH-(m-Br- | H | H | CH₃ | 3.286 min | |
| | Ph) | | | | m/z = 520.0 | |
| | | | | | [M + H]⁺ | |
| 83 | 2-C(O)NH₂ | H | H | CH₃ | 2.220 min | |
| | | | | | m/z = 364.1 | |
| | | | | | [M + H]⁺ | |
| 84 | 2-C(O)NH—CH(CH₃)(CH₂—O—CH₃) | H | H | CH₃ | 2.520 min | |
| | | | | | m/z = 436.2 | |
| | | | | | [M + H]⁺ | |
| 85 | 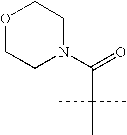 | H | H | CH₃ | 2.416 min m/z = 434.2 [M + H]⁺ | |
| 86 | 2-C(O)O—CH₂C≡CH | H | H | CH₃ | 3.003 min | |
| | | | | | m/z = 403.1 | |
| | | | | | [M + H]⁺ | |
| 87 | 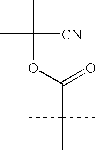 | H | H | CH₃ | 3.084 min m/z = 432.1 [M + H]⁺ | |
| 88 | 2-Br | 4,5-(—O—CH₂—O—) | | CH₃ | 3.115 min | |
| | | | | | m/z = | |
| | | | | | 445.0 | |
| | | | | | [M + H]⁺ | |
| 89 | 2-Br | 4-F | H | CH₃ | 3.189 min | isomer |
| | | | | | m/z = | 1 |
| | | | | | 417.0 | |
| | | | | | [M + H]⁺ | |
| 90 | 2-Br | 4-F | H | CH₃ | 3.197 min | isomer |
| | | | | | m/z = | 2 |
| | | | | | 417.0 | |
| | | | | | [M + H]⁺ | |
| 91 | 2-CN | 4,5-(—O—CH₂—O—) | | CH₃ | 2.711 min | |
| | | | | | m/z = | |
| | | | | | 412.1 | |
| | | | | | [M + Na]⁺ | |
| 92 | 2-CN | 4-F | H | CH₃ | 2.848 min | |
| | | | | | m/z = | |
| | | | | | 386.1 | |
| | | | | | [M + Na]⁺ | |
| 93 | 2-Br | H | H | CH₃ | 3.086 min | |
| | | | | | m/z = | |
| | | | | | 399.0 | |
| | | | | | [M + H]⁺ | |
| 94 | 2-CN | 4-CH₃ | H | CH₃ | 2.940 min | |
| | | | | | m/z = | |
| | | | | | 360.1 | |
| | | | | | [M + H]⁺ | |
| 95 | 2-C(S)NH₂ | H | H | CH₃ | 2.634 min | |
| | | | | | m/z = 380.1 | |
| | | | | | [M + H]⁺ | |
| 96 | 2-CN | H | H | CH₃ | 2.816 min | |
| | | | | | m/z = | |
| | | | | | 346.4 | |
| | | | | | [M + H]⁺ | |
| | | | | | 209° C. | |
| 97 | 2-OH | 5-NO₂ | H | CH₃ | 2.709 min | |
| | | | | | m/z = | |
| | | | | | 382.1 | |
| | | | | | [M + H]⁺ | |
| 98 | 2-CN | 5-NO₂ | H | CH₃ | 3.029 min | isomer |
| | | | | | m/z = | 1 |
| | | | | | 391.1 | |
| | | | | | [M + H]⁺ | |
| 99 | 2-CN | 5-NO₂ | H | CH₃ | 2.982 min | isomer |
| | | | | | m/z = | 2 |
| | | | | | 391.1 | |
| | | | | | [M + H]⁺ | |
| 100 | 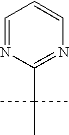 | H | H | CH₃ | 2.660 min m/z = 399.5 [M + H]⁺ | isomer 1 |
| 101 | 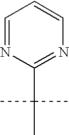 | H | H | CH₃ | 2.736 min m/z = 399.5 [M + H]⁺ | isomer 2 |
| 102 | 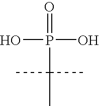 | H | H | CH₃ | 2.089 min m/z = 401.4 [M + H]⁺ 105° C. | |
| 103 | 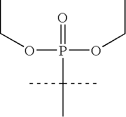 | H | H | CH₃ | 2.780 min m/z = 457.5 [M + H]⁺ 115° C. | isomer 1 |
| 104 | 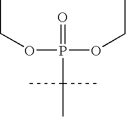 | H | H | CH₃ | 2.845 min m/z = 457.5 [M + H]⁺ | isomer 2 |
| 105 | 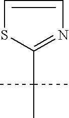 | H | H | CH₃ | 3.074 min m/z = 404.1 [M + H]⁺ | isomer 1 |
| 106 | 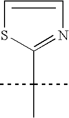 | H | H | CH₃ | 2.886 min m/z = 404.1 [M + H]⁺ | isomer 2 |
| 107 | 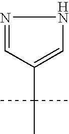 | H | H | CH₃ | 2.563 min m/z = 387.5 [M + H]⁺ | isomer 1 |
| 108 | 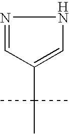 | H | H | CH₃ | 2.700 min m/z = 387.5 [M + H]⁺ | isomer 2 |
| 109 | 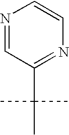 | H | H | CH₃ | 2.680 min m/z = 399.1 [M + H]⁺ | isomer 1 |
| 110 | 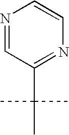 | H | H | CH₃ | 2.785 min m/z = 399.1 [M + H]⁺ | isomer 2 |
| 111 | 2-NO₂ | 6-Br | H | CH₃ | 3.289 min | isomer |
| | | | | | m/z = 444.0 | 1 |
| | | | | | [M + H]⁺ | |
| | | | | | 115° C. | |
| 112 | 2-NO₂ | 6-Br | H | CH₃ | 3.333 min | isomer |
| | | | | | m/z = 444.0 | 2 |
| | | | | | [M + H]⁺ | |
| | | | | | 130° C. | |
| 113 | 2-CN | 6-NH₂ | H | CH₃ | 2.839 min | |
| | | | | | m/z = | |
| | | | | | 361.1 | |
| | | | | | [M + H]⁺ | |
| | | | | | 193° C. | |
| 114 | 2-CN | 5-F | 6-OH | CH₃ | 2.716 min | |
| | | | | | m/z = | |
| | | | | | 380.1 | |
| | | | | | [M + H]⁺ | |
| | | | | | 205° C. | |
| 115 | 2-CN | 5-F | 6-F | CH₃ | 3.153 min | |
| | | | | | m/z = | |
| | | | | | 382.1 | |
| | | | | | [M + H]⁺ | |
| | | | | | 175° C. | |
| 116 | 2-CN | 6-CN | H | CH₃ | 2.903 min | isomer |
| | | | | | m/z = | 1 |
| | | | | | 371.4 | |
| | | | | | [M + H]⁺ | |
| | | | | | 225° C. | |
| 117 | 2-CN | 6-CN | H | CH₃ | 2.790 min | isomer |
| | | | | | m/z = | 2 |
| | | | | | 371.4 | |
| | | | | | [M + H]⁺ | |
| | | | | | 235° C. | |
| 118 | 2-CN | 6-Br | H | CH₃ | 3.122 min | |
| | | | | | m/z = | |
| | | | | | 424.4 | |
| | | | | | [M]⁺ | |
| | | | | | 175-180° C. | |
| 119 | 2-Br | 5-F | 6-F | CH₃ | 3.488 min | |
| | | | | | m/z = | |
| | | | | | 435.0 | |
| | | | | | [M]⁺ | |
| 120 | 2-CN | 6-CH═CH₂ | H | CH₃ | 3.143 min | isomer |
| | | | | | m/z = | 1 |
| | | | | | 372.1 | |
| | | | | | [M + H]⁺ | |
| 121 | 2-CN | 6-CH═CH₂ | H | CH₃ | 3.261 min | isomer |
| | | | | | m/z = | 2 |
| | | | | | 372.0 | |
| | | | | | [M + H]⁺ | |
| 122 | 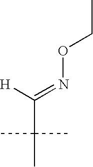 | H | H | CH₃ | 3.343 min m/z = 392.5 [M + H]⁺ | |
| 123 | 2-NHC(O)CH₃ | 6-Br | H | CH₃ | 3.035 min | |
| | | | | | m/z = 456.2 | |
| | | | | | [M + H]⁺ | |
| 124 | 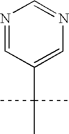 | H | H | CH₃ | 2.654 min m/z = 399.1 [M + H]⁺ | |
| 125 | 2-CN | 6-NHC(O)CH₃ | H | CH₃ | 2.676 min | |
| | | | | | m/z = 403.5 | |
| | | | | | [M + H]⁺ | |
| 126 | 2-CN | 6-NHC(O)NH(Ph) | H | CH₃ | 3.514 min | |
| | | | | | m/z = 480.5 | |
| | | | | | [M + H]⁺ | |
| 127 | 2-CN | 6-N(CH₃)₂ | H | CH₃ | 3.171 min | |
| | | | | | m/z = 389.5 | |
| | | | | | [M + H]⁺ | |
| | | | | | 58° C. | |
| 128 | 2-NH—SO₂—NHCH(CH₃)₂ | H | H | CH₃ | 3.129 min | |
| | | | | | m/z = 482.0 | |
| | | | | | [M + H]⁺ | |
| | | | | | 103° C. | |
| 129 | 2-CH═CH₂ | H | H | CH₃ | 3.259 min | isomer |
| | | | | | m/z = 347.1 | 1 |
| | | | | | [M + H]⁺ | |
| 130 | 2-CH═CH₂ | H | H | CH₃ | 3.262 min | isomer |
| | | | | | m/z = 347.1 | 2 |
| | | | | | [M + H]⁺ | |
| 131 | 2-CN | 4-Cl | 5-Cl | CH₃ | 3.421 min | |
| | | | | | m/z = 413.0 | |
| | | | | | [M]⁺ | |
| 132 | 2-Br | 5-Cl | 6-Cl | CH₃ | 3.744 min | |
| | | | | | m/z = 468.8 | |
| | | | | | [M]⁺ | |
| 133 | 2-CN | 5-Cl | 6-Cl | CH₃ | 3.430 min | isomer |
| | | | | | m/z = 413.9 | 1 |
| | | | | | [M]⁺ | |
| 134 | 2-CN | 5-Cl | 6-Cl | CH₃ | 3.408 min | isomer |
| | | | | | m/z = 414.0 | 2 |
| | | | | | [M]⁺ | |
| 135 | 2-Phenyl | H | H | CH₃ | 3.534 min | |
| | | | | | m/z = 397.5 | |
| | | | | | [M + H]⁺ | |
| 136 | 2-CN | H | H | CH₂—CH═CH₂ | 3.198 | |
| | | | | | m/z = 372.5 | |
| | | | | | [M + H]⁺ | |
| 137 | 2-CN | H | H | C(O)—(C₆H₅) | 3.648 | |
| | | | | | m/z = 436.5 | |
| | | | | | [M + H]⁺ | |
| 138 | 2-CN | H | H | CH₂—OH | 2.524 | |
| | | | | | m/z = 362.4 | |
| | | | | | [M + H]⁺ | |
| 139 | 2-CN | H | H | CH₂—C(O)—OCH₃ | 3.233 | |
| | | | | | m/z = 403.7 | |
| | | | | | [M + H]⁺ | |
| 140 | 2-CN | H | H | C(O)—CH₃ | 3.322 | |
| | | | | | m/z = 374.0 | |
| | | | | | [M + H]⁺ | |
| 141 | 2-SO₂CH₃ | H | H | CH₃ | m/z = 399.4 | isomer |
| | | | | | [M + H]⁺ | 1 |
| 142 | 2-SO₂CH₃ | H | H | CH₃ | m/z = 399.2 | isomer |
| | | | | | [M + H]⁺ | 2 |
| 143 | 2-NO₂ | 6-CH═CH₂ | H | CH₃ | m/z = 392.3 | isomer |
| | | | | | [M + H]⁺ | 1 |
| 144 | 2-NO₂ | 6-CH═CH₂ | H | CH₃ | m/z = 392.3 | isomer |
| | | | | | [M + H]⁺ | 2 |
| 145 | 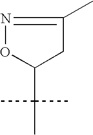 | H | H | CH₃ | m/z = 404.4 [M + H]⁺ | |
| 146 | 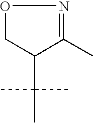 | H | H | CH₃ | m/z = 404.4 [M + H]⁺ | |
| 147 | 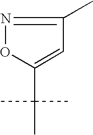 | H | H | CH₃ | m/z = 402.4 [M + H]⁺ 98° C. | isomer 1 |
| 148 | 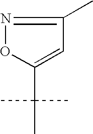 | H | H | CH₃ | m/z = 402.4 [M + H]⁺ 112° C. | isomer 2 |
| 149 | 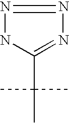 | H | H | CH₃ | m/z = 389.4 [M + H]⁺ | isomer 1 |
| 150 | 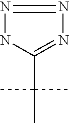 | H | H | CH₃ | m/z = 389.4 [M + H]⁺ | isomer 2 |
| 151 | 2-CN | 6-CF₃ | H | CH₃ | 3.212 | isomer |
| | | | | | m/z = 414.5 | 1 |
| | | | | | [M]⁺ | |
| 152 | 2-CN | 6-CF₃ | H | CH₃ | 3.243 | isomer |
| | | | | | m/z = 414.5 | 2 |
| | | | | | [M]⁺ | |
| 153 | 2-Br | 6-CF₃ | H | CH₃ | 3.522 | |
| | | | | | m/z = 469.4 | |
| | | | | | [M + H]⁺ | |
| 154 | 2-CN | 5-Cl | 6-Cl | H | 3.131 min | |
| | | | | | m/z = 422.0 | |
| | | | | | [M + Na]⁺ | |
| 155 | 2-Br | 5-Cl | 6-Cl | C(O)—CH₃ | 4.146 min | |
| | | | | | m/z = 518.8 | |
| | | | | | [M + Na]⁺ | |
| 156 | 2-Br | 5-Cl | 6-Cl | H | 3.484 min | |
| | | | | | m/z = 454.8 | |
| | | | | | [M + H]⁺ | |
| 157 | 2-CN | 6-Cl | H | CH₃ | 3.070 | isomer |
| | | | | | m/z = 380.4 | 1 |
| | | | | | [M + H]⁺ | |
| 158 | 2-CN | 6-Cl | H | CH₃ | 3.117 | isomer |
| | | | | | m/z = 380.4 | 2 |
| | | | | | [M + H]⁺ | |

Example No. Example Number
m.p. melting point
Ph phenyl
i-Pr isopropyl

TABLE 3

| | |
|---|---|
| | (I) |
| 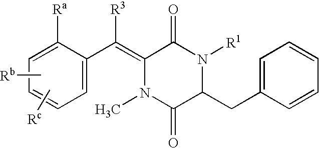 | |

| Ex- | | | | | | | |
|---|---|---|---|---|---|---|---|
| ample | | | | | | RT HPLC/MS | |
| No. | R^{a} | R^{b} | R^{c} | R³ | R¹ | or m.p. | Isomer |
| 159 | 2-NO₂ | H | H | Br | CH₃ | 3.306 min | |
| | | | | | | m/z = 446.0 | |
| | | | | | | [M + H]⁺ | |
| 160 | 2-CN | H | H | Br | CH₃ | 3.113 min | |
| | | | | | | m/z = 424.4 | |
| | | | | | | [M]⁺ | |
| 161 | 2-NO₂ | H | H | CH═CH₂ | CH₃ | 3.172 min | |
| | | | | | | m/z = 392.1 | |
| | | | | | | [M + H]⁺ | |
| 162 | 2-NO₂ | H | H | CH(CH₃)₂ | CH₃ | 3.369 min | isomer |
| | | | | | | m/z = 408.2 | 1 |
| | | | | | | [M + H]⁺ | |
| 163 | 2-NO₂ | H | H | CH(CH₃)₂ | CH₃ | 3.096 min | isomer |
| | | | | | | m/z = 408.1 | 2 |
| | | | | | | [M + H]⁺ | |
| 164 | 2-Br | H | H | CN | CH₃ | 3.180 min | |
| | | | | | | m/z = 424.4 | |
| | | | | | | [M]⁺ | |
| 165 | 2-Br | H | H | Br | CH₃ | 3.578 min | isomer |
| | | | | | | m/z = 478.9 | 1 |
| | | | | | | [M + H]⁺ | |
| 166 | 2-Br | H | H | Br | CH₃ | 3.643 min | isomer |
| | | | | | | m/z = 478.9 | 2 |
| | | | | | | [M + H]⁺ | |

Example No. Example Number
m.p. melting point

TABLE 4

| | |
|---|---|
| | (I) |
| 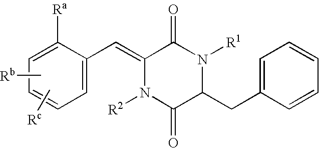 | |

| Ex- | | | | | | |
|---|---|---|---|---|---|---|
| ample | | | | | | RT HPLC/MS |
| No. | R^{a} | R^{b} | R^{c} | R² | R¹ | or m.p. |
| 167 | 2-NO₂ | H | H | CH₂CH₃ | C(O)CH₃ | 3.578 min |
| | | | | | | m/z = 408.1 |
| | | | | | | [M + H]⁺ |
| 168 | 2-Br | H | H | C(O)CH₃ | C(O)CH₃ | 3.812 min |
| | | | | | | m/z = 456.9 |
| | | | | | | [M + H]⁺ |
| 169 | 2-Br | H | H | CH₂—(C₆H₅) | CH₂—(C₆H₅) | 4.385 min |
| | | | | | | m/z = 552.9 |
| | | | | | | [M + H]⁺ |
| 170 | 2-Br | H | H | CH₂—CH₃ | CH₂—CH₃ | 3.637 min |
| | | | | | | m/z = 428.9 |
| | | | | | | [M + H]⁺ |
| 171 | 2-Br | H | H | CH₂—CH═CH₂ | CH₂—CH═CH₂ | 3.879 min |
| | | | | | | m/z = 453.0 |
| | | | | | | [M + H]⁺ |
| 172 | 2-CN | H | H | CH₂—(C₆H₅) | CH₂—(C₆H₅) | 3.995 min |
| | | | | | | m/z = 498.1 |
| | | | | | | [M + H]⁺ |
| 173 | 2-CN | H | H | CH₂—CH₃ | CH₂—CH₃ | 3.223 min |
| | | | | | | m/z = 374.1 |
| | | | | | | [M + H]⁺ |
| 174 | 2-CN | H | H | CH₂—CH═CH₂ | CH₂—CH═CH₂ | 3.469 min |
| | | | | | | m/z = 398.1 |
| | | | | | | [M + H]⁺ |
| 175 | 2-Br | H | H | CH₂—(C₆H₅) | C(O)CH₃ | 4.235 min |
| | | | | | | m/z = 504.9 |
| | | | | | | [M + H]⁺ |
| | | | | | | 98° C. |
| 176 | 2-Br | H | H | CH₂—CH═CH₂ | C(O)CH₃ | 4.010 min |
| | | | | | | m/z = 454.9 |
| | | | | | | [M + H]⁺ |
| 177 | 2-Br | H | H | CH₂—CH₃ | C(O)CH₃ | 3.921 min |
| | | | | | | m/z = 441.4 |
| | | | | | | [M + H]⁺ |
| 178 | 2-Br | H | H | CH₂—(C₆H₅) | H | 3.620 min |
| | | | | | | m/z = 463.4 |
| | | | | | | [M + H]⁺ |
| 179 | 2-Br | H | H | CH₂—CH═CH₂ | H | 3.322 min |
| | | | | | | m/z = 410.9 |
| | | | | | | [M + H]⁺ |
| | | | | | | 78° C. |
| 180 | 2-Br | H | H | CH₂—CH₃ | H | 3.216 min |
| | | | | | | m/z = 401.0 |
| | | | | | | [M + H]⁺ |
| 181 | 2-CN | H | H | CH₂—(C₆H₅) | H | 3.218 min |
| | | | | | | m/z = 408.0 |
| | | | | | | [M + H]⁺ |
| 182 | 2-CN | H | H | CH₂—CH═CH₂ | H | 2.929 min |
| | | | | | | m/z = 358.4 |
| | | | | | | [M + H]⁺ |
| 183 | 2-CN | H | H | CH₂—CH₃ | H | 2.830 min |
| | | | | | | m/z = 346.4 |
| | | | | | | [M + H]⁺ |

Example No. Example Number
m.p. melting point

TABLE 5

| Example | | RT HPLC/MS | |
|---|---|---|---|
| No. | Name | or m.p. | Isomer |
| 184 | 3-benzyl-1,4-dimethyl-6-(4-nitro-1H- | 2.743 min, m/z = 405.2 [M + H]⁺ | isomer 1 |
| | indol-3-ylmethylene)piperazine-2,5-dione | 159° C. | |
| 185 | 3-benzyl-1,4-dimethyl-6-(4-nitro-1H- | 2.608 min, m/z = 405.1 [M + H]⁺ | isomer 2 |
| | indol-3-ylmethylene)piperazine-2,5-dione | 238° C. | |
| 186 | tert-butyl 3-(5-benzyl-1,4-dimethyl-3,6- | 3.776 min, m/z = 505.0 [M + H]⁺ | isomer 1 |
| | dioxopiperazin-2-ylidenemethyl)-4-nitro- | | |
| | indole-1-carboxylate | | |
| 187 | tert-butyl 3-(5-benzyl-1,4-dimethyl-3,6- | 4.243 min, m/z = 505.5 [M + H]⁺ | isomer 2 |
| | dioxopiperazin-2-ylidenemethyl)-4-nitro- | | |
| | indole-1-carboxylate | | |
| 188 | 1-methyl-6-(2-nitrobenzylidene)-3- | 2.579 min, m/z = 357.9 [M + H]⁺ | isomer 1 |
| | thiophen-2-ylmethylpiperazine-2,5-dione | | |
| 188 | 1-methyl-6-(2-nitrobenzylidene)-3- | 2.579 min, | isomer 1 |
| | thiophen-2-ylmethylpiperazine-2,5-dione | m/z = 357.9 [M + H]⁺ | |
| 189 | 1-methyl-6-(2-nitrobenzylidene)-3- | 2.622 min, | isomer 2 |
| | thiophen-2-ylmethylpiperazine-2,5-dione | m/z = 358.0 [M + H]⁺ | |
| 190 | 3-benzyl-6-(5-chloro-1-methyl-3- | 3.108 min, | |
| | trifluoromethyl-1H-pyrazol-4-yl- | m/z = 427.1 [M + H]⁺ | |
| | methylene)-1,4-dimethylpiperazine-2,5- | | |
| | dione | | |
| 191 | 3-benzyl-1,4-dimethyl-6-(2-nitro- | 2.730 min, | isomer 1 |
| | thiophen-3-ylmethylene)piperazine-2,5- | m/z = 372.1 [M + H]⁺ | |
| | dione | | |
| 192 | 3-benzyl-1,4-dimethyl-6-(2-nitro- | 2.784 min, | isomer 2 |
| | thiophen-3-ylmethylene)piperazine-2,5- | m/z = 372.1 [M + H]⁺ | |
| | dione | | |
| 193 | 3-benzyl-1,4-dimethyl-6-(3-nitro- | 2.756 min, | isomer 1 |
| | thiophen-2-ylmethylene)piperazine-2,5- | m/z = 372.0 [M + H]⁺ | |
| | dione | | |
| 194 | 3-benzyl-1,4-dimethyl-6-(3-nitro- | 2.899 min, | isomer 2 |
| | thiophen-2-ylmethylene)piperazine-2,5- | m/z = 372.1 [M + H]⁺ | |
| | dione | | |
| 195 | 1,4-dimethyl-3-(2-nitrobenzylidene)-6- | 2.884 min, | |
| | thiophen-2-ylmethylpiperazine-2,5-dione | m/z = 372.1 [M + H]⁺ | |
| 196 | 3-benzyl-1,4-dimethyl-6-(3-nitropyridin-4- | 2.425 min, | isomer 1 |
| | ylmethylene)piperazine-2,5-dione | m/z = 367.1 [M + H]⁺ | |
| | | 180° C. | |
| 197 | 3-benzyl-1,4-dimethyl-6-(3-nitropyridin-4- | 2.418 min, | isomer 2 |
| | ylmethylene)piperazine-2,5-dione | m/z = 367.1 [M + H]⁺ | |
| | | 175° C. | |
| 198 | 3-benzyl-1,4-dimethyl-6-(3-nitropyridin-2- | 2.606 min, | |
| | ylmethylene)piperazine-2,5-dione | m/z = 367.1 [M + H]⁺ | |
| | | 165° C. | |
| 199 | 3-benzyl-1,4-dimethyl-6-(4-bromo-1H- | 2.845 min, | |
| | indol-3-ylmethylene)piperazine-2,5-dione | m/z = 440.0 [M + H]⁺ | |
| | | 236° C. | |
| 200 | 3-benzyl-1,4-dimethyl-6-(4-cyano-1H- | 2.553 min, | isomer 1 |
| | indol-3-ylmethylene)piperazine-2,5-dione | m/z = 385.2 [M + H]⁺ | |
| 201 | 3-benzyl-1,4-dimethyl-6-(4-cyano-1H- | 2.553 min, | isomer 2 |
| | indol-3-ylmethylene)piperazine-2,5-dione | m/z = 385.2 [M + H]⁺ | |
| 202 | 3-benzyl-1,4-dimethyl-6-(4- | 3.887 min, | |
| | trimethylsilanylethynyl-1H-indol-3- | m/z = 456.1 [M + H]⁺ | |
| | ylmethylene)piperazine-2,5-dione | | |
| 203 | 2-(1,4-dimethyl-3,6-dioxo-5-thiophen-2- | 2.743 min, | |
| | ylmethylpiperazin-2-ylidene- | m/z = 352.1 [M + H]⁺ | |
| | methyl)benzonitrile | | |
| 204 | 3-benzyl-6-(2-bromopyridin-3- | 2.615 min, | |
| | ylmethylene)-1,4-dimethylpiperazine-2,5- | m/z = 400.0 [M]⁺ | |
| | dione | | |
| 205 | 3-benzyl-6-(2-cyanopyridin-3- | 2.485 min, | |
| | ylmethylene)-1,4-dimethylpiperazine-2,5- | m/z = 347.4 [M]⁺ | |
| | dione | 218° C. | |
| 206 | 3-benzyl-6-(4-iodopyridin-3-yl- | 3.029 min, | |
| | methylene)-1,4-dimethylpiperazine-2,5- | m/z = 445.9 [M + H]⁺ | |
| | dione | | |
| 207 | 2-[5-(4-fluorobenzyl)-1,4-dimethyl-3,6- | 2.843 min, | |
| | dioxopiperazin-2-ylidene- | m/z = 364.0 [M + H]⁺ | |
| | methyl]benzonitrile | | |
| 208 | 3-benzyl-6-(5-bromo-3H-imidazol-4- | 200° C. | isomer 1 |
| | ylmethylene)-1,4-dimethylpiperazine-2,5- | | |
| | dione | | |
| 209 | 3-benzyl-6-(5-bromo-3H-imidazol-4- | m/z = 389.1 [M]⁺ | isomer 2 |
| | ylmethylene)-1,4-dimethylpiperazine-2,5- | | |
| | dione | | |
| 210 | 3-benzyl-6-(5-cyano-3H-imidazol-4- | 170° C. | |
| | ylmethylene)-1,4-dimethylpiperazine-2,5- | | |
| | dione | | |
| 211 | 3-benzyl-6-(5-cyano-3H-imidazol-4- | m/z = 336.2 [M + H]⁺ | isomer 2 |
| | ylmethylene)-1,4-dimethylpiperazin-2,5- | | |
| | dione | | |
| 212 | 3-benzyl-6-(5-brom-3H-[1,2,3]triazol-4- | m/z = 390.0 [M]⁺ | isomer 1 |
| | ylmethylene)-1,4-dimethylpiperazin-2,5- | | |
| | dione | | |
| 213 | 3-benzyl-6-(5-brom-3H-[1,2,3]triazol-4- | m/z = 392.0 [M ]⁺ | isomer 2 |
| | ylmethylene)-1,4-dimethylpiperazin-2,5- | | |
| | dione | | |
| 214 | 3-(2-Iodobenzylidene)-1,4-dimethyl-6- | 3.303 min | |
| | (thiophen-2-ylmethyl)piperazin-2,5-dione | m/z = 453.0 [M + H]⁺ | |

Ex. No. Example Number
m.p. melting point

### Part B

### USE EXAMPLES

The herbicidal activity of the compounds of the formula I was demonstrated by greenhouse experiments:

The culture containers used were plastic flowerpots containing loamy sand with approximately 3.0% of humus as the substrate. The seeds of the test plants were sown separately for each species.

For the pre-emergence treatment, the active ingredients, which had been suspended or emulsified in water, were applied directly after sowing by means of finely distributing nozzles. The containers were irrigated gently to promote germination and growth and subsequently covered with transparent plastic hoods until the plants had rooted. This cover causes uniform germination of the test plants, unless this has been impaired by the active ingredients.

For the post-emergence treatment, the test plants were first grown to a height of 3 to 15 cm, depending on the plant habit, and only then treated with the active ingredients which had been suspended or emulsified in water. For this purpose, the test plants were either sown directly and grown in the same containers, or they were first grown separately as seedlings and transplanted into the test containers a few days prior to treatment.

Depending on the species, the plants were kept at 10-25° C. or 20-35° C. The test period extended over 2 to 4 weeks. During this time, the plants were tended, and their response to the individual treatments was evaluated.

Evaluation was carried out using a scale from 0 to 100. 100 means no emergence of the plants, or complete destruction of at least the aerial moieties, and 0 means no damage, or normal course of growth. Good herbicidal activity is given for values of at least 70 and very good herbicidal activity is given for values of at least 85.

The plants used in the greenhouse experiments belonged to the following species:

| Scientific name | Common name |
|---|---|
| Lulium perenne (LOLMU) | annual ryegrass |
| Amaranthus retroflexus | pig weed |
| (AMARE) | |
| Abutilon theophrasti (ABUTH) | velvet leaf |
| Apera spica-venti (APESV) | windgrass |
| Avena fatua (AVEFA) | wild oat |
| Echinochloa crus galli | barnyard grass |
| (ECHCG) | |
| Setaria faberi (SETFA) | giant foxtail |
| Setaria viridis (SETVI) | green foxtail |

The compounds of Examples 1, 13, 16, 44, 47, 48, 49, 51, 52, 129, 132, 160 and 170 exhibit good to very good herbicidal activity when applied by the post-emergence method.

The compounds of Examples 44 and 160, applied by the post-emergence method at an application rate of 3 kg/ha, exhibit good herbicidal activity against ABUTH.

The compounds of Examples 1, 16 and 129, applied by the post-emergence method at an application rate of 1 kg/ha, exhibit very good herbicidal activity against AMARE. The compound of Example 13, applied by the post-emergence method at an application rate of 0.5 kg/ha, exhibits very good herbicidal activity against AMARE.

The compound of Example 132, applied by the post-emergence method at an application rate of 3 kg/ha, exhibits very good herbicidal activity against AVEFA.

The compound of Example 1, applied by the post-emergence method at an application rate of 1 kg/ha, exhibits very good herbicidal activity against LOLMU.

The compounds of Examples 44, 47/48 (mixture), 49, 51 and 160, applied by the post-emergence method at an application rate of 3 kg/ha, exhibit very good herbicidal activity, and the compounds of Examples 52 and 132 exhibit good herbicidal activity against SETFA. The compound of Example 170, applied by the post-emergence method at an application rate of 1 kg/ha, exhibits good herbicidal activity against SETFA.

The compounds of Examples 38, 40, 45, 54, 62, 73, 100, 101, 109, 110, 119, 124, 137, 140, 147, 148, 191, 198, 204, 206, 214 exhibit good to very good herbicidal activity when applied by the pre-emergence method.

The compound of Example 73, applied by the pre-emergence method at an application rate of 3 kg/ha, exhibits good herbicidal activity against ABUTH.

The compound of Example 137, applied by the pre-emergence method at an application rate of 1 kg/ha, exhibits good herbicidal activity against AMARE.

The compound of Example 206, applied by the pre-emergence method at an application rate of 1 kg/ha, exhibits good herbicidal activity against AVEFA.

The compounds of Examples 38, 45, 54, 124, 140, 147 and 198, applied by the pre-emergence method at an application rate of 1 kg/ha, exhibit very good herbicidal activity against APSEV. The compounds of Examples 119 and 191, applied by the pre-emergence method at an application rate of 0.5 kg/ha, exhibit good herbicidal activity against APSEV.

The compounds of Examples 38, 45, 100/101 (mixture) and 109/110 (mixture), applied by the pre-emergence method at an application rate of 1 kg/ha, exhibit very good herbicidal activity, and the compound 147 exhibits good herbicidal activity against ECHCG. The compounds of Examples 40 and 204, applied by the pre-emergence method at an application rate of 3 kg/ha, exhibit very good herbicidal activity, and the compound of Example 214 exhibits good herbicidal activity against ECHCG.

The compounds of Examples 109/110 (mixture) and 140, applied by the pre-emergence method at an application rate of 1 kg/ha, exhibit very good herbicidal activity against SETFA. The compound of Example 62, at an application rate of 3 kg/ha, and the compound of Example 206, at an application rate of 2 kg/ha, each exhibit good herbicidal activity against SETFA.

The compounds of Examples 100/101 (mixture), applied by the pre-emergence method at an application rate of 1 kg/ha, exhibit very good herbicidal activity against SETIT. The compounds of Examples 40 and 204, applied by the pre-emergence method at an application rate of 3 kg/ha, exhibit very good herbicidal activity against SETIT. The compound of Example 214, applied by the pre-emergence method at an application rate of 3 kg/ha, exhibits good herbicidal activity against SETIT.

1-20. (canceled)

21. A method for controlling unwanted vegetation comprising treating a plant, a plant seed, and/or a plant habitat with a herbicidally effective amount of at least one piperazine compound of formula (I) 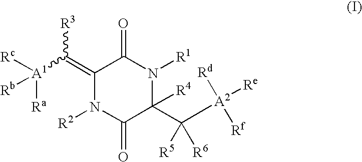
or an agriculturally useful salt thereof, wherein:
R¹ and R²
are, independently of one another cyano, C₁-C₆-alkyl, C₃-C₆-cycloalkyl, C₃-C₆-alkenyl, C₃-C₆-cycloalkenyl, C₃-C₆-alkynyl, C₃-C₆-cycloalkynyl, phenyl, phenyl-(C₁-C₆)-alkyl, heterocyclyl, heterocyclyl-(C₁-C₆)-alkyl; phenyl-[C₁-C₆-alkoxycarbonyl]-(C₁-C₆)-alkyl; phenylheterocyclyl-(C₁-C₆)-alkyl; or
COR²¹, wherein
R²¹ is hydrogen, C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, hydroxyl, C₁-C₆-alkoxy, C₃-C₆-alkenyloxy, C₃-C₆-alkynyloxy, amino, C₁-C₆-alkylamino, [di-(C₁-C₆)-alkyl]amino, C₃-C₆-alkenylamino, C₃-C₆-alkynylamino, C₁-C₆-alkylsulfonylamino, N—(C₂-C₆-alkenyl)-N—(C₁-C₆-alkyl)amino, N—(C₂-C₆-alkynyl)-N—(C₁-C₆-alkyl)amino, N—(C₁-C₆-alkoxy)-N—(C₁-C₆-alkyl)amino, N—(C₂-C₆-alkenyl)-N—(C₁-C₆-alkoxy)amino, N—(C₂-C₆-alkynyl)-N—(C₁-C₆-alkoxy)amino, phenyl, phenylamino, phenoxy, naphthyl, heterocyclyl; or
N²²R²³, wherein
R²² and R²³ are, independently of one another, hydrogen, C₁-C₆-alkyl, C₃-C₆-cycloalkyl, C₃-C₆-alkenyl, C₃-C₆-cycloalkenyl, C₃-C₆-alkynyl, C₃-C₆-cycloalkynyl, C₁-C₆-alkylcarbonyl; or
OR²⁴, wherein
R²⁴ is C₁-C₆-alkyl, C₃-C₆-cycloalkyl, C₃-C₆-alkenyl, C₃-C₆-cycloalkenyl, C₃-C₆-alkynyl, C₃-C₆-cycloalkynyl, phenyl, phenyl-(C₁-C₆)-alkyl; or
SO₂R²⁵, wherein
R²⁵ is C₁-C₆-alkyl or phenyl;
wherein, when R¹ and R² are aliphatic, cyclic, or aromatic substituents, said aliphatic, cyclic, or aromatic substituents are optionally partially or fully halogenated and/or are optionally mono-, di, or tri-substituted with cyano, hydroxyl, C₁-C₄-alkyl, C₁-C₄-haloalkyl, C₃-C₆-cycloalkyl, C₁-C₄-alkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl, C₁-C₄-alkylcarbonyloxy, or combinations thereof, and
wherein R¹ is optionally hydrogen;
R³ is hydrogen, halogen, cyano, C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, phenyl, phenyl-(C₁-C₆)-alkyl, heterocyclyl, heterocyclyl-(C₁-C₆)-alkyl, phenyl-[C₁-C₆-alkoxycarbonyl]-(C₁-C₆)-alkyl, phenylheterocyclyl-(C₁-C₆)-alkyl, or a radical COR²⁶, NR²⁷R²⁸, OR²⁹, SO₂R³⁰, or N(OR³¹)R³², wherein
R²⁶ is hydrogen, C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, hydroxyl, C₁-C₆-alkoxy, C₃-C₆-alkenyloxy, C₃-C₆-alkynyloxy, amino, C₁-C₆-alkylamino, [di-(C₁-C₆)-alkyl]amino, C₃-C₆-alkenylamino, C₃-C₆-alkynylamino, C₁-C₆-alkylsulfonylamino, N—(C₂-C₆-alkenyl)-N—(C₁-C₆-alkyl)amino, N—(C₂-C₆-alkynyl)-N—(C₁-C₆-alkyl)amino, N—(C₁-C₆-alkoxy)-N—(C₁-C₆-alkyl)amino, N—(C₂-C₆-alkenyl)-N—(C₁-C₆-alkoxy)amino, N—(C₂-C₆-alkynyl)-N—(C₁-C₆-alkoxy)amino, phenyl, phenylamino, phenoxy, naphthyl, or heterocyclyl;
R²⁷ and R²⁸
are, independently of one another, hydrogen, C₁-C₆-alkyl, aryl, or heteroaryl;
R²⁹ is C₁-C₆-alkyl;
R³⁰ is C₁-C₆-alkyl or phenyl;
R³¹ is hydrogen, C₁-C₆-alkyl, phenyl, or phenyl-(C₁-C₆)-alkyl;
R³² is C₁-C₆-alkyl, phenyl, or phenyl-(C₁-C₆)-alkyl;
wherein, when R³, R²⁶, R²⁷, R²⁸, R²⁹, R³⁰, R³¹, and R³² are aliphatic, cyclic, or aromatic substituents, said aliphatic, cyclic, or aromatic substituents are optionally partially or fully halogenated and/or are optionally mono-, di, or tri-substituted with cyano, hydroxyl, C₁-C₄-alkyl, C₁-C₄-haloalkyl, C₃-C₆-cycloalkyl, C₁-C₄-alkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl, C₁-C₄-alkylcarbonyloxy, or combinations thereof;
R⁴, R⁵, and R⁶
are, independently of one another, hydrogen, hydroxyl, C₁-C₆-alkyl, or C₁-C₆-alkoxy, wherein the aliphatic moieties of these substituents are optionally partially or fully halogenated and/or are optionally mono-, di, or tri-substituted with cyano, hydroxyl, C₁-C₄-alkyl, C₁-C₄-haloalkyl, C₃-C₆-cycloalkyl, C₁-C₄-alkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl, C₁-C₄-alkylcarbonyloxy, or combinations thereof;
A¹ is aryl or heteroaryl;
A² is aryl or heteroaryl, with the proviso that A² is not indolyl;
R^{a} is halogen, cyano, nitro, C₁-C₆-alkyl, C₃-C₆-cycloalkyl, C₂-C₆-alkenyl, C₃-C₆-cycloalkenyl, C₄-C₁₀-alkadienyl, C₂-C₆-alkynyl, [tri-(C₁-C₆)-alkylsilyl]-(C₂-C₆)-alkynyl, C₃-C₆-cycloalkynyl, C₁-C₆-alkylthio, C₁-C₆-alkylsulfinyl, aryl, phenyl-(C₁-C₆)-alkyl, phenyl-(C₂-C₆)-alkenyl, phenylsulfonyl-(C₁-C₆)-alkyl, heterocyclyl, heterocyclyl-(C₁-C₆)-alkyl, or phenyl-[C₁-C₆-alkoxycarbonyl]-(C₁-C₆)-alkyl, or
Z¹P(O)(OR⁹)₂ or Z²B(OR¹⁰)₂, wherein
R⁹ and R¹⁰ are each, independently of one another, hydrogen or C₁-C₆-alkyl and both R¹⁰ moieties in Z²B(OR¹⁰)₂ together optionally define a C₂-C₄-alkylene chain; or
Z³COR¹¹, wherein
R¹¹ is hydrogen, C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, hydroxyl, C₁-C₆-alkoxy, C₃-C₆-alkenyloxy, C₃-C₆-alkynyloxy, amino, C₁-C₆-alkylamino, [di-(C₁-C₆)-alkyl]amino, C₁-C₆-alkoxyamino, [di-(C₁-C₆)-alkoxy]amino, C₁-C₆-alkylsulfonylamino, C₁-C₆-alkylaminosulfonylamino, [di-(C₁-C₆)-alkylamino]sulfonylamino, C₃-C₆-alkenylamino, C₃-C₆-alkynylamino, N—(C₂-C₆-alkenyl)-N—(C₁-C₆₆-alkyl)amino, N—(C₂-C₆-alkynyl)-N—(C₁-C₆-alkyl)amino, N—(C₁-C₆-alkoxy)-N—(C₁-C₆-alkyl)amino, N—(C₂-C₆-alkenyl)-N—(C₁-C₆-alkoxy)amino, N—(C₂-C₆-alkynyl)-N—(C₁-C₆-alkoxy)amino, phenyl, phenoxy, phenylamino, naphthyl, or heterocyclyl; or
Z⁴NR¹²R¹³, wherein
R¹² and R¹³ are, independently of one another, hydrogen, C₁-C₆-alkyl, C₃-C₆-cycloalkyl, C₃-C₆-alkenyl, C₃-C₆-cycloalkenyl, C₃-C₆-alkynyl, C₃-C₆-cycloalkynyl, C₁-C₆-alkylcarbonyl, C₃-C₆-cycloalkylcarbonyl, [di-(C₁-C₆)-alkylamino]carbonyl, C₁-C₆-alkoxycarbonyl, C₁-C₆-alkoxycarbonyl-(C₁-C₆)-alkyl, C₁-C₆-alkylsulfonyl, C₁-C₆-alkylaminosulfonyl, [di-(C₁-C₆)-alkylamino]sulfonyl, phenylcarbonyl, phenylaminocarbonyl, phenylsulfonyl, phenylsulfonylaminocarbonyl, or heterocyclylcarbonyl; or
Z⁵CH═N—O—R¹⁴, wherein R¹⁴ is hydrogen or C₁-C₆-alkyl; or
Z⁶OR¹⁵, wherein
R¹⁵ is hydrogen, C₁-C₆-alkyl, C₃-C₆-cycloalkyl, C₃-C₆-alkenyl, C₃-C₆-cycloalkenyl, C₃-C₆-alkynyl, C₃-C₆-cycloalkynyl, C₁-C₆-alkylcarbonyl, C₁-C₆-alkoxycarbonyl-(C₁-C₆)-alkyl, [di-(C₁-C₆)-alkoxycarbonyl]-(C₁-C₆)-alkyl, phenyl, or phenyl-(C₁-C₆)-alkyl; or
Z⁷SO₂R¹⁶, wherein R¹⁶ is C₁-C₆-alkyl or phenyl;
wherein
Z¹, Z², Z³, Z⁴, Z⁶, and Z⁷
are, independently of one another, a bond, —CH₂—, —CH₂—CH₂—, —O—OH(R¹⁷)—, —S—CH(R¹⁸)—, —S(O)—CH(R¹⁹)—, or —SO₂CH(R²⁰)—, wherein R¹⁷, R¹⁸, R¹⁹, and R²⁰ are, independently of one another, hydrogen or C₁-C₆-alkyl; and
wherein, when R^{a} is an aliphatic, cyclic, or aromatic substituent, said aliphatic, cyclic, or aromatic substituent is optionally partially or fully halogenated and/or is optionally mono-, di, or tri-substituted with cyano, hydroxyl, C₁-C₄-alkyl, C₁-C₄-haloalkyl, C₃-C₆-cycloalkyl, C₁-C₄-alkoxy, C₁-C₄-haloalkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl, C₁-C₄-alkylcarbonyloxy, or combinations thereof;
R^{b}, R^{c}, R^{d}, R^{e}, and R^{f}
are, independently of one another, hydrogen or a substituent as defined in R^{a}; and wherein any two of R^{a}, R^{b}, or R^{c} attached to adjacent ring atoms of A¹ or any two of R^{d}, R^{e} or R^{f} attached to adjacent ring atoms of A² are optionally straight-chain C₃-C₆-alkylene optionally partially or fully halogenated and optionally mono-, di, or tri-substituted with cyano, hydroxyl, C₁-C₄-alkyl, C₁-C₄-haloalkyl, C₃-C₆-cycloalkyl, C₁-C₄-alkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl, C₁-C₄-alkylcarbonyloxy, or combinations thereof, wherein one CH₂ group in C₃-C₆-alkylene is optionally replaced by a carbonyl group, a thiocarbonyl group, or a sulfonyl group and wherein one or two non-adjacent CH₂ groups in C₃-C₆-alkylene are optionally replaced by oxygen, sulfur, or a group NR³⁴, wherein R³⁴ is a substituent as defined in R¹².

22. The method of claim 21, wherein R^{a} is attached in the ortho-position to the point of attachment of A¹.

23. The method of claim 21, wherein A¹ is bicyclic aryl or hetaryl.

24. The method of claim 21, wherein A¹ and A² are, independently of one another, selected from the group consisting of phenyl, naphthyl, furyl, thienyl, pyrrolyl, pyrazolyl, thiazolyl, isothiazolyl, oxazolyl, isoxazolyl, triazolyl, tetrazolyl, pyridinyl, pyridazinyl, pyrimidinyl, pyrazinyl, triazinyl, and tetrazinyl.

25. The method of claim 21, wherein A¹ and A² are, independently of one another, selected from the group consisting of phenyl, furyl, thienyl, triazolyl, tetrazolyl, and pyridinyl.

26. The method of claim 21, wherein A¹ is phenyl or pyridinyl.

27. The method of claim 21, wherein A² is phenyl or thienyl.

28. The method of claim 21, wherein
R^{a} is selected from the group consisting of halogen, cyano, nitro, C₁-C₆-alkyl, C₂-C₆-alkenyl, C₂-C₆-alkynyl, C₃-C₆-cycloalkyl, [tri-(C₁-C₆)-alkylsilyl]-C₂-C₆-alkynyl, Z¹P(O)(OR¹)₂, Z¹COR¹¹, Z⁴NR¹²R¹³, Z⁵CH═N—O—R¹⁴, Z⁶OR¹⁵, Z⁷SO₂R¹⁶, C₁-C₆-alkylthio, C₁-C₆-alkylsulfinyl, aryl, and heterocyclyl, wherein
Z¹ is a bond or CH₂ and each R⁹ is, independently of another, hydrogen or C₁-C₆-alkyl;
Z³ is a bond and R¹¹ is hydrogen, C₁-C₆-alkyl, hydroxyl, C₁-C₆-alkoxy, C₃-C₆-alkenyloxy, C₃-C₆-alkynyloxy, amino, C₁-C₆-alkylamino, [di-(C₁-C₆)-alkyl]amino, C₁-C₆-alkoxyamino, (C₁-C₆)-alkoxy-(C₁-C₆)-alkylamino, C₁-C₆-alkylsulfonylamino, C₁-C₆-alkylaminosulfonylamino, [di-(C₁-C₆)-alkylamino]sulfonylamino, phenyl, phenoxy, phenylamino, naphthyl, or heterocyclyl;
Z⁴ is a bond or CH₂ and R¹² and R¹³ are, independently of one another, hydrogen, C₁-C₆-alkyl, C₃-C₆-cycloalkyl, C₃-C₆-alkenyl, C₃-C₆-alkynyl, C₁-C₆-alkylcarbonyl, [di-(C₁-C₆)-alkylamino]carbonyl, C₁-C₆-alkoxycarbonyl, C₁-C₆-alkylsulfonyl, phenylcarbonyl, phenylsulfonyl, or heterocyclylcarbonyl;
Z⁵ is a bond and R¹⁴ is hydrogen or C₁-C₆-alkyl;
Z⁶ is a bond or CH₂ and R¹⁵ is hydrogen, C₁-C₆-alkyl, C₃-C₆-cycloalkyl, C₃-C₆-alkenyl, C₃-C₆-alkynyl, C₁-C₆-alkylcarbonyl, C₁-C₆-alkoxycarbonyl-(C₁-C₆)-alkyl, phenyl, or phenyl-(C₁-C₆)-alkyl; and
Z⁷ is a bond and R¹⁶ is C₁-C₆-alkyl or phenyl;
and wherein
R^{b}, R^{c}, R^{d}, R^{e}, and R^{f}
are, independently of one another, hydrogen or are as defined in R^{a}, and
wherein, when R^{a}, R^{b}, R^{c}, R^{d}, R^{e}, and R^{f} are aliphatic, cyclic, or aromatic substituents, said aliphatic, cyclic, or aromatic substituents are optionally partially or fully halogenated.

29. The method of claim 21, wherein R¹ is hydrogen or C₁-C₆-alkyl and R² is C₁-C₆-alkyl; wherein C₁-C₆-alkyl in R¹ and R² is optionally partially or fully halogenated.

30. The method of claim 21, wherein R³ is hydrogen, halogen, C₁-C₆-alkyl, or halo-C₁-C₆-alkyl.

31. The method of claim 21, wherein R⁴, R⁵, and R⁶ are hydrogen.

32. The method of claim 21, wherein the center of chirality in the piperazine ring has the (S)-configuration.

33. The method of claim 21, wherein the exo double bond at the piperazine ring has the (Z)-conformation.

34. A composition comprising a herbicidally effective amount of at least one piperazine compound of formula (I) 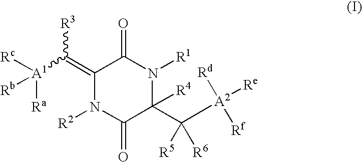
or an agriculturally useful salt thereof, wherein:
R¹ and R²
are, independently of one another cyano, C₁-C₆-alkyl, C₃-C₆-cycloalkyl, C₃-C₆-alkenyl, C₃-C₆-cycloalkenyl, C₃-C₆-alkynyl, C₃-C₆-cycloalkynyl, phenyl, phenyl-(C₁-C₆)-alkyl, heterocyclyl, heterocyclyl-(C₁-C₆)-alkyl; phenyl-[C₁-C₆-alkoxycarbonyl]-(C₁-C₆)-alkyl; phenylheterocyclyl-(C₁-C₆)-alkyl; or
COR²¹, wherein
R²¹ is hydrogen, C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, hydroxyl, C₁-C₆-alkoxy, C₃-C₆-alkenyloxy, C₃-C₆-alkynyloxy, amino, C₁-C₆-alkylamino, [di-(C₁-C₆)-alkyl]amino, C₃-C₆-alkenylamino, C₃-C₆-alkynylamino, C₁-C₆-alkylsulfonylamino, N—(C₂-C₆-alkenyl)-N—(C₁-C₆-alkyl)amino, N—(C₂-C₆-alkynyl)-N—(C₁-C₆-alkyl)amino, N—(C₁-C₆-alkoxy)-N—(C₁-C₆-alkyl)amino, N—(C₂-C₆-alkenyl)-N—(C₁-C₆-alkoxy)amino, N—(C₂-C₆-alkynyl)-N—(C₁-C₆-alkoxy)amino, phenyl, phenylamino, phenoxy, naphthyl, heterocyclyl; or
NR²²R²³, wherein
R² and R²³ are, independently of one another, hydrogen, C₁-C₆-alkyl, C₃-C₆-cycloalkyl, C₃-C₆-alkenyl, C₃-C₆-cycloalkenyl, C₃-C₆-alkynyl, C₃-C₆-cycloalkynyl, C₁-C₆-alkylcarbonyl; or
OR²⁴, wherein
R²⁴ is C₁-C₆-alkyl, C₃-C₆-cycloalkyl, C₃-C₆-alkenyl, C₃-C₆-cycloalkenyl, C₃-C₆-alkynyl, C₃-C₆-cycloalkynyl, phenyl, phenyl-(C₁-C₆)-alkyl; or
SO₂R²⁵, wherein
R²⁵ is C₁-C₆-alkyl or phenyl;
wherein, when R¹ and R² are aliphatic, cyclic, or aromatic substituents, said aliphatic, cyclic, or aromatic substituents are optionally partially or fully halogenated and/or are optionally mono-, di, or tri-substituted with cyano, hydroxyl, C₁-C₄-alkyl, C₁-C₄-haloalkyl, C₃-C₆-cycloalkyl, C₁-C₄-alkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl, C₁-C₄-alkylcarbonyloxy, or combinations thereof, and
wherein R¹ is optionally hydrogen;
R³ is hydrogen, halogen, cyano, C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, phenyl, phenyl-(C₁-C₆)-alkyl, heterocyclyl, heterocyclyl-(C₁-C₆)-alkyl, phenyl-[C₁-C₆-alkoxycarbonyl]-(C₁-C₆)-alkyl, phenylheterocyclyl-(C₁-C₆)-alkyl, or a radical COR²⁶, NR²⁷R²⁸, OR²⁹, SO₂R¹⁰, or N(OR³¹)R³², wherein
R²⁶ is hydrogen, C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, hydroxyl, C₁-C₆-alkoxy, C₃-C₆-alkenyloxy, C₃-C₆-alkynyloxy, amino, C₁-C₆-alkylamino, [di-(C₁-C₆)-alkyl]amino, C₃-C₆-alkenylamino, C₃-C₆-alkynylamino, C₁-C₆-alkylsulfonylamino, N—(C₂-C₆-alkenyl)-N—(C₁-C₆-alkyl)amino, N—(C₂-C₆-alkynyl)-N—(C₁-C₆-alkyl)amino, N—(C₁-C₆-alkoxy)-N—(C₁-C₆-alkyl)amino, N—(C₂-C₆-alkenyl)-N—(C₁-C₆-alkoxy)amino, N—(C₂-C₆-alkynyl)-N—(C₁-C₆-alkoxy)amino, phenyl, phenylamino, phenoxy, naphthyl, or heterocyclyl;
R²⁷ and R²⁸
are, independently of one another, hydrogen, C₁-C₆-alkyl, aryl, or heteroaryl;
R²⁹ is C₁-C₆-alkyl;
R³⁰ is C₁-C₆-alkyl or phenyl;
R³¹ is hydrogen, C₁-C₆-alkyl, phenyl, or phenyl-(C₁-C₆)-alkyl;
R³² is C₁-C₆-alkyl, phenyl, or phenyl-(C₁-C₆)-alkyl;
wherein, when R³, R²⁶, R²⁷, R²⁸, R²⁹, R³⁰, R³¹, and R³² are aliphatic, cyclic, or aromatic substituents, said aliphatic, cyclic, or aromatic substituents are optionally partially or fully halogenated and/or are optionally mono-, di, or tri-substituted with cyano, hydroxyl, C₁-C₄-alkyl, C₁-C₄-haloalkyl, C₃-C₆-cycloalkyl, C₁-C₄-alkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl, C₁-C₄-alkylcarbonyloxy, or combinations thereof;
R⁴, R⁵, and R⁶
are, independently of one another, hydrogen, hydroxyl, C₁-C₆-alkyl, or C₁-C₆-alkoxy, wherein the aliphatic moieties of these substituents are optionally partially or fully halogenated and/or are optionally mono-, di, or ti-substituted with cyano, hydroxyl, C₁-C₄-alkyl, C₁-C₄-haloalkyl, C₃-C₆-cycloalkyl, C₁-C₄-alkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl, C₁-C₄-alkylcarbonyloxy, or combinations thereof;
A¹ is aryl or heteroaryl;
A² is aryl or heteroaryl, with the proviso that A² is not indolyl;
R^{a} is halogen, cyano, nitro, C₁-C₆-alkyl, C₃-C₆-cycloalkyl, C₂-C₆-alkenyl, C₃-C₆-cycloalkenyl, C₄-C₁₀-alkadienyl, C₂-C₆-alkynyl, [tri-(C₁-C₆)-alkylsilyl]-(C₂-C₆)-alkynyl, C₃-C₆-cycloalkynyl, C₁-C₆-alkylthio, C₁-C₆-alkylsulfinyl, aryl, phenyl-(C₁-C₆)-alkyl, phenyl-(C₂-C₆)-alkenyl, phenylsulfonyl-(C₁-C₆)-alkyl, heterocyclyl, heterocyclyl-(C₁-C₆)-alkyl, or phenyl-[C₁-C₆-alkoxycarbonyl]-(C₁-C₆)-alkyl, or
Z¹P(O)(OR⁹)₂ or Z²B(OR¹⁰)₂, wherein
R⁹ and R¹⁰ are each, independently of one another, hydrogen or C₁-C₆-alkyl and both R¹⁰ moieties in Z²B(OR¹⁰)₂ together optionally define a C₂-C₄-alkylene chain; or
Z³COR¹¹, wherein
R¹¹ is hydrogen, C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, hydroxyl, C₁-C₆-alkoxy, C₃-C₆-alkenyloxy, C₃-C₆-alkynyloxy, amino, C₁-C₆-alkylamino, [di-(C₁-C₆)-alkyl]amino, C₁-C₆-alkoxyamino, [di-(C₁-C₆)-alkoxy]amino, C₁-C₆-alkylsulfonylamino, C₁-C₆-alkylaminosulfonylamino, [di-(C₁-C₆)-alkylamino]sulfonylamino, C₃-C₆-alkenylamino, C₃-C₆-alkynylamino, N—(C₂-C₆-alkenyl)-N—(C₁-C₆-alkyl)amino, N—(C₂-C₆-alkynyl)-N—(C₁-C₆-alkyl)amino, N—(C₁-C₆-alkoxy)-N—(C₁-C₆-alkyl)amino, N—(C₂-C₆-alkenyl)-N—(C₁-C₆-alkoxy)amino, N—(C₂-C₆-alkynyl)-N—(C₁-C₆-alkoxy)amino, phenyl, phenoxy, phenylamino, naphthyl, or heterocyclyl; or
Z⁴NR¹²R¹³, wherein
R¹² and R¹³ are, independently of one another, hydrogen, C₁-C₆-alkyl, C₃-C₆-cycloalkyl, C₃-C₆-alkenyl, C₃-C₆-cycloalkenyl, C₃-C₆-alkynyl, C₃-C₆-cycloalkynyl, C₁-C₆-alkylcarbonyl, C₃-C₆-cycloalkylcarbonyl, [di-(C₁-C₆)-alkylamino]carbonyl, C₁-C₆-alkoxycarbonyl, C₁-C₆-alkoxycarbonyl-(C₁-C₆)-alkyl, C₁-C₆-alkylsulfonyl, C₁-C₆-alkylaminosulfonyl, [di-(C₁-C₆)-alkylamino]sulfonyl, phenylcarbonyl, phenylaminocarbonyl, phenylsulfonyl, phenylsulfonylaminocarbonyl, or heterocyclylcarbonyl; or
Z⁵CH═N—O—R¹⁴, wherein R¹⁴ is hydrogen or C₁-C₆-alkyl; or
Z⁶OR¹⁵, wherein
R¹⁵ is hydrogen, C₁-C₆-alkyl, C₃-C₆-cycloalkyl, C₃-C₆-alkenyl, C₃-C₆-cycloalkenyl, C₃-C₆-alkynyl, C₃-C₆-cycloalkynyl, C₁-C₆-alkylcarbonyl, C₁-C₆-alkoxycarbonyl-(C₁-C₆)-alkyl, [di-(C₁-C₆)-alkoxycarbonyl]-(C₁-C₆)-alkyl, phenyl, or phenyl-(C₉-C₆)-alkyl; or
Z⁷SO₂R¹⁶, wherein R¹⁶ is C₁-C₆-alkyl or phenyl;
wherein
Z¹, Z², Z³, Z⁴, Z⁵, Z⁶, and Z⁷
are, independently of one another, a bond, —CH₂—, —CH₂—CH₂—, —O—CH(R¹⁷)—, —S—CH(R¹⁸)—, —S(O)—CH(R¹⁹)—, or —SO₂CH(R²⁰)—, wherein R¹⁷, R¹⁸, R¹⁹, and R²⁰ are, independently of one another, hydrogen or C₁-C₆-alkyl; and
wherein, when R^{a} is an aliphatic, cyclic, or aromatic substituent, said aliphatic, cyclic, or aromatic substituent is optionally partially or fully halogenated and/or is optionally mono-, di, or tri-substituted with cyano, hydroxyl, C₁-C₄-alkyl, C₁-C₄-haloalkyl, C₃-C₆-cycloalkyl, C₁-C₄-alkoxy, C₁-C₄-haloalkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl, C₁-C₄-alkylcarbonyloxy, or combinations thereof;
R^{b}, R^{c}, R^{d}, R^{e}, and R^{f}
are, independently of one another, hydrogen or a substituent as defined in R^{a}; and wherein any two of R^{a}, R^{b}, or R^{c} attached to adjacent ring atoms of A¹ or any two of R^{d}, R^{e}, or R^{f} attached to adjacent ring atoms of A² are optionally straight-chain C₃-C₆-alkylene optionally partially or fully halogenated and optionally mono-, di, or tri-substituted with cyano, hydroxyl, C₁-C₄-alkyl, C₁-C₄-haloalkyl, C₃-C₆-cycloalkyl, C₁-C₄-alkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl, C₁-C₄-alkylcarbonyloxy, or combinations thereof, where one CH₂ group in C₃-C₆-alkylene is optionally replaced by a carbonyl group, a thiocarbonyl group, or a sulfonyl group and wherein one or two non-adjacent CH₂ groups in C₃-C₆-alkylene are optionally replaced by oxygen, sulfur, or a group NR³⁴, wherein R³⁴ is a substituent as defined in R¹²; and
auxiliaries customary for formulating crop protection agents.

35. A process for preparing the composition of claim 34 comprising mixing a herbicidally effective amount of at least one piperazine compound of the formula (I) or an agriculturally useful salt thereof and auxiliaries customary for formulating crop protection agents.

36. A piperazine compound of general formula (I) 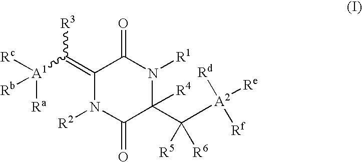
or an agriculturally useful salt thereof, wherein:
R¹ and R²
are, independently of one another cyano, C₁-C₆-alkyl, C₃-C₆-cycloalkyl, C₃-C₆-alkenyl, C₃-C₆-cycloalkenyl, C₃-C₆-alkynyl, C₃-C₆-cycloalkynyl, phenyl, phenyl-(C₁-C₆)-alkyl, heterocyclyl, heterocyclyl-(C₁-C₆)-alkyl; phenyl-[C₁-C₆-alkoxycarbonyl]-(C₁-C₆)-alkyl; phenylheterocyclyl-(C₁-C₆)-alkyl; or
COR²¹, wherein
R²¹ is hydrogen, C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, hydroxyl, C₁-C₆-alkoxy, C₃-C₆-alkenyloxy, C₃-C₆-alkynyloxy, amino, C₁-C₆-alkylamino, [di-(C₁-C₆)-alkyl]amino, C₃-C₆-alkenylamino, C₃-C₆-alkynylamino, C₁-C₆-alkylsulfonylamino, N—(C₂-C₆-alkenyl)-N—(C₁-C₆-alkyl)amino, N—(C₂-C₆-alkynyl)-N—(C₁-C₆-alkyl)amino, N—(C₁-C₆-alkoxy)-N—(C₁-C₆-alkyl)amino, N—(C₂-C₆-alkenyl)-N—(C₁-C₆-alkoxy)amino, N—(C₂-C₆-alkynyl)-N—(C₁-C₆-alkoxy)amino, phenyl, phenylamino, phenoxy, naphthyl, heterocyclyl; or
NR²²R²³, wherein
R²² and R²³ are, independently of one another, hydrogen, C₁-C₆-alkyl, C₃-C₆-cycloalkyl, C₃-C₆-alkenyl, C₃-C₆-cycloalkenyl, C₃-C₆-alkynyl, C₃-C₆-cycloalkynyl, C₁-C₆-alkylcarbonyl; or
OR²⁴, wherein
R²⁴ is C₁-C₆-alkyl, C₃-C₆-cycloalkyl, C₃-C₆-alkenyl, C₃-C₆-cycloalkenyl, C₃-C₆-alkynyl, C₃-C₆-cycloalkynyl, phenyl, phenyl-(C₁-C₆)-alkyl; or
SO₂R²⁵, wherein
R²⁵ is C₁-C₆-alkyl or phenyl;
wherein, when R¹ and R² are aliphatic, cyclic, or aromatic substituents, said aliphatic, cyclic, or aromatic substituents are optionally partially or fully halogenated and/or are optionally mono-, di, or tri-substituted with cyano, hydroxyl, C₁-C₄-alkyl, C₁-C₄-haloalkyl, C₃-C₆-cycloalkyl, C₁-C₄-alkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl, C₁-C₄-alkylcarbonyloxy, or combinations thereof, and
wherein R¹ is optionally hydrogen;
R³ is hydrogen, halogen, cyano, C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, phenyl, phenyl-(C₁-C₆)-alkyl, heterocyclyl, heterocyclyl-(C₁-C₆)-alkyl, phenyl-[C₁-C₆-alkoxycarbonyl]-(C₁-C₆)-alkyl, phenylheterocyclyl-(C₁-C₆)-alkyl, or a radical COR²⁶, NR²⁷R²⁸, OR²⁹, SO₂R³⁰, or N(OR³¹)R¹², wherein
R²⁶ is hydrogen, C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, hydroxyl, C₁-C₆-alkoxy, C₃-C₆-alkenyloxy, C₃-C₆-alkynyloxy, amino, C₁-C₆-alkylamino, [di-(C₁-C₆)-alkyl]amino, C₃-C₆-alkenylamino, C₃-C₆-alkynylamino, C₁-C₆-alkylsulfonylamino, N—(C₂-C₆-alkenyl)-N—(C₁-C₆-alkyl)amino, N—(C₂-C₆-alkynyl)-N—(C₁-C₆-alkyl)amino, N—(C₁-C₆-alkoxy)-N—(C₁-C₆-alkyl)amino, N—(C₂-C₆-alkenyl)-N—(C₁-C₆-alkoxy)amino, N—(C₂-C₆-alkynyl)-N—(C₁-C₆-alkoxy)amino, phenyl, phenylamino, phenoxy, naphthyl, or heterocyclyl;
R²⁷ and R²⁸
are, independently of one another, hydrogen, C₁-C₆-alkyl, aryl, or heteroaryl;
R²⁹ is C₁-C₆-alkyl;
R³⁰ is C₁-C₆-alkyl or phenyl;
R³¹ is hydrogen, C₁-C₆-alkyl, phenyl, or phenyl-(C₁-C₆)-alkyl;
R³² is C₁-C₆-alkyl, phenyl, or phenyl-(C₁-C₆)-alkyl;
wherein, when R³, R²⁶, R²⁷, R²⁸, R²⁹, R³⁰, R³¹, and R³² are aliphatic, cyclic, or aromatic substituents, said aliphatic, cyclic, or aromatic substituents are optionally partially or fully halogenated and/or are optionally mono-, di, or tri-substituted with cyano, hydroxyl, C₁-C₄-alkyl, C₁-C₄-haloalkyl, C₃-C₆-cycloalkyl, C₁-C₄-alkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl, C₁-C₄-alkylcarbonyloxy, or combinations thereof;
R⁴, R⁵, and R⁶
are, independently of one another, hydrogen, hydroxyl, C₁-C₆-alkyl, or C₁-C₆-alkoxy, wherein the aliphatic moieties of these substituents are optionally partially or fully halogenated and/or are optionally mono-, di, or tri-substituted with cyano, hydroxyl, C₁-C₄-alkyl, C₁-C₄-haloalkyl, C₃-C₆-cycloalkyl, C₁-C₄-alkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl, C₁-C₄-alkylcarbonyloxy, or combinations thereof;
A¹ is aryl or heteroaryl;
A² is aryl or heteroaryl, with the proviso that A² is not indolyl;
R^{a} is halogen, cyano, nitro, C₁-C₆-alkyl, C₃-C₆-cycloalkyl, C₂-C₆-alkenyl, C₃-C₆-cycloalkenyl, C₄-C₁₀-alkadienyl, C₂-C₆-alkynyl, [tri-(C₁-C₆)-alkylsilyl]-(C₂-C₆)-alkynyl, C₃-C₆-cycloalkynyl, C₁-C₆-alkylthio, C₁-C₆-alkylsulfinyl, aryl, phenyl-(C₁-C₆)-alkyl, phenyl-(C₂-C₆)-alkenyl, phenylsulfonyl-(C₁-C₆)-alkyl, heterocyclyl, heterocyclyl-(C₁-C₆)-alkyl, or phenyl-[C₁-C₆-alkoxycarbonyl]-(C₁-C₆)-alkyl, or
Z¹P(O)(OR⁹)₂ or Z²B(OR¹⁰)₂, wherein
R⁹ and R¹⁰ are each, independently of one another, hydrogen or C₁-C₆-alkyl and both R¹⁰ moieties in Z²B(OR¹⁰)₂ together optionally define a C₂-C₄-alkylene chain; or
Z³COR¹¹, wherein
R¹¹ is hydrogen, C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, hydroxyl, C₁-C₆-alkoxy, C₃-C₆-alkenyloxy, C₃-C₆-alkynyloxy, amino, C₁-C₆-alkylamino, [di-(C₁-C₆)-alkyl]amino, C₁-C₆-alkoxyamino, [di-(C₁-C₆)-alkoxy]lamino, C₁-C₆-alkylsulfonylamino, C₁-C₆-alkylaminosulfonylamino, [di-(C₁-C₆)-alkylamino]sulfonylamino, C₃-C₆-alkenylamino, C₃-C₆-alkynylamino, N—(C₂-C₆-alkenyl)-N—(C₁-C₆-alkyl)amino, N—(C₂-C₆-alkynyl)-N—(C₁-C₆-alkyl)amino, N—(C₁-C₆-alkoxy)-N—(C₁-C₆-alkyl)amino, N—(C₂-C₆-alkenyl)-N—(C₁-C₆-alkoxy)amino, N—(C₂-C₆-alkynyl)-N—(C₁-C₆-alkoxy)amino, phenyl, phenoxy, phenylamino, naphthyl, or heterocyclyl; or
Z⁴NR¹²R¹³, wherein
R¹² and R¹³ are, independently of one another, hydrogen, C₁-C₆-alkyl, C₃-C₆-cycloalkyl, C₃-C₆-alkenyl, C₃-C₆-cycloalkenyl, C₃-C₆-alkynyl, C₃-C₆-cycloalkynyl, C₁-C₆-alkylcarbonyl, C₃-C₆-cycloalkylcarbonyl, [di-(C₁-C₆)-alkylamino]carbonyl, C₁-C₆-alkoxycarbonyl, C₁-C₆-alkoxycarbonyl-(C₁-C₆)-alkyl, C₁-C₆-alkylsulfonyl, C₁-C₆-alkylaminosulfonyl, [di-(C₁-C₆)-alkylamino]sulfonyl, phenylcarbonyl, phenylaminocarbonyl, phenylsulfonyl, phenylsulfonylaminocarbonyl, or heterocyclylcarbonyl; or
Z⁵CH═N—O—R¹⁴, wherein R¹⁴ is hydrogen or C₁-C₆-alkyl; or
Z⁶OR¹⁵, wherein
R¹⁵ is hydrogen, C₁-C₆-alkyl, C₃-C₆-cycloalkyl, C₃-C₆-alkenyl, C₃-C₆-cycloalkenyl, C₃-C₆-alkynyl, C₃-C₆-cycloalkynyl, C₁-C₆-alkylcarbonyl, C₁-C₆-alkoxycarbonyl-(C₁-C₆)-alkyl, [di-(C₁-C₆)-alkoxycarbonyl]-(C₁-C₆)-alkyl, phenyl, or phenyl-(C₁-C₆)-alkyl; or
Z⁷SO₂R¹⁶, wherein R¹⁶ is C₁-C₆-alkyl or phenyl;
wherein
Z¹, Z², Z³, Z⁴, Z⁵, Z⁶, and Z⁷
are, independently of one another, a bond, —CH₂—, —CH₂—CH₂—, —O—CH(R¹⁷)—, —S—CH(R¹⁸)—, —S(O)—CH(R¹⁹)—, or —SO₂CH(R²⁰)—, wherein R¹⁷, R¹⁸, R¹⁹, and R²⁰ are, independently of one another, hydrogen or C₁-C₆-alkyl; and
wherein, when R^{a} is an aliphatic, cyclic, or aromatic substituent, said aliphatic, cyclic, or aromatic substituent is optionally partially or fully halogenated and/or is optionally mono-, di, or tri-substituted with cyano, hydroxyl, C₁-C₄-alkyl, C₁-C₄-haloalkyl, C₃-C₆-cycloalkyl, C₁-C₄-alkoxy, C₁-C₄-haloalkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl, C₁-C₄-alkylcarbonyloxy, or combinations thereof;
R^{b}, R^{c}, R^{d}, R^{e}, and R^{f}
are, independently of one another, hydrogen or a substituent as defined in R^{a}; and wherein any two of R^{a}, R^{b}, or R^{c} attached to adjacent ring atoms of A¹ or any two of R^{d}, R^{e}, or R^{f} attached to adjacent ring atoms of A² are optionally straight-chain C₃-C₆-alkylene optionally partially or fully halogenated and optionally mono-, di, or tri-substituted with cyano, hydroxyl, C₁-C₄-alkyl, C₁-C₄-haloalkyl, C₃-C₆-cycloalkyl, C₁-C₄-alkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl, C₁-C₄-alkylcarbonyloxy, or combinations thereof, where one CH₂ group in C₃-C₆-alkylene is optionally replaced by a carbonyl group, a thiocarbonyl group, or a sulfonyl group and wherein one or two non-adjacent CH₂ groups in C₃-C₆-alkylene are optionally replaced by oxygen, sulfur, or a group NR³⁴, wherein R³⁴ is a substituent as defined in R¹²;
with the proviso that compounds of formula (I) and agriculturally useful salts thereof wherein
A¹ is phenyl and A² is 4-imidazolyl or A¹ is 4-imidazolyl and A² is phenyl;
R¹ is hydrogen; R² is methyl; R³, R⁴, R⁵, and R⁶ are hydrogen; the moiety A¹(R^{a}R^{b}R^{c}) is 4-methoxyphenyl; and the moiety A²(R^{d}R^{e}R^{f}) is phenyl; and
A¹ is phenyl; R¹ and R² are methyl; R³, R⁴, R⁵ and R⁶ are hydrogen; R^{a} is benzyloxy attached in the 3-position; R^{b} and R^{c} are hydrogen; and the moiety A²(R^{d}R^{c}R^{f}) is phenyl or 3-nitrophenyl; and
A¹ is phenyl; R¹ is hydrogen, acetyl, or isopropyloxycarbonyl; R² is hydrogen or benzyl; R³, R⁴, R⁵, and R⁶ are hydrogen; R^{a} is benzyloxy attached in the 2-position; R^{b} and R^{c} together are a moiety OCH₂—O attached to the carbon atoms in the 4- and 5-positions of phenyl; and the moiety A²(R^{d}R^{e}R^{f}) is 3-methyl-4-methoxyphenyl; and
R¹ is isopropyloxycarbonyl; R² is benzyl; R³, R⁴, R⁵, and R⁶ are hydrogen; and the moieties A¹(R^{a}R^{b}R^{c}) and A²(R^{d}R^{e}R^{f}) are each 3,4,5-trimethoxyphenyl;
are excluded.

37. A process for preparing the piperazine compound of claim 36 comprising reacting a compound of formula (II) 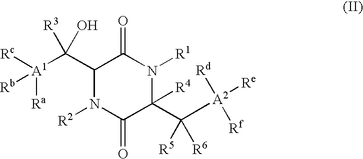
wherein A¹, A², R¹, R², R³, R⁴, R⁵, R⁶, R^{a}, R^{b}, R^{c}, R^{d}, R^{e}, and R^{f} are as defined for the compound of formula (I) in claim 21,
to obtain a compound of formula (III) 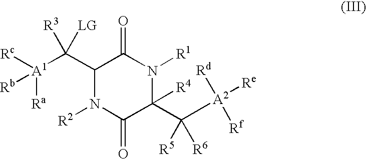
wherein LG is a leaving group,
and either eliminating the compound H-LG from the compound of formula (III), to obtain the compound of formula (I); or
when LG is OH, dehydrating the compound of formula (III), optionally in the presence of a dehydrating agent, to obtain the compound of formula (I).

38. The process of claim 37, wherein LG is selected from the group consisting of 4-toluenesulfonyloxy, trifluoromethanesulfonyloxy, and methanesulfonyloxy.

39. The process of claim 37, where said dehydrating agent is selected from group consisting of (1) the system triphenylphosphine/diethyl azodicarboxylate and (2) the Burgess reagent.
